(19) United States
(12) Patent Application Publication
(10) Pub. No.: US 2024/0076689 A1
(43) Pub. Date: Mar. 7, 2024

(54) VIRAL VECTORS AND NUCLEIC ACIDS FOR REGULATED GENE THERAPY

(71) Applicant: Boehringer Ingelheim International GmbH, Ingelheim am Rhein (DE)

(72) Inventors: John Park, Ingelheim am Rhein (DE); Philipp MÜLLER, Ingelheim am Rhein (DE); Sebastian KREUZ, Ingelheim am Rhein (DE); Benjamin STROBEL, Ingelheim am Rhein (DE); Matthias DÜCHS, Ingelheim am Rhein (DE); Matthias KLUGMANN, Ingelheim am Rhein (DE); Stefan MICHELFELDER, Ingelheim am Rhein (DE); Dragica BLAZEVIC, Ingelheim am Rhein (DE); Romona KRATZER, Ingelheim am Rhein (DE)

(21) Appl. No.: 17/999,122

(22) PCT Filed: May 17, 2021
(86) PCT No.: PCT/EP2021/063034

§ 371 Date: Nov. 17, 2022

(30) Foreign Application Priority Data
May 17, 2020 (EP) 20175116.1

(51) Int. Cl. C12N 15/86; A61K 48/00; C12N 15/63

(52) U.S. Cl. CPC C12N 15/86; A61K 48/0058; A61K 48/0066; C12N 15/635; C12N 2750/14143; C12N 2750/14171; C12N 2830/003

(57) ABSTRACT

This invention generally relates to the field of somatic gene therapy. The invention provides a nucleic acid construct comprising a transgene encoding a therapeutic protein, a tetracycline-responsive aptazyme sequence, and inverted terminal repeats (ITRs). The nucleic acid construct can be transferred to a subject in need thereof in the form of a viral vector, in particular an adeno-associated virus (AAV) vector. The Tet-responsive aptazyme sequence allows for a tightly controlled expression of the transgene in the subject, thereby avoiding toxic side effects. The nucleic acid construct and the viral vectors comprising same are particularly useful in the treatment of proliferative diseases like cancer.

### FIELD OF THE INVENTION

This invention generally relates to the field of somatic gene therapy. The invention provides a nucleic acid construct comprising a transgene encoding a therapeutic protein, a tetracycline (Tet)-responsive aptazyme sequence, and inverted terminal repeats (ITRs). The nucleic acid construct can be transferred to a subject in need of the encoded therapeutic protein in the form of a viral vector, particularly an adeno-associated virus (AAV) vector. The Tet-responsive aptazyme sequence allows for a tightly controlled expression of the transgene in the subject, thereby avoiding toxic side effects of the therapeutic protein. The nucleic acid construct and the viral vectors comprising same are particularly useful in the treatment of proliferative diseases like cancer.

### BACKGROUND OF THE INVENTION

Clinical trials using recombinant first-generation adeno-associated virus (AAV) vectors have contributed significantly to the further advancement of gene therapy by achieving important milestones, such as the first market approved AAV-based therapies (Russell et al. 2017; Jiang et al. 2018; Kumar et al. 2016). At the same time, these trials identified vector elements, whose optimization has the potential to further improve efficacy, tissue specificity and safety, e.g., by engineering vector capsids and promoter/enhancer elements (Grimm & Büning, 2017; Sarcar et al. 2019). Respective approaches are further strengthened by the aspiration to extend next-generation gene therapies beyond the field of inherited rare diseases towards acquired diseases and larger patient populations. To address potential safety issues and account for the natural variability in patients' disease biology and therapeutic response, a particularly desirable feature of gene therapy vectors would be a system that allows to control and precisely induce gene expression.

Specifically, it is envisioned that patients are able to switch on the expression of an AAV-delivered therapeutic by the temporal intake of a small-molecule drug. Such a therapeutic AAV-based system is depicted in FIG. 1. Conceptually, such a system would allow to fine-tune expression levels according to an individual patient's needs, improve the safety profile of transgenes with a narrow therapeutic window or provide a safety switch to mitigate the risk of unwanted immune responses against foreign therapeutic proteins.

Achieving controllable gene expression is highly desirable, as evident from different protein-based systems that have been investigated recently, including destabilizing domains and inducible promoters as the most advanced approaches (Santiago et al. 2018; Barrett et al. 2018). These transcriptional control systems, including the Rheoswitch system, Mifepristone, and classic Tet-ON/OFF promoter systems, suffer from a common drawback: The require the expression of DNA-binding proteins that enable transcription after being activated by their cognate ligands (Chiocca et al., 2019; Wang et al., 2004; Gonzalez-Aparicio et al., 2011; Vanrell et al., 2011; Das et al., 2016). These DNA-binding proteins bear an immunogenic risk by representing T-cell epitopes. Attempts to address this for the Tet-promoter control system by engineering versions without certain T cell epitopes for HLA0201 (the most common human HLA serotype), revealed that if that is even possible—as the protein still has to retain specific binding to both the Tet repressor AND to tetracycline—there will be other humans with other serotypes that may still present epitopes from the resulting protein since there will be no immune tolerance to this foreign protein (Ginhoux et al., 2004). These data show that in order to achieve the full potential of gene therapy technologies, genetic switches with wide dynamic ranges that control transgene expression without the requirement of additional protein components are required.

In this context, so-called artificial riboswitches have been described as attractive building blocks for gene expression control systems that function independently of co-expressed regulatory proteins or fused destabilizing protein domains. Artificial riboswitches (or aptazymes) are DNA-encodable fusions of a ligand-binding RNA aptamer and a ribozyme, which enable to control messenger RNA (mRNA) integrity by conditional mRNA self-cleavage. As shown in FIG. 1, when placed into either the 5′- or 3′-untranslated region (UTR) of an expression construct, autocatalytic ribozyme self-cleavage results in 5′-cap or 3′-poly(A) tail loss, respectively, thereby inducing mRNA degradation and shutdown of gene expression. Allosteric control over ribozyme cleavage is achieved by fusing the ribozyme to an aptamer domain, whose structural re-arrangement upon binding of its cognate ligand alters the global riboswitch architecture. This prevents its ability to self-cleave, thereby enabling gene expression (“ON-switch” type). Naturally occurring bacteria-, plant- or virus-derived riboswitches control endogenous gene expression in response to cellular cues by steric hindrance of polymerase, ribosome or splicing activity (Berens et al. 2015). Thus, engineered riboswitches represent a prime example for synthetic biology, i.e. the optimization and re-purposing of naturally occurring mechanisms for therapeutic applications (Ausländer & Fussenegger, 2013; Kitada et al, 2018).

Using Theophylline, tetracycline (Tet), Guanine or protein-responsive hammerhead or Hepatitis Delta Virus (HDV)-based riboswitches, principal functionality in cell culture has been demonstrated, yet mostly using OFF-switches (Kumar et al. 2009; Ketzer et al. 2012; Ketzer et al. 2014; Nomura et al (2013); Wei & Smolke, 2015; Bloom et al. 2015; Kennedy et al. 2014). In contrast, exploration of riboswitch function in animals has been scarce. One early study in mice showed direct (i.e. non-allosteric) inhibition of a ribozyme by an RNA-binding compound (Yen et al. 2004). Another demonstrated riboswitch-mediated transgene regulation in ex vivo manipulated cells after transplantation into mice (Chen et al. 2010). In the context of viral vectors, it was previously shown that a recombinant AAV vector equipped with a guanine-HDV switch that enabled conditional shutdown of various genes' expression in vitro allowed for robust gene expression in mice, in the absence of exogenous guanine (Strobel et al. 2015b). In addition, one recent study demonstrated approximately seven-fold Tet-riboswitch-mediated downregulation of AAV-mediated reporter gene expression in the gastrocnemius muscle of mice (Zhong et al. 2016). Tight regulation of self-cleaving activity in the context of an engineered AAV-delivered aptazyme was also reported by employing steric-blocking antisense oligonucleotide resulting in an ON-fashion of transgene expression (Zhong et al., 2020). However, versatility of this approach is hampered by the poor bioavailability of antisense oligonucleotides compared to small molecule ligands.

Whereas ligand-induced suppression of gene expression might in principle find applications as a safety switch, e.g. in oncolytic therapy, a far more attractive option for many therapeutic applications would be the ability to induce therapeutic gene expression only in response to a ligand. However, the only viral vector and ON-riboswitch-based study available to date achieved very modest effects of at best two-fold induction of GFP expression in the eye of mice (Reid et al. 2018), but only the 3×-L2Bulgel8tc riboswitch demonstrated a significant increase (2-fold) in GFP expression compared to baseline levels in vivo (p<0.05, paired test). WO2018/165536 describes the effects of K19 in cell culture, see FIG. 10 and probably erroneously referring to FIG. 9B-D instead of FIG. 10B-D.

In summary, there is a need for a clinically applicable inducible system fulfilling the desired criteria of being efficient, non-immunogenic, small and transgene-independent. Preferably, this system should provide an expression induction which covers a broad range of therapeutic protein expression, i.e. ideally 0-100% of a conventional, riboswitch-free construct. In addition, it should render possible the fine-tuning of expression levels by ligand dose adjustment. Finally, it should allow for repeated ON and OFF switching.

### SUMMARY OF THE INVENTION

The present invention provides nucleic acid constructs and viral vectors that comprise a transgene and a tetracycline-responsive aptazyme which allows for a controlled expression of the transgene. The tetracycline-responsive aptazyme preferably is the aptazyme “K19” previously described Beilstein et al. 2015. The aptazyme comprises the tetracycline aptamer (Berens et al. 2001) and the full-length hammerhead ribozyme N79 from Schistosoma mansoni (Yen et al. 2004). In has been found herein that when used in an expression cassette and delivered by a viral vector, such as an AAV vector, the K19 aptazyme effectively controls and dose-dependently induces AAV-mediated transgene expression by providing or retracting tetracycline in an animal in vivo.

Prior to the present invention, therapeutic applicability of a Tet-responsive aptazyme in eukaryotes has been hampered by (a) lack of sufficient expression induction to cover a broad range of therapeutic protein expression, i.e. ideally 0-100% of a conventional, riboswitch-free construct; (b) lack of ligand dose dependency to fine-tune therapeutic protein expression levels in vivo and (c) uncertainty whether this approach also allows for repeated ON and OFF switching. The present invention has overcome these obstacles.

As described below, the functionality of the K19 Tet riboswitch was first established herein in the context of AAV vector expression cassettes in different cellular cultures, exploring the potency of different designs and chronological aspects of inducibility. Following an in vivo pharmacokinetic (PK) study for Tet, the riboswitch performance was investigated in liver, lung, muscle and heart of mice by simultaneous AAV-mediated secretion of a liver-restricted tool antibody of the diabody type, and a ubiquitously expressed cellular luciferase. It was surprisingly found that the K19 riboswitch construct repeatedly induced reporter antibody expression in a dose-dependent and highly dynamic manner, by administering or retracting Tet treatment. The K19 Tet RNA switch construct was subsequently tested with the therapeutically relevant single-chain IL-12 gene enabling expression of fully bioactive IL-12. Tet-induced IL-12 levels and background levels in vitro were comparable for murine and human single-chain IL-12. Surprisingly, the range of Tet-induced IL-12 levels and leakiness of the system were similar to the reporter gene data observed in vivo. Following liver targeting after systemic delivery of hepatotropic AAV, IL-12 plasma levels induced by a single Tet application increased and dropped to baseline within 24 hrs. IL-12 induction could be repeated by a Tet rechallenge nine days after the first challenge and yielded clinically meaningful cytokine levels without toxicity. In a separate set of pharmacokinetic (PK) and safety experiments in naive mice, we successfully titrated the AAV vector dose so that a twice daily Tet application over five consecutive days resulted in safe and sustained inducible IL-12 expression. In contrast, IL-12 was not detectable in vector-dose matched animals receiving no Tet. The identical treatment regimen was then applied to study the pharmacokinetic and pharmacodynamic (PK/PD) of local and inducible IL-12 immunotherapy in a model of hepatocellular carcinoma (HCC). We observed comparable PK of inducible IL-12 in tumor-bearing and naive mice and recorded near complete remission that coincided with an influx of T-cells in the tumor nodules.

The nucleic acid constructs and viral vectors of the invention therefore allow to fine-tune the expression levels of therapeutic proteins in vivo by adjusting the dose of the Tet ligand in a rib oswitch context. Moreover, the aptazyme-mediated control over the transgene expression following AAV-mediated gene delivery enables repeated, i.e. dynamic ON-OFF switching. This renders the nucleic acid constructs and viral vectors of the invention particularly suitable for use in a clinical setting, as the system could be repeatedly switched on until full tumor remission, as well as in the case of tumor relapse, even several months after delivery of the system.

### BRIEF DESCRIPTION OF THE DRAWINGS

FIG. 1 shows the mode of action of aptazyme riboswitches as a gene expression control system for gene therapy. Left panel: When encoded in the 3′-UTR of an expression construct, riboswitch auto-cleavage leads to a loss of the poly(A) tail, which triggers degradation of the mRNA, thereby preventing protein translation (OFF-state). Upon binding of the cognate ligand via its aptamer domain, the riboswitch undergoes a conformational change, which prevents auto-cleavage activity. The mRNA therefore remains intact and is translated into protein (ON-state). Right panel: A patient receives a recombinant AAV gene therapy vector, encoding a therapeutic gene of interest (GOI) under the control of a riboswitch. In absence of the riboswitch ligand, expression is switched off or reduced to basal levels due to riboswitch auto-cleavage activity. Upon intake of the expressioninducing drug, gene expression is temporarily induced. By adjusting the drug dose, expression levels can be fine-tuned, e.g., to increase therapeutic expression (as shown) according to an individual patient's needs or to reduce expression levels to mitigate risks associated with a narrow therapeutic window or immune responses targeted towards the therapeutic protein.

FIG. 2 shows the evaluation of K19 riboswitch function in cellular systems. (a) Schematic design of eGFP expression constructs, harboring the tetracycline (Tet)-responsive riboswitch at different positions within the 5′- or 3′-untranslated region (UTR). (b) Tet dose-dependent induction of eGFP expression in HEK-293 cells transfected with the expression constructs shown in (a), assessed by direct fluorescence measurements, 24 h after Tet addition. Regulation was also assessed by (c) fluorescence microscopy and imaging and (d) Western blotting for selected constructs. (e) Tet dose-dependent induction of sNLuc expression in HEK-293 cells. inact=inactive, non-cleaving ribozyme control; act=active ribozyme. Vinc=vinculin. N=3 biological replicates. Representative images are shown in (c). Mean±SD.

FIG. 3 shows data on the K19 riboswitch kinetics in cellular systems. (a) HEK-293 cells were transfected with plasmids harboring either an active or inactive K19 switch and incubated for 24 h before addition of 50 μM Tet to induce eGFP expression. Induction was monitored over time on the mRNA level by qPCR as well as via direct GFP fluorescence detection and Western Blotting. (b) 24 h after HEK-293 cell transfection with active K19 riboswitch-harboring sNLuc expression plasmids, culture media was replaced by either Tet-free or Tet-containing media and sNLuc induction was measured in the cell supernatant. Expression changes were further monitored by qPCR for up to 8 h (dashed lines). (c) 24 h after transfection and growth in presence of Tet, media was changed to either Tet-free or Tet-containing media and the relative decrease in sNLuc was measured in the supernatant and via qPCR on the mRNA level (dashed lines). inact=inactive, non-cleaving ribozyme control; act=active ribozyme. Vinc=vinculin. N=3 biological replicates (b, c). (a) shows one representative experiment with N=3 sample replicates out of three similar studies. Mean±SD. **p<0.01; ***p<0.001.

FIG. 4 summarizes results for the tetracycline 24 h-pharmacokinetics measured by HPLC-MS/MS. (a) Mice received 54 mg/kg Tet-HCl via i.p. administration and Tet plasma concentration was measured over time. Inset: logarithmic scale and calculated Tet elimination half-life. (b) Tet plasma and tissue exposure determined 2 hours after i.p. administration of 54 mg/kg Tet. Tissue exposure levels relative to plasma exposure is depicted in (c). (d) PK non-parametric modeling of a three times a day (8 h intervals) i.p. dose of 100 mg/kg Tet based on the 24 h-PK data from (a). Solid line: mean, dotted lines: SD; dashed line: 7 μM (=approximate trough levels). N=3 animals; Mean±SD.

FIG. 5 shows the results from determining the K19 riboswitch functionality in liver, heart, muscle and lung of mice. (a) AAV vector expression cassette design and experimental setup. Mice received a mixture of AAV9 mediating liver-directed anti-FITC scFv antibody (aFITC) expression and AAV9 encoding a cellular ubiquitously expressed Nano-luciferase (cNLuc) at a dose of 5×10¹⁰ vg/mouse per vector. 100 mg/kg Tet or vehicle treatment and blood (B) plasma sampling were conducted at the indicated time points, where plasma was always sampled immediately prior to Tet administration. Tissue (T) lysates were prepared at the end of the study. (b) Tet dose-dependent induction of aFITC and cNLuc expression in transfected HepG2 cells, 24 h after Tet addition. (c) aFITC expression induction measured after repeated Tet dosing in plasma samples over time, versus vehicle treatment. (d) Plasma activity of liver enzymes AST, ALT and GLDH measured at the end of the study. (e) Tet-dependent induction of cNLuc reporter expression measured in tissue lysates obtained at the end of the study. (f) aFITC expression in HepG2 cells transfected with increasing amounts of either CMV- or LP1-aFITC plasmid constructs and conditional stimulation with 50 μM Tet. Fold changes in expression upon Tet stimulation are depicted. The data marked in dashed boxes is compared side-by-side in (g). inact=inactive, non-cleaving ribozyme control; act=active ribozyme. N=3 biological replicates (b); N=8 animals (c, d, e), except from untreated (N=5); N=6 replicates (f, g). Mean±SD (b, d, e, f, g) or SEM (c). *p<0.05, **p<0.01, ***p<0.001, as indicated or relative to vehicle.

FIG. 6 depicts the results from assessing K19 riboswitch-induced expression levels relative to a conventional construct. (a) AAV vector expression cassette designs and experimental setup. (b) Mice received 5×10¹⁰ vg of AAV9-LP1-aFITC vector, either harboring or not the K19 riboswitch. Two weeks after i.v. administration of AAVs, mice received a single 100 mg/kg dose of Tet (arrowhead) and aFITC plasma levels were measured over a time frame of 24 h. Absolute aFITC levels and the fold change in expression relative to vehicle treatment are shown. N=4 animals per group. Mean±SEM. *p<0.05, ***p<0.001, as indicated or relative to vehicle.

FIG. 7 illustrates results for dose-dependency, repeated induction and PK/PD relationships in mice. (a) AAV vector expression cassette design and experimental setup. Mice received 5×10¹⁰ vg of AAV9-LP1-aFITC vectors, either containing or lacking the K19 riboswitch. Two weeks after AAV administration and baseline sampling, Tet (3, 10, 30, 90 mg/kg) or vehicle was administered and aFITC expression was measured in blood (B) plasma samples over time. One week after the first Tet treatment, mice received a second dose to re-induce expression. At time points of Tet treatment, plasma sampling was performed immediately before Tet administration. (b) aFITC expression induction measured in plasma samples over time, depicted as expression relative to the riboswitch-free control construct (upper graph) and as fold change in expression, relative to the averaged expression detected for vehicle treatment (lower graph). (c) qPCR-based measurements of aFITC mRNA expression, relative to vehicle treatment (left graph) and corresponding AAV vector genomes (right graph), detected in liver tissue at the end of the study. (d) Plasma activity of liver enzymes AST, ALT and GLDH measured at the end of the study. (e) Total Tet plasma concentration over time. (f) Riboswitch-induced aFITC expression 8 h after Tet administration as a function of Tet plasma exposure detected at 4 h after administration. A three-parametric “Agonist vs. response” curve fit was generated using GraphPad Prism. LLOQ=lower limit of quantification. Arrowheads depict time points of Tet administration. N=8 animals per group, except from untreated (N=5). Mean±SD (c, d) or SEM (b). *p<0.05, **p<0.01, ***p<0.001, as indicated or relative to vehicle.

FIG. 8 shows in vitro induction of mIL-12 in a human liver cell line (Hep G2) transduced with AAV9 carrying the sequence of murine IL-12 (mIL-12) under the control of either an active (mIL-12 switch active) or inactive Riboswitch (mIL12_switch_inactive). After stimulation with tetracycline the active switch induces an increase in mIL-12 production of 6.4-fold reaching 19% of the constitutively active expression levels mediated by the inactive switch. N=3 biological replicates. Mean±SD.

FIG. 9 gives an overview of the design of the mIL12 expression study in vivo. A total of 23 female C57B1/6 mice either received NaCl or 5×10⁹ or 5×10¹⁰ or 5×10¹¹ vector genomes (vg) of the AAV9 vector harboring the construct with the inactive switch under the control of the liver specific LP1 promotor, (mIL-12_switch_inactive) via intravenous administration. Weight of the animals was monitored daily for calculation of weight loss. At the end of the experiment, plasma and liver samples were collected for measurement of systemic IL-12 levels and for histological analysis of immune cell influx into the liver.

FIG. 10 depicts the development of body weight of the animals during the mIL-12 expression study, given as average per group. Due to weight loss the experiment had to be stopped at different time points; namely on Day 7 for group 4 (receiving 5×10¹¹ vg), on Day 9 for the group 3 (receiving 5×10¹⁰ vg) and on day 11 for the remaining animals of group 1 (Vehicle) and group 2 (receiving 5×10⁰⁹ vg).

FIG. 11 shows that the obtained level of mIL-12 in the expression study increase proportional to the dose of administered vector. The levels of murine IL-12 were measured in plasma after administration of AAV9 vector mIL-12_switch_inactive. The blood was collected at the end of study (day 7, 9, 11) and the respective day of blood sampling is shown in the graph. Plasma was collected via puncture of retro-bulbar sinus in the anesthetizedanimals and murine IL-12 was measured via electrochemiluminescence multiplex assay measurement. Data are presented as mean±SD.

FIG. 12 gives an overview of the design of the tetracycline-induced mIL-12 in vivo time course study. A total of 25 female C57B1/6 mice either received NaCl (group 1), 5×10⁹ vector genomes (vg) of mIL-12_switch_inactive vector (group 2), 5×10⁹ vg of mIL-12_switch_active vector (group 3), 5×10⁹ vg of mIL12_switch_active vector+10 mg/kg tetracycline or 5×10⁹ vg of mIL-12_switch_active vector+30 mg/kg tetracycline. The respective AAV9 vectors were administered intravenously on day 0, tetracycline was given twice; on day 5 and on day 14 with both tetracycline application time points labeled as t=0 h. Weight of the animals was monitored daily for calculation of weight loss. Animals were sacrificed on day 14, 8 h post second tetracycline administration. At the end of the experiment, plasma and the liver tissue samples were collected for measurement of murine IL-12 levels and for analysis of vector genomes in the liver tissue.

FIG. 13 depicts (a) levels of tetracycline measured in plasma collected at the end of the study and (b) viral genomes measured in DNA extracted from homogenized liver tissue and quantified via qPCR. Data are presented as mean±SD. ****p<0.001, as indicated.

FIG. 14 illustrates longitudinal changes in the bodyweight development of treated animals. Only treatment with 5×10⁹ AAV9_LP1_muIL12_inactive vector, inducing constant expression of IL-12, leads to a loss of bodyweight. On Day 11, three animals had to be excluded from the study, as they showed the lowest bodyweight. Therefore, group 2 showed a constant decrease of bodyweight over the course of the experiment, which, however, due to the exclusion of the three animals with the lowest bodyweight is not reflected in the curve of group 2 after day 11.

FIG. 15 shows the tetracycline-induced time dependent induction of the IL-12p70 expression, measured in the plasma via electrochemiluminescence multiplex assay measurement. (a) Group 2, which was treated with 5×10⁹ AAV9_LP1_muIL12_switch_inactive vector (vector with an inactive switch that constitutively expresses IL-12), over time shows a constant increase in IL-12. (b) Depicted are the levels of IL-12 of all groups on the final day of the experiment, day 14, 8 h after the second tetracycline administration. The levels show that the activation of the switch with 30 mg/kg of tetracycline induces a 4.7fold increase of IL-12 compared to group 3 that received no tetracycline. (c) The levels of IL-12 in correlation to the time point after administration of tetracycline, given as fold changes of mIL12 vs. time. *p<0.05, ***p<0.001, relative to the indicated group. Data are presented as mean±SD.

FIG. 16 illustrates effects of different doses of AAV9 inducing unregulated expression of mIL-12. (a) Design of the mIL-12 expression study in vivo. Mice received i.v. injections of either 5×10¹¹ or 5×10¹⁰ or 5×10⁹ vector genomes (vg) of the AAV9 vector harboring the murine IL-12 construct with the inactive K19 switch under the control of the liver-specific LP1 promoter, or buffer. (b) Animals were monitored daily for calculation of changes in body weight. (c) At the end of the experiment for each group, plasma samples were collected for measurement of IL-12 levels. Due to weight loss, the experiment had to be stopped at different time points; namely on day 7 for group 4 (receiving 5×10¹¹ vg), on day 9 for the group 3 (receiving 5×10¹⁰ vg) and on day 11 for the remaining animals of group 1 (control buffer) and group 2 (receiving 5×10⁹ vg). Blood was collected via puncture of retro-bulbar sinus in the anesthetized animals at the end of study (day 7, 9, 11) and IL-12 was measured via electrochemiluminescence multiplex assay measurement. Data are presented as mean±SD. N=6 animals per group.

FIG. 17 describes a PK study of Tet-induced mIL-12 expression. (a) Mice received 5×10⁹ vector genomes (vg) of either AAV9-LP1-mIL-12-inactive-switch, AAV9-LP1mIL-12-switch+30 mg/kg Tet, AAV9-LP1-mIL-12-switch+10 mg/kg Tet, AAV9-LP1mIL-12-switch+0 mg/kg Tet, or buffer. The respective AAV9 vectors were administered i. v. on day 0. Tet was given twice: on day 5 and on day 14 both with Tet application time point t=0 h. Animals were euthanized on day 14, at 8h post Tet administration. At the end of the experiment, plasma and liver tissue samples were collected for measurement of murine IL-12 levels and for analysis of vector genomes in the liver tissue. (b) Viral genomes were measured in DNA extracted from homogenized liver tissue and quantified via qPCR. (c) Tet plasma concentration was determined at the end of the study. (d) Tetdependent induction of IL-12 expression was measured in the plasma via electrochemiluminescence multiplex assay measurement. The levels of IL-12 in correlation to the time point after Tet administration, given as fold-changes of IL-12 compared to IL-12 levels without Tet. (e) IL-12 of all groups on day 14, i.e. the last day of the experiment, at 8 h after the Tet re-challenge. Induction with 30 mg/kg Tet induced a 4.7-fold increase of IL-12 compared to the group that received the same vector dose but no Tet. (f) IL-12 PK in mice that received 5×10⁹ vg AAV9-LP1-mIL-12-inactive-switch, showed a rapid onset of IL-12 expression on day 2 that reached a plateau by day 14. N=5 animals per group.

FIG. 18 describes a dose-finding study to determine PK and safety. (a) AAV vector expression cassette design and experimental setup; at the start of the experiment all mice received an i.v. application of either 5×10⁹ vg of AAV9-LP1-mIL-12-switch_inactive (*), 5×10⁹ vg, 5×10⁸ vg, 5×10⁷ of AAV9-LP1_mIL-12_switch_active, 5×10⁹ vg of AAV9LP1-aFITC-switch-active, or saline buffer. 30 mg/kg of Tet was given i.p. twice daily from day 7 to day 11 in the indicated groups (+Tet). Blood (B) was collected on day 7, 11, 14 and on the final day of the experiment, day 21. (b) Longitudinal changes in the body weight development of treated animals. Measurement of IL-12 on (c) day 7, (d) day 9, and (e) day 14. Plasma activity of liver enzymes (f) AST, (g) ALT and (h) GLDH measured at the end of the study. N=4-5 animals per group. (i) Measurement of IFNγ levels in plasma on day 14. LLOD=lower limit of detection,+=animals were either euthanized prior to blood sampling or due to ethical reasons no samples were taken.

FIG. 19 describes a dose-finding study in tumor-bearing mice. (a) AAV vector expression cassette design and experimental setup. At the start of the experiment all mice received Hepal-6 tumor cells via intrasplenic application. On day 2, all mice received an i.v. application of either 5×10⁹ vg of AAV9-LP1-mIL-12-inactive-switch, 5×10⁹ vg or 5×10⁸ vg of AAV9-LP1-mIL-12-switch, buffer, or 5×10⁹ vg of AAV9-LP1-aFITC-switch as AAV control. 30 mg/kg of Tet was given i.p. twice daily from day 7 to day 11 in the indicated groups (+Tet). Blood (B) was collected on day 7, 11, 14 and on day 18, the final day of the experiment. (b) Viral genomes were measured in DNA extracted from homogenized liver tissue and quantified via ddPCR. (c) Levels of IL-12 over the course of the experiment. The fold increase of the two groups receiving 5×10⁸ vg of AAV9-LP1IL-12-switch with and without Tet induction are shown. (d) Whole body images of luciferase signals assessed on the final day of the experiment with quantitative analysis of luciferase activity. (e) Liver weight of animals on day 18. Plasma activity of liver enzymes (f) AST, (g) ALT and (h) GLDH measured at the end of the study. N=4-12 animals per group.

FIG. 20 depicts immunohistochemical staining of livers from tumor-bearing mice from the experiment shown in FIG. 19. (a) Representative images of Hematoxylin (nuclei) and CD45-stained liver sections and quantification of CD45+ liver area for all animals. (b) Representative images of Hematoxylin and Eosin (H&E)-stained liver sections and quantification of liver tumor area for all animals. N=11-12 animals per group scale bar represent 500 μm, all pictures are taken at the same magnification.

FIG. 21 illustrates the Tet-dependent induction of human IL-12 in vitro. HEK293 were transfected with LP1-promoter driven human IL-12 (hIL-12) expression plasmids with an active or inactive riboswitch (pLP1-hIL-12-switch or pLP1-hIL-12-inactive-switch). After addition of Tet, for pLP1-hIL-12-switch, IL-12 production increased 5.6-fold, and reached 25% of the constitutively active expression levels of pLP1-hIL-12-inactive-switch. N=3 biological replicates, mean is presented as ±SD.

FIG. 22 shows bioactivity of human and mouse IL-12 constructs. The experiment was conducted with IL-12 in the p35-linker-p40 and p40-linker-p35 orientation. HEK293 cells were transfected with mouse IL-12 or human IL-12 expression plasmids, and supernatants were tested on HEK-Blue™ IL-12 cells. It could be confirmed that both the p40-linker-p35 as well as the p35-linker-p40 orientations of single chain IL-12 result in bioactive IL-12.

FIG. 23 shows the subsequences of the plasmids used, and in particular the regions between ITRs.

### DETAILED DESCRIPTION OF THE INVENTION

In a first aspect, the present invention relates to a nucleic acid construct comprising (i) a transgene encoding one or more therapeutic proteins, (ii) at least one tetracycline-responsive aptazyme sequence, and (iii) inverted terminal repeats (ITRs). The nucleic acid construct can comprise or consist of either RNA or DNA. Preferably, the nucleic acid construct will comprise or consist of DNA. The construct may be in linear or circular form, e.g. in the form of a plasmid. In a preferred embodiment, the nucleic acid construct of the invention comprises or consists of single-stranded or double-stranded DNA. In a particularly preferred embodiment, the nucleic acid construct of the invention consists of single-stranded DNA.

The nucleic acid construct of the invention comprises a transgene which codes for one or more therapeutic proteins. As used herein, therapeutic proteins include all types of proteins that exert, upon administration, a therapeutic benefit to a patient suffering from a disease or condition. Therapeutic proteins include proteins which are active immediately after their translation, as well as proteins which are first produced as in an inactive form and are activated after cleavage by a protease or peptidase, e.g. proteins produced with a signal peptide. Preferably, the transgene codes for a mammalian protein, more preferably a human protein. The protein is therapeutically active which means that its delivery to a subject effectively reduces or inhibits the severity of a disease or pathological condition. Preferably, the therapeutically active protein is a protein whose constitutive expression in the subject would lead to toxic or other significant side effects, so that a tight control of the expression is required. Toxic and other significant side effects caused by expression of the protein may include severe conditions caused by strong and persistent activation of the immune response, including cachexia, fever, chills, fatigue, arthromyalgia and/or headache.

In a preferred embodiment of the invention, the transgene encodes one or more immunoregulatory proteins. Immunoregulatory proteins in the sense of the instant disclosure include, but are not limited to, antibodies, such as Ipilimumab or anti-PD1 antibodies, antibody fragments, cytokines, such as interleukins, interferons, lymphokines, and pro-inflammatory and pro-apoptotic members of the tumor necrosis factor (TNF)/tumor necrosis factor receptor (TNFR) superfamily. Further included are T cell engagers, immune checkpoint inhibitors, agonists such as anti-CD137, anti-CD28, or anti-CD40 combinations of any of the above. In a preferred embodiment, the immunoregulatory protein encoded by the transgene is an interleukin selected from the group consisting of IL-4, IL-6, IL-10, IL-11, IL-12, IL-13, IL-23, IL-27, and IL-33. It is particularly preferred that the immunoregulatory protein in the construct of the invention is IL-12, preferably human IL-12.

It is known that IL-12 plays an important role in regulating both the innate and adaptive immune response. Apart from inducing potent anti-cancer effects, it synergizes with several other cytokines for increased immunoregulatory activities. A gene transfer of IL-12 would circumvent the safety issues caused by exposure to bolus doses of recombinant cytokine protein. If the gene encoding IL-12 is delivered locally, it is possible to achieve a beneficial concentration in the desired tissue, with diminished systemic exposure and hence, toxicity (Berraondo et al., 2018; Chiocca et al., 2019). Vectorized IL-12 delivery using gene shuttles with tissue tropism enables systemic delivery followed by local IL-12 production and, attraction and activation of T-cells in a paracrine fashion. IFNγ conveys beneficial anti-tumoral effects of IL-12 by modifying the tumor microenvironment. However, IFNγ is also the main mediator of the toxic effects of IL-12 and, over time, turns on immunoregulatory mechanisms, such as PD-L1 and IDO-1 expression, which mediate adaptive resistance to immunotherapy (Berraondo et al., 2018). Therefore, a genetically encoded, regulatable IL-12 expression is highly beneficial, as it indirectly also controls the level of IFNγ release from natural killer cells.

Natural IL-12 is a heterodimeric protein consisting of one p35 subunit (the alpha chain) and one p40 subunit (the beta chain). The two subunits are covalently linked via a disulfide bridge and form the biologically active 70 kDa dimer. Simultaneous expression of the alpha and beta chain required for production of active IL-12, has been achieved by bicistronic expression using an internal ribosomal entry site (IRES) or by expressing both subunits individually. However, the IRES strategy results in unequal expression of the individual subunits resulting in a bias towards p40 homodimers that display inhibitory p70 signaling. Moreover, two full expression cassettes entailing all necessary cis-acting modules exceed the packaging limit of AAV. Therefore, it is preferred to express for research purposes in mice a bioactive single chain murine IL-12 fusion protein sequence identical to mIL-12.p40.Δp35 as described by Lieschke et al. (mIL-12 hereinafter). In this construct, the p40 subunit is linked by a (Gly-Ser) linker to the p35 subunit from which the first 22 amino acids were deleted. The analogous human IL-12 fusion protein, with a p40-G6S-p35 configuration, also has been reported to retain high in vitro bioactivity (Lieschke et al., 1997; Zhang et al., 2011). For use in human cells, e.g. in cell tissues or human individuals, the corresponding protein of human origin (denoted “hIL-12”, see e.g. SEQ ID No. 6) can be used and is preferred herein. Human IL-12 is not cross-reactive for mouse cells. Consequently, mIL-12 was employed in the mouse experiments described below (for the mature sequence see SEQ ID No. 12, the precursor sequence with signal peptide is encoded by Seq ID NO:11, for the region in the plasmid flanked by ITRs see SEQ ID NO:29). Human IL-12 can be used in human tissue accordingly as an example for single chain IL-12 that works in human tissue. The term single chain IL-12 shall mean that the function of the IL-12 heterodimer is realized by one fusion protein.

In a preferred embodiment, the nucleic acid construct of the invention comprises a transgene encoding single chain IL-12. It is preferred that for the single chain IL-12, one or more of the conditions (A) to (AAAAA) are met:

(A) The single chain IL-12 shows at least 80% sequence identity to
i. the amino acid sequence of SEQ ID No. 1 over the length of SEQ ID No. 1, or
ii. the amino acid sequence SEQ ID No 2 over the length of SEQ ID No. 2, or
iii. the amino acid sequence SEQ ID No 3 over the length of SEQ ID No. 3, or
iv. the amino acid sequence SEQ ID No 4 over the length of SEQ ID No. 4, or
v. the amino acid sequence SEQ ID No 5 over the length of SEQ ID No. 5, or
vi. the amino acid sequence SEQ ID No 6 over the length of SEQ ID No. 6, or
vii. the amino acid sequence SEQ ID No 34 over the length of SEQ ID No 34, or
viii. the amino acid sequence SEQ ID No 35 over the length of SEQ ID No 35, or
ix. the amino acid sequence SEQ ID No 36 over the length of SEQ ID No 36, or
x. the amino acid sequence SEQ ID No 37 over the length of SEQ ID No 37, or
xi. the amino acid sequence SEQ ID No 38 over the length of SEQ ID No 38, or
xii. the amino acid sequence SEQ ID No 39 over the length of SEQ ID No 39, or
xiii. the amino acid sequence SEQ ID No 40 over the length of SEQ ID No 40, or
xiv. the amino acid sequence SEQ ID No 41 over the length of SEQ ID No 41.

(AA) The single chain IL-12 shows at least 90% sequence identity to
i. the amino acid sequence of SEQ ID No. 1 over the length of SEQ ID No. 1, or
ii. the amino acid sequence SEQ ID No 2 over the length of SEQ ID No. 2, or
iii. the amino acid sequence SEQ ID No 3 over the length of SEQ ID No. 3, or
iv. the amino acid sequence SEQ ID No 4 over the length of SEQ ID No. 4, or
v. the amino acid sequence SEQ ID No 5 over the length of SEQ ID No. 5, or
vi. the amino acid sequence SEQ ID No 6 over the length of SEQ ID No. 6.
vii. the amino acid sequence SEQ ID No 34 over the length of SEQ ID No 34, or
viii. the amino acid sequence SEQ ID No 35 over the length of SEQ ID No 35, or
ix. the amino acid sequence SEQ ID No 36 over the length of SEQ ID No 36, or
x. the amino acid sequence SEQ ID No 37 over the length of SEQ ID No 37, or
xi. the amino acid sequence SEQ ID No 38 over the length of SEQ ID No 38, or
xii. the amino acid sequence SEQ ID No 39 over the length of SEQ ID No 39, or
xiii. the amino acid sequence SEQ ID No 40 over the length of SEQ ID No 40, or
xiv. the amino acid sequence SEQ ID No 41 over the length of SEQ ID No 41.

(AAA) The single chain IL-12 shows at least 95% sequence identity to
i. the amino acid sequence of SEQ ID No. 1 over the length of SEQ ID No. 1, or
ii. the amino acid sequence SEQ ID No 2 over the length of SEQ ID No. 2, or
iii. the amino acid sequence SEQ ID No 3 over the length of SEQ ID No. 3, or
iv. the amino acid sequence SEQ ID No 4 over the length of SEQ ID No. 4, or
v. the amino acid sequence SEQ ID No 5 over the length of SEQ ID No. 5, or
vi. the amino acid sequence SEQ ID No 6 over the length of SEQ ID No. 6, or
vii. the amino acid sequence SEQ ID No 34 over the length of SEQ ID No 34, or
viii. the amino acid sequence SEQ ID No 35 over the length of SEQ ID No 35, or
ix. the amino acid sequence SEQ ID No 36 over the length of SEQ ID No 36, or
x. the amino acid sequence SEQ ID No 37 over the length of SEQ ID No 37, or
xi. the amino acid sequence SEQ ID No 38 over the length of SEQ ID No 38, or
xii. the amino acid sequence SEQ ID No 39 over the length of SEQ ID No 39, or
xiii. the amino acid sequence SEQ ID No 40 over the length of SEQ ID No 40, or
xiv. the amino acid sequence SEQ ID No 41 over the length of SEQ ID No 41.

(AAAA) The single chain IL-12 shows at least 98% sequence identity to
i. the amino acid sequence of SEQ ID No. 1 over the length of SEQ ID No. 1, or
ii. the amino acid sequence SEQ ID No 2 over the length of SEQ ID No. 2, or
iii. the amino acid sequence SEQ ID No 3 over the length of SEQ ID No. 3, or
iv. the amino acid sequence SEQ ID No 4 over the length of SEQ ID No. 4, or
v. the amino acid sequence SEQ ID No 5 over the length of SEQ ID No. 5, or
vi. the amino acid sequence SEQ ID No 6 over the length of SEQ ID No. 6.
vii. the amino acid sequence SEQ ID No 34 over the length of SEQ ID No 34, or
viii. the amino acid sequence SEQ ID No 35 over the length of SEQ ID No 35, or
ix. the amino acid sequence SEQ ID No 36 over the length of SEQ ID No 36, or
x. the amino acid sequence SEQ ID No 37 over the length of SEQ ID No 37, or
xi. the amino acid sequence SEQ ID No 38 over the length of SEQ ID No 38, or
xii. the amino acid sequence SEQ ID No 39 over the length of SEQ ID No 39, or
xiii. the amino acid sequence SEQ ID No 40 over the length of SEQ ID No 40, or
xiv. the amino acid sequence SEQ ID No 41 over the length of SEQ ID No 41.

(AAAAA) The single chain IL-12 shows 100% sequence identity to
i. the amino acid sequence of SEQ ID No. 1 over the length of SEQ ID No. 1, or
ii. the amino acid sequence SEQ ID No 2 over the length of SEQ ID No. 2, or
iii. the amino acid sequence SEQ ID No 3 over the length of SEQ ID No. 3, or
iv. the amino acid sequence SEQ ID No 4 over the length of SEQ ID No. 4, or
v. the amino acid sequence SEQ ID No 5 over the length of SEQ ID No. 5, or
vi. the amino acid sequence SEQ ID No 6 over the length of SEQ ID No. 6.
vii. the amino acid sequence SEQ ID No 34 over the length of SEQ ID No 34, or
viii. the amino acid sequence SEQ ID No 35 over the length of SEQ ID No 35, or
ix. the amino acid sequence SEQ ID No 36 over the length of SEQ ID No 36, or
x. the amino acid sequence SEQ ID No 37 over the length of SEQ ID No 37, or
xi. the amino acid sequence SEQ ID No 38 over the length of SEQ ID No 38, or
xii. the amino acid sequence SEQ ID No 39 over the length of SEQ ID No 39, or
xiii. the amino acid sequence SEQ ID No 40 over the length of SEQ ID No 40, or
xiv. the amino acid sequence SEQ ID No 41 over the length of SEQ ID No 41.

For each case (A) to (AAAAA), it is preferred that the single chain IL-12 shows the recited level of identity for the reference sequence specified under (iii) or (iv). More preferably, the single chain IL-12 shows the recited level of identity for a sequence according to SEQ ID No. 3 and to a sequence according to SEQ ID No. 4 over the full length of SEQ ID 3 and 4, respectively.

Preferably, the single chain IL-12 comprises one or more sequences selected from the group consisting of SEQ ID No. 1, 2, 3, 4, 5, and 6. More preferably, the human single chain IL-12 comprises both a sequence according to SEQ ID No. 3 and a sequence according to SEQ ID No. 4. Most preferably, the single chain IL-12 comprises both a sequence of SEQ ID No. 3 and a sequence of SEQ ID No. 4, wherein the sequence of SEQ ID No. 3 is followed by the sequence of SEQ ID No. 4, see e.g. SEQ ID No. 1, 2, 5, 6, optionally separated by a linker sequence such as (G 4 5) 3 (i.e. GGGGSGGGGSGGGGS) or G 4 5 (i.e. GGGGS) or G6s (i.e. GGGGGGS) (see e.g. SEQ ID No. 1, 2, 5, 6, 34, 35, 36, 37, 38, 39, 40, or 41). To allow or facilitate secretion, the single chain human IL-12 protein encoded by the transgene of the nucleic acid construct [which comprises a transgene encoding one or more therapeutic proteins, at least one tetracycline-responsive aptazyme sequence, and inverted terminal repeats (ITRs)] should preferably comprise an N-terminal signal sequence, such as the authentic signal sequence (e.g. SEQ ID No. 33 in SEQ ID No. 2, 6) or a signal sequence that stems from a different secreted protein (see e.g. the amino acid sequence encoded by the sequence SEQ ID No. 13) or an artificial signal sequence having the same function to allow cleavage by the signal peptidase. Such single chain IL-12 proteins are known in the art, see EP 3 211 000 B1 (sequence referred to as SEQ ID No. 6 therein) and US 10,646,549 B2 (sequence referred to as SEQ ID No. 48 therein). In another preferred embodiment, the single chain IL-12 comprises the sequence of SEQ ID No. 75.

In a particularly preferred embodiment, the nucleic acid construct of the invention comprises a transgene that encodes a single chain IL-12 comprising the sequence of SEQ ID No. 3, the sequence of SEQ ID No. 4, a linker sequence between the sequence of SEQ ID No. 3 and the sequence of SEQ ID No. 4, and an N-terminal signal sequence that provides for secretion of the single chain IL-12.

As used herein, the terms “identical” or “percent identity,” in the context of two or more nucleic acid or polypeptide sequences, refer to two or more sequences or subsequences that are the same in length and/or have a specified percentage of nucleotides or amino acid residues that are the same, when compared and aligned for maximum correspondence.

To determine the percent identity, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity =number of identical positions/total number of positions (e.g., overlapping positions)×100). In some embodiments, the two sequences that are compared are the same length after gaps are introduced within the sequences, as appropriate (e.g., excluding additional sequence extending beyond the sequences being compared).

The term “% sequence identity to the amino acid sequence of SEQ ID No. X over the length of SEQ ID No. X” means that the alignment should cover the entire length of the sequence of SEQ ID No. X (the reference sequence). In case the algorithms mentioned below do not render an alignment of the entire length of the reference sequence with the test sequence, but only over a subsequence of said reference sequence, amino acid residues within the reference sequence that do not have an identical counterpart on the test sequence are calculated as mismatch. The percent identity score given by said algorithm is then adjusted: If the algorithm yields K identical amino acids over an alignment length of L amino acids, and yields a percent identity of K/L*100, the term L is replaced by the number amino acids of the reference sequence if that number is higher than L. For instance, if the test sequence has one amino acid at the N-terminus less than the reference sequence SEQ ID No. 2 (but is otherwise identical except for this difference), the percent identity is 517/518*100%≈99.8%. The same applies vice versa to nucleic acid sequences.

The determination of percent identity or percent similarity between two sequences can be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and)(BLAST programs of Altschul et al., 1990, J. Mol. Biol. 215:403-410. BLAST nucleotide searches can be performed with the NBLAST program, score=100, wordlength=12, to obtain nucleotide sequences homologous to a nucleic acid encoding a protein of interest. BLAST protein searches can be performed with the) (BLAST program, score=50, wordlength=3, to obtain amino acid sequences homologous to a protein of interest. To obtain gapped alignments for comparison purposes, Gapped BLAST can be utilized as described in Altschul et al., 1997, Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g.,) (BLAST and NBLAST) can be used. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti, 1994, Comput. Appl. Biosci. 10:3-5; and FASTA described in Pearson and Lipman, 1988, Proc. Natl. Acad. Sci. USA 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. Alternatively, protein sequence alignment may be carried out using the CLUSTAL W algorithm, as described by Higgins et al., 1996, Methods Enzymol. 266:383-402.

An alignment can easily be produced, e.g. by using the following link: https://blast.ncbi.nlm.nih.gov/Blast.cgi?PAGE=Proteins&PROGRAM=blastp&BLAST_PROGRAMS=blas tp&PAGE TYPE=BlastSearch&BLAST_SPEC=blast2seq&DATABASE=n/a&QUERY=&SUBJECTS=

For the purpose of calculating the percent identity, an alignment between test sequence and reference sequence (selected form the group consisting of SEQ ID No. 1, SEQ ID No. 2, SEQ ID No. 3, SEQ ID No. 4, SEQ ID No. 5 and SEQ ID No. 6, respectively) is chosen among possible alignments produced by the mentioned algorithm that gives the highest identity score.

The single chain IL-12 protein preferably exhibits in an assay according to example (1.14) an immune-stimulating activity of at the same order of magnitude or better than the activity of commercially available bioactive human IL-12 consisting of two subunits linked via a disulfide bond with known half-maximum activity generally observed at concentrations of 100 to 400 pg/mL (Gately et al., 1995).

This table shows sequences mentioned in the text. In case of inconsistencies with the sequence listing, the sequences shown in the table are the authentic sequences. For explanations see Table 3.

TABLE 1

| Seq. ID No. | |
|---|---|
| Seq. ID No. 1 | IWELKKDVYVVELDWYPDAPGEMVVLTCDTPEEDGITWTLDQSSEVLGSGKTLTIQVK |
| | EFGDAGQYTCHKGGEVLSHSLLLLHKKEDGIWSTDILKDQKEPKNKTFLRCEAKNYSGRFTCWWLTTIST |
| | DLTFSVKSSR |
| | GSSDPQGVTCGAATLSAERVRGDNKEYEYSVECQEDSACPAAEESLPIEVMVDAVHKLKYENYTSSFFIR |
| | DIIKPDPPKN |
| | LQLKPLKNSRQVEVSWEYPDTWSTPHSYFSLTFCVQVQGKSKREKKDRVFTDKTSATVICRKNASISVRA |
| | QDRYYSSSWS |
| | EWASVPCSGGGGSGGGGSGGGGSRNLPVATPDPGMFPCLHHSQNLLRAVSNMLQKARQTLEFYPCTSEE |
| | IDHEDITKDKT |
| | STVEACLPLELTKNESCLNSRETSFITNGSCLASRKTSFMMALCLSSIYEDLKMYQVEFKTMNAKLLMDPK |
| | RQIFLDQNM |
| | LAVIDELMQALNFNSETVPQKSSLEEPDFYKTKIKLCILLHAFRIRAVTIDRVMSYLNAS |
| SEQ ID No. 2 | MCHQQLVISWFSLVFLASPLVAIWELKKDVYVVELDWYPDAPGEMVVLTCDTPEEDGITWTLDQSSEVL |
| | GSGKTLTIQVK |
| | EFGDAGQYTCHKGGEVLSHSLLLLHKKEDGIWSTDILKDQKEPKNKTFLRCEAKNYSGRFTCWWLTTIST |
| | DLTFSVKSSR |
| | GSSDPQGVTCGAATLSAERVRGDNKEYEYSVECQEDSACPAAEESLPIEVMVDAVHKLKYENYTSSFFIR |
| | DIIKPDPPKN |
| | LQLKPLKNSRQVEVSWEYPDTWSTPHSYFSLTFCVQVQGKSKREKKDRVFTDKTSATVICRKNASISVRA |
| | QDRYYSSSWS |
| | EWASVPCSGGGGSGGGGSGGGGSRNLPVATPDPGMFPCLHHSQNLLRAVSNMLQKARQTLEFYPCTSEE |
| | IDHEDITKDKT |
| | STVEACLPLELTKNESCLNSRETSFITNGSCLASRKTSFMMALCLSSIYEDLKMYQVEFKTMNAKLLMDPK |
| | RQIFLDQNM |
| | LAVIDELMQALNFNSETVPQKSSLEEPDFYKTKIKLCILLHAFRIRAVTIDRVMSYLNAS |
| SEQ ID No. 3 | IWELKKDVYVVELDWYPDAPGEMVVLTCDTPEEDGITWTLDQSSEVLGSGKTLTIQVK |
| | EFGDAGQYTCHKGGEVLSHSLLLLHKKEDGIWSTDILKDQKEPKNKTFLRCEAKNYSGRFTCWWLTTIST |
| | DLTFSVKSSR |
| | GSSDPQGVTCGAATLSAERVRGDNKEYEYSVECQEDSACPAAEESLPIEVMVDAVHKLKYENYTSSFFIR |
| | DIIKPDPPKN |
| | LQLKPLKNSRQVEVSWEYPDTWSTPHSYFSLTFCVQVQGKSKREKKDRVFTDKTSATVICRKNASISVRA |
| | QDRYYSSSWS |
| | EWASVPCS |
| SEQ ID No. 4 | RNLPVATPDPGMFPCLHHSQNLLRAVSNMLQKARQTLEFYPCTSEEIDHEDITKDKT |
| | STVEACLPLELTKNESCLNSRETSFITNGSCLASRKTSFMMALCLSSIYEDLKMYQVEFKTMNAKLLMDPK |
| | RQIFLDQNM |
| | LAVIDELMQALNFNSETVPQKSSLEEPDFYKTKIKLCILLHAFRIRAVTIDRVMSYLNAS |
| SEQ ID No. 5 | IWELKKDVYVVELDWYPDAPGEMVVLTCDTPEEDGITWTLDQSSEVLGSGKTLTIQVK |
| | EFGDAGQYTCHKGGEVLSHSLLLLHKKEDGIWSTDILKDQKEPKNKTFLRCEAKNYSGRFTCWWLTTIST |
| | DLTFSVKSSR |
| | GSSDPQGVTCGAATLSAERVRGDNKEYEYSVECQEDSACPAAEESLPIEVMVDAVHKLKYENYTSSFFIR |
| | DIIKPDPPKN |
| | LQLKPLKNSRQVEVSWEYPDTWSTPHSYFSLTFCVQVQGKSKREKKDRVFTDKTSATVICRKNASISVRA |
| | QDRYYSSSWS |
| | EWASVPCSGGGGGGSRNLPVATPDPGMFPCLHHSQNLLRAVSNMLQKARQTLEFYPCTSEEIDHEDITKD |
| | KTSTVEACLP |
| | LELTKNESCLNSRETSFITNGSCLASRKTSFMMALCLSSIYEDLKMYQVEFKTMNAKLLMDPKRQIFLDQN |
| | MLAVIDELM |
| | QALNFNSETVPQKSSLEEPDFYKTKIKLCILLHAFRIRAVTIDRVMSYLNAS |
| SEQ ID No. 6 | MCHQQL VISWFSL VFLASPLVA |
| | IWELKKDVYVVELDWYPDAPGEMVVLTCDTPEEDGITWTLDQSSEVLGSGKTLTIQVK |
| | EFGDAGQYTCHKGGEVLSHSLLLLHKKEDGIWSTDILKDQKEPKNKTFLRCEAKNYSGRFTCWWLTTIST |
| | DLTFSVKSSR |
| | GSSDPQGVTCGAATLSAERVRGDNKEYEYSVECQEDSACPAAEESLPIEVMVDAVHKLKYENYTSSFFIR |
| | DIIKPDPPKN |
| | LQLKPLKNSRQVEVSWEYPDTWSTPHSYFSLTFCVQVQGKSKREKKDRVFTDKTSATVICRKNASISVRA |
| | QDRYYSSSWS |
| | EWASVPCSGGGGGGSRNLPVATPDPGMFPCLHHSQNLLRAVSNMLQKARQTLEFYPCTSEEIDHEDITKD |
| | KTSTVEACLP |
| | LELTKNESCLNSRETSFITNGSCLASRKTSFMMALCLSSIYEDLKMYQVEFKTMNAKLLMDPKRQIFLDQN |
| | MLAVIDELM |
| | QALNFNSETVPQKSSLEEPDFYKTKIKLCILLHAFRIRAVTIDRVMSYLNAS |
| SEQ ID No. 33 | MCHQQLVISWFSL VFLASPLVA |
| SEQ ID No. 34 | IWELKKDVYVVELDWYPDAPGEMVVLTCDTPEEDGITWTLDQSSEVLGSGKTLTIQVK |
| | EFGDAGQYTCHKGGEVLSHSLLLLHKKEDGIWSTDILKDQKEPKNKTFLRCEAKNYSGRFTCWWLTTIST |
| | DLTFSVKSSR |
| | GSSDPQGVTCGAATLSAERVRGDNKEYEYSVECQEDSACPAAEESLPIEVMVDAVHKLKYENYTSSFFIR |
| | DIIKPDPPKN |
| | LQLKPLKNSRQVEVSWEYPDTWSTPHSYFSLTFCVQVQGKSKREKKDRVFTDKTSATVICRKNASISVRA |
| | QDRYYSSSWS |
| | EWASVPCS |
| | GGGGSRNLPVATPDPGMFPCLHHSQNLLRAVSNMLQKARQTLEFYPCTSEEIDHEDITKDKTSTVEACLP |
| | LELTKNESCLNSRETSFITNGSCLASRKTSFMMALCLSSIYEDLKMYQVEFKTMNAKLLMDPKRQIFLDQN |
| | MLAVIDELM |
| | QALNFNSETVPQKSSLEEPDFYKTKIKLCILLHAFRIRAVTIDRVMSYLNAS |
| SEQ ID No. 35 | MCHQQLVISWFSL VFLASPLVA |
| | IWELKKDVYVVELDWYPDAPGEMVVLTCDTPEEDGITWTLDQSSEVLGSGKTLTIQVK |
| | EFGDAGQYTCHKGGEVLSHSLLLLHKKEDGIWSTDILKDQKEPKNKTFLRCEAKNYSGRFTCWWLTTIST |
| | DLTFSVKSSR |
| | GSSDPQGVTCGAATLSAERVRGDNKEYEYSVECQEDSACPAAEESLPIEVMVDAVHKLKYENYTSSFFIR |
| | DIIKPDPPKN |
| | LQLKPLKNSRQVEVSWEYPDTWSTPHSYFSLTFCVQVQGKSKREKKDRVFTDKTSATVICRKNASISVRA |
| | QDRYYSSSWS |
| | EWASVPCS |
| | GGGGSRNLPVATPDPGMFPCLHHSQNLLRAVSNMLQKARQTLEFYPCTSEEIDHEDITKDKTSTVEACLP |
| | LELTKNESCLNSRETSFITNGSCLASRKTSFMMALCLSSIYEDLKMYQVEFKTMNAKLLMDPKRQIFLDQN |
| | MLAVIDELM |
| | QALNFNSETVPQKSSLEEPDFYKTKIKLCILLHAFRIRAVTIDRVMSYLNAS |
| SEQ ID No. 36 | RNLPVATPDPGMFPCLHHSQNLLRAVSNMLQKARQTLEFYPCTSEEIDHEDITKDKT |
| | STVEACLPLELTKNESCLNSRETSFITNGSCLASRKTSFMMALCLSSIYEDLK- |
| | MYQVEFKTMNAKLLMDPKRQIFLDQNM |
| | LAVIDELMQALNFNSETVPQKSSLEEPDFYKTKIKLCILLHAFRIRAVTIDRVMSYLNAS |
| | GGGGSGGGGSGGGGS |
| | IWELKKDVYVVELDWYPDAPGEMVVLTCDTPEEDGITWTLDQSSEVLGSGKTLTIQVK |
| | EFGDAGQYTCHKGGEVLSHSLLLLHKKEDGIWSTDILKDQKEPKNKTFLRCEAK- |
| | NYSGRFTCWWLTTISTDLTFSVKSSR |
| | GSSDPQGVTCGAATLSAERVRGDNKEYEYSVECQEDSACPAAEESLPIEVMVDAVHKLKYEN- |
| | YTSSFFIRDIIKPDPPKN |
| | LQLKPLKNSRQVEVSWEYPDTWSTPHSYFSLTFCVQVQGKSKREKKDRVFTDKTSAT- |
| | VICRKNASISVRAQDRYYSSSWS |
| | EWASVPCS |
| SEQ ID No. 37 | MCHQQL VISWFSL VFLASPLVA |
| | RNLPVATPDPGMFPCLHHSQNLLRAVSNMLQKARQTLEFYPCTSEEIDHEDITKDKT |
| | STVEACLPLELTKNESCLNSRETSFITNGSCLASRKTSFMMALCLSSIYEDLK- |
| | MYQVEFKTMNAKLLMDPKRQIFLDQNM |
| | LAVIDELMQALNFNSETVPQKSSLEEPDFYKTKIKLCILLHAFRIRAVTIDRVMSYLNAS |
| | GGGGSGGGGSGGGGS |
| | IWELKKDVYVVELDWYPDAPGEMVVLTCDTPEEDGITWTLDQSSEVLGSGKTLTIQVK |
| | EFGDAGQYTCHKGGEVLSHSLLLLHKKEDGIWSTDILKDQKEPKNKTFLRCEAK- |
| | NYSGRFTCWWLTTISTDLTFSVKSSR |
| | GSSDPQGVTCGAATLSAERVRGDNKEYEYSVECQEDSACPAAEESLPIEVMVDAVHKLKYEN- |
| | YTSSFFIRDIIKPDPPKN |
| | LQLKPLKNSRQVEVSWEYPDTWSTPHSYFSLTFCVQVQGKSKREKKDRVFTDKTSAT- |
| | VICRKNASISVRAQDRYYSSSWS |
| | EWASVPCS |
| SEQ ID No. 38 | RNLPVATPDPGMFPCLHHSQNLLRAVSNMLQKARQTLEFYPCTSEEIDHEDITKDKT |
| | STVEACLPLELTKNESCLNSRETSFITNGSCLASRKTSFMMALCLSSIYEDLK- |
| | MYQVEFKTMNAKLLMDPKRQIFLDQNM |
| | LAVIDELMQALNFNSETVPQKSSLEEPDFYKTKIKLCILLHAFRIRAVTIDRVMSYLNAS |
| | GGGGGGS |
| | IWELKKDVYVVELDWYPDAPGEMVVLTCDTPEEDGITWTLDQSSEVLGSGKTLTIQVK |
| | EFGDAGQYTCHKGGEVLSHSLLLLHKKEDGIWSTDILKDQKEPKNKTFLRCEAK- |
| | NYSGRFTCWWLTTISTDLTFSVKSSR |
| | GSSDPQGVTCGAATLSAERVRGDNKEYEYSVECQEDSACPAAEESLPIEVMVDAVHKLKYEN- |
| | YTSSFFIRDIIKPDPPKN |
| | LQLKPLKNSRQVEVSWEYPDTWSTPHSYFSLTFCVQVQGKSKREKKDRVFTDKTSAT- |
| | VICRKNASISVRAQDRYYSSSWS |
| | EWASVPCS |
| SEQ ID No. 39 | MCHQQLVISWFSL VFLASPLVA |
| | RNLPVATPDPGMFPCLHHSQNLLRAVSNMLQKARQTLEFYPCTSEEIDHEDITKDKT |
| | STVEACLPLELTKNESCLNSRETSFITNGSCLASRKTSFMMALCLSSIYEDLK- |
| | MYQVEFKTMNAKLLMDPKRQIFLDQNM |
| | LAVIDELMQALNFNSETVPQKSSLEEPDFYKTKIKLCILLHAFRIRAVTIDRVMSYLNAS |
| | GGGGGGS |
| | IWELKKDVYVVELDWYPDAPGEMVVLTCDTPEEDGITWTLDQSSEVLGSGKTLTIQVK |
| | EFGDAGQYTCHKGGEVLSHSLLLLHKKEDGIWSTDILKDQKEPKNKTFLRCEAK- |
| | NYSGRFTCWWLTTISTDLTFSVKSSR |
| | GSSDPQGVTCGAATLSAERVRGDNKEYEYSVECQEDSACPAAEESLPIEVMVDAVHKLKYEN- |
| | YTSSFFIRDIIKPDPPKN |
| | LQLKPLKNSRQVEVSWEYPDTWSTPHSYFSLTFCVQVQGKSKREKKDRVFTDKTSAT- |
| | VICRKNASISVRAQDRYYSSSWS |
| | EWASVPCS |
| SEQ ID No. 40 | RNLPVATPDPGMFPCLHHSQNLLRAVSNMLQKARQTLEFYPCTSEEIDHEDITKDKT |
| | STVEACLPLELTKNESCLNSRETSFITNGSCLASRKTSFMMALCLSSIYEDLK- |
| | MYQVEFKTMNAKLLMDPKRQIFLDQNM |
| | LAVIDELMQALNFNSETVPQKSSLEEPDFYKTKIKLCILLHAFRIRAVTIDRVMSYLNAS |
| | GGGGS |
| | IWELKKDVYVVELDWYPDAPGEMVVLTCDTPEEDGITWTLDQSSEVLGSGKTLTIQVK |
| | EFGDAGQYTCHKGGEVLSHSLLLLHKKEDGIWSTDILKDQKEPKNKTFLRCEAK- |
| | NYSGRFTCWWLTTISTDLTFSVKSSR |
| | GSSDPQGVTCGAATLSAERVRGDNKEYEYSVECQEDSACPAAEESLPIEVMVDAVHKLKYEN- |
| | YTSSFFIRDIIKPDPPKN |
| | LQLKPLKNSRQVEVSWEYPDTWSTPHSYFSLTFCVQVQGKSKREKKDRVFTDKTSAT- |
| | VICRKNASISVRAQDRYYSSSWS |
| | EWASVPCS |
| SEQ ID No. 41 | MCHQQLVISWFSL VFLASPLVA |
| | RNLPVATPDPGMFPCLHHSQNLLRAVSNMLQKARQTLEFYPCTSEEIDHEDITKDKT |
| | STVEACLPLELTKNESCLNSRETSFITNGSCLASRKTSFMMALCLSSIYEDLK- |
| | MYQVEFKTMNAKLLMDPKRQIFLDQNM |
| | LAVIDELMQALNFNSETVPQKSSLEEPDFYKTKIKLCILLHAFRIRAVTIDRVMSYLNAS |
| | GGGGS |
| | IWELKKDVYVVELDWYPDAPGEMVVLTCDTPEEDGITWTLDQSSEVLGSGKTLTIQVK |
| | EFGDAGQYTCHKGGEVLSHSLLLLHKKEDGIWSTDILKDQKEPKNKTFLRCEAK- |
| | NYSGRFTCWWLTTISTDLTFSVKSSR |
| | GSSDPQGVTCGAATLSAERVRGDNKEYEYSVECQEDSACPAAEESLPIEVMVDAVHKLKYEN- |
| | YTSSFFIRDIIKPDPPKN |
| | LQLKPLKNSRQVEVSWEYPDTWSTPHSYFSLTFCVQVQGKSKREKKDRVFTDKTSAT- |
| | VICRKNASISVRAQDRYYSSSWS |
| | EWASVPCS |

The nucleic acid construct of the invention also comprises one or more tetracycline-responsive aptazyme sequences. As used herein, the term “aptazyme sequence” includes both an RNA, i.e. the aptazyme sequence itself, and the DNA encoding such RNA. An aptazyme as used herein refers to an RNA molecule that combines ribozyme and aptamer functionalities. An aptazyme normally comprises a first and a second RNA sequence which have been fused to each other. The first RNA sequence has ribozyme activity, i.e. it catalyzes the cleavage of an RNA molecule. Preferably, the first RNA sequence catalyzes a self-cleavage reaction which means that it provides for an intramolecular RNA cleavage within the ribozyme part of the aptazyme. The second RNA sequence of the aptazyme has aptamer functionality, i.e. it is capable of binding to a target molecule due to a stable three-dimensional structure. The first RNA sequence having ribozyme activity and the second RNA sequence having aptamer functionality are fused such that the ribozyme activity of the first RNA sequence is influenced by the binding of the second RNA sequence to its cognate ligand. In this way, the aptazyme can control the integrity of a messenger RNA (mRNA) by conditional mRNA self-cleavage.

According to the invention, the aptazyme is tetracycline-responsive which means that the aptamer sequence of the aptazyme specifically binds to tetracycline and reacts to such binding by a change in the three-dimensional structure. By changing the structure of the aptamer sequence, the activity of the ribozyme sequence is modulated, i.e. either increased or decreased. In a preferred embodiment, tetracycline binding by the aptazyme decreases, and preferably completely prevents, RNA cleavage by the ribozyme, thereby providing for an increased expression of the nucleic acid construct of the invention by mRNA stabilization.

Hence, the at least one tetracycline-responsive aptazyme preferably induces or enhances expression of the transgene upon tetracycline binding. In a particularly preferred embodiment, expression levels of a DNA construct of the invention are at least 2-fold, at least 3-fold, at least 4-fold, at least 5-fold, at least 6-fold, at least 7-fold, at least 8-fold, at least 9-fold, at least 10-fold, at least 12-fold, or at least 14-fold, higher in the presence of an effective amount of tetracycline compared to the absence of tetracycline. More specifically, it is preferred that the nucleic acid construct of the invention, after delivery into a test subject, results in an at least 4-fold, preferably at least 6-fold, at least 8-fold, or at least 9-fold, higher expression level of the transgene compared to baseline level 8 hours after administration of 30 mg tetracycline per kg bodyweight to said subject. The test subject is a human, non-human primate or a mouse preferably a mouse.

In a preferred embodiment, the nucleic acid construct comprises a transgene encoding single chain IL-12, preferably human single chain IL-12, at least one tetracycline-responsive aptazyme sequence which comprises the sequence of SEQ ID NO:9 or SEQ ID NO:10 and ITRs derived from AAV2 such as the sequences according to Seq ID 8, 43, 44. 49. The construct preferably also comprises the liver-specific promoter LP1 such as the sequences according to SEQ ID NOs: 42 or 72. The nucleic acid construct preferably comprises any of the sequences set forth in SEQ ID NOs:29, 30, 31,46, 47, 50, 51, 57, 58, 59, 60, 61, 62, 63, 64, 65 or 66, or a complement of any of these. The nucleic acid construct may be double stranded.

Within the nucleic acid construct of the invention, the at least one tetracycline-responsive aptazyme can be located either 5′ or 3′ of the transgene. It is however preferred that the at least one tetracycline-responsive aptazyme is located 3′ of the transgene, e.g. in the 3′ UTR region of the transgene. The nucleic acid construct of the invention may also comprise more than one tetracycline-responsive aptazyme, such as 2, 3, 4 or 5 of these aptazymes. If the construct comprises two tetracycline-responsive aptazymes, it is preferred that these are both located 3′ of the transgene, e.g. in the 3′ UTR region. Such arrangement is referred to herein as 3′3′ construct.

In a particularly preferred embodiment, the tetracycline-responsive aptazyme is the aptazyme “K19” previously described by Beilstein et al. 2015. The aptazyme comprises the tetracycline aptamer (Berens et al. 2001) and the full-length hammerhead ribozyme N79 from Schistosoma mansoni (Yen et al. 2004). The sequence of the aptazyme K19 is provided in SEQ ID NO:10 herein. The respective DNA sequence encoding the aptazyme K19 is provided in SEQ ID NO:9. Accordingly, in one embodiment of the invention, the tetracycline-responsive aptazyme sequence comprises or consists of the sequence set forth in any of SEQ ID NOs: 9 or 10.

The nucleic acid construct further comprises inverted terminal repeat (ITR) sequences. An ITR normally comprises of a first upstream nucleotide sequence which is followed by a second downstream nucleotide sequence which is the reverse complement of the first upstream nucleotide sequence. The intervening sequence of nucleotides (if any) between the first upstream and the second downstream nucleotide sequence can be of any length.

ITR sequences naturally occur in the genome of AAV and retroviruses where they are involved in packaging of the nucleic acid into viral capsids. Preferably, the ITR sequences of the nucleic acid construct of the invention comprises flank the transgene and the aptazyme, which means that the transgene and the aptazyme are located between the ITR sequences. It is preferred that the two ITR sequences that flank the transgene and the aptazyme are each about 140-145 bp in length. In a preferred embodiment, the ITR sequences in the nucleic acid construct of the invention are derived from an adeno-associated virus, preferably from AAV2, AAV8, or AAV9. It is particularly preferred that the ITR sequences comprise or consist of the ITR sequence set forth in SEQ ID NO: 8.

The ITR sequences usually both have a length between 130 and 145 nucleotides. At least one of which may be considerably shorter (Zhou, Tian et al., 2017). It is preferred that the two ITR sequences that flank the transgene and the aptazyme are each about 145 bp in length. In a preferred embodiment, the ITR sequences in the nucleic acid construct of the invention are derived from an adeno-associated virus, preferably from AAV2 (Wilmott et al., 2019; Samulski et al., 1983, Zhou et al., 2017). It is particularly preferred that the ITR sequences comprise or consist of the ITR sequence set forth in SEQ ID NO: 8, 43, 49, 50. It is understood that the ITRs have to be arranged in a certain way to exhibit their function: For the AAV2 wild-type ITR sequences according to Wilmott et al., 2019 the following set up is preferred:

(ITR right 3′-downstream)
(Seq ID 48)
5′aggaacccctagtgatggagttggccactccctctctgcgcgctcgct
cgctcactgaggccgcccgggcaaagcccgggcgtcgggcgacctttggt
cgcccggcctcagtgagcgagcgagcgcgcagagagggagtggccaa′3
Revcomp: (ITR left 5′-upstream)
(Seq ID 43)
5′ttggccactccctctctgcgcgctcgctcgctcactgaggccgggcga
ccaaaggtcgcccgacgcccgggctttgcccgggcggcctcagtgagcga
gcgagcgcgcagagagggagtggccaactccatcactaggggttcct′3

In the scientific literature, the view shown for ITR right 3′-downstream is more common.

The construct comprising the transgene, the tetracycline-responsive aptazyme, and the ITRs can in principle have any size. Preferably, the size will be such that it can be packaged into the capsid of a viral vector. A skilled person will readily be able to select the size in consideration of the packaging capacity of the viral vector at hand. For example, if the nucleic acid construct, e.g. a single-stranded DNA, shall be used in combination with an AAV vector, the size of the construct should be below 4.7 kb which is the maximum size that is effectively packaged into an AAV vector. In a preferred embodiment of the invention, the nucleic acid construct is between 0.5 kb and 4.5 kb in size, such as between 0.75 kb and 4.0 kb, between 1.0 kb and 3.5 kb, between 1.5 kb and 3.0 kb, or between 2.0 kb and 2.5 kb. In a particularly preferred embodiment, the nucleic acid construct is a DNA molecule having a size between 0.5 kb and 4.5 kb, between 0.75 kb and 4.0 kb, between 1.0 kb and 3.5 kb, between 1.5 kb and 3.0 kb, or between 2.0 kb and 2.5 kb.

The nucleic acid construct may further comprise a promoter that drives the expression of the one or more transgenes. The promoter will be selected dependent on the intended use of the construct and the putative site of transgene expression. For example, where expression of the transgene in the liver is desired, a promoter having a high activity in liver tissue will be selected, such as the liver-specific promoter LP1. Similarly, if the transgene is to be expressed in tumor tissue, a tumor-specific promoter will be used, such as the alpha fetoprotein (AFP) promoter. Accordingly, in a preferred embodiment, the nucleic acid construct of the invention comprises a liver-specific promoter or a tumor-specific promoter.

As used herein, liver-specific promoters include the LP1 promoter, the transthyretin (TTR) promoter, A1AT promoter, and the thyroxine binding globulin (TPG) promoter (Greig et al., 2017), hybrid liver-specific promoter (HLP), human thyroxine-binding globulin (TBG), transthyretin (TTR), human alpha 1-antitrypsin (hAAT) promoter combined with liver-specific apolipoprotein E (ApoE) enhancer, synthetic liver-specific promoters (Okuyama et al., 1996; Cabrera-Pérez et al. 2019; EP2698163A1, WO2020104424). Tumor-specific promoters include the alpha fetoprotein (AFP) promoter (Shi, et al. 2004), the CEA promoter (Cao et al. 1998; Lan et al. 1997) and the Mucl promoter (Chen et al. 1995; Tai et al. 1999), and the hTERT promoter (Quante et al. 2005). Differential transcriptional regulation of human telomerase in a cellular model representing important genetic alterations in esophageal squamous carcinogenesis, Carcinogenesis vol.26 no.11 pp. 1879-1889). In an even more preferred embodiment, the nucleic acid construct of the invention comprises a promoter that is selected from the group of the human cytomegalovirus (CMV) promoter, the liver-specific promoter LP1, the tumor-specific alpha fetoprotein (AFP) promoter, and the human telomerase reverse transcriptase (hTERT) promoter.

As a further component, the nucleic acid construct of the invention may comprise a poly(A) signal. A poly(A) signal is a sequence motif which is recognized by the RNA cleavage complex, a multi-protein complex that cleaves the mRNA at the end of the transcription process. In a subsequent step, a tail of adenosine monophosphate residues is added to the 3′ end of a mRNA in a reaction catalyzed by the enzyme polyadenylate polymerase. The resulting poly(A) tail is in involved in nuclear export, translation, and stability of mRNA. Poly(A) signals are well known to a skilled person. Most human polyadenylation signals contain the sequence AAUAAA. Thus, in a preferred embodiment the nucleic acid construct of the invention comprises the sequence AAUAAA. In yet another preferred embodiment the nucleic acid construct comprises a synthetic poly(A) signal as described (Levitt et al., 1989). In yet another preferred embodiment the nucleic acid construct comprises the SV40 poly(A) signal depicted in SEQ ID NO: 7.

Preferably, the nucleic acid construct of the invention is a DNA construct comprising a transgene expression cassette. The expression cassette comprises a promoter which is operably linked to a transgene encoding a therapeutic protein, a sequence encoding an aptazyme upstream or downstream of the transgene, a polyadenylation signal, and ITR sequences at the 3′ and 5′ end.

In another aspect, the present invention relates to a transgene expression cassette comprising a promoter, a transgene encoding one or more therapeutic proteins, and at least one tetracycline-responsive aptazyme sequence. The promoter preferably is a liverspecific promoter, such as the liver-specific LP1 promoter, or a tumor-specific promoter as described hereinabove. The liver specific LP1 promotor may comprise an intron, see Seq ID 42. An example without SV40 intron is shown in Seq ID 72,

Preferably, the transgene expression cassette will comprise or consist of DNA. The expression cassette may be in linear or circular form, e.g. in the form of a plasmid. In a preferred embodiment, the transgene expression cassette of the invention comprises or consists of single-stranded or double-stranded DNA. In a particularly preferred embodiment, transgene expression cassette of the invention consists of single-stranded DNA.

The transgene expression cassette may further comprise a poly(A) signal, such as a SV40 poly(A) signal as described hereinabove. Thus, in a preferred embodiment the transgene expression cassette of the invention comprises the sequence AAUAAA. In yet another preferred embodiment the transgene expression cassette comprises a synthetic poly(A) signal. In yet another preferred embodiment the transgene expression cassette comprises the SV40 poly(A) signal depicted in SEQ ID NO: 7.

The transgene expression cassette comprises a transgene encoding one or more immunoregulatory proteins. As described hereinabove, immunoregulatory proteins include, but are not limited to, antibodies, such as Ipilimumab or anti-PD1 antibodies, antibody fragments, cytokines, such as interleukins, interferons, lymphokines, and pro-inflammatory and pro-apoptotic members of the tumor necrosis factor (TNF)/tumor necrosis factor receptor (TNFR) superfamily. Immunoregulatory proteins further include, but are not limited to, T cell engagers, immune checkpoint inhibitors, agonists such as anti-CD137, anti-CD28, or anti-CD40 combinations of any of the above. In a preferred embodiment, the immunoregulatory protein encoded by the transgene is an interleukin selected from the group consisting of IL-4, IL-6, IL-10, IL-11, IL-12, IL-13, IL-23, IL-27, and IL-33. It is particularly preferred that the immunoregulatory protein in the transgene expression cassette of the invention is IL-12, preferably human IL-12. Preferably, the single chain IL-12 comprises one or more of the sequences selected from the group consisting of SEQ ID Nos. 1-6 or 34 to 41. Further examples can be found in Table 4 below.

The transgene expression cassette of the invention comprises at least one tetracycline-responsive aptazyme sequence. As described herein above, the at least one tetracycline-responsive aptazyme can be located either 5′ or 3′ of the transgene. It is however preferred that the at least one tetracycline-responsive aptazyme is located 3′ of the transgene, e.g. in the 3′ UTR region of the transgene. The transgene expression cassette of the invention may also comprise more than one tetracycline-responsive aptazyme, such as 2, 3, 4 or 5 of these aptazymes. It is particularly preferred that the tetracycline-responsive aptazyme comprises or consists of the sequence set forth in any of SEQ ID NOs: 9 or 10.

The at least one tetracycline-responsive aptazyme sequence preferably induces or enhances expression of the transgene upon tetracycline binding. In a particularly preferred embodiment, expression levels of a DNA construct of the invention are at least 2-fold, at least 3-fold, at least 4-fold, at least 5-fold, at least 6-fold, at least 7-fold, at least 8-fold, at least 9-fold, at least 10-fold, at least 12-fold, or at least 14-fold, higher in the presence of an effective amount of tetracycline compared to the absence of tetracycline. More specifically, it is preferred that the transgene expression cassette of the invention, after delivery into a subject, results in an at least 4-fold, preferably at least 6-fold, at least 8-fold, or at least 9-fold, higher expression level of the transgene compared to baseline level 8 hours after administration of 30 mg tetracycline per kg bodyweight to said subject. The subject is preferably a mouse.

In a preferred embodiment, the present invention provides a transgene expression cassette comprising a transgene encoding single chain IL-12, preferably human single chain IL-12, at least one tetracycline-responsive aptazyme sequence which comprises the sequence of SEQ ID NO:9 or SEQ ID NO:10 and ITRs derived from AAV2. The expression cassette preferably also comprises the liver-specific promoter LP1. The expression cassette preferably comprises any of the sequences set forth in SEQ ID NOs: SEQ ID NOs:29, 30, 31, 46, 47, 50, 51, 57, 58, 59, 60, 61, 62, 63, 64, 65 or 66, without the flanking ITRs, preferably any of sequences set forth in SEQ ID NO: 73 or 74.

In another aspect, the invention relates to a viral vector comprising a capsid and a packaged nucleic acid, wherein the packaged nucleic acid comprises a nucleic acid construct or a transgene expression cassette as defined herein above, preferably a DNA construct. The viral vector can be selected dependent on the tissue to be transduced. Non-limiting examples of viral vectors that can be used in accordance with the invention include lentivirus vectors, adenovirus vectors, adeno-associated virus vectors (AAV vectors), and paramyxovirus vectors. Among these, the AAV vectors are particularly preferred, especially those with an AAV-2, AAV-8 or AAV-9 serotype. The viral vectors may comprise capsid proteins that have been modulated to include an amino acid sequence that provides for selective binding to a target tissue, such as liver tissue or lung tissue (see for example WO 2015/018860).

The nucleic acid constructs or transgene expression cassettes of the invention are particularly useful for the treatment of cancer diseases, in particular liver cancer. After systemic injection, the nucleic acid constructs or transgene expression cassettes of the invention locally deliver a regulatable transgene expression cassette, e.g. by use of a tissue-tropic AAV that targets towards a chosen cancerous organ (e.g. liver), induce local expression of the therapeutic protein in the cancerous organ, subsequent activation of T cells and other immune cells, and tumor elimination. The nucleic acid constructs or transgene expression cassettes can be applied to eliminate primary tumors, as well as secondary tumors (i.e. metastases) which are located in the cancerous organ provided with the regulated expression cassette. T cells and other immune cells primed locally through the described system can migrate with the blood stream to distant sites of the body, and induce abscopal anti-tumor responses towards cancer lesions that are located outside the cancerous organ that had been provided with the nucleic acid constructs or transgene expression cassettes.

It is known that many cancer patients die not as a result of their primary tumors, but rather of metastases resulting from those primary tumors (Dillekas et al., 2019). The formation of metastasis is a complex process that depends on both the circulation from the primary tumor and the properties of the target organ, such as its propensity to suppress the immune system. Several tumor types frequently metastasize to the liver, including colorectal cancer (Valderrama-Treviño et al., 2017), lung cancer and melanoma. For example, hepatic metastasis formation correlates with diminished immunotherapy efficacy in patients with cancer (Yu et al., 2021). Due to the location of the metastases in liver, their size, the amounts of liver metastases, residual normal liver, and additional hepatic disease, 85% of these patients are not eligible for surgery (Jemal et al., 2002), representing a very high medical need. The nucleic acid constructs or transgene expression cassettes of the invention therefore represent an important contribution by providing a treatment option for these patients.

For the treatment of cancers residing in the liver, hepatocytes represent an ideal target cell population in order to transduce them for release of IL-12 in the proximity of the tumor. AAV vectors have an excellent safety and efficacy profile documented in over 180 clinical trials (Paulk, 2020) and have been used widely for systemic liver gene delivery due to their natural hepatotropism (Wang et al., 2019). As such, AAV vectors encapsidating the IL-12 gene combined with a riboswitch cassette for toggleable control, would represent an ideal platform for the regulatable IL-12 gene therapy of liver cancers.

In another aspect, the invention relates to a nucleic acid construct or transgene expression cassette as defined herein above or a viral vector according as defined herein above for use in medicine. Specifically, the nucleic acid constructs, transgene expression cassettes and viral vectors are contemplated for use in a method of treating a proliferative disease, such as fibrosis or a cancer disease. Cancer diseases that can be treated by the nucleic acid constructs, transgene expression cassettes and viral vectors of the invention comprise liver cancer, brain cancer, pancreatic cancer, colorectal cancer, esophageal cancer, gastric cancer, hepatocellular cancer, anal cancer, breast cancer, cervical cancer, ovarian cancer, endometrial cancer, prostate cancer, testicular cancer, vulvar cancer, skin cancer, urogenital cancer, renal cancer, bladder cancer, head and neck cancer, oropharyngeal cancer, laryngeal cancer, non-small cell lung cancer, small cell lung cancer. In a particularly preferred embodiment of the invention, the nucleic acid constructs, transgene expression cassettes or viral vectors are for use in a method of treating or preventing liver cancer, such as hepatocellular carcinoma (HCC) or cholangiocarcinoma. In another preferred embodiment, the nucleic acid construct, transgene expression cassette or viral vector of the invention is used for treating colorectal cancer.

It is particularly preferred according to the invention that the nucleic acid construct, the transgene expression cassette or the viral vector of the invention is used to treat a patient that has one or more cancer lesions located in the liver. The lesions may result from a primary liver cancer or from a secondary liver cancer. As used herein, secondary liver cancer is understood to refer to metastasis in the liver that result from a primary tumor other than a liver tumor.

If the nucleic acid construct or the transgene expression cassette of the invention is administered to a subject in the form of a viral vector, it is preferred that the viral vector is administered in an amount corresponding to a dose of virus in the range of 1.0×10¹⁰ to 1.0×10¹⁴ vg/kg (virus genomes per kg body weight), although a range of 1.0×10¹¹ to 1.0×10¹² vg/kg is more preferred, and a range of 5.0×10¹¹ to 5.0×10¹² vg/kg is still more preferred, and a range of 1.0×10¹² to 5.0×10¹¹ is still more preferred. A virus dose of approximately 2.5×10¹² vg/kg is most preferred. The amount of the viral vector to be administered, such as the AAV vector according to the invention, for example, can be adjusted according to the strength of the expression of the one or more transgenes.

In another aspect, the invention provides a cell which comprises a nucleic acid construct, a transgene expression cassette or a viral vector as defined herein above.

In yet another aspect, the invention provides a pharmaceutical composition comprising a nucleic acid construct, a transgene expression cassette or a viral vector as defined herein above in combination with a pharmaceutical-acceptable carrier or diluent.

In yet another aspect, the invention provides a method of treating a proliferative disease comprising administering to a patient in need thereof a therapeutically effective amount of a nucleic acid construct, transgene expression cassette or a viral vector as defined herein above. Preferably, the proliferative disease to be treated is a fibrosis or cancer disease. Cancer diseases that can be treated by the nucleic acid constructs, transgene expression cassettes and viral vectors of the invention have been discussed elsewhere herein. The treatment of liver cancer is particularly preferred.

In yet another aspect, the invention relates to the use of a nucleic acid construct, transgene expression cassette or viral vector of the invention for the manufacture of a medicament for treating a proliferative disease, such as a cancer disease.

### Examples

### 1. Materials and Methods

### 1.1 Expression Constructs

Riboswitch or control plasmid constructs were cloned using available constructs based on CMV or LP1 promoters and an enhanced GFP (eGFP) transgene as wells as a SV40 poly(A) signal. For packaging into recombinant AAV vectors, all plasmids were equipped with AAV2 ITRs. All AAVs applied in in vivo experiments had an 11nt deletion in the left ITR (see SEQ ID NO: 46 and analogous constructs). Cellular and secreted NanoLuciferase genes were derived from pNL1.1 and pNL1.3 vectors purchased from Promega. The anti-FITC tandem scFv construct was constructed based on published sequences (Vaughan et al. 1996) and synthesized at Life technologies. The K19 riboswitch sequence was derived from Beilstein (Beilstein et al. 2015) and cloned into the reporter constructs flanked by (CAAA) 3 spacer sequences. The sequences encoding murine or human single chain IL-12 were derived from the published sequence mIL-12.p40.L.Ap35 (Lieschke et al., 1997). The human IgG signal peptide was then introduced by PCR and cloned into pCR-TOPOP3.3. Restriction enzyme mediated subcloning of the continuous sequence encoding the signal peptide and single chain IL-12 replaced the reporter genes in the respective AAV plasmids.

### 1.2 Cellular Assays

HEK293H and HepG2 cells were cultured in DMEM+GlutaMAX+10% FCS at 37° C. 30,000 HEK293 cells per 96-well were seeded 24 h prior to transfection using the Lipofectamine-3000 kit with 35 ng DNA, 0.07 μL P3000, 0.15 μL Lipofectamine-3000 and 10 μL Opti-MEM per well. Master mixes were prepared and up-scaled according to growth area for bigger culture formats. Transfection optimization for HepG2 resulted in 50,000 cells being seeded and transfected using 70 ng DNA, 0.14 μL P3000 and 0.15 μL Lipofectamine-3000 per 96-well. 10,000. Unless stated differently, tetracycline (Tet-HCl, Sigma-Aldrich) was added to cells 1-2 h after transfection and simultaneously to AAV addition in case of transduction. Tet was stored as frozen 2 mM stock solution in water in light-protected single-use aliquots and serially diluted in water prior to its addition (10 μL per 96-well) to the cells.

### 1.3 Production of Recombinant AAV Vectors

AAVs were produced in transiently transfected HEK293 cells and quantified by qPCR as described (Strobel et al. 2015a). Briefly, HEK293H cells were cultivated in DMEM+GlutaMAX media supplemented with 10% fetal calf serum. Three days before transfection, the cells were seeded in 15 cm tissue culture plates to reach 70-80% confluency on the day of transfection. For transfection, 0.5 μg total DNA per cm 2 of culture area were mixed with 1/10 culture volume of 300 mM CaCl₂ as well as all plasmids required for AAV production in an equimolar ratio. The plasmid constructs were as follows: One plasmid encoding the AAV cap gene (Strobel et al., 2015a); the AAV cis-plasmid containing the expression cassette flanked by ITRs; a pHelper plasmid (AAV Helper-free system, Agilent). The plasmid CaCl₂ mix was then added dropwise to an equal volume of 2× HBS buffer (50 mM HEPES, 280 mM NaCl, 1.5 mM Na₂HPO₄), incubated for 2 min at room temperature and added to the cells. After 5-6 h of incubation, the culture medium was replaced by fresh medium. The transfected cells were grown at 37° C. for a total of 72 h. Cells were detached by addition of EDTA to a final concentration of 6.25 mM and pelleted by centrifugation at room temperature and 1000× g for 10 min. The cells were then resuspended in lysis buffer (50 mM Tris, 150 mM NaCl, 2 mM MgCl₂, pH 8.5).

AAV vectors were purified essentially as previously described (Strobel et al., 2015a): For iodixanol gradient based purification, cells harvested from up to 40 plates were dissolved in 8 mL lysis buffer. Cells were then lysed by three freeze/thaw cycles using liquid nitrogen and a 37° C. water bath. For each initially transfected plate, 100 units Benzonase nuclease (Merck) were added to the mix and incubated for 1 h at 37° C. After pelleting cell debris for 15 min at 2500× g, the supernatant was transferred to a 39 mL Beckman Coulter Quick-Seal tube and an iodixanol (OptiPrep, Sigma Aldrich) step gradient was prepared by layering 8 mL of 15%, 6 mL of 25%, 8 mL of 4% and 5 mL of 58% iodixanol solution diluted in PBS-MK (lx PBS, 1 mM MgCl₂, 2.5 mM KCl) below the cell lysate. NaCl had previously been added to the 15% phase at 1 M final concentration. 1.5 μL of 0.5% phenol red had been added per mL to the 15% and 25% iodixanol solutions and 0.5 μL had been added to the 58% phase to facilitate easier distinguishing of the phase boundaries within the gradient. After centrifugation in a 70Ti rotor for 2 h at 63000 rpm and 18° C., the tube was punctured at the bottom. The first five milliliters (corresponding to the 58% phase) were then discarded, and the following 3.5 mL, containing AAV vector particles, were collected. PBS was added to the AAV fraction to reach a total volume of 15 mL and ultrafiltered/concentrated using Merck Millipore Amicon Ultra-15 centrifugal filter units with a MWCO of 100 kDa. After concentration to ˜1 mL, the retentate was filled up to 15 mL and concentrated again. This process was repeated three times in total. Glycerol was added to the preparation at a final concentration of 10%. After sterile filtration using the Merck Millipore Ultrafree-CL filter tubes, the AAV product was aliquoted and stored at −80° C.

### 1.4 AAV In Vivo Experiments

9-12 weeks old C57BL/6 mice with a body weight of 19-21 g were purchased from Charles River laboratories. AAVs were diluted to the desired concentration in PBS and administered into the tail vein in a volume of 100 μL per mouse, under light isoflurane anesthesia. For preparation of a 100 mg/kg tetracycline solution, 20 mg tetracycline-HCl were dissolved in 400 μL 25% 2-hydroxypropyl beta-cyclodextrin solution (HP-β-CD, Sigma Aldrich) +600 μL PBS and adjusted to about pH 6 by addition of 35 μL 1M NaOH. For lower doses, this solution was serially diluted in PBS+HP-β-CD. Tet solutions were prepared immediately before i.p. administration (200 μL per mouse). At the respective time points, 20 μL blood were sampled by puncturing the vena saphena and collected using K3-EDTA Microvette POCT 20 μL capillary microtubes (Sarstedt) followed by centrifugation. Blood plasma was used for quantitative anti-FITC and tetracycline measurements. At the final blood draw, additional serum samples were prepared for the assessment of liver enzymes. Organs of interest were dissected and snap-frozen in liquid nitrogen for DNA/RNA extraction or the preparation of protein tissue lysates. For experiments in tumor-bearing mice, luciferase-expressing Hepal-6 tumor cells (1.0×10⁶ cells in 50 μL PBS) were injected into the spleen of each mouse under anesthesia and allowed to migrate into the liver for 5 min via vena lienalis. Thereafter the spleen was resected. Mice received the analgeticum Meloxicam (1.0 mg/kg in 10.0 ml/kg) subcutaneously 1-2 h before surgery and 24 h later. Body weight and tumor growth were monitored. Tumor volumes were determined by in vivo bioluminescence using an IVIS® Lumina III bioluminescence imaging system (Perkin Elmer) with a CCD-camera. For this purpose, 150 mg/kg (7.5 mL/kg) D-Luciferin in aqua dest. was injected i.p. 8 min before anesthetization. Light emission was measured 10 min post injection. Tumor-bearing mice were block-randomized according to tumor sizes measured by the in vivo biolumines-cence imaging of the same day. For block-randomization, a robust automated random number generation within individual blocks was used (MS-Excel 2016).

### 1.5 Reporter Protein Assessments

eGFP expression was assessed by fluorescence microscopy or direct fluorescence detection using the Molecular Devices SpextraMax i3x with a MiniMax 300 imaging unit. Nano-Luciferase measurements were performed using the Promega Nano-Glo Luciferase assay as per manufacturer's instructions. If required, appropriate sample dilutions were identified prior to assessment. A detailed description of the anti-FITC ELISA setup and measurement is provided further below.

### 1.6 Expression and Purification of Anti-FITC Protein (ELISA Standard)

HEK293 cells were transfected using the calcium phosphate method as described for AAV production, using 30 μg of CMV-aFITC expression plasmid per 15-cm culture dish. 48 h after transfection, the culture supernatant was harvested and centrifuged at 400×g for 5 min. 45 mL supernatant were then mixed with 60 μL of anti-VS beads and purified using the “VS-tagged protein purification kit Ver.2” (3317, MBL) as per instructions. Following protein elution, V5 elution peptide was removed by ultrafiltration. Therefore, 40 μL protein eluate were added to a PBS-pre-equilibrated Vivaspin 500 column (VS0101, Sartorius) and filled up to 500 μL using PBS. Following centrifugation at 15000×g for approximately 2 min to reach a retentate volume of 50 μL, PBS was again added to 500 μL and centrifuged again. This process was repeated three times in total. After retentate recovery, anti-FITC protein was aliquoted and frozen at −20° C. Protein concentration was determined using NanoDrop-One measurements at 280 nm and calculations based on a protein size of 57 kDa and a molar extinction coefficient of 116,240 M⁻¹ cm⁻¹.

### 1.7 Anti-FITC ELISA

A standard MSD plate (L15XA-1) was coated with 30 μL of BSA-FITC (A23015, Molecular Probes) and diluted to 0.25 μg/mL in PBS under shaking for 5 min at 750 rpm. After incubation at 4° C. overnight (or 1 h at room temperature (RT)), the plate was washed three times using 300 μL/well wash buffer (PBS+0.05% Tween-20). 150 μL of blocking solution (3% Blocker A (R93BA-2, MSD) in PBS) were then added and incubated for 1 h at 750 rpm and RT. After washing three times, 25 μL of each sample, stand- and (diluted in 1% Blocker A in PBS) or blank were added per well and incubated for 1 h at 750 rpm and RT. Detection antibody (biotinylated rabbit anti-V5, ab18617, Abcam) was diluted to 1 μg/mL in 1% Blocker A in PBS and SULFO-tag labeled streptavidin (R32AD-5, MSD) was diluted to 0.5 μg/mL in 1% Blocker A in PBS. After washing the plate three times, 25 μL of antibody and streptavidin dilutions were added simultaneously to each well and incubated for 1 h at 750 rpm and RT. Following three washing steps, 150 μL/well of 2× Read Buffer T (R92TC-2, MSD) diluted in water were added per well. The plate was then read using an MSD Sector Imager 600.

### 1.8 IL-12, IFNγ ELISA

Expression of IL-12 and IFNγ were analyzed using the Mouse IL-12p70 or the Proinflammatory Panel 1 Mouse Kit (K152ARB, K15048D, MSD) according to the manufacturer's instructions. Lowest standard of provided IL12p70 was taken as lower limit of detection (LLOD).

### 1.9 Preparation of Protein Tissue Lysates

Flash frozen tissue samples were homogenized in 100 μL MSD lysis buffer (R6OTX-2), using a Precellys-24 homogenizer and ceramic (KT03961-1-009.2, VWR) or metal bead tubes (KT03961-1-001.2) at 6000 rpm for 30 sec. Homogenates were immediately placed on ice, followed by addition of additional 900 μL lysis buffer. A second round of homogenization was then carried out. Samples were again cooled on ice and centrifuged for 10 min at 20,000×g. 700 μL of supernatant were recovered and protein concentration was determined using a BCA assay (Promega). Homogenates were stored at −80° C.

### 1.10 DNA and RNA Isolation

Tissue samples were flash frozen immediately after dissection. For DNA and RNA isolation, samples were homogenized in 900 μL RLT buffer (79216, Qiagen), using a Precellys-24 homogenizer and ceramic bead tubes (KT03961-1-009.2, VWR) at 6000 rpm for 30 sec. Afterwards, samples were immediately placed on ice. 350 μL Phenolchloroform-isoamyl alcohol (77617, Sigma Aldrich) were then added to 700 μL homogenate in a Phase Lock gel tube and mixed by shaking. Following centrifugation for 5 min at 16000×g, 350 μL Chloroform-isoamyl alcohol (25666, Sigma-Aldrich) were added and the mixture was shaken again. After 3 min of incubation at RT and centrifugation for 5 min at 12000×g, the upper (aqueous) phase was collected and pipetted into a deep well plate placed on dry ice. After processing of all samples, DNA and RNA were purified, using the AllPrep DNA/RNA 96 kit (80311, Qiagen) as per instructions, including the optional “on-column DNase digestion” step. RNA from cell cultures was isolated by pelleting cells, followed by lysis in 350 μL RLT buffer and purification using the RNeasy mini kit (74104, Qiagen).

1.11 Analysis of Gene Expression and AAV Vector Genomes (qPCR and ddPCR)

For gene expression analysis, equal amounts of RNA were reverse transcribed to cDNA using the High-capacity cDNA RT kit (4368814, Thermo Fisher) as per instructions. qRT-PCR reactions were set up using the QuantiFast Probe RT-PCR kit (204456, Qiagen) and primers specifically binding the K19 riboswitch sequence or the anti-FITC gene. Expression was normalized to RNA polymerase II housekeeper expression. AAV vector genomes were detected using extracted DNA either for ddPCR or qPCR. For qPCR a standard curve was generated by serial dilutions of the respective expression plasmid. qPCR runs were performed on an Applied Biosystems ViiA 7 Real-Time PCR System. For ddPCR, Automated Droplet Generator, QX200 Droplet Digital PCR System, and QX200 Droplet Reader (all Bio-rad) were applied.

### 1.12 Pharmacokinetic and Exposure Measurements

Pharmacokinetic of tetracycline was investigated in 12 weeks old (approximately 30 g body weight) male C57BL/6 mice purchased from Janvier Labs. A Tet solution was administered i.p. at an administration volume of 10 mL/kg and a dose of 54 mg/kg. The Tet solution contained 10% 2-hydroxypropyl beta-cyclodextrin and was adjusted to pH 6. Serial blood sampling was performed via puncture of the saphenous vein into K3-EDTA coated vials. A maximum volume of 20 μL blood was collected per sampling time point. Plasma samples were prepared by centrifugation. For tissue distribution, intraperitoneal dosing was performed as described above in the same animals at day 3. The mice were sacrificed two hours after Tet administration and subsequently the brain, liver, kidney, heart, lungs, both eyes, a piece of leg muscle and a blood sample were collected. Tissue weights were recorded and all samples were stored at −20° C. prior to bioanalysis. Plasma protein was precipitated with acetonitrile. Tissue samples were transferred to Precellys vials and three parts of acetonitrile/methanol (1:1) and one part of water was added for the homogenization step. All samples were centrifuged prior to bioanalysis. Compound concentrations were determined by high performance liquid chromatography coupled with tandem mass spectrometry.

### 1.13 Assessment of AST, ALT and GLDH Enzyme Activity

All measurements were performed using the Konelab PRIME 60 and test kits from Thermo Scientific (following the Konelab Chemistry Information Manual 12A/2003, March 2003) and spectrophotometrical assessment at 340 nm. Aspartate aminotransferase (AST) activity was measured by an enzymatic rate method (Schumann et al. 2002a) without pyridoxal-5′-phosphate for AST activation. Alanine aminotransferase (ALT) activity was measured by an enzymatic rate method based on the IFCC method (Schumann et al. 2002b). without adding pyridoxal-5′-phosphate. The removal of NADH was measured spectrophotometrically at 340 nm. Glutamate dehydrogenase (GLDH) activity was measured by an enzymatic rate method, using a kit supplied by Roche Diagnostics.

### 1.14 IL-12 In Vitro Bioactivity Reporter Assays

A bioassay is employed to measure human or mouse IL-12 bioactivity as a function of the proliferation of phytohaemagglutinin (PHA)-activated human lymphoblasts, as described by Gately et al., 1995.

### Basic Protocol 1: Antibody-Capture Bioassay for IL-12 Activity.

Briefly, this functional assay is based on the ability of IL-12 to stimulate proliferation of PHA-activated T lymphoblasts (“PHA blasts”). In this assay, IL-12 that has been bound to immobilized anti-IL-12 antibody stimulates proliferation of PHA-activated human lymphoblasts. Human or mouse IL-12 is captured from IL-12-containing culture fluid or serum by anti—human IL-12 or anti—mouse IL-12 antibody adsorbed to the wells of an EIA (enzyme immunoassay) plate. The test fluid is then washed from the wells and replaced with a PHA-activated human lymphoblast suspension. The lymphoblasts proliferation in response to the captured IL-12 is measured. Commercially available bioactive human IL-12 recombinant protein consisting of two subunits linked via a disulphide bond (for example Thermo Fisher Scientific; Cat. #PHC1124) is used as a standard.

As an alternative, a commercial IL-12 Bioassay (Promega GmbH; Cat.#J3042) can also be employed. This is a bioluminescent cell-based assay designed to measure IL-12 stimulation or inhibition and is performed according to manufacturer's instructions. Briefly, the IL-12 Bioassay consists of a genetically engineered human cell line that expresses a luciferase reporter driven by a response element (RE). When IL-12 binds to IL-12R it transduces intracellular signals resulting in luminescence. The bioluminescent signal is detected and quantified using Bio-Glo™ Luciferase Assay System (Cat.#G7940, G7941) and a standard luminometer.

As another alternative, a HEK-Blue™ assay can be used for showing IL-12 bioactivity in vitro. HEK-Blue™ IL-12 cells (InvivoGen, #hkb-i112) are designed to detect bioactive human and murine IL-12. The human embryonic kidney HEK293-based cell line expresses the human genes for the IL-12 receptor and the genes of the IL-12 signaling pathway into line, and a STAT4-inducible SEAP reporter gene. Cell surface ligand binding triggers a signaling cascade activating STAT-4 and production of the reporter protein secreted alkaline phosphatase (SEAP). SEAP can be detected in the supernatant using QUANTI-Blue™ Solution according to manufacturer's instructions. To show in vitro bioactivity of IL-12 expressed from expression plasmids, AAV plasmids or AAV vectors, cells are cultured, transfected with plasmids or transduced with AAV vectors, and a reporter assay carried out according to manufacturer information.

### 1.15 Statistics

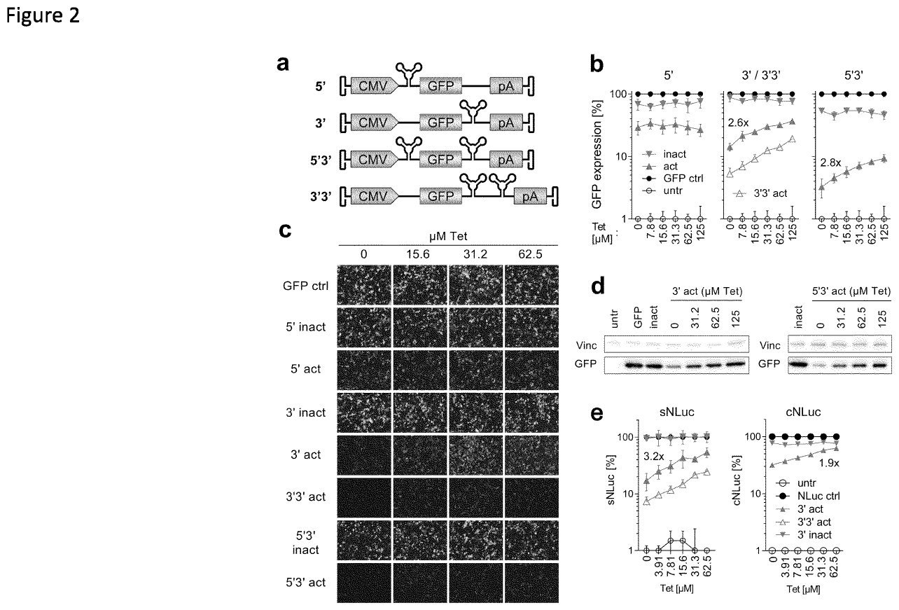

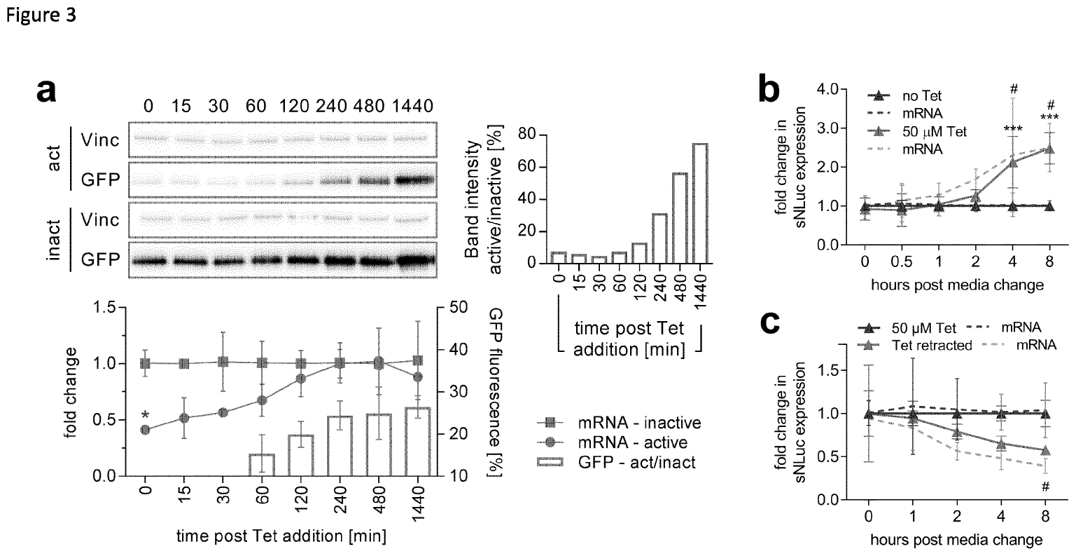

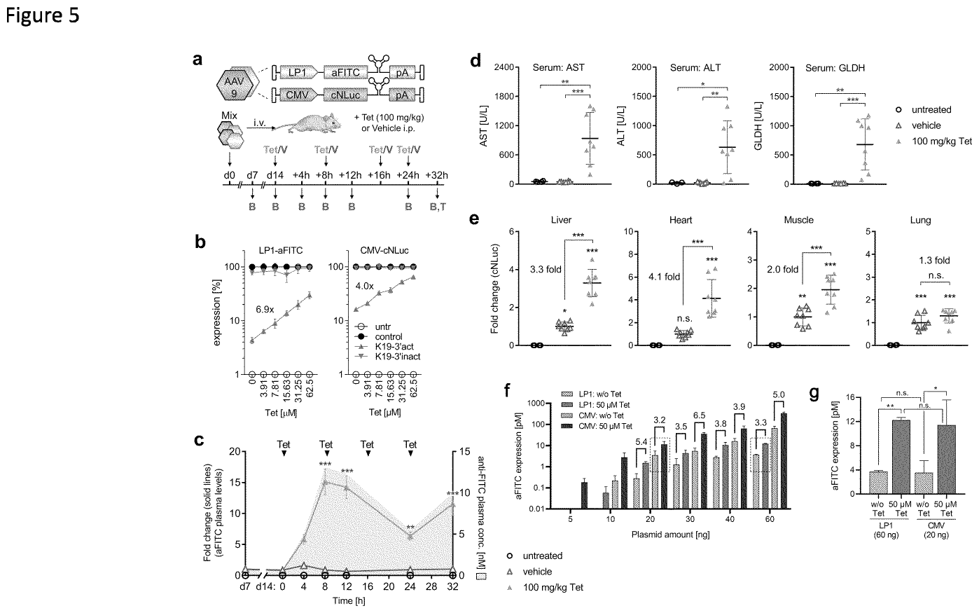

Statistical calculations were performed using GraphPad Prism V7.03. FIGS. 2b, e: Two-way ANOVA, controlled for multiple testing (MT) by Dunnett's test. FIGS. 3a, b, c, 5c: Two-way ANOVA, considering matched design (time), Sidak's MT test. FIGS. 5d, e, g, 7c, d: One-way ANOVA, Tukey's MT test. 6b, 7b: Two-way ANOVA, considering matched design (time), Tukey's MT test. P-values were derived based on two-tailed tests, assuming data is normally distributed.

### 1.16 Histology and Immunohistochemistry

Tissue samples of rat liver were fixed in 4% PFA and paraffin embedded (formalin fixed and paraffin embedded, FFPE). 3 μm thick sections of FFPE tissue on super frost plus slides were deparaffinised and rehydrated by serial passage through changes of xylene and graded ethanol for H&E and immunohistochemistry staining.

H&E staining was performed according to standard protocols (Romeis, Mikroskopische Technik; Hrsg. P. Böck; Urban and Schwarzenberg; München, Wien, Baltimore; 19. Auflage; 2015; pp 201; ISBN: 978-3-642-55189-5)

For Immunohistochemistry, antigen retrieval was performed by incubating the sections in Leica Bond Enzyme solution (Bond Enzyme Pre-treatment Kit, Cat#35607) for 5 minutes. Sections were incubated with an anti-CD45 antibody (abcam, ab10558, rabbit polyclonal). The antibody was diluted (1:400) with Leica Primary Antibody Diluent (AR9352; Leica Biosystems, Nussloch, Germany) and incubated for 30 min at room temperature. Bond Polymer Refine Detection, (Cat#37072) was used for detection (3,3′ Diaminobenzidine as chromogen, DAB) and counterstaining (hematoxylin). Staining was performed on the automated Leica IHC Bond-III platform (Leica Biosystems, Nussloch, Germany). Microscopic assessment of samples was conducted with a Zeiss Axiolmager M2 microscope and ZEN slidescan software (Zeiss, Oberkochen, Germany).

### 1.17 Image Analysis

Tumor size was calculated using the image processing software HALO 3.1. A classifier based on DenseNET (Huang et al., 2017) was trained with 16 sample regions from background, healthy and cancerous tissue.

For quantitative analysis anti-CD45 stained sections of the liver were scanned with an Axio Scan.Z1 whole slide scanner (Carl Zeiss Microscopy GmbH, Jena, Germany) using an 20× objective (0.22 μm/px) in bright field illumination. The percentage of the anti-CD45-positive cells was calculated using the image processing software HALO 3.1 with CytoNuclear v2.0.9 module (Indica Labs, Corrales, NM, USA). Cell count analysis was then restricted to ‘normal’ tissue, segmented in a pre-processing step using a built-in classifier (QC Slide). The analysis module uses color deconvolution to split signals of hematoxylin and DAB. Parameters for cell detection in the hematoxylin image and thresholds for positive DAB staining intensities in the cytoplasm were optimized manually. The summed percentage of strongly and moderately stained DAB positive cells were used in the quantitative analysis.

### 2. Results

To evaluate functionality of the K19 aptazyme in the AAV vector context, K19 was cloned into a plasmid containing AAV2 inverted terminal repeats (ITRs) and a CMV promoter-driven eGFP gene. K19 was either positioned 5′ upstream, 3′ downstream or at both positions relative to the eGFP gene (FIG. 2a). Twenty-four hours after transfection of HEK293 cells and subsequent addition of increasing doses of tetracycline (Tet), eGFP fluorescence was measured (FIG. 2b) and imaged (FIG. 2c). Whereas the 5′-design resulted in a general decrease in eGFP signal but lacked regulatability, possibly due to impeded ribosomal access and altered translation due to the artificial start codon within the switch sequence, the 3′-design allowed for dose-dependent induction of eGFP from 14% in the absence to 36% in the presence of Tet, relative to a constitutive, aptazyme-free control construct. An additional control plasmid, harboring a catalytically inactive K19 switch, expressed steady levels of about 90% of constitutive signal. Interestingly, the 5′3′-construct integrated the features of the 5′- and 3′-designs, as it showed similar switching behavior as the 3′-construct, however, at overall decreased expression levels. Based on the functional 3′-design, a tandem construct was further explored with two K19 aptazymes positioned in series (3′3′), which allowed for similarly potent expression control at overall reduced expression levels, i.e. from ˜5% to 19%. This finding is in accordance with results previously obtained for other switches (Ketzer et al. 2012; Beilstein et al. 2015). All results were confirmed by Western blotting (FIG. 2d). In addition to eGFP, successful regulation was also confirmed using an additional transgene, i.e. a secreted Nano luciferase (sNLuc), which confirmed successful dose-dependent induction of about three-fold using the 3′-design, and overall reduced signal with the 3′3′-tandem construct (FIG. 2e), which therefore was not considered further. Also, the intracellular NLuc variant (cNLuc), was successfully regulated by the switch, albeit at lower potency, which is likely a result of the intracellular accumulation of the reporter protein, resulting in a higher background signal (FIG. 2e).

To deepen the insight into temporal gene expression regulation, an mRNA-focused kinetics experiment was conducted. Therefore, a qPCR probe spanning the aptazyme auto-cleavage site was designed, allowing direct assessment of eGFP mRNA cleavage. Twenty-four hours after HEK293 cell transfection with plasmids containing either the active or inactive K19 switch, media were changed, baseline samples were taken and Tet was added to all remaining cultures, before being lysed at several time points to obtain RNA and protein for gene expression analysis. Slight but steadily increasing eGFP expression induction was seen from 15 min after Tet addition and full induction was observed after four hours on the mRNA level (FIG. 3b). This was paralleled by an increase in direct eGFP fluorescence signal and protein from two and four hours after addition of Tet, respectively. To corroborate these results, Tet-mediated regulation of sNLuc was also explored over time. Therefore, following HEK293 transfection and incubation for 24 h, media was changed to either Tet-free or Tet-containing media and sNLuc was detected in the cell supernatant. Similar to the eGFP data, a Tet-induced sNLuc increase was detected 2 h after addition, which reached saturation at about 4-8 h (FIG. 3c). Moreover, when Tet was retracted from cells previously grown in presence of Tet, a relative decrease in sNLuc expression was observed, therefore demonstrating reversibility (FIG. 3d). While our assays focused on functional riboswitch downstream effects (i.e. protein output), which are additionally influenced by continuous de novo transcription, mRNA degradation as well as protein translation, stability and turnover, actual ribozyme cleavage rates can be obtained from assays using naked RNA, as previously performed for K19 (Beilstein et al. 2015).

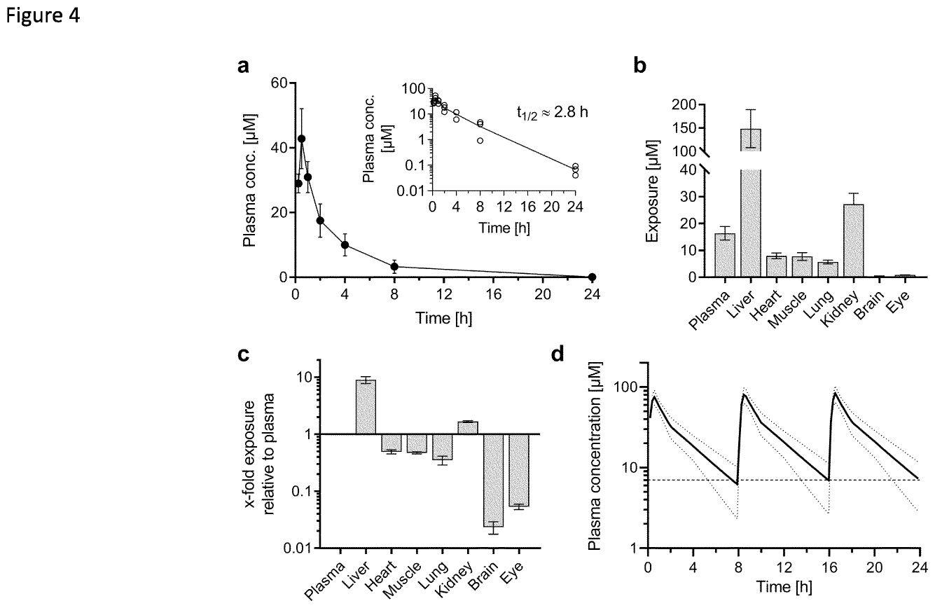

Because the tetracycline aptamer domain of the K19 aptazyme specifically binds Tet but not Doxycycline, preparation for the evaluation of the riboswitch system in mice included Tet pharmacokinetic (PK) studies, for which preclinical in vivo data is scarce. Following administration of 54 mg/kg i.p., peak plasma concentrations of 42 μM at 30 mins and residual levels of 3.3 μM at 8 h were measured, corresponding to a half-life of approx. 2.8 h (FIG. 4a). Moreover, exposure in various mouse organs was determined 2 h after Tet administration, revealing total concentrations of 16.4 μM in plasma, 5.7 μM in lung, 7.8 μM in muscle, 8.0 μM in heart, 27.2 μM in kidney and 149 μM in the liver (FIG. 4b, c). Only little exposure was detected in the brain (0.42 μM) and eyes (0.88 μM). To estimate which plasma concentrations can be achieved by multiple administration of Tet, PK non-parametric modeling was performed. The modeling approach suggested that i.p. administration of 100 mg/kg Tet three times a day (8 h intervals) would result in plasma trough levels of approximately 7 μM (FIG. 4d).

Next, alternative reporter proteins were tested that would allow measuring switching performance and kinetics in vivo, ideally in a multiplexed fashion. We decided for a secreted anti-Fluorescein isothiocyanate (aFITC) tandem single chain variable fragment (scFv) antibody (Vaughan et al. 1996; Honegger et al. 2005) under the control of the liver-specific LP1 promoter (Nathwani 2006) and a CMV promoter expressed, non-secreted Nano-Luciferase (cNLuc) and cloned appropriate expression constructs and controls (FIG. 5a). For anti-FITC scFv analysis, we first established an MSD ELISA assay, which, upon optimization of coating and detection antibody concentration, allowed for robust anti-FITC scFv measurements at 0.1 pM sensitivity. As expected, functionality assessment in the hepatocyte cell line HepG2 demonstrated that both constructs allowed for Tet-dependent gene expression induction, whereas expression remained unaltered when using control constructs (FIG. 5b).

Previous studies have tested aptazyme designs in the muscle and eye of mice, however, either OFF-designs were used (Zhong et al. 2016) or the ON-designs led to very modest effects (Reid et al. 2018). Moreover, aptazyme functionality across different organs has not been studied so far. Therefore, an experiment was designed herein to explore ONswitch potency and functionality across organs in a simultaneous fashion. Specifically, advantage was taken of the broad transduction pattern of recombinant AAV9 following i.v. administration (Zincarelli et al. 2008) to simultaneously express and study regulation of 1) intracellularly expressed cNLuc in the liver, lung, heart and muscle tissue and 2) the transcriptionally liver-targeted, secreted aFITC antibody, by measuring its levels in plasma. Therefore, mixtures of AAV9-CMV-cNLuc-K19 (mediating ubiquitous expression) and AAV9-LP1-aFITC-K19 (mediating liver specific expression) were administered to mice (1x10 11 vg in total per animal, N=8 animals per group). Two weeks after AAV administration, a total of four 100 mg/kg doses of Tet was administered in 8 h intervals to induce expression from the aptazyme constructs (see scheme in FIG. 5a). Blood was drawn before and at multiple time points after induction, and tissue lysates for cNLuc protein analyses were prepared 8 h after the last Tet dose. First anti-FITC antibody plasma levels were analyzed. Basal expression was similar on day 7 (mean=0.86 nM, standard deviation (SD)=0.37) and day 14 (0.72 nM, SD=0.18) in all AAV-treated animals, demonstrating that the plateau of AAV-mediated expression had been reached (FIG. 5c). Intriguingly, upon administration of a single dose of 100 mg/kg Tet, anti-FITC expression levels were strongly induced by the aptazyme construct, reaching 38% induction at 4 h and peak induction levels (=100%) at 8 h post dosing, corresponding to 5.8- and yet unprecedented 15.1-fold increases at 4 and 8 h over averaged vehicle control values, respectively (FIG. 5c). While strong induction was mediated by the first Tet dose, the following three administrations did not further enhance expression. Instead, a decrease of absolute aFITC levels and less pronounced expression induction (6.3- to 11.5-fold) were observed. Elevated AST, liver-specific ALT and liver mitochondria-derived GLDH plasma activity measured 8 h after the last Tet dose indicated liver injury specifically induced by Tet, explaining this observation (FIG. 5d). While Tet-mediated liver enzyme elevation is a well-known side effect (Choi et al. 2015), no other signs of toxicity were observed in Tet-treated animals in the instant study.

In addition to the potent induction of aFITC, successful regulation was also observed for the intracellular expression of cNLuc driven by a CMV promoter. In the liver, a 3.3-fold increase upon Tet treatment was observed by measuring luciferase activity in tissue lysates (FIG. 5e). Moreover, expression was induced 4.1-fold, 2-fold and 1.3-fold in heart, muscle and lung, respectively. While the observed differences in gene expression induction in the liver using LP1-mediated anti-FITC antibody (15.1-fold) and CMV-driven cNLuc expression (3.3-fold) likely lie in the fact that anti-FITC scFv is secreted and therefore constantly cleared, whereas cNLuc is accumulating in the cell, we also considered promoter strength as an influencing factor. Therefore, switching efficiency was assessed for LP1 and CMV promoter constructs in HepG2 cells. The results showed that although CMV promoter strength in general was 5- to 15-fold higher than that of LP1 (median: 10.3-fold difference), similar induction of anti-FITC expression was observed upon Tet stimulation (range: 3.2-6.5-fold, mean CMV: 4.4-fold, mean LP1: 4.1-fold), independent of the amount of transcript expressed (FIG. 5f). Moreover, when plasmid levels that led to equal basal transcriptional output were compared, switching efficiency was indistinguishable between CMV- and LP1-constructs (FIG. 5g). These results suggest that the observed differences in vivo are due to the use of intracellular versus secreted reporters and largely independent of the promoter used.

Although impeded by the fact that by the time of induction, basal cNLuc expression had already proceeded for two weeks, leading to intracellular accumulation and less pronounced induction, our results clearly demonstrate that gene expression induction by a riboswitch is feasible in different organs in mice. Another interesting finding in this regard was that total Tet exposure in the liver was approximately 18-fold higher than in the heart and muscle, but nevertheless, CMV promoter-driven intracellular cNLuc expression was similarly induced by the switch (3.3-, 4.1- and 2-fold in liver, heart and muscle, respectively). Given that AAV9 transduction efficiency is similar in liver and heart (Zincarelli et al. 2008), these results might suggest that cardiac expression could be particularly well regulated by a riboswitch, possibly due to higher transcriptional and/or mRNA degradation activity. While systematic follow-up studies are required to prove this particular hypothesis, our data support the general assumption that the potency of riboswitch-controlled gene regulation might be partly dependent on the cellular context.

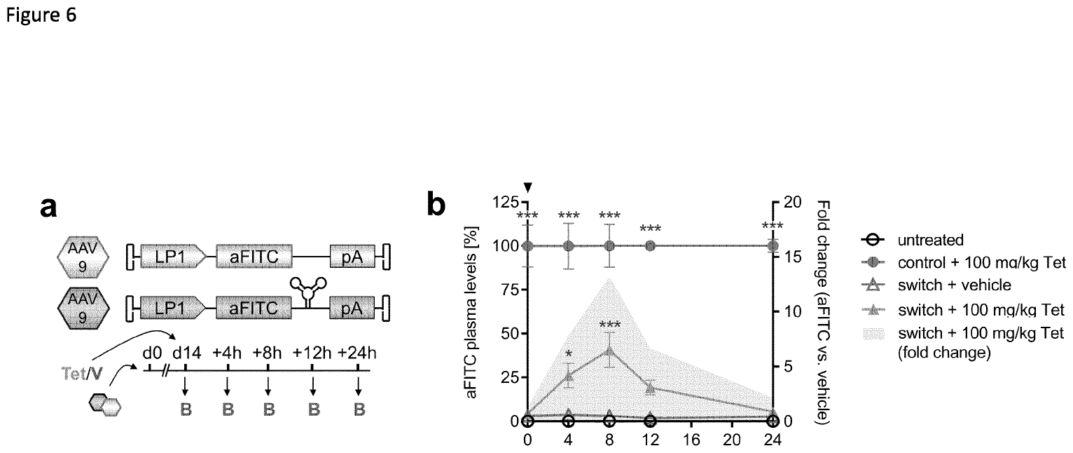

Having demonstrated functionality of the Tet switch in an in vivo setting, we examined how Tet-induced expression levels compare to those of a conventional riboswitch-free construct, mediating constitutive expression. We therefore re-assessed expression induction in a simple follow-up experiment, including an AAV9-LP1-aFITC control vector and a single Tet trigger (100 mg/kg) to induce expression in mice (FIG. 6a, N=4 animals per group). The results showed that the riboswitch repressed transgene expression to 3.1% of constitutive control levels, whereas maximal, 13.2-fold induction reached 40.1% at 8 h after Tet administration (FIG. 6b). Expression levels decreased to half-maximal levels 12 h after induction and returned to baseline at 24 h, nicely demonstrating reversibility upon ligand clearance. Our results therefore proof the ability to temporarily induce gene expression to levels in a relevant dimension, as defined by a constitutive control construct.

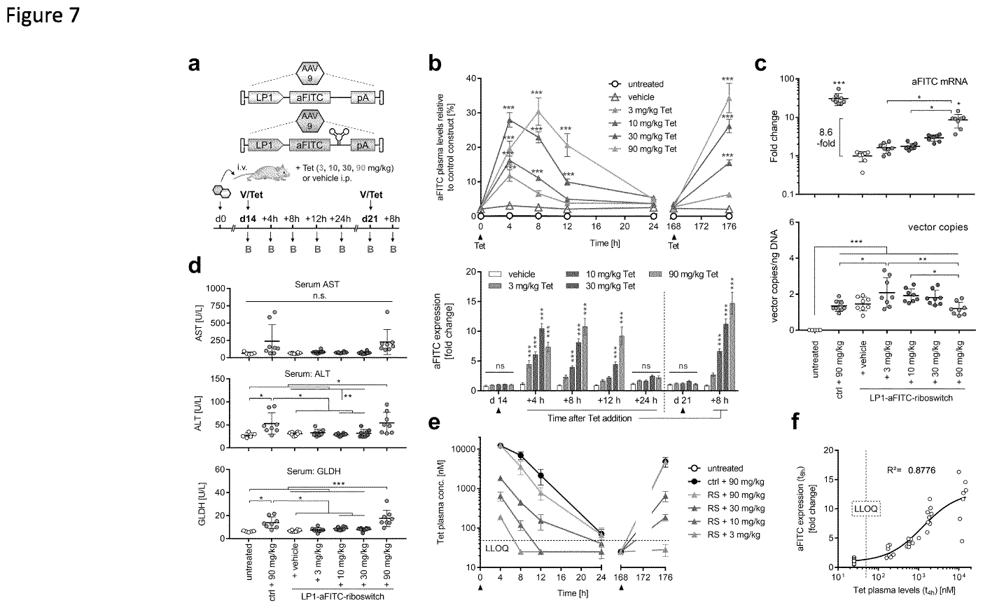

One expected feature of riboswitch vectors, which, however, has not been proven so far, is the potential to fine-tune expression levels in vivo by adjusting the dose of ligand. Moreover, aptazymes should in principle allow for repeated, i.e. dynamic ON-OFF switching, yet also this aspect has not been experimentally proven in animals so far. Therefore, we finally investigated the degree and kinetics of reporter expression induction by four different (3, 10, 30, 90 mg/kg) single-dose Tet administrations (N=8 animals per group) and further explored the possibility to re-stimulate expression one week after the first induction. Pharmacokinetic (PK) measurements further enabled to investigate associated PK/PD relationships. For this experiment, we again made use of the AAV9LP1-aFITC vectors (FIG. 7a) at the previously used vector dose of 2.5×10¹² vg/kg, which, notably, equals the maximal dose used in AAV-based, liver-directed hemophilia B trials in the clinic (Manno et al. 2006; Nathwani et al. 2011; Nathwani et al. 2014). The results showed that two weeks after recombinant AAV administration (=t₀ₕ in FIG. 7b), anti-FITC antibody expression in animals receiving riboswitch vectors was repressed to 2.5% of the levels of the riboswitch-free control construct (set 100%) (FIG. 7b). However, upon a single administration of increasing Tet doses (3, 10, 30, 90 mg/kg), anti-FITC expression was rapidly induced to dose-dependent peak expression levels of approximately 12, 16, 28 and 30% of control, respectively. While with 3, 10 and 30 mg/kg, maxima were reached 4 h after administration, maximal expression at the 90 mg/kg dose was only reached at 8 h. Moreover, also the duration of transgene induction was dose-dependent, with a return to baseline occurring more rapidly at lower doses. In all cases, however, expression had largely returned to baseline levels by 24 h after Tet administration at the latest.

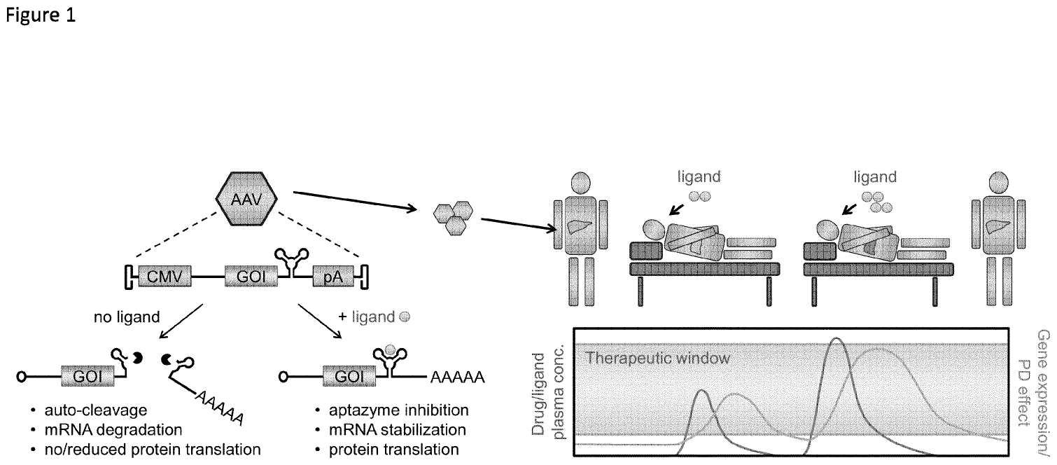

To assure complete Tet clearance and to simulate a treatment-free phase with desired expression shutdown, proofing persistent riboswitch activity (compare FIG. 1), it was waited for one week before re-administration of Tet. As expected, by day 21, transgene expression had fully returned to baseline, showing equal repression as one week before, i.e. 2.8% of control levels (FIG. 7b). Importantly, upon re-administration, expression was induced in the same dose-dependent fashion as seen previously, reaching a maximum of 34% of control levels (i.e. 14.7-fold induction) at the highest ligand dose (FIG. 7b). Tetracycline dose-dependent expression induction was finally also validated on the mRNA level and similar AAV vector genome counts were detected in all AAV-treated animals, with minor fluctuations (FIG. 7c) that did not impact data interpretation. Nevertheless, normalization of mRNA levels to the corresponding vector genomes further decreased intra-group fluctuations.

In contrast to the previous experiment, using multiple dosing (FIG. 5d), in the current experiment serum liver enzyme activation was only moderately increased at the highest Tet dose (FIG. 7d). In fact, serum activity of AST, ALT and GLDH was 4-, 11- and 38-fold lower than upon multiple dosing. Accordingly, anti-FITC peak and control expression levels as well as the degree of induction remained stable throughout the experiment, indicative of good tolerability.

Pharmacokinetic and -dynamic (PD) measurements finally allowed for correlation assessment between Tet plasma levels (FIG. 7e) and observed aFITC expression induction (FIG. 7b). The best nonlinear three-parametric fit (R²=0.8776) was observed for the Tet levels measured at t₄ₕ and expression induction at t₈ₕ (FIG. 7f), indicative of a time delay due to intracellular Tet uptake, de novo anti-FITC mRNA and protein synthesis as well as -turnover. In summary, our results establish important proof for the possibility to control viral vector-mediated gene expression by ligand-controlled riboswitches in a dose-dependent and highly dynamic fashion in mice.

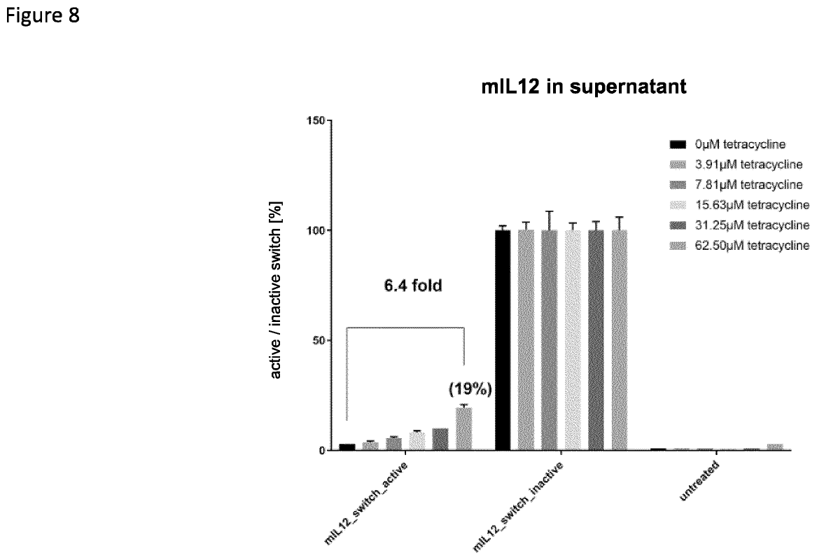

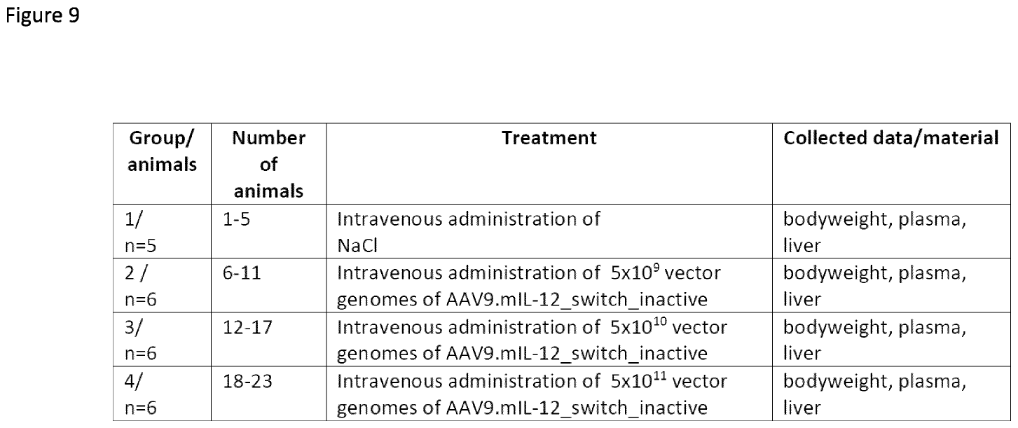

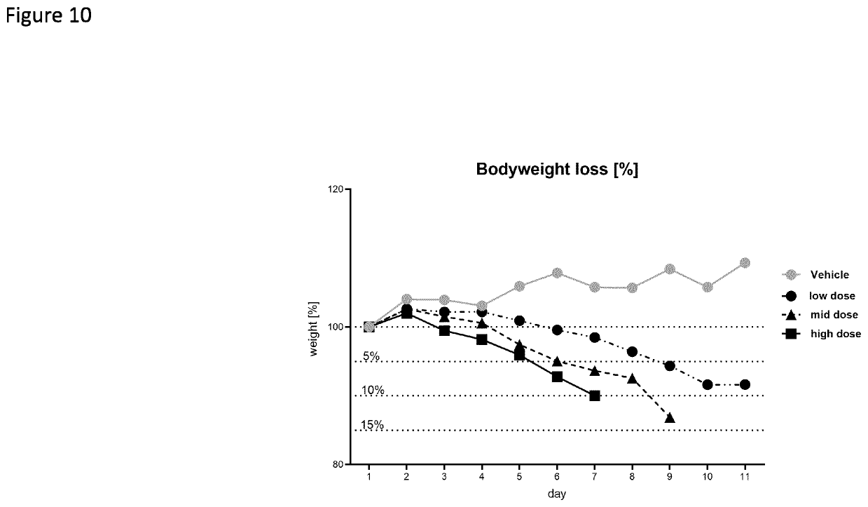

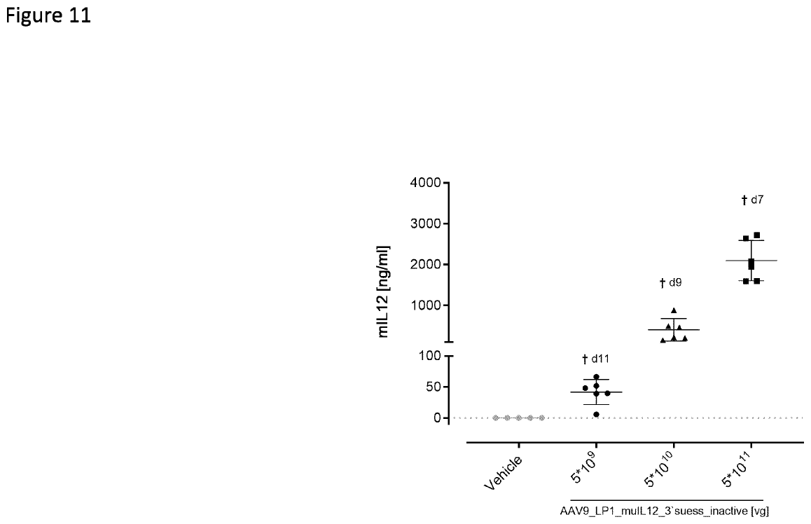

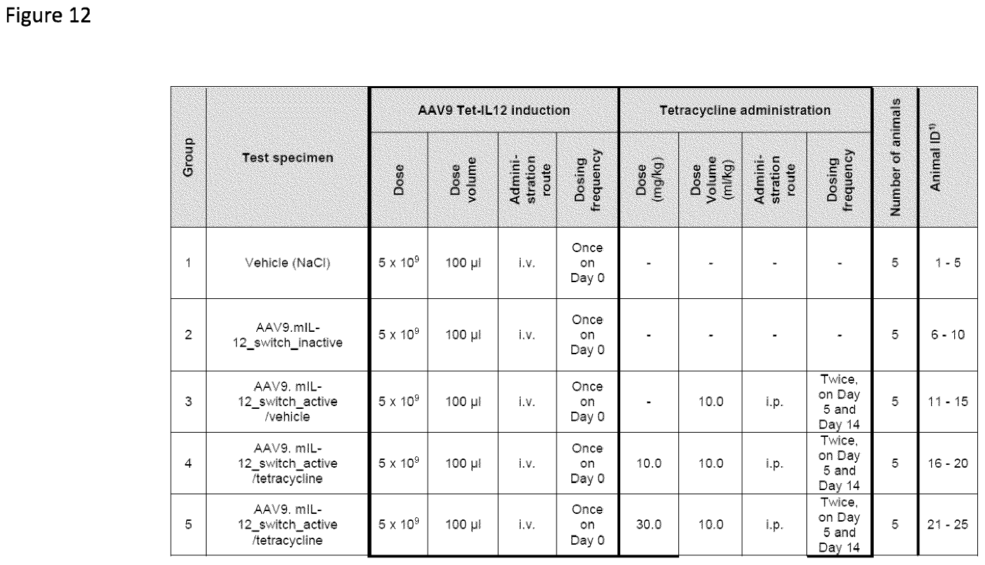

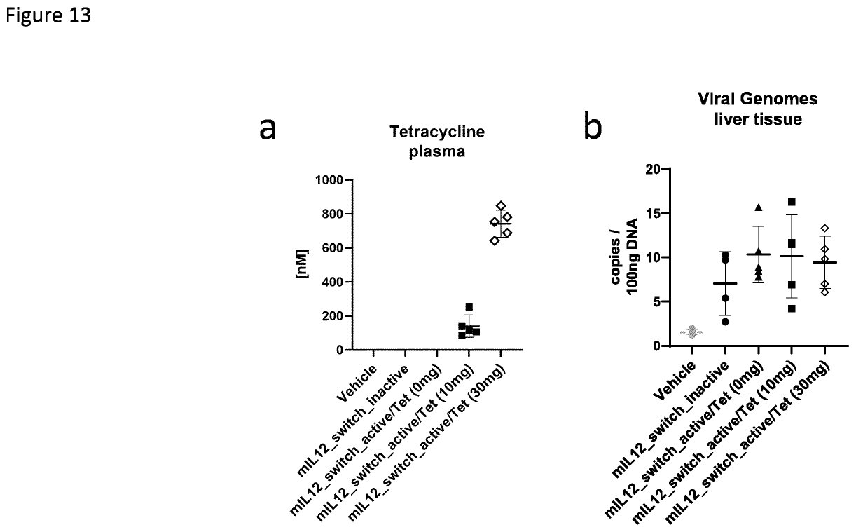

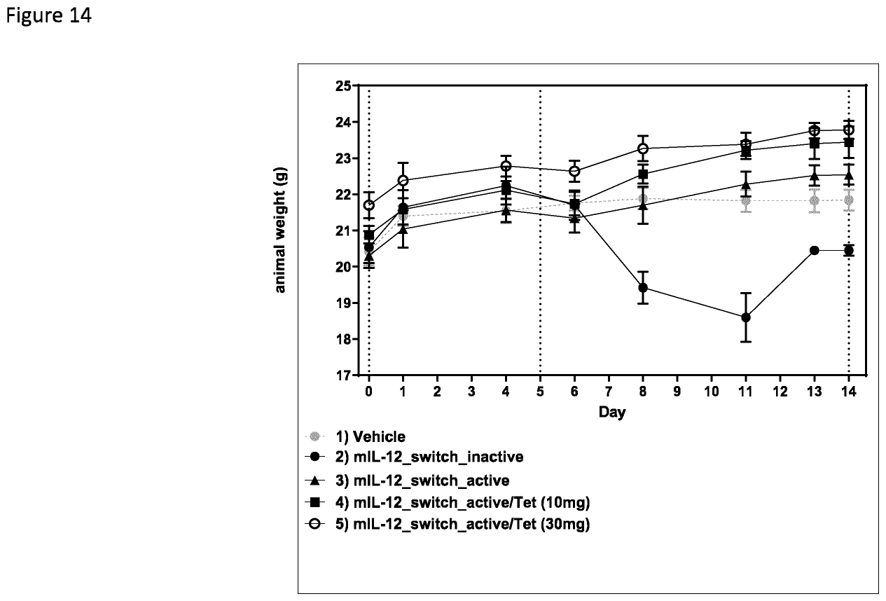

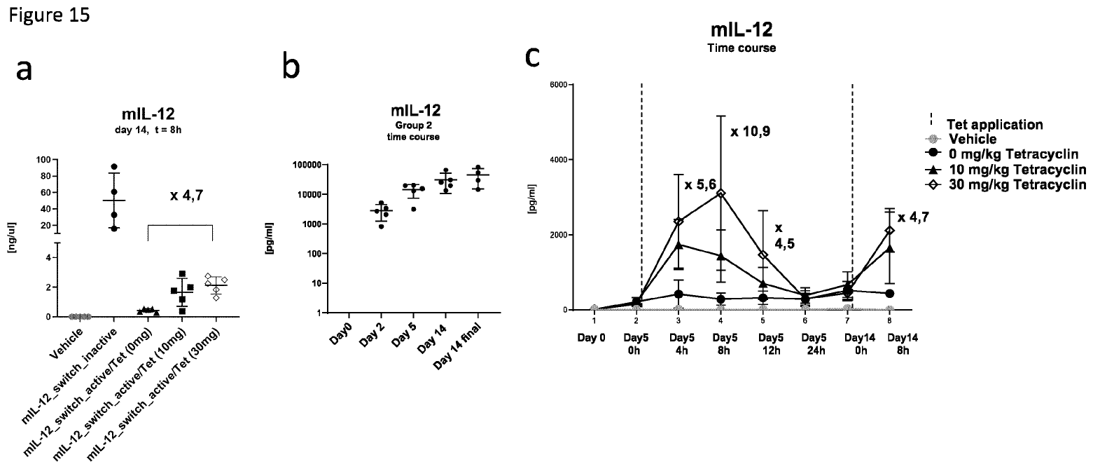

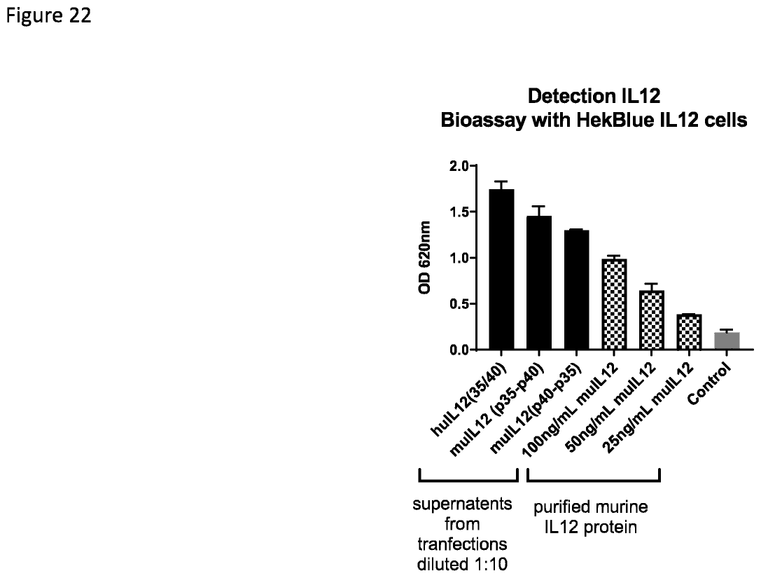

Following successful proof of concept in the context of reporter genes, the reporter gene was next replaced by the IL-12 gene encoding murine single chain IL-12 and packaged as AAV9. Transduction of HepG2 cells with the active K19-IL-12 vector carrying the liver-specific LP1 promoter revealed 3% background levels of IL-12 in the supernatant and a 6.4-fold induction at the highest Tet dose (FIG. 8). The experiment in FIG. 8 was conducted with IL-12 in the p40-linker-p35 orientation. In FIG. 22, we transfected HEK293 cells with expression plasmids (pOptiVEC, Thermo Fisher Scientific) for mouse and human IL-12 constructs and could confirm that both the p40-linker-p35 as well as the p35-linker-p40 orientations of single chain IL-12 result in bioactive IL-12 (FIG. 22). The successful in vitro biopotency data were the basis for a dose-finding study using i.v. delivery of the AAV9 harboring the constitutive mIL-12-inactive construct to naïve C57B1/6 mice at three different doses (5×10⁹, 5×10¹⁰, 5×10¹¹ vg) (FIG. 9). Weight loss was monitored as primary endpoint for the expected side effects of high IL-12 in the circulation as a consequence of sustained hepatocyte-derived transgene expression. A dose-dependent rapid drop in body weight occurred in all AAV.IL-12 groups that led to termination of the in-life phase at day 7, day 9 and day 11 for the low, mid and high dose animals, respectively (FIG. 10). In contrast, the vehicle control group gained weight. IL-12 levels in plasma of the treatment groups collected at the final day of the in-life phase showed dose dependency with 48 ng/mL in the low dose group (FIG. 11). Baseline IL-12 levels were below the detection threshold in controls. In summary, side effects such as body weight loss can be interpreted to be a function of pathological circulating levels IL-12 levels that originate from hepatocytes. The low vector dose was selected to be used for all vectors nominated to be assessed for a PD study on Tet-induced IL-12 expression in naïve mice (n=5 per group). The study design (FIG. 12) included three groups of mice receiving AAV9.LP1_mIL12_switch active and two challenges with saline, Tet (10 mg/kg) or Tet 30 mg/kg) at 5 and 14 days following AAV delivery. Control groups received no vector or the constitutive AAV9.mIL-12 switch inactive and no Tet. At 8 hrs after the Tet re-challenge on day 14, Tet concentrations in plasma were determined at 100 nM and 750 nM in the 10 mg and 30 mg Tet groups (FIG. 13a). The transduction efficiency in the livers of AAV-treatment groups was determined to be similar across the groups (FIG. 13b). Body weight monitoring over the duration of 14 day revealed a drop only in the constitutive AAV9.mIL-12_switch_inactive group (FIG. 14), reproducing the previous results with this vector (FIG. 10). Again, IL-12 plasma levels in this group were determined to be 50 ng/mL suggesting that these sustained IL-12 quantities are not tolerated (FIG. 15a). A time course of IL-12 levels in the plasma of this group shows substantial cytokine amounts as early as day 2 following vector delivery (FIG. 15b). This quick kinetic suggests that gene therapy even using single-stranded AAV vector genomes is feasible in a rapid an aggressive HCC model. The plasma IL-12 levels in the animals dosed with the Tet-responsive AAV9.LP1_mIL12_switch active vector showed Tet-dose dependent induction. 30 mg/mL Tet induced almost 11-fold after 8 hrs over background levels (Day 5, 0 hrs) (FIG. 15c). Following the fast on-kinetic, IL-12 levels had returned to baseline after 24 hrs. While the Tet-re-challenge at day 14 induced IL-12 at a somewhat lower level of 4.7-fold, the absolute IL-12 concentrations ranged between 2-3 ng/mL. This level is well in the expected therapeutic window and lacks obvious signs of side effects. Moreover, taking away Tet virtually eliminates detectable IL-12 expression.

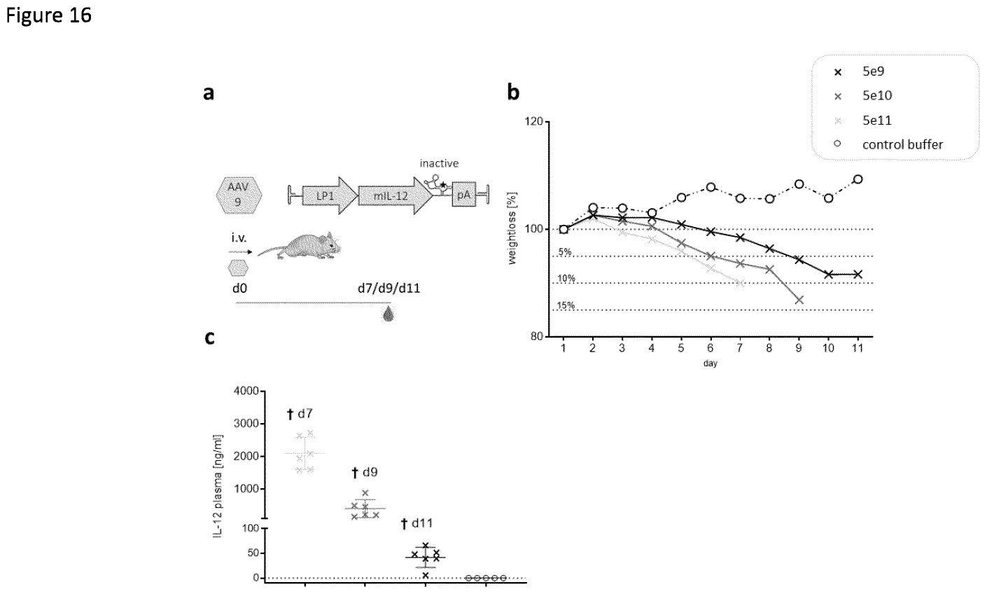

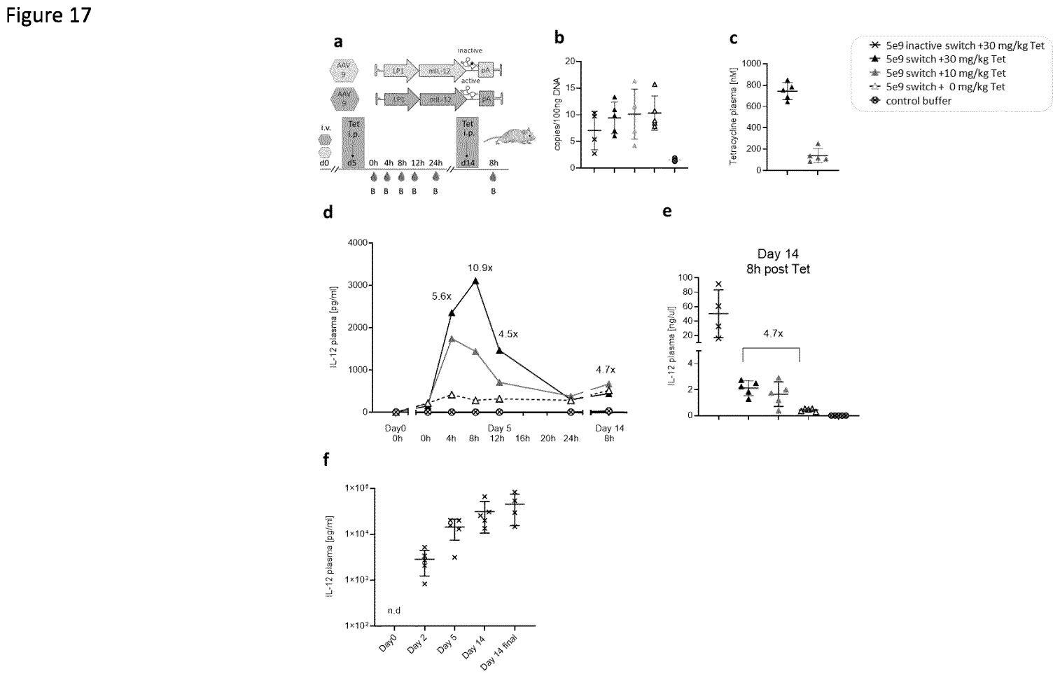

The following FIGS. 16-17 include graphs that are also presented in the preceding FIGS. 9-15. These graphs are depicted in a slightly different way, but are based on the same set of data.

The dose-finding study using i.v. delivery of the AAV9 harboring the constitutive mIL-12-K19 inactive construct to naïve C57BL/6 mice at three different doses (5×10⁹, 5×10¹⁰, 5×10¹¹ vg) is shown in FIG. 16. Transgene expression was restricted to liver by the use of the LP1 promoter (FIG. 16a). Weight loss was monitored as primary endpoint for the expected side effects of high IL-12 in the circulation as a consequence of sustained hepatocyte-derived transgene expression. A dose-dependent rapid drop in body weight occurred in all AAV.IL-12 groups that led to termination of the in-life phase at day 7, day 9 and day 11 for the low, mid and high dose animals, respectively (FIG. 16b). In contrast, the buffer control group gained weight. IL-12 levels in plasma of the treatment groups collected at the final day of the in-life phase showed dose dependency with 48 ng/mL in the low dose group (FIG. 16c). Baseline IL-12 levels were below the detection threshold in controls. In summary, side effects such as body weight loss can be interpreted as a function of toxicity caused by circulating levels IL-12 levels that originate from hepatocytes.

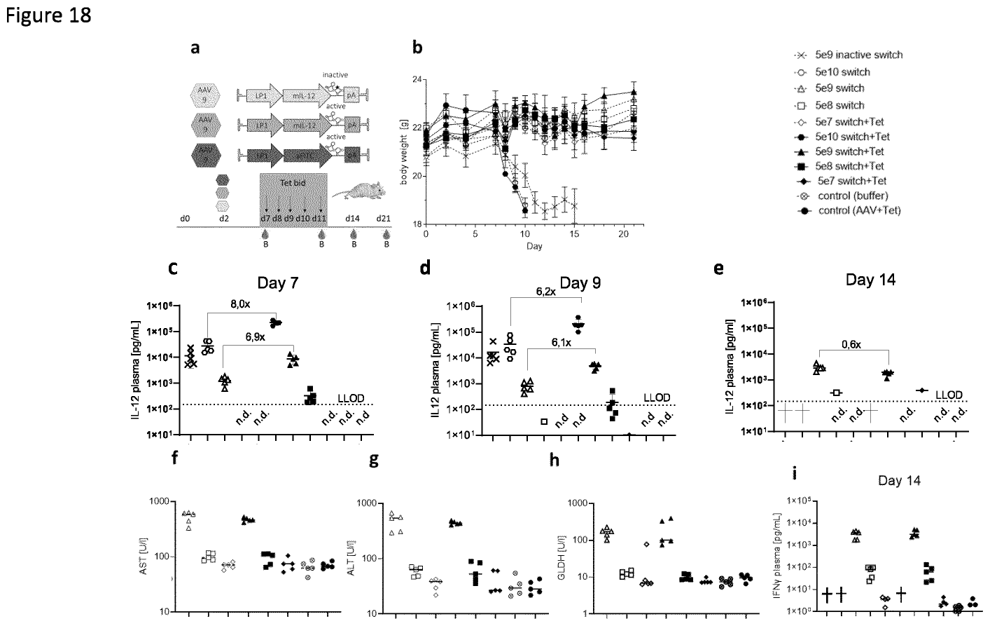

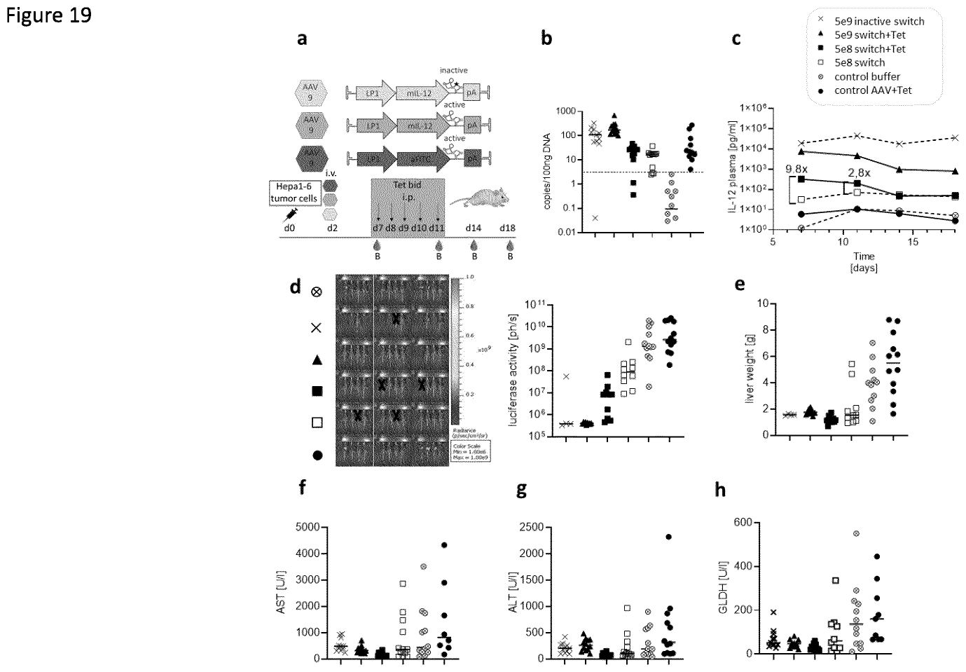

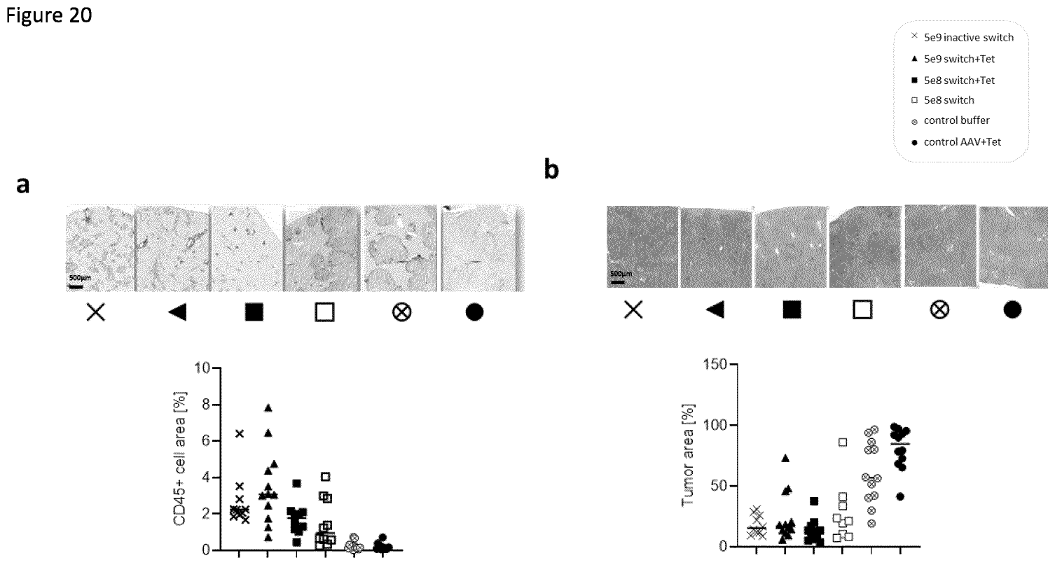

The low vector dose was selected to be used for all vectors nominated to be assessed for a PD study on Tet-induced IL-12 expression in naive mice (N=5 per group). The study design (FIG. 17a) included three groups of mice receiving AAV9.LP1-mIL-12-switch and two challenges with buffer, Tet (10 mg/kg) or Tet 30 mg/kg) at 5 and 14 days following AAV delivery. Control groups received no vector or the constitutive AAV9.mIL-12-inactive-switch_ and no Tet. The transduction efficiency in the livers of AAV-treatment groups was determined to be similar across the groups (FIG. 17b). At 8 h after the Tet re-challenge on day 14, Tet concentrations in plasma were determined to be 100 nM and 750 nM in the 10 mg and 30 mg Tet groups (FIG. 17c). The plasma IL-12 levels in the animals dosed with the Tet-responsive AAV9.LP1-mIL-12-switch vector showed Tet-dose dependent induction (FIG. 17d). 30 mg/mL Tet induced almost 11-fold after 8 h over background levels (Day 5, 0 h). Following the fast on-kinetic, IL-12 levels had returned to baseline after 24 h. While the Tet-re-challenge at day 14 induced IL-12 at a somewhat lower level of 4.7-fold, the absolute IL-12 concentrations ranged between 2-3 ng/mL. This level is well in the expected therapeutic window and lacks obvious signs of side effects. Moreover, taking away Tet virtually eliminated detectable IL-12 expression. Again, IL-12 plasma levels in this group was determined to be 50 ng/mL suggesting that these sustained IL-12 quantities are not tolerated (FIG. 17e). A time course of IL-12 levels in the plasma of this group shows substantial cytokine amounts as early as day 2 following vector delivery (FIG. 17f). This rapid kinetic of AAV-mediated transgene expression suggests that gene therapy even using single-stranded AAV vector genomes is feasible in a rapid an aggressive HCC model. Expression levels increased between day 2 and the plateau at day 14. This observation explained the slight rise in background IL-12 levels observed in animals receiving the IL-12 active vector without Tet (FIG. 17d,e). We then performed a comprehensive dose-finding study to determine PK and safety of Tet-switch-controlled IL-12 expression using sustained Tet challenges (FIG. 18a). The study design entailed delivery of the AAV9-LP1-mIL-12-switch vector at doses from 5×10⁷-5×10¹⁰ vg/mouse. Half the animals of each group were subjected to twice daily Tet applications for 5 consecutive days, while the other half received no Tet. The aim of this study was to identify a vector dose that allowed Tet-dependent sustained induction of relevant IL-12 levels in plasma during the Tet challenge but return to low or no background levels before the end of the experiment. A secondary endpoint was to monitor potential weight loss and liver enzymes as a measure of toxicity. In fact, we observed that the longitudinal body weight development was normal for all groups except the two highest AAV9-LP1-mIL-12-switch dose groups and inactive switch group, suggesting that IL-12 levels in most groups, and Tet in general, was well tolerated (FIG. 18b). IL-12 levels showed vector dose-dependency (FIG. 18c,d). Importantly, in the 5×10⁸ vg group active switch group, IL-12 levels had returned to background 3 days after the final Tet exposure (FIG. 18e) and showed normal levels of liver enzymes (FIG. 18f,g,h). Importantly, the 5×10⁸ vg active switch group showed elevated levels of IFN gamma, the key effector of IL-12-induced T-cell activation (FIG. 18i). In summary, the AAV9-LP1-mIL-12-switch_dose of 5×10⁹ vg was identified as the maximum tolerated dose, based on the absence of weight loss, while the dose of 5×10⁸ vg had the best PD and safety. These doses were then nominated in a PD study using a mouse model of HCC. The study design (FIG. 19a) was adopted from the dose finding study. Two cohorts of mice received AAV9-LP1-mIL-12-switch_in a dose of 5×10⁸ vg, one received Tet, the other one did not. The same vector was administered at 5×10⁹ vg and Tet. A dose-matched group received the benchmark vector AAV9-LP1-mIL-12_-inactive-switch in order to likely achieve remission even though adverse effects were expected based on our previous studies. At the end of the study, we confirmed dose-dependent transduction efficiency across all vector-treated groups (FIG. 19b). The range of Tet-induced IL-12 regulation compared to the dose-matched control group was 9.8-fold at the beginning of the Tet treatment regimen and dropped to background levels after the final challenge (FIG. 19c). The IL-12 background levels were generally higher than in tumor-free mice, suggesting increased endogenous IL-12 production in the tumor model. Whole body imaging was used to quantify luciferase signal intensity from the engrafted Hepal-6 cells as a correlate of tumor size (FIG. 19d). Of note, both the benchmark inactive switch group and the high dose active switch group showed benefits in reducing tumor size but also toxicity reflected by losing animals. In fact, remission was observed that appeared to show IL-12-responsiveness. The liver weight, assessed at the end of the study, was in agreement with the imaging data (FIG. 19e). Liver enzyme measurements were performed but not conclusive (FIG. 19f,g,h), suggesting the requirement of other criteria for drug tolerability in this disease model. The same is true for body weight development which was recorded, however was confounded by the abundant tumor growth that masked body weight loss and hence toxicity (not shown). Importantly, the IL-12 immunotherapy was paralleled by homing of CD45+ cells to the tumor nodules and reduction of tumor area in the liver indicating the successful realization of the concept to turn a cold tumor hot (FIG. 20a,b).

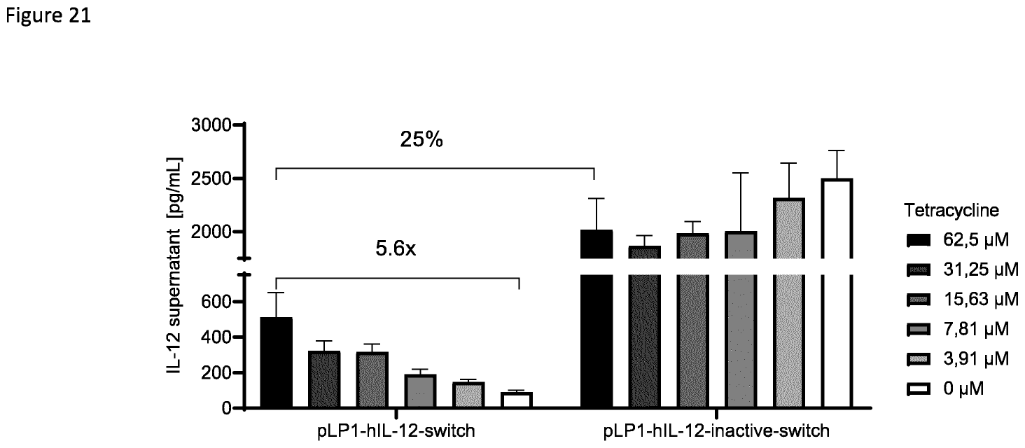

In addition, we showed in HEK293 cells that the K19 riboswitch responds to Tet by inducing human IL-12 (5.6-fold) in a drug-dose-dependent manner (FIG. 21), comparable to the 6.4-fold AAV-mediated expression of Tet-induced mIL-12, illustrated in FIG. 8.

Finally, using HEK-Blue™ IL-12 cells, we showed bioactivity of human single chain IL-12 contained in supernatants of HEK293 cell that had been transfected with plasmids encoding human single chainIL-12 (FIG. 22). This bioassay revealed bioactivity was comparable between murine and human IL-12. Moreover, bioactivity did not depend on the order of p35 and p40 in the single-chain hIL-12 protein.

In summary, these data suggest that IL-12 gene therapy can be tightly controlled in a spatio-temporal manner for a safe and efficient immunomodulatory effect using a rational combination of AAV serotype, vector dose, Tet dosing regimen and target organ. The IL-12 data confirm the potential to fine tune expression levels of a therapeutic protein in vivo by adjusting the dose of ligand in a riboswitch context. Moreover, the aptazyme-mediated control over IL-12 expression enables for repeated, i.e. dynamic ON-OFF switching.

### LIST OF REFERENCES

Altschul et al., 1997, Nucleic Acids Res. 25:3389-3402

Ausländer, S., and Fussenegger, M. (2013) From gene switches to mammalian designer cells: present and future prospects, Trends Biotechnol. 31, 155-168

Barrett, J. A., Cai, H., Miao, J., Khare, P. D., Gonzalez, P., Dalsing-Hernandez, J., Sharma, G., Chan, T., Cooper, L. J. N., and Lebel, F. (2018) Regulated intratumoral expression of IL-12 using a RheoSwitch Therapeutic System((R)) (RTS((R))) gene switch as gene therapy for the treatment of glioma, Cancer Gene Ther 25, 106-116

Beilstein, K., Wittmann, A., Grez, M., and Suess, B. (2015) Conditional Control of Mammalian Gene Expression by Tetracycline-Dependent Hammerhead Ribozymes, ACS synthetic biology, 4, 526-534

Berens, C., Thain, A., and Schroeder, R. (2001) A tetracycline-binding RNA aptamer, Bioorg Med Chem 9, 2549-2556

Berens, C., Groher, F., and Suess, B. (2015) RNA aptamers as genetic control devices: the potential of riboswitches as synthetic elements for regulating gene expression, Biotechnology Journal 10, 246-257

Berraondo, P., Etxeberria, I., Ponz-Sarvise, M., and Melero, I. (2018) Revisiting Interleukin-12 as a Cancer Immunotherapy Agent, Clin Cancer Res 24, 2716-2718

Bloom, R. J., Winkler, S. M., and Smolke, C. D. (2015) Synthetic feedback control using an RNAi-based gene-regulatory device, J Biol Eng 9, 5

Cao G. et al. 1998. Comparison of carcinoembryonic antigen promoter regions isolated from human colorectal carcinoma and normal adjacent mucosa to induce strong tumor specific gene expression. Int J Cancer 78:242-247

Chen L. et al. 1995. Breast cancer selective gene expression and therapy mediated by recombinant adenoviruses containing the DF3/MUC1 promoter. J. Clin. Invest. 96:2775-2782

Chen, Y. Y., Jensen, M. C., and Smolke, C. D. (2010) Genetic control of mammalian T-cell proliferation with synthetic RNA regulatory systems, Proc. Natl. Acad. Sci. U.S.A 107, 8531-8536

Chiocca E A, Yu J S, Lukas R V, Solomon I H, Ligon K L, Nakashima H, Triggs D A, Reardon D A, Wen P, Stopa B M, Naik A, Rudnick J, Hu J L, Kumthekar P, Yamini B, Buck J Y, Demars N, Barrett J A, Gelb A B, Zhou J, Lebel F, Cooper L J N. (2019). Regulatable interleukin-12 gene therapy in patients with recurrent high-grade glioma: Results of a phase 1 trial. Sci Transl Med.11(505).

Choi, Y. J., Lee, C. H., Lee, K. Y., Jung, S. H., and Lee, B. H. (2015) Increased hepatic Fatty Acid uptake and esterification contribute to tetracycline-induced steatosis in mice, Toxicol Sci 145, 273-282

Cohen J. (1995). IL-12 Deaths: Explanation and a Puzzle. Science 270:908.

Das A T, Tenenbaum L, Berkhout B. (2016). Tet-On Systems For Doxycycline-inducible Gene Expression. Curr Gene Ther. 16(3):156-67.

Dillekas H, Rogers M S, Straume O. Are 90% of deaths from cancer caused by metastases? (2019). Cancer Med. 8(12):5574-5576.

Gately, M. K, Chizzonite R., Presky D. H. (1995). Measurement of human and mouse interleukin-12. Curr Protoc Immunol 1995;6(16):1-15.

Ginhoux F, Turbant S, Gross DA, Poupiot J, Marais T, Lone Y, Lemonnier FA, Firat H, Perez N, Danos 0, Davoust J. (2004). HLA-A*0201-restricted cytolytic responses to the rtTA transactivator dominant and cryptic epitopes compromise transgene expression induced by the tetracycline on system. Mol Ther. 10(2):279-89.

Gonzalez-Aparicio M, Alzuguren P, Mauleon I, Medina-Echeverz J, Hervas-Stubbs S, Mancheno U, Berraondo P, Crettaz J, Gonzalez-Aseguinolaza G, Prieto J, Hernandez-Alcoceba R. (2011). Oxaliplatin in combination with liver-specific expression of interleukin 12 reduces the immunosuppressive microenvironment of tumours and eradicates metastatic colorectal cancer in mice. Gut 60(3):341-9.

Greig J A, Wang Q, Reicherter A L, Chen S J, Hanlon A L, Tipper C H, Clark K R, Wadsworth S, Wang L, Wilson J M. (2017). Characterization of Adeno-Associated Viral Vector-Mediated Human Factor VIII Gene Therapy in Hemophilia A Mice. Hum Gene Ther. 2017 28:392-402

Grimm, D., and Büning, H. (2017) Small But Increasingly Mighty: Latest Advances in AAV Vector Research, Design, and Evolution, Human Gene Therapy 28, 1075-1086

Higgins et al., 1996, Methods Enzymol. 266:383-402

Honegger, A., Spinelli, S., Cambillau, C., and Plückthun, A. (2005) A mutation designed to alter crystal packing permits structural analysis of a tight-binding fluorescein-scFv complex, Protein Sci. 14, 2537-2549

Huang, G.; Liu, Z.; van der Maaten, L.; Weinberger, K. Q. (2017). Densely Connected Convolutional Networks. In Proceedings of the IEEE Conference on Computer Vision and Pattern Recognition, Honolulu, HI, USA, 21-26 Jul. 2017.

Jemal, A., Thomas, A., Murray, T., Thun, M. (2002). Cancer statistics, 2002. CA Cancer J Clin. 52(1):23-47.

Jiang, D. J., Xu, C. L., and Tsang, S. H. (2018) Revolution in Gene Medicine Therapy and Genome Surgery, Genes (Basel) 9, 575

Kamimura K, Yokoo T, Abe H, Terai S. (2019). Gene Therapy for Liver Cancers: Current Status from Basic to Clinics. Cancers (Basel). 11(12):1865.

Karlin and Altschul, 1990, Proc. Natl. Acad. Sci. USA 87:2264-2268

Karlin and Altschul, 1993, Proc. Natl. Acad. Sci. USA 90:5873-5877

Kennedy, A. B., Vowles, J. V., d'Espaux, L., and Smolke, C. D. (2014) Proteinresponsive ribozyme switches in eukaryotic cells, Nucleic Acids Research. 19, 12306-12321

Ketzer, P., Haas, S. F., Engelhardt, S., Hartig, J. S., and Nettelbeck, D. M. (2012) Synthetic riboswitches for external regulation of genes transferred by replication-deficient and oncolytic adenoviruses, Nucleic Acids Research 40, e167

Ketzer, P., Kaufmann, J. K., Engelhardt, S., Bossow, S., von Kalle, C., Hartig, J. S., Ungerechts, G., and Nettelbeck, D. M. (2014) Artificial riboswitches for gene expression and replication control of DNA and RNA viruses, Proc. Natl. Acad. Sci. U.S.A 111, E554-562

Kitada, T., DiAndreth, B., Teague, B., and Weiss, R. (2018) Programming gene and engineered-cell therapies with synthetic biology, Science 359

Kumar, D., An, C.-I., and Yokobayashi, Y. (2009) Conditional RNA interference mediated by allosteric ribozyme, J. Am. Chem. Soc. 131, 13906-13907

Kumar, S. R., Markusic, D. M., Biswas, M., High, K. A., and Herzog, R. W. (2016) Clinical development of gene therapy: results and lessons from recent successes, Mol Ther Methods Clin Dev 3, 16034

Lan K H. et al. 1997. In vivo selective gene expression and therapy mediated by adenoviral vectors for human carcinoembryonic antigen-producing gastric carcinoma. Cancer Res. 57(19):4279-84

Leonard, J. P., Sherman, M. L., Fisher, G. L., Buchanan, L. J., Larsen, G., Atkins, M. B., Sosman, J. A., Dutcher, J. P., Vogelzang, N. J., and Ryan, J. L. (1997) Effects of singledose interleukin-12 exposure on interleukin-12-associated toxicity and interferon-gamma production, Blood 90, 2541-2548

Levitt, N., Briggs, D., Gil, A., Proudfoot, N. J. (1989). Definition of an efficient synthetic poly(A) site. Genes Dev. 3(7):1019-25.

Lieschke G J I, Rao P K, Gately M K, Mulligan R C. Bioactive murine and human interleukin-12 fusion proteins which retain antitumor activity in vivo. Nat Biotechnol. 1997 January;15(1):35-40.

Lo C H, Chang C M, Tang S W, Pan W Y, Fang C C, Chen Y, Wu P Y, Chen K Y, Ma H I, Xiao X, Tao M H. (2010). Differential antitumor effect of interleukin-12 family cytokines on orthotopic hepatocellular carcinoma. J Gene Med. 12(5):423-34.

Manno, C. S., Pierce, G. F., Arruda, V. R., Glader, B., Ragni, M., Rasko, J. J., Rasko, J., Ozelo, M. C., Hoots, K., Blatt, P., Konkle, B., Dake, M., Kaye, R., Razavi, M., Zajko, A., Zehnder, J., Rustagi, P. K., Nakai, H., Chew, A., Leonard, D., Wright, J. F., Lessard, R. R., Sommer, J. M., Tigges, M., Sabatino, D., Luk, A., Jiang, H., Mingozzi, F., Couto, L., Ertl, H. C., High, K. A., and Kay, M. A. (2006) Successful transduction of liver in hemophilia by AAV-Factor IX and limitations imposed by the host immune response, Nature Medicine 12, 342-347.

Nathwani A C, Gray J T, Ng C Y, Zhou J, Spence Y, Waddington S N, Tuddenham E G, Kemball-Cook G, McIntosh J, Boon-Spijker M, Mertens K, Davidoff A M. (2006) Self-complementary adeno-associated virus vectors containing a novel liver-specific human factor IX expression cassette enable highly efficient transduction of murine and nonhuman primate liver, Blood 107, 2653-2661

Nathwani, A. C., Tuddenham, E. G. D., Rangarajan, S., Rosales, C., McIntosh, J., Linch, D. C., Chowdary, P., Riddell, A., Pie, A. J., Harrington, C., O'Beirne, J., Smith, K., Pasi, J., Glader, B., Rustagi, P., Ng, C. Y. C., Kay, M. A., Zhou, J., Spence, Y., Morton, C. L., Allay, J., Coleman, J., Sleep, S., Cunningham, J. M., Srivastava, D., Basner-Tschakarjan, E., Mingozzi, F., High, K. A., Gray, J. T., Reiss, U. M., Nienhuis, A. W., and Davidoff, A. M. (2011) Adenovirus-associated virus vector-mediated gene transfer in hemophilia B, N. Engl. J. Med. 365, 2357-2365

Nathwani, A. C., Reiss, U. M., Tuddenham, E. G. D., Rosales, C., Chowdary, P., McIntosh, J., Della Peruta, M., Lheriteau, E., Patel, N., Raj, D., Riddell, A., Pie, J., Rangarajan, S., Bevan, D., Recht, M., Shen, Y.-M., Halka, K. G., Basner-Tschakarj an, E., Mingozzi, F., High, K. A., Allay, J., Kay, M. A., Ng, C. Y. C., Zhou, J., Cancio, M., Morton, C. L., Gray, J. T., Srivastava, D., Nienhuis, A. W., and Davidoff, A. M. (2014) Long-term safety and efficacy of factor IX gene therapy in hemophilia B, N. Engl. J. Med. 371, 1994-2004

Nomura, Y., Zhou, L., Miu, A., and Yokobayashi, Y. (2013) Controlling mammalian gene expression by allosteric hepatitis delta virus ribozymes, ACS synthetic biology 2, 684-689

Okuyama T, Huber RM, Bowling W, et al. Liver-directed gene therapy: a retroviral vector with a complete LTR and the ApoE enhancer-alpha 1-antitrypsin promoter dramatically increases expression of human alpha 1-antitrypsin in vivo. Human Gene Therapy. 1996 March;7(5):637-645. DOI: 10.1089/hum.1996.7.5-637.

Paulk, N. (2020). Gene Therapy: It's Time to Talk about High-Dose AAV. The deaths of two children with X-linked myotubular myopathy in the ASPIRO trial prompts a reexamination of vector safety. Genetic Engineering & Biotechnology News 40(9):14-16.

Pearson and Lipman, 1988, Proc. Natl. Acad. Sci. USA 85:2444-8

Cabrera-Pérez R, Vila-Julia F, Hirano M, Mingozzi F, Torres-Torronteras J, Marti R. Alpha-1-Antitrypsin Promoter Improves the Efficacy of an Adeno-Associated Virus Vector for the Treatment of Mitochondrial Neurogastrointestinal Encephalomyopathy. Hum Gene Ther. 2019 August;30(8):985-998. doi: 10.1089/hum.2018.217. Epub 2019 Apr 24. PMID: 30900470; PMCID: PMC7647930.

Qian C, Liu X Y, Prieto J. (2006). Therapy of cancer by cytokines mediated by gene therapy approach. Cell Res. 16(2):182-8.

Michael Quante, Steffen Heeg, Alexander von Werder, Gitta Goessel, Christine Fulda, Michaela Doebele, Hiroshi Nakagawa, Roderick Beijersbergen, Hubert E Blum, Oliver G Opitz (2005). Differential transcriptional regulation of human telomerase in a cellular model representing important genetic alterations in esophageal squamous carcinogenesis, Carcinogenesis vol.26 no.11 pp. 1879-1889

Reid, C. A., Nettesheim, E. R., Connor, T. B., and Lipinski, D. M. (2018) Development of an inducible anti-VEGF rAAV gene therapy strategy for the treatment of wet AMD, Sci Rep 8, 11763

Russell, S., Bennett, J., Wellman, J. A., Chung, D. C., Yu, Z.-F., Tillman, A., Wittes, J., Pappas, J., Elci, O., McCague, S., Cross, D., Marshall, K. A., Walshire, J., Kehoe, T. L., Reichert, H., Davis, M., Raffini, L., George, L. A., Hudson, F. P., Dingfield, L., Zhu, X., Haller, J. A., Sohn, E. H., Mahaj an, V. B., Pfeifer, W., Weckmann, M., Johnson, C., Gewaily, D., Drack, A., Stone, E., Wachtel, K., Simonelli, F., Leroy, B. P., Wright, J. F., High, K. A., and Maguire, A. M. (2017) Efficacy and safety of voretigene neparvovec (AAV2-hRPE65v2) in patients with RPE65-mediated inherited retinal dystrophy: a randomised, controlled, open-label, phase 3 trial, Lancet 390, 849-860

Samulski R. J., Srivastava A., Berns K. I., Muzyczka, N. (1983). Rescue of adeno-associated virus from recombinant plasmids: gene correction within the terminal repeats of AAV. Cell 33(1):135-43.

Santiago, C. P., Keuthan, C. J., Boye, S. L., Boye, S. E., Imam, A. A., and Ash, J. D. (2018) A Drug-Tunable Gene Therapy for Broad-Spectrum Protection against Retinal Degeneration, Mol Ther 26, 2407-2417.

Sarcar, S., Tulalamba, W., Rincon, M. Y., Tipanee, J., Pham, H. Q., Evens, H., Boon, D., Samara-Kuko, E., Keyaerts, M., Loperfido, M., Berardi, E., Jarmin, S., In't Veld, P., Dickson, G., Lahoutte, T., Sampaolesi, M., De Bleser, P., VandenDriessche, T., and Chuah, M. K. (2019) Next-generation muscle-directed gene therapy by in silico vector design, Nat Commun 10, 492

Schumann et al. 2002a IFCC Primary Reference Procedures for the Measurement of Catalytic Activity Concentrations of Enzymes at 37° C., Clin Chem Lab Med 2002; 40(7):718-724© 2002 by Walter de Gruyter·Berlin·New York

Schumann et al. 2002b, IFCC Primary Reference Procedures for the Measurement of Catalytic Activity Concentrations of Enzymes at 37° C. Clin Chem Lab Med 2002; 40(6):643-648© 2002 by Walter de Gruyter·Berlin·New York

Sheridan, C. (2015). Mayo clinician reports on patient death in oncolytic virus trial. https://www.bioworld.com/articles/396511-mayo-clinician-reports-on-patient-death-in-oncolytic-virus-trial?v=preview.

Yu-Jun Shi, Jian-Ping Gong, Chang-An Liu, Xu-Hong Li, Ying Mei, Can Mi, and YanYing Huo (2004). Construction of a targeting adenoviral vector carrying AFP promoter for expressing EGFP gene in AFP producing hepatocarcinoma cell, World J Gastroenterol. 2004 Jan. 15; 10(2): 186-189.

Strobel, B., Miller, F. D., Rist, W., and Lamla, T. (2015a) Comparative analysis of cesium chloride- and iodixanol-based purification of recombinant Adeno-associated virus (AAV) vectors for preclinical applications, Hum Gene Ther Methods 26, 147-57

Strobel, B., Klauser, B., Hartig, J. S., Lamla, T., Gantner, F., and Kreuz, S. (2015b) Riboswitch-mediated Attenuation of Transgene Cytotoxicity Increases Adeno-associated Virus Vector Yields in HEK-293 Cells, Molecular Therapy 23, 1582-1591

Tang, A., Harding, F. (2019). The challenges and molecular approaches surrounding interleukin-2-based therapeutics in cancer. Cytokine: X1. 1:100001

Tai YT. et al. 1999. In vivo cytotoxicity of ovarian cancer cells through tumor-selective expression of the BAX gene. Cancer Res. 59(9):2121-6.

Torellis and Robotti, 1994, Comput. Appl. Biosci. 10:3-5

Valderrama-Treviño, A. I., Barrera-Mera, B., Ceballos-Villalva, J. C., and Montalvo-Javé, E. E. (2017). Hepatic Metastasis from Colorectal Cancer. Euroasian J Hepatogastroenterol. 7: 166-175.

Vanrell L, Di Scala M, Blanco L, Otano I, Gil-Farina I, Baldim V, Paneda A, Berraondo P, Beattie S G, Chtarto A, Tenenbaum L, Prieto J, Gonzalez-Aseguinolaza G. (2011). Development of a liver-specific Tet-on inducible system for AAV vectors and its application in the treatment of liver cancer. Mol Ther. 19(7):1245-53.

Vaughan, T. J., Williams, A. J., Pritchard, K., Osbourn, J. K., Pope, A. R., Earnshaw, J. C., McCafferty, J., Hodits, R. A., Wilton, J., and Johnson, K. S. (1996) Human antibodies with sub-nanomolar affinities isolated from a large non-immunized phage display library, Nature Biotechnology 14, 309-314

Wang, L., Hernandez-Alcoceba, R., Shankar, V., Zabala, M., Kochanek, S., Sangro, B., Kramer, M. G., Prieto, J., Qian, C. (2004). Prolonged and inducible transgene expression in the liver using gutless adenovirus: a potential therapy for liver cancer. Gastroenterology. 126(1):278-89.

Wang, D., Tai, P. W. L., Gao, G. (2019). Adeno-associated virus vector as a platform for gene therapy delivery. Nat. Rev. Drug. Disc.18:358-378.

Wei, K. Y., and Smolke, C. D. (2015) Engineering dynamic cell cycle control with synthetic small molecule-responsive RNA devices, J Biol Eng 9, 21.

Wilmott P., Lisowski L., Alexander I. E., Logan G. J. (2019). A User's Guide to the Inverted Terminal Repeats of Adeno-Associated Virus Hum Gene Ther Methods. 30(6):206-213.

Yen, L., Svendsen, J., Lee, J. -S., Gray, J. T., Magnier, M., Baba, T., D'Amato, R. J., and Mulligan, R. C. (2004) Exogenous control of mammalian gene expression through modulation of RNA self-cleavage, Nature 431, 471-476

Yu J, Green M D, Li S, Sun Y, Journey S N, Choi J E, Rizvi S M, Qin A, Waninger J J, Lang X, Chopra Z, El Naqa I, Zhou J, Bian Y, Jiang L, Tezel A, Skvarce J, Achar R K, Sitto M, Rosen BS, Su F, Narayanan S P, Cao X, Wei S, Szeliga W, Vatan L, Mayo C, Morgan M A, Schonewolf C A, Cuneo K, Kryczek I, Ma V T, Lao C D, Lawrence T S, Ramnath N, Wen F, Chinnaiyan A M, Cieslik M, Alva A, Zou W. (2021). Liver metastasis restrains immunotherapy efficacy via macrophage-mediated T cell elimination. Nat Med. 27(1):152-164.

Zhang, L., Morgan, R. A., Beane J. D., Zheng Z., Dudley M. E., Kassim S. H., Nahvi A. V., Ngo L. T., Sherry R. M., Phan G. Q., Hughes M. S., Kammula U. S., Feldman S. A., Toomey M. A., Kerkar S. P., Restifo N. P., Yang J. C., Rosenberg S. A. (2011) Improving adoptive T cell therapy by targeting and controlling IL-12 expression to the tumor environment. Molecular Therapy 19, 751-759

Zhong, G., Wang, H., Bailey, C. C., Gao, G., and Farzan, M. (2016) Rational design of aptazyme riboswitches for efficient control of gene expression in mammalian cells, Elife 5:e18858

Zhong (2020) A reversible RNA on-switch that controls gene expression of AAV-delivered therapeutics in vivo Nat Biotechnol. 2020 February;38(2):169-175. doi: 10.1038/541587-019-0357-y. Epub 2019 Dec. 23

Zhou Q, Tian W, Liu C, Lian Z, Dong X, Wu X. (2017). Deletion of the B-B′ and C-C′ regions of inverted terminal repeats reduces rAAV productivity but increases transgene expression. Sci Rep. 7(1):5432.

Zou, W. (2005) Immunosuppressive networks in the tumour environment and their therapeutic relevance, Nat Rev Cancer 5, 263-274

Zincarelli, C., Soltys, S., Rengo, G., and Rabinowitz, J. E. (2008) Analysis of AAV Serotypes 1-9 Mediated Gene Expression and Tropism in Mice After Systemic Injection, Molecular Therapy 16, 1073-1080

Qingzhang Zhou, Wenhong Tian, Qingzhang Zhou, Wenhong Tian, Deletion of the B-B′ and C-C′ regions of inverted terminal repeats reduces rAAV productivity but increases transgene expression, Sci Rep. 2017 Jul. 14;7(1):5432. doi: 10.1038/s41598-017-04054-4

Shin, Yue, and Duan Recombinant Adeno-Associated Viral Vector Production and Purification Methods Mol Biol. 2012; 798: 267-284.doi: 10.1007/978-1-61779-343-1_15

This table shows sequences in FASTA format mentioned in the text. In case of inconsistencies with the sequence listing, the sequences shown in the table are the authentic sequences.

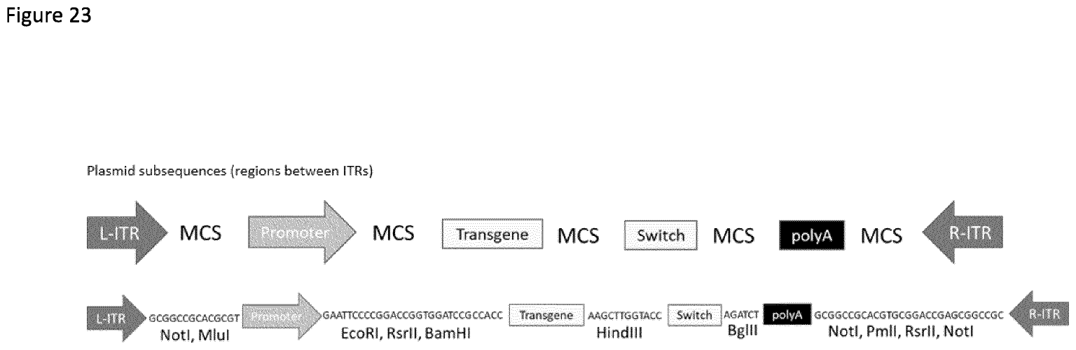

TABLE 2
>SV40 poly(A) (SEQ ID. No. 7)
cttgtttattgcagcttataatggttacaaataaagcaatagcatcacaaatttcacaaataaagcatttttttcactgcattcta
gttgtggtttgtccaaactcatcaatgtatcttaacgcggccg
>AAV2 ITR (SEQ ID. No. 8)
cctgcaggcagctgcgcgctcgctcgctcactgaggccgcccgggcaaagcccgggcgtcgggcgacctttggtcgcccggcc
tcagtgagcgagcgagcgcgcagagagggagtggccaactccatcactaggggttcct
>K19 riboswitch DNA (described in Beilstein et al., 2014) (SEQ ID No. 9)
GGCGCGTCCTGGATTCGTGGTAAAACATACCAGATTTCGATCTGGAGAGGTGAAGAATACGAC
CACCTACTACATCCAGCTGATGAGTCCCAAATAGGACGAAACGCGCT
>K19 riboswitch RNA (SEQ ID. No. 10)
GGCGCGUCCUGGAUUCGUGGUAAAACAUACCAGAUUUCGAUCUGGAGAGGUGAAGAAUA
CGACCACCUACUACAUCCAGCUGAUGAGUCCCAAAUAGGACGAAACGCGCU
>Nucleotide sequence encoding functional mIL-12 single chain (human IgG signal pep-
tide, p40 subunit, (Gly4Ser)3 linker, p35 subunit (SEQ ID No. 11)
atgggctggtcctgcatcattctgtttctggtggccacagccaccggtgtccactctatgtgggaactcgagaaggacgtgtac
gtggtggaagtggactggacacctgatgctccaggcgagacagtgaacctgacctgtgacacacccgaagaggacgacatc
acctggacaagcgatcagagacacggcgtgatcggcagcggcaagaccctgacaatcaccgtgaaagagtttctggacgcc
ggccagtacacctgtcacaaaggcggagagacactgtcccacagccatctgctgctgcacaagaaagagaacggcatctggt
ccaccgagatcctgaagaacttcaagaacaagaccttcctgaagtgcgaggcccctaactacagcggcagattcacatgtag
ctggctggtgcagagaaacatggacctgaagttcaacatcaagtcctccagcagcagccccgacagcagagctgttacatgt
ggcatggctagcctgagcgccgagaaagtgacactggaccagagagactacgagaagtacagcgtgtcctgccaagaggac
gtgacctgtcctacagccgaggaaacactgcctatcgagctggccctggaagccagacagcagaacaaatacgagaactac
tctaccagcttcttcatccgggacatcatcaagcccgatcctccaaagaacctgcagatgaagcctctgaagaacagccaggt
cgaggtgtcctgggagtaccctgactcttggagcacccctcacagctacttcagcctgaaattcttcgtgcgcatccagcgcaa
gaaagaaaagatgaaggaaaccgaggaaggctgcaaccagaagggcgccttcctggtcgaaaagacctctaccgaggtgc
agtgcaaaggcggcaatgtctgtgtgcaggcccaggataggtactacaacagcagctgcagcaagtgggcctgcgtgccatg
tagagttagaagcggaggcggaggaagtggtggcggaggttctggcggcggtggaagtagagttatccctgtgtctggccct
gccagatgcctgtctcagagcagaaacctgctgaaaaccaccgacgacatggtcaagaccgccagagagaagctgaagcac
tacagctgcaccgccgaggacatcgaccacgaggatatcacaagggaccagaccagcacactgaaaacctgcctgcctctg
gaactgcataagaacgagagctgcctggccacaagagagacaagcagcaccacaagaggcagctgtctgcctcctcagaa
aaccagcctgatgatgacactgtgcctgggcagcatctacgaggatctgaagatgtaccagaccgagttccaggccatcaac
gccgctctgcagaaccacaaccaccagcagatcatcctggataagggcatgctggtggctatcgacgagctgatgcagagcc
tgaaccacaatggcgagacactgagacagaagcctccagtcggagaggccgatccttacagagtgaagatgaagctgtgca
tcctgctgcacgccttcagcaccagagtggtcaccatcaacagagtgatgggctacctgagtagtgcatga
>Protein sequence of functional mIL-12 single chain (p40 subunit, (Gly4Ser)3 linker, p35
subunit) without signal peptide (SEQ ID. No. 12)
MWELEKDVYVVEVDWTPDAPGETVNLTCDTPEEDDITWTSDQRHGVIGSGKTLTITVKEFLDAGQ
YTCHKGGETLSHSHLLLHKKENGIWSTEILKNFKNKTFLKCEAPNYSGRFTCSWLVQRNMDLKFNIKS
SSSSPDSRAVTCGMASLSAEKVTLDQRDYEKYSVSCQEDVTCPTAEETLPIELALEARQQNKYENYST
SFFIRDIIKPDPPKNLQMKPLKNSQVEVSWEYPDSWSTPHSYFSLKFFVRIQRKKEKMKETEEGCNQ
KGAFLVEKTSTEVQCKGGNVCVQAQDRYYNSSCSKWACVPCRVRSGGGGSGGGGSGGGGSRVIP
VSGPARCLSQSRNLLKTTDDMVKTAREKLKHYSCTAEDIDHEDITRDQTSTLKTCLPLELHKNESCLAT
RETSSTTRGSCLPPQKTSLMMTLCLGSIYEDLKMYQTEFQAINAALQNHNHQQIILDKGMLVAIDEL
MQSLNHNGETLRQKPPVGEADPYRVKMKLCILLHAFSTRVVTINRVMGYLSSA
> human IgG signal peptide (SEQ ID No. 13)
atgggctggtcctgcatcattctgtttctggtggccacagccaccggtgtccactct
>murine IL-12 beta chain (p40 subunit) (SEQ ID No. 14)
atgtgggaactcgagaaggacgtgtacgtggtggaagtggactggacacctgatgctccaggcgagacagtgaacctgacct
gtgacacacccgaagaggacgacatcacctggacaagcgatcagagacacggcgtgatcggcagcggcaagaccctgaca
atcaccgtgaaagagtttctggacgccggccagtacacctgtcacaaaggcggagagacactgtcccacagccatctgctgct
gcacaagaaagagaacggcatctggtccaccgagatcctgaagaacttcaagaacaagaccttcctgaagtgcgaggcccc
taactacagcggcagattcacatgtagctggctggtgcagagaaacatggacctgaagttcaacatcaagtcctccagcagc
agccccgacagcagagctgttacatgtggcatggctagcctgagcgccgagaaagtgacactggaccagagagactacgag
aagtacagcgtgtcctgccaagaggacgtgacctgtcctacagccgaggaaacactgcctatcgagctggccctggaagcca
gacagcagaacaaatacgagaactactctaccagcttcttcatccgggacatcatcaagcccgatcctccaaagaacctgca
gatgaagcctctgaagaacagccaggtcgaggtgtcctgggagtaccctgactcttggagcacccctcacagctacttcagcc
tgaaattcttcgtgcgcatccagcgcaagaaagaaaagatgaaggaaaccgaggaaggctgcaaccagaagggcgccttcc
tggtcgaaaagacctctaccgaggtgcagtgcaaaggcggcaatgtctgtgtgcaggcccaggataggtactacaacagcag
ctgcagcaagtgggcctgcgtgccatgtagagttaga
>murine IL-12 alpha chain (p35 subunit) (SEQ ID No. 15)
agagttatccctgtgtctggccctgccagatgcctgtctcagagcagaaacctgctgaaaaccaccgacgacatggtcaagac
cgccagagagaagctgaagcactacagctgcaccgccgaggacatcgaccacgaggatatcacaagggaccagaccagca
cactgaaaacctgcctgcctctggaactgcataagaacgagagctgcctggccacaagagagacaagcagcaccacaagag
gcagctgtctgcctcctcagaaaaccagcctgatgatgacactgtgcctgggcagcatctacgaggatctgaagatgtaccag
accgagttccaggccatcaacgccgctctgcagaaccacaaccaccagcagatcatcctggataagggcatgctggtggcta
tcgacgagctgatgcagagcctgaaccacaatggcgagacactgagacagaagcctccagtcggagaggccgatccttaca
gagtgaagatgaagctgtgcatcctgctgcacgccttcagcaccagagtggtcaccatcaacagagtgatgggctacctgagt
agtgcatga
>(Gly4Ser)3 linker (SEQ ID No. 16)
agcggaggcggaggaagtggtggcggaggttctggcggcggtggaagt
Primer Sequences
>K19-riboswitch FW (SEQ ID No. 17)
GCGTCCTGGATTCGTGGTAA
>K19-riboswitch RV (SEQ ID No. 18)
GCTGGATGTAGTAGGTGGTCGTATT
>K19-riboswitch probe (SEQ ID No. 19)
ATTTCGATCTGGAGAGGTG
>anti-FITC gene FW (SEQ ID No.20)
TCTGCGCGTGGAAGATACAG
>anti-FITC gene RV (SEQ ID No. 21)
CAATATCCGGAGGAGTCGTAGCT
>anti-FITC gene probe (SEQ ID No. 22)
TGTGTATTATTGCGCTAGGC
> polr2a FW (SEQ ID No. 23)
GCCAAAGACTCCTTCACTCACTGT
> polr2a RV (SEQ ID No. 24)
TTCCAAGCGGCAAAGAATGT
> polr2a probe (SEQ ID No. 25)
TGGCTCTTTCAGCATCTCGTGCAGATT
> POLR2A FW (SEQ ID No. 26)
GCAAGCGGATTCCATTTGG
> POLR2A RV (SEQ ID No. 27)
TCTCAGGCCCGTAGTCATCCT
> POLR2A probe (SEQ ID No. 28)
AAGCACCGGACTCTGCCTCACTTCATC
Plasmid subsequences (regions between ITRs) see FIG. 23
>pAAV.LP1-mIL-12-3′-riboswitch (SEQ ID No. 29)
cctgcaggcagctgcgcgctcgctcgctcactgaggccgcccgggcaaagcccgggcgtcgggcgacctttggtcgcccggcc
tcagtgagcgagcgagcgcgcagagagggagtggccaactccatcactaggggttcctgcggccgcacgcgttcgaccccct
aaaatgggcaaacattgcaagcaaacagcaaacacacagccctccctgcctgctgaccttggagctggggcagaggtcaga
gacctctctgggcccatgccacctccaacatccactcgaccccttggaatttcggtggagaggagcagaggttgtcctggcgtg
gtttaggtagtgtgagaggggaatgactcctttcggtaagtgcagtggaagctgtacactgcccaggcaaagcgtccgggcag
cgtaggcgggcgactcagatcccagccagtggacttagcccctgtttgctcctccgataactggggtgaccttggttaatattca
ccagcagcctcccccgttgcccctctggatccactgcttaaatacggacgaggacagggccctgtctcctcagcttcaggcacc
accactgacctgggacagtgaatccggactctaagagaattccccggaccggtggatccgccaccatgggctggtcctgcatc
attctgtttctggtggccacagccaccggtgtccactctatgtgggaactcgagaaggacgtgtacgtggtggaagtggactgg
acacctgatgctccaggcgagacagtgaacctgacctgtgacacacccgaagaggacgacatcacctggacaagcgatcag
agacacggcgtgatcggcagcggcaagaccctgacaatcaccgtgaaagagtttctggacgccggccagtacacctgtcaca
aaggcggagagacactgtcccacagccatctgctgctgcacaagaaagagaacggcatctggtccaccgagatcctgaaga
acttcaagaacaagaccttcctgaagtgcgaggcccctaactacagcggcagattcacatgtagctggctggtgcagagaaa
catggacctgaagttcaacatcaagtcctccagcagcagccccgacagcagagctgttacatgtggcatggctagcctgagcg
ccgagaaagtgacactggaccagagagactacgagaagtacagcgtgtcctgccaagaggacgtgacctgtcctacagccg
aggaaacactgcctatcgagctggccctggaagccagacagcagaacaaatacgagaactactctaccagcttcttcatccg
ggacatcatcaagcccgatcctccaaagaacctgcagatgaagcctctgaagaacagccaggtcgaggtgtcctgggagtac
cctgactcttggagcacccctcacagctacttcagcctgaaattcttcgtgcgcatccagcgcaagaaagaaaagatgaagga
aaccgaggaaggctgcaaccagaagggcgccttcctggtcgaaaagacctctaccgaggtgcagtgcaaaggcggcaatgt
ctgtgtgcaggcccaggataggtactacaacagcagctgcagcaagtgggcctgcgtgccatgtagagttagaagcggaggc
ggaggaagtggtggcggaggttctggcggcggtggaagtagagttatccctgtgtctggccctgccagatgcctgtctcagag
cagaaacctgctgaaaaccaccgacgacatggtcaagaccgccagagagaagctgaagcactacagctgcaccgccgagg
acatcgaccacgaggatatcacaagggaccagaccagcacactgaaaacctgcctgcctctggaactgcataagaacgaga
gctgcctggccacaagagagacaagcagcaccacaagaggcagctgtctgcctcctcagaaaaccagcctgatgatgacac
tgtgcctgggcagcatctacgaggatctgaagatgtaccagaccgagttccaggccatcaacgccgctctgcagaaccacaa
ccaccagcagatcatcctggataagggcatgctggtggctatcgacgagctgatgcagagcctgaaccacaatggcgagaca
ctgagacagaagcctccagtcggagaggccgatccttacagagtgaagatgaagctgtgcatcctgctgcacgccttcagca
ccagagtggtcaccatcaacagagtgatgggctacctgagtagtgcatgaaagcttggtacccaaacaaacaaaggcgcgtc
ctggattcgtggtaaaacataccagatttcgatctggagaggtgaagaatacgaccacctactacatccagctgatgagtccc
aaataggacgaaacgcgctcaaacaaacaaaagatctcttgtttattgcagcttataatggttacaaataaagcaatagcatc
acaaatttcacaaataaagcatttttttcactgcattctagttgtggtttgtccaaactcatcaatgtatcttaacgcggccgcac
gtgcggaccgagcggccgcaggaacccctagtgatggagttggccactccctctctgcgcgctcgctcgctcactgaggccgg
gcgaccaaaggtcgcccgacgcccgggctttgcccgggcggcctcagtgagcgagcgagcgcgcagctgcctgcagg
> pAAV.LP1-aFITC-3′-riboswitch (SEQ ID No. 30)
cctgcaggcagctgcgcgctcgctcgctcactgaggccgcccgggcaaagcccgggcgtcgggcgacctttggtcgcccggcc
tcagtgagcgagcgagcgcgcagagagggagtggccaactccatcactaggggttcctgcggccgcacgcgttcgaccccct
aaaatgggcaaacattgcaagcaaacagcaaacacacagccctccctgcctgctgaccttggagctggggcagaggtcaga
gacctctctgggcccatgccacctccaacatccactcgaccccttggaatttcggtggagaggagcagaggttgtcctggcgtg
gtttaggtagtgtgagaggggaatgactcctttcggtaagtgcagtggaagctgtacactgcccaggcaaagcgtccgggcag
cgtaggcgggcgactcagatcccagccagtggacttagcccctgtttgctcctccgataactggggtgaccttggttaatattca
ccagcagcctcccccgttgcccctctggatccactgcttaaatacggacgaggacagggccctgtctcctcagcttcaggcacc
accactgacctgggacagtgaatccggactctaagagaattccccggaccggtggatccgccaccatggactggacctggcg
ggtgttctgtctgctggctgtggctcctggcgcccactctcaggtgcagctggtggaatctggcggcaacctggtgcagcctgg
cggctctctgagactgtcttgtgccgccagcggcttcaccttcggcagcttcagcatgagctgggtgcgacaggctccaggcgg
aggactggaatgggtggcaggcctgagcgccagaagcagcctgacacactacgccgacagcgtgaagggcagattcaccat
cagcagagacaacgccaagaacagcgtgtacctgcagatgaacagcctgagggtggaagataccgccgtgtactactgtgc
cagaagaagctacgacagcagcggctactggggccacttctacagctacatggacgtgtggggccagggcaccctcgtgaca
gtgtctagtggcggaggcggaagtggcggcggaggatcagggggaggcggatctcagtctgtgctgacccagcctagcagc
gtgtccgctgctcctggccagaaagtgaccatcagctgcagcggcagcaccagcaacatcggcaacaactacgtgtcctggt
atcagcagcaccccggcaaggcccccaagctgatgatctacgacgtgtccaagaggcccagcggcgtgcccgatagattcag
cggctctaagagcggcaacagcgccagcctggacatcagcggcctgcagtctgaggacgaggccgactattactgcgccgcc
tgggacgacagcctgtccgagttcctgttcggcaccggcaccaagctgacagtgctgggagggggaggatctggcgggggag
gctcacaggtgcagctggtggaaagcggcggaaatctggtgcagccagggggcagcctgagactgagctgtgccgcttccgg
ctttacctttggctccttctccatgtcctgggtgcgccaggcacctgggggcggactggaatgggtggccggactgtctgccag
aagctctctgacccactatgctgactctgtgaagggccggttcacaatctcccgggataacgctaagaactctgtgtacctgca
gatgaactctctgcgcgtggaagatacagctgtgtattattgcgctaggcggagctacgactcctccggatattggggacactt
ttactcttatatggatgtgtgggggcagggaacactcgtgaccgtgtcaagcggaggcggcggaagcgggggagggggatct
gggggcggaggcagtcagagtgtgctgacacagcccagctccgtgtctgccgccccaggacagaaagtgacaatctcctgct
ccggctccacctccaatatcggaaacaattatgtgtcttggtatcagcagcatcctgggaaggctcctaaactgatgatctatg
atgtgtctaaacggccttccggcgtgccagacaggttctccggaagcaagtccggcaactccgcctctctggacatctccggac
tgcagagcgaggatgaggctgactactattgtgctgcttgggacgactccctgagcgagtttctgtttggaacagggacaaaa
ctgaccgtgctgggcggcagcggaggcaagcctatccctaatcctctgctgggcctggacagcacctgaaagcttggtaccca
aacaaacaaaggcgcgtcctggattcgtggtaaaacataccagatttcgatctggagaggtgaagaatacgaccacctacta
catccagctgatgagtcccaaataggacgaaacgcgctcaaacaaacaaaagatctcttgtttattgcagcttataatggttac
aaataaagcaatagcatcacaaatttcacaaataaagcatttttttcactgcattctagttgtggtttgtccaaactcatcaatg
tatcttaacgcggccgcacgtgcggaccgagcggccgcaggaacccctagtgatggagttggccactccctctctgcgcgctc
gctcgctcactgaggccgggcgaccaaaggtcgcccgacgcccgggctttgcccgggcggcctcagtgagcgagcgagcgc
gcagctgcctgcagg
> pAAV.CMV-cNluc-3′-riboswitch (SEQ ID No. 31)
cctgcaggcagctgcgcgctcgctcgctcactgaggccgcccgggcaaagcccgggcgtcgggcgacctttggtcgcccggcc
tcagtgagcgagcgagcgcgcagagagggagtggccaactccatcactaggggttcctgcggccgcacgcgtctagttatta
atagtaatcaattacggggtcattagttcatagcccatatatggagttccgcgttacataacttacggtaaatggcccgcctgg
ctgaccgcccaacgacccccgcccattgacgtcaataatgacgtatgttcccatagtaacgccaatagggactttccattgacg
tcaatgggtggagtatttacggtaaactgcccacttggcagtacatcaagtgtatcatatgccaagtacgccccctattgacgt
caatgacggtaaatggcccgcctggcattatgcccagtacatgaccttatgggactttcctacttggcagtacatctacgtatta
gtcatcgctattaccatggtgatgcggttttggcagtacatcaatgggcgtggatagcggtttgactcacggggatttccaagtc
tccaccccattgacgtcaatgggagtttgttttgcaccaaaatcaacgggactttccaaaatgtcgtaacaactccgccccattg
acgcaaatgggcggtaggcgtgtacggtgggaggtctatataagcagagctcgtttagtgaaccgtcagatcgcctggagac
gccatccacgctgttttgacctccatagaagacaccgggaccgatccagcctccgcggattcgaacatcgattgaattccccgg
accggtggatccgccaccatggtcttcacactcgaagatttcgttggggactggcgacagacagccggctacaacctggacca
agtccttgaacagggaggtgtgtccagtttgtttcagaatctcggggtgtccgtaactccgatccaaaggattgtcctgagcgg
tgaaaatgggctgaagatcgacatccatgtcatcatcccgtatgaaggtctgagcggcgaccaaatgggccagatcgaaaaa
atttttaaggtggtgtaccctgtggatgatcatcactttaaggtgatcctgcactatggcacactggtaatcgacggggttacgc
cgaacatgatcgactatttcggacggccgtatgaaggcatcgccgtgttcgacggcaaaaagatcactgtaacagggaccct
gtggaacggcaacaaaattatcgacgagcgcctgatcaaccccgacggctccctgctgttccgagtaaccatcaacggagtg
accggctggcggctgtgcgaacgcattctggcgtaaaagcttggtacccaaacaaacaaaggcgcgtcctggattcgtggta
aaacataccagatttcgatctggagaggtgaagaatacgaccacctactacatccagctgatgagtcccaaataggacgaaa
cgcgctcaaacaaacaaaagatctcttgtttattgcagcttataatggttacaaataaagcaatagcatcacaaatttcacaa
ataaagcatttttttcactgcattctagttgtggtttgtccaaactcatcaatgtatcttaacgcggccgcacgtgcggaccgagc
ggccgcaggaacccctagtgatggagttggccactccctctctgcgcgctcgctcgctcactgaggccgggcgaccaaaggtc
gcccgacgcccgggctttgcccgggcggcctcagtgagcgagcgagcgcgcagctgcctgcagg
> pAAV.CMV-GFP-3′-riboswitch (SEQ ID No. 32)
cctgcaggcagctgcgcgctcgctcgctcactgaggccgcccgggcaaagcccgggcgtcgggcgacctttggtcgcccggcc
tcagtgagcgagcgagcgcgcagagagggagtggccaactccatcactaggggttcctgcggccgcacgcgtctagttatta
atagtaatcaattacggggtcattagttcatagcccatatatggagttccgcgttacataacttacggtaaatggcccgcctgg
ctgaccgcccaacgacccccgcccattgacgtcaataatgacgtatgttcccatagtaacgccaatagggactttccattgacg
tcaatgggtggagtatttacggtaaactgcccacttggcagtacatcaagtgtatcatatgccaagtacgccccctattgacgt
caatgacggtaaatggcccgcctggcattatgcccagtacatgaccttatgggactttcctacttggcagtacatctacgtatta
gtcatcgctattaccatggtgatgcggttttggcagtacatcaatgggcgtggatagcggtttgactcacggggatttccaagtc
tccaccccattgacgtcaatgggagtttgttttgcaccaaaatcaacgggactttccaaaatgtcgtaacaactccgccccattg
acgcaaatgggcggtaggcgtgtacggtgggaggtctatataagcagagctcgtttagtgaaccgtcagatcgcctggagac
gccatccacgctgttttgacctccatagaagacaccgggaccgatccagcctccgcggattcgaacatcgattgaattccccgg
accggtggatccgccaccatggtgagcaagggcgaggagctgttcaccggggtggtgcccatcctggtcgagctggacggcg
acgtaaacggccacaagttcagcgtgtccggcgagggcgagggcgatgccacctacggcaagctgaccctgaagttcatctg
caccaccggcaagctgcccgtgccctggcccaccctcgtgaccaccctgacctacggcgtgcagtgcttcagccgctaccccg
accacatgaagcagcacgacttcttcaagtccgccatgcccgaaggctacgtccaggagcgcaccatcttcttcaaggacgac
ggcaactacaagacccgcgccgaggtgaagttcgagggcgacaccctggtgaaccgcatcgagctgaagggcatcgacttc
aaggaggacggcaacatcctggggcacaagctggagtacaactacaacagccacaacgtctatatcatggccgacaagcag
aagaacggcatcaaggtgaacttcaagatccgccacaacatcgaggacggcagcgtgcagctcgccgaccactaccagcag
aacacccccatcggcgacggccccgtgctgctgcccgacaaccactacctgagcacccagtccgccctgagcaaagacccca
acgagaagcgcgatcacatggtcctgctggagttcgtgaccgccgccgggatcactctcggcatggacgagctgtacaagtcc
ggccggactcagatttcgagctcaagttttgaattttagaagcttggtacccaaacaaacaaaggcgcgtcctggattcgtggt
aaaacataccagatttcgatctggagaggtgaagaatacgaccacctactacatccagctgatgagtcccaaataggacgaa
acgcgctcaaacaaacaaaagatctcttgtttattgcagcttataatggttacaaataaagcaatagcatcacaaatttcaca
aataaagcatttttttcactgcattctagttgtggtttgtccaaactcatcaatgtatcttaacgcggccgcacgtgcggaccga
gcggccgcaggaacccctagtgatggagttggccactccctctctgcgcgctcgctcgctcactgaggccgggcgaccaaagg
tcgcccgacgcccgggctttgcccgggcggcctcagtgagcgagcgagcgcgcagctgcctgcagg
>hIL-12 first orientation, additional linker (SEQ ID No. 34-35), see Table 1
>hIL-12 second orientation (SEQ ID No. 36-41), see Table 1
>LP1-promoter/SV40-intron (SEQ ID No. 42
CCCTAAAATGGGCAAACATTGCAAGCAGCAAACAGCAAACACACAGCCCTCCCTGCCTGCTGAC
CTTGGAGCTGGGGCAGAGGTCAGAGACCTCTCTGGGCCCATGCCACCTCCAACATCCACTCGAC
CCCTTGGAATTTCGGTGGAGAGGAGCAGAGGTTGTCCTGGCGTGGTTTAGGTAGTGTGAGAGG
GTCCGGGTTCAAAACCACTTGCTGGGTGGGGAGTCGTCAGTAAGTGGCTATGCCCCGACCCCG
AAGCCTGTTTCCCCATCTGTACAATGGAAATGATAAAGACGCCCATCTGATAGGGAATGACTCC
TTTCGGTAAGTGCAGTGGAAGCTGTACACTGCCCAGGCAAAGCGTCCGGGCAGCGTAGGCGGG
CGACTCAGATCCCAGCCAGTGGACTTAGCCCCTGTTTGCTCCTCCGATAACTGGGGTGACCTTG
GTTAATATTCACCAGCAGCCTCCCCCGTTGCCCCTCTGGATCCACTGCTTAAATACGGACGAGGA
CAGGGCCCTGTCTCCTCAGCTTCAGGCACCACCACTGACCTGGGACAGTGAATCCGGACTCTAA
GGTAAATATAAAATTTTTAAGTGTATAATGTGTTAAACTACTGATTCTAATTGTTTCTCTATTTTA
GATTCCAACCTTTGGAACTGA
>WT AAV2 ITR (SEQ ID No. 43)
ttggccactccctctctgcgcgctcgctcgctcactgaggccgggcgaccaaaggtcgcccgacgcccgggctttgcccgggc
ggcctcagtgagcgagcgagcgcgcagagagggagtggccaactccatcactaggggttcct
>AAV ITRAC (SEQ ID No. 44)
aggaacccctagtgatggagttggccactccctctctgcgcgctcgctcgctcactgaggccgggcgaccaaaggtcgcccga
cgcccgggcggcctcagtgagcgagcgagcgcgcagctgcctgcagg
>K19 riboswitch-inactive DNA (SEQ ID No. 45)
GGCGCGTCCTGGATTCGTGGTAAAACATACCAGATTTCGATCTGGAGAGGTGAAGAATACGAC
CACCTACTACATCCAGCTGATGAGTCCCAAATAGGACGAGACGCGCT
>pAAV.LP1-mIL-12-3′-riboswitch (SEQ ID No. 46) - sequence according to Seq ID 29 as con-
firmed by confirmatory sequencing having an 11 nt deletion in the left ITR
CCTGCAGGCAGCTGCGCGCTCGCTCGCTCACTGAGGCCGCCCGGGCGTCGGGCGACCTTTGGT
CGCCCGGCCTCAGTGAGCGAGCGAGCGCGCAGAGAGGGAGTGGCCAACTCCATCACTAGGGG
TTCCTGCGGCCGCACGCGTTCGACCCCCTAAAATGGGCAAACATTGCAAGCAAACAGCAAACAC
ACAGCCCTCCCTGCCTGCTGACCTTGGAGCTGGGGCAGAGGTCAGAGACCTCTCTGGGCCCATG
CCACCTCCAACATCCACTCGACCCCTTGGAATTTCGGTGGAGAGGAGCAGAGGTTGTCCTGGCG
TGGTTTAGGTAGTGTGAGAGGGGAATGACTCCTTTCGGTAAGTGCAGTGGAAGCTGTACACTG
CCCAGGCAAAGCGTCCGGGCAGCGTAGGCGGGCGACTCAGATCCCAGCCAGTGGACTTAGCCC
CTGTTTGCTCCTCCGATAACTGGGGTGACCTTGGTTAATATTCACCAGCAGCCTCCCCCGTTGCC
CCTCTGGATCCACTGCTTAAATACGGACGAGGACAGGGCCCTGTCTCCTCAGCTTCAGGCACCA
CCACTGACCTGGGACAGTGAATCCGGACTCTAAGAGAATTCCCCGGACCGGTGGATCCGCCACC
ATGGGCTGGTCCTGCATCATTCTGTTTCTGGTGGCCACAGCCACCGGTGTCCACTCTATGTGGG
AACTCGAGAAGGACGTGTACGTGGTGGAAGTGGACTGGACACCTGATGCTCCAGGCGAGACA
GTGAACCTGACCTGTGACACACCCGAAGAGGACGACATCACCTGGACAAGCGATCAGAGACAC
GGCGTGATCGGCAGCGGCAAGACCCTGACAATCACCGTGAAAGAGTTTCTGGACGCCGGCCAG
TACACCTGTCACAAAGGCGGAGAGACACTGTCCCACAGCCATCTGCTGCTGCACAAGAAAGAG
AACGGCATCTGGTCCACCGAGATCCTGAAGAACTTCAAGAACAAGACCTTCCTGAAGTGCGAG
GCCCCTAACTACAGCGGCAGATTCACATGTAGCTGGCTGGTGCAGAGAAACATGGACCTGAAG
TTCAACATCAAGTCCTCCAGCAGCAGCCCCGACAGCAGAGCTGTTACATGTGGCATGGCTAGCC
TGAGCGCCGAGAAAGTGACACTGGACCAGAGAGACTACGAGAAGTACAGCGTGTCCTGCCAA
GAGGACGTGACCTGTCCTACAGCCGAGGAAACACTGCCTATCGAGCTGGCCCTGGAAGCCAGA
CAGCAGAACAAATACGAGAACTACTCTACCAGCTTCTTCATCCGGGACATCATCAAGCCCGATC
CTCCAAAGAACCTGCAGATGAAGCCTCTGAAGAACAGCCAGGTCGAGGTGTCCTGGGAGTACC
CTGACTCTTGGAGCACCCCTCACAGCTACTTCAGCCTGAAATTCTTCGTGCGCATCCAGCGCAAG
AAAGAAAAGATGAAGGAAACCGAGGAAGGCTGCAACCAGAAGGGCGCCTTCCTGGTCGAAAA
GACCTCTACCGAGGTGCAGTGCAAAGGCGGCAATGTCTGTGTGCAGGCCCAGGATAGGTACTA
CAACAGCAGCTGCAGCAAGTGGGCCTGCGTGCCATGTAGAGTTAGAAGCGGAGGCGGAGGAA
GTGGTGGCGGAGGTTCTGGCGGCGGTGGAAGTAGAGTTATCCCTGTGTCTGGCCCTGCCAGAT
GCCTGTCTCAGAGCAGAAACCTGCTGAAAACCACCGACGACATGGTCAAGACCGCCAGAGAGA
AGCTGAAGCACTACAGCTGCACCGCCGAGGACATCGACCACGAGGATATCACAAGGGACCAGA
CCAGCACACTGAAAACCTGCCTGCCTCTGGAACTGCATAAGAACGAGAGCTGCCTGGCCACAA
GAGAGACAAGCAGCACCACAAGAGGCAGCTGTCTGCCTCCTCAGAAAACCAGCCTGATGATGA
CACTGTGCCTGGGCAGCATCTACGAGGATCTGAAGATGTACCAGACCGAGTTCCAGGCCATCAA
CGCCGCTCTGCAGAACCACAACCACCAGCAGATCATCCTGGATAAGGGCATGCTGGTGGCTATC
GACGAGCTGATGCAGAGCCTGAACCACAATGGCGAGACACTGAGACAGAAGCCTCCAGTCGG
AGAGGCCGATCCTTACAGAGTGAAGATGAAGCTGTGCATCCTGCTGCACGCCTTCAGCACCAGA
GTGGTCACCATCAACAGAGTGATGGGCTACCTGAGTAGTGCATGAAAGCTTGGTACCCAAACA
AACAAAGGCGCGTCCTGGATTCGTGGTAAAACATACCAGATTTCGATCTGGAGAGGTGAAGAA
TACGACCACCTACTACATCCAGCTGATGAGTCCCAAATAGGACGAAACGCGCTCAAACAAACAA
AAGATCTCTTGTTTATTGCAGCTTATAATGGTTACAAATAAAGCAATAGCATCACAAATTTCACA
AATAAAGCATTTTTTTCACTGCATTCTAGTTGTGGTTTGTCCAAACTCATCAATGTATCTTAACGC
GGCCGCACGTGCGGACCGAGCGGCCGCAGGAACCCCTAGTGATGGAGTTGGCCACTCCCTCTC
TGCGCGCTCGCTCGCTCACTGAGGCCGGGCGACCAAAGGTCGCCCGACGCCCGGGCTTTGCCC
GGGCGGCCTCAGTGAGCGAGCGAGCGCGCAGCTGCCTGCAGG
>pAAV.LP1-mIL-12-3′-riboswitch (SEQ ID No. 47) - Sequence according to Seq ID 29 but
with the wt AAV2 ITR sequences instead of those shown in Seq ID 29
ttggccactccctctctgcgcgctcgctcgctcactgaggccgggcgaccaaaggtcgcccgacgcccgggctttgcccgggc
ggcctcagtgagcgagcgagcgcgcagagagggagtggccaactccatcactaggggttccTGCGGCCGCACGCGT
TCGACCCCCTAAAATGGGCAAACATTGCAAGCAAACAGCAAACACACAGCCCTCCCTGCCTGCT
GACCTTGGAGCTGGGGCAGAGGTCAGAGACCTCTCTGGGCCCATGCCACCTCCAACATCCACTC
GACCCCTTGGAATTTCGGTGGAGAGGAGCAGAGGTTGTCCTGGCGTGGTTTAGGTAGTGTGAG
AGGGGAATGACTCCTTTCGGTAAGTGCAGTGGAAGCTGTACACTGCCCAGGCAAAGCGTCCGG
GCAGCGTAGGCGGGCGACTCAGATCCCAGCCAGTGGACTTAGCCCCTGTTTGCTCCTCCGATAA
CTGGGGTGACCTTGGTTAATATTCACCAGCAGCCTCCCCCGTTGCCCCTCTGGATCCACTGCTTA
AATACGGACGAGGACAGGGCCCTGTCTCCTCAGCTTCAGGCACCACCACTGACCTGGGACAGT
GAATCCGGACTCTAAGAGAATTCCCCGGACCGGTGGATCCGCCACCATGGGCTGGTCCTGCATC
ATTCTGTTTCTGGTGGCCACAGCCACCGGTGTCCACTCTATGTGGGAACTCGAGAAGGACGTGT
ACGTGGTGGAAGTGGACTGGACACCTGATGCTCCAGGCGAGACAGTGAACCTGACCTGTGACA
CACCCGAAGAGGACGACATCACCTGGACAAGCGATCAGAGACACGGCGTGATCGGCAGCGGC
AAGACCCTGACAATCACCGTGAAAGAGTTTCTGGACGCCGGCCAGTACACCTGTCACAAAGGC
GGAGAGACACTGTCCCACAGCCATCTGCTGCTGCACAAGAAAGAGAACGGCATCTGGTCCACC
GAGATCCTGAAGAACTTCAAGAACAAGACCTTCCTGAAGTGCGAGGCCCCTAACTACAGCGGC
AGATTCACATGTAGCTGGCTGGTGCAGAGAAACATGGACCTGAAGTTCAACATCAAGTCCTCCA
GCAGCAGCCCCGACAGCAGAGCTGTTACATGTGGCATGGCTAGCCTGAGCGCCGAGAAAGTGA
CACTGGACCAGAGAGACTACGAGAAGTACAGCGTGTCCTGCCAAGAGGACGTGACCTGTCCTA
CAGCCGAGGAAACACTGCCTATCGAGCTGGCCCTGGAAGCCAGACAGCAGAACAAATACGAGA
ACTACTCTACCAGCTTCTTCATCCGGGACATCATCAAGCCCGATCCTCCAAAGAACCTGCAGATG
AAGCCTCTGAAGAACAGCCAGGTCGAGGTGTCCTGGGAGTACCCTGACTCTTGGAGCACCCCTC
ACAGCTACTTCAGCCTGAAATTCTTCGTGCGCATCCAGCGCAAGAAAGAAAAGATGAAGGAAA
CCGAGGAAGGCTGCAACCAGAAGGGCGCCTTCCTGGTCGAAAAGACCTCTACCGAGGTGCAGT
GCAAAGGCGGCAATGTCTGTGTGCAGGCCCAGGATAGGTACTACAACAGCAGCTGCAGCAAGT
GGGCCTGCGTGCCATGTAGAGTTAGAAGCGGAGGCGGAGGAAGTGGTGGCGGAGGTTCTGGC
GGCGGTGGAAGTAGAGTTATCCCTGTGTCTGGCCCTGCCAGATGCCTGTCTCAGAGCAGAAAC
CTGCTGAAAACCACCGACGACATGGTCAAGACCGCCAGAGAGAAGCTGAAGCACTACAGCTGC
ACCGCCGAGGACATCGACCACGAGGATATCACAAGGGACCAGACCAGCACACTGAAAACCTGC
CTGCCTCTGGAACTGCATAAGAACGAGAGCTGCCTGGCCACAAGAGAGACAAGCAGCACCACA
AGAGGCAGCTGTCTGCCTCCTCAGAAAACCAGCCTGATGATGACACTGTGCCTGGGCAGCATCT
ACGAGGATCTGAAGATGTACCAGACCGAGTTCCAGGCCATCAACGCCGCTCTGCAGAACCACA
ACCACCAGCAGATCATCCTGGATAAGGGCATGCTGGTGGCTATCGACGAGCTGATGCAGAGCC
TGAACCACAATGGCGAGACACTGAGACAGAAGCCTCCAGTCGGAGAGGCCGATCCTTACAGAG
TGAAGATGAAGCTGTGCATCCTGCTGCACGCCTTCAGCACCAGAGTGGTCACCATCAACAGAGT
GATGGGCTACCTGAGTAGTGCATGAAAGCTTGGTACCCAAACAAACAAAGGCGCGTCCTGGAT
TCGTGGTAAAACATACCAGATTTCGATCTGGAGAGGTGAAGAATACGACCACCTACTACATCCA
GCTGATGAGTCCCAAATAGGACGAAACGCGCTCAAACAAACAAAAGATCTCTTGTTTATTGCAG
CTTATAATGGTTACAAATAAAGCAATAGCATCACAAATTTCACAAATAAAGCATTTTTTTCACTG
CATTCTAGTTGTGGTTTGTCCAAACTCATCAATGTATCTTAACGCGGCCGCACGTGCGGACCGA
GCGGCCGCAggaacccctagtgatggagttggccactccctctctgcgcgctcgctcgctcactgaggccgcccgggcaa
agcccgggcgtcgggcgacctttggtcgcccggcctcagtgagcgagcgagcgcgcagagagggagtggccaa
> AAV.LP1-hIL-12-3′-riboswitch (Seq ID No. 50): signal peptide, IL12 p40-(G4S)3-
p35; carrying the 4 nt deletion in the ITR
CCTGCAGGCAGCTGCGCGCTCGCTCGCTCACTGAGGCCGCCCGGGCAAAGCCCGGGCGTCGGG
CGACCTTTGGTCGCCCGGCCTCAGTGAGCGAGCGAGCGCGCAGAGAGGGAGTGGCCAACTCCA
TCACTAGGGGTTCCTGCGGCCGCACGCGTTCGACCCCCTAAAATGGGCAAACATTGCAAGCAAA
CAGCAAACACACAGCCCTCCCTGCCTGCTGACCTTGGAGCTGGGGCAGAGGTCAGAGACCTCTC
TGGGCCCATGCCACCTCCAACATCCACTCGACCCCTTGGAATTTCGGTGGAGAGGAGCAGAGGT
TGTCCTGGCGTGGTTTAGGTAGTGTGAGAGGGGAATGACTCCTTTCGGTAAGTGCAGTGGAAG
CTGTACACTGCCCAGGCAAAGCGTCCGGGCAGCGTAGGCGGGCGACTCAGATCCCAGCCAGTG
GACTTAGCCCCTGTTTGCTCCTCCGATAACTGGGGTGACCTTGGTTAATATTCACCAGCAGCCTC
CCCCGTTGCCCCTCTGGATCCACTGCTTAAATACGGACGAGGACAGGGCCCTGTCTCCTCAGCTT
CAGGCACCACCACTGACCTGGGACAGTGAATCCGGACTCTAAGAGAATTCCCCGGACCGGTGG
ATCCGCCACCATGGGCTGGTCCTGCATCATTCTGTTTCTGGTGGCCACAGCCACCGGCGTGCACT
CTATTTGGGAGCTGAAGAAAGACGTGTACGTGGTCGAGCTGGACTGGTATCCTGATGCTCCCG
GCGAAATGGTGGTGCTGACCTGTGATACACCCGAAGAGGACGGCATCACATGGACACTGGATC
AGTCTAGCGAGGTGCTCGGCAGCGGCAAGACACTGACCATCCAAGTGAAAGAGTTTGGCGACG
CCGGCCAGTACACATGTCACAAAGGCGGAGAGGTGCTGAGCCATTCTCTGCTGCTGCTCCACAA
GAAAGAGGATGGCATTTGGAGCACCGACATCCTGAAGGACCAGAAAGAGCCCAAGAACAAGA
CCTTCCTGAGATGCGAGGCCAAGAACTACAGCGGCAGATTCACCTGTTGGTGGCTGACCACCAT
CAGCACCGATCTGACCTTCAGCGTGAAGTCCAGCAGAGGCAGCTCTGATCCTCAAGGCGTTACA
TGTGGCGCCGCTACACTGTCTGCCGAAAGAGTGCGGGGCGACAACAAAGAGTACGAGTACAGC
GTCGAGTGCCAAGAGGATTCTGCCTGTCCTGCCGCCGAGGAATCTCTGCCTATCGAAGTGATGG
TGGACGCCGTGCACAAGCTGAAGTACGAGAACTACACCAGCAGCTTTTTCATCCGGGACATCAT
CAAGCCCGATCCTCCAAAGAACCTGCAGCTGAAGCCCCTGAAGAACAGCAGACAGGTGGAAGT
GTCTTGGGAGTACCCCGACACATGGTCTACCCCTCACAGCTACTTCAGCCTGACCTTCTGTGTGC
AAGTGCAGGGCAAGTCCAAGCGCGAGAAGAAAGATCGGGTGTTCACCGACAAGACCAGCGCC
ACCGTGATCTGCAGAAAGAACGCCAGCATCAGCGTGCGCGCTCAGGATAGGTACTACAGCAGC
TCTTGGAGCGAGTGGGCCTCTGTTCCTTGTTCTGGCGGCGGAGGAAGCGGAGGCGGAGGATCT
GGTGGTGGTGGAAGTAGAAACCTGCCAGTGGCTACCCCTGATCCGGGCATGTTTCCTTGTCTGC
ACCACAGCCAGAACCTGCTGAGAGCCGTGTCCAATATGCTGCAGAAGGCCCGGCAGACCCTTG
AGTTCTACCCTTGCACCAGCGAGGAAATCGACCACGAGGACATCACCAAGGATAAGACCAGCA
CCGTGGAAGCCTGCCTGCCTCTGGAACTGACCAAGAACGAGTCCTGCCTGAACAGCCGGGAAA
CCAGCTTCATCACCAATGGCAGCTGTCTGGCCAGCAGAAAGACCTCCTTCATGATGGCCCTGTG
CCTGAGCAGCATCTACGAGGACCTGAAGATGTATCAGGTCGAGTTCAAGACCATGAACGCCAA
GCTGCTGATGGACCCCAAGAGACAGATTTTCCTCGACCAGAACATGCTGGCCGTGATCGATGAA
CTGATGCAGGCCCTGAACTTCAACAGCGAGACAGTGCCCCAGAAGTCCTCTCTGGAAGAACCCG
ACTTCTACAAGACCAAGATCAAGCTGTGCATCCTGCTGCACGCCTTCAGAATCAGGGCCGTGAC
CATCGACAGAGTGATGAGCTATCTGAACGCCAGCTGAAAGCTTGGTACCCAAACAAACAAAGG
CGCGTCCTGGATTCGTGGTAAAACATACCAGATTTCGATCTGGAGAGGTGAAGAATACGACCAC
CTACTACATCCAGCTGATGAGTCCCAAATAGGACGAAACGCGCTCAAACAAACAAAAGATCTCT
TGTTTATTGCAGCTTATAATGGTTACAAATAAAGCAATAGCATCACAAATTTCACAAATAAAGCA
TTTTTTTCACTGCATTCTAGTTGTGGTTTGTCCAAACTCATCAATGTATCTTAACGCGGCCGCACG
TGCGGACCGAGCGGCCGCAGGAACCCCTAGTGATGGAGTTGGCCACTCCCTCTCTGCGCGCTC
GCTCGCTCACTGAGGCCGGGCGACCAAAGGTCGCCCGACGCCCGGGCTTTGCCCGGGCGGCCT
CAGTGAGCGAGCGAGCGCGCAGCTGCCTGCAGG
> AAV.LP1-hIL-12-3′-riboswitch (Seq ID No. 51) signal peptide, IL12 p35-(G4S)3-
p40; carrying the 4 nt deletion in the ITR
CCTGCAGGCAGCTGCGCGCTCGCTCGCTCACTGAGGCCGCCCGGGCAAAGCCCGGGCGTCGGG
CGACCTTTGGTCGCCCGGCCTCAGTGAGCGAGCGAGCGCGCAGAGAGGGAGTGGCCAACTCCA
TCACTAGGGGTTCCTGCGGCCGCACGCGTTCGACCCCCTAAAATGGGCAAACATTGCAAGCAAA
CAGCAAACACACAGCCCTCCCTGCCTGCTGACCTTGGAGCTGGGGCAGAGGTCAGAGACCTCTC
TGGGCCCATGCCACCTCCAACATCCACTCGACCCCTTGGAATTTCGGTGGAGAGGAGCAGAGGT
TGTCCTGGCGTGGTTTAGGTAGTGTGAGAGGGGAATGACTCCTTTCGGTAAGTGCAGTGGAAG
CTGTACACTGCCCAGGCAAAGCGTCCGGGCAGCGTAGGCGGGCGACTCAGATCCCAGCCAGTG
GACTTAGCCCCTGTTTGCTCCTCCGATAACTGGGGTGACCTTGGTTAATATTCACCAGCAGCCTC
CCCCGTTGCCCCTCTGGATCCACTGCTTAAATACGGACGAGGACAGGGCCCTGTCTCCTCAGCTT
CAGGCACCACCACTGACCTGGGACAGTGAATCCGGACTCTAAGAGAATTCCCCGGACCGGTGG
ATCCGCCACCATGGGCTGGTCCTGCATCATTCTGTTTCTGGTGGCCACAGCCACCGGCGTGCACT
CTAGAAACCTGCCAGTGGCTACCCCTGATCCGGGCATGTTTCCTTGTCTGCACCACAGCCAGAAC
CTGCTGAGAGCCGTGTCCAATATGCTGCAGAAGGCCCGGCAGACCCTTGAGTTCTACCCTTGCA
CCAGCGAGGAAATCGACCACGAGGACATCACCAAGGATAAGACCAGCACCGTGGAAGCCTGCC
TGCCTCTGGAACTGACCAAGAACGAGTCCTGCCTGAACAGCCGGGAAACCAGCTTCATCACCAA
TGGCAGCTGTCTGGCCAGCAGAAAGACCTCCTTCATGATGGCCCTGTGCCTGAGCAGCATCTAC
GAGGACCTGAAGATGTATCAGGTCGAGTTCAAGACCATGAACGCCAAGCTGCTGATGGACCCC
AAGAGACAGATTTTCCTCGACCAGAACATGCTGGCCGTGATCGATGAACTGATGCAGGCCCTGA
ACTTCAACAGCGAGACAGTGCCCCAGAAGTCCTCTCTGGAAGAACCCGACTTCTACAAGACCAA
GATCAAGCTGTGCATCCTGCTGCACGCCTTCAGAATCAGGGCCGTGACCATCGACAGAGTGATG
AGCTATCTGAACGCCAGCGGCGGCGGAGGAAGCGGAGGCGGAGGATCTGGTGGTGGTGGAA
GTATTTGGGAGCTGAAGAAAGACGTGTACGTGGTCGAGCTGGACTGGTATCCTGATGCTCCCG
GCGAAATGGTGGTGCTGACCTGTGATACACCCGAAGAGGACGGCATCACATGGACACTGGATC
AGTCTAGCGAGGTGCTCGGCAGCGGCAAGACACTGACCATCCAAGTGAAAGAGTTTGGCGACG
CCGGCCAGTACACATGTCACAAAGGCGGAGAGGTGCTGAGCCATTCTCTGCTGCTGCTCCACAA
GAAAGAGGATGGCATTTGGAGCACCGACATCCTGAAGGACCAGAAAGAGCCCAAGAACAAGA
CCTTCCTGAGATGCGAGGCCAAGAACTACAGCGGCAGATTCACCTGTTGGTGGCTGACCACCAT
CAGCACCGATCTGACCTTCAGCGTGAAGTCCAGCAGAGGCAGCTCTGATCCTCAAGGCGTTACA
TGTGGCGCCGCTACACTGTCTGCCGAAAGAGTGCGGGGCGACAACAAAGAGTACGAGTACAGC
GTCGAGTGCCAAGAGGATTCTGCCTGTCCTGCCGCCGAGGAATCTCTGCCTATCGAAGTGATGG
TGGACGCCGTGCACAAGCTGAAGTACGAGAACTACACCAGCAGCTTTTTCATCCGGGACATCAT
CAAGCCCGATCCTCCAAAGAACCTGCAGCTGAAGCCCCTGAAGAACAGCAGACAGGTGGAAGT
GTCTTGGGAGTACCCCGACACATGGTCTACCCCTCACAGCTACTTCAGCCTGACCTTCTGTGTGC
AAGTGCAGGGCAAGTCCAAGCGCGAGAAGAAAGATCGGGTGTTCACCGACAAGACCAGCGCC
ACCGTGATCTGCAGAAAGAACGCCAGCATCAGCGTGCGCGCTCAGGATAGGTACTACAGCAGC
TCTTGGAGCGAGTGGGCCTCTGTTCCTTGTTCTTGAAAGCTTGGTACCCAAACAAACAAAGGCG
CGTCCTGGATTCGTGGTAAAACATACCAGATTTCGATCTGGAGAGGTGAAGAATACGACCACCT
ACTACATCCAGCTGATGAGTCCCAAATAGGACGAAACGCGCTCAAACAAACAAAAGATCTCTTG
TTTATTGCAGCTTATAATGGTTACAAATAAAGCAATAGCATCACAAATTTCACAAATAAAGCATT
TTTTTCACTGCATTCTAGTTGTGGTTTGTCCAAACTCATCAATGTATCTTAACGCGGCCGCACGTG
CGGACCGAGCGGCCGCAGGAACCCCTAGTGATGGAGTTGGCCACTCCCTCTCTGCGCGCTCGCT
CGCTCACTGAGGCCGGGCGACCAAAGGTCGCCCGACGCCCGGGCTTTGCCCGGGCGGCCTCAG
TGAGCGAGCGAGCGCGCAGCTGCCTGCAGG
>human-immunoglobulin-signal-sequence (Seq ID No. 52)
mgwsciilflvatatgvhs
>signal-sequence-p35-p40-hulL12 (Seq ID No. 53)
mgwsciilflvatatgvhsrnlpvatpdpgmfpclhhsqnllravsnmlqkarqtlefypctseeidhedit
kdktstveaclpleltknesclnsretsfitngsclasrktsfmmalclssiyedlkmyqvefktmnakllm
dpkrqifldqnmlavidelmqalnfnsetvpqkssleepdfyktkiklcillhafriravtidrvmsylna
sggggggggsggggsiwelkkdvyvveldwypdapgemvvltcdtpeedgitwtldqssevlgsgktltiq
vkefgdagqytchkggevlshsllllhkkedgiwstdilkdqkepknktflrceaknysgrftcwwlttistd
ltfsvkssrgssdpqgvtcgaatlsaervrgdnkeyeysvecqedsacpaaeeslpievmvdavhklkyeny
tssffirdiikpdppknlqlkplknsrqvevsweypdtwstphsyfsltfcvqvqgkskrekkdrvftdktsa
tvicrknasisvraqdryyssswsewasvpcs
>p35-p40-huIL12 (after signal sequence cleavage) (Seq ID No. 54)
rnlpvatpdpgmfpclhhsqnllravsnmlqkarqtlefypctseeidheditkdktstveaclpleltknes
clnsretsfitngsclasrktsfmmalclssiyedlkmyqvefktmnakllmdpkrqifldqnmlavidelmq
alnfnsetvpqkssleepdfyktkiklcillhafriravtidrvmsylnasggggsggggggggsiwelkkdv
yvveldwypdapgemvvltcdtpeedgitwtldqssevlgsgktltiqvkefgdagqytchkggevlshsllllh
kkedgiwstdilkdqkepknktflrceaknysgrftcwwlttistdltfsvkssrgssdpqgvtcgaatlsa
ervrgdnkeyeysvecqedsacpaaeeslpievmvdavhklkyenytssffirdiikpdppknlqlkplknsrq
vevsweypdtwstphsyfsltfcvqvqgkskrekkdrvftdktsatvicrknasisvraqdryyssswsewasvpcs
>signal-sequence-p35-p40-hulL12-GS_linker (Seq ID No. 55)
mgwsciilflvatatgvhsrnlpvatpdpgmfpclhhsqnllravsnmlqkarqtlefypctseeidhedit
kdktstveaclpleltknesclnsretsfitngsclasrktsfmmalclssiyedlkmyqvefktmnakllm
dpkrqifldqnmlavidelmqalnfnsetvpqkssleepdfyktkiklcillhafriravtidrvmsylna
sggggggggsggggsiwelkkdvyvveldwypdapgemvvltcdtpeedgitwtldqssevlgsgktltiq
vkefgdagqytchkggevlshsllllhkkedgiwstdilkdqkepknktflrceaknysgrftcwwlttistd
ltfsvkssrgssdpqgvtcgaatlsaervrgdnkeyeysvecqedsacpaaeeslpievmvdavhklkyeny
tssffirdiikpdppknlqlkplknsrqvevsweypdtwstphsyfsltfcvqvqgkskrekkdrvftdktsa
tvicrknasisvraqdryyssswsewasvpcsGGGGSGGGS
>p35-p40-huIL12-GS_linker (after signal sequence cleavage) (Seq ID No. 56)
rnlpvatpdpgmfpclhhsqnllravsnmlqkarqtlefypctseeidheditkdktstveaclpleltknes
clnsretsfitngsclasrktsfmmalclssiyedlkmyqvefktmnakllmdpkrqifldqnmlavidelmq
alnfnsetvpqkssleepdfyktkiklcillhafriravtidrvmsylnasggggsggggsggggsiwelkkdv
yvveldwypdapgemvvltcdtpeedgitwtldqssevlgsgktltiqvkefgdagqytchkggevlshsllllh
kkedgiwstdilkdqkepknktflrceaknysgrftcwwlttistdltfsvkssrgssdpqgvtcgaatlsa
ervrgdnkeyeysvecqedsacpaaeeslpievmvdavhklkyenytssffirdiikpdppknlqlkplknsrq
vevsweypdtwstphsyfsltfcvqvqgkskrekkdrvftdktsatvicrknasisvraqdryyssswsewasvpcs
GGGGSGGGS
> AAV.LP1-hIL-12-3′-riboswitch (Seq ID No. 57): signal peptide, IL12 p40-(G4S)3-p35;
AAV2-WT ITR
ttggccactccctctctgcgcgctcgctcgctcactgaggccgggcgaccaaaggtcgcccgacgcccgggctttgcccgggc
ggcctcagtgagcgagcgagcgcgcagagagggagtggccaactccatcactaggggttccTGCGGCCGCACGCGTT
CGACCCCCTAAAATGGGCAAACATTGCAAGCAAACAGCAAACACACAGCCCTCCCTGCCTGCTG
ACCTTGGAGCTGGGGCAGAGGTCAGAGACCTCTCTGGGCCCATGCCACCTCCAACATCCACTCG
ACCCCTTGGAATTTCGGTGGAGAGGAGCAGAGGTTGTCCTGGCGTGGTTTAGGTAGTGTGAGA
GGGGAATGACTCCTTTCGGTAAGTGCAGTGGAAGCTGTACACTGCCCAGGCAAAGCGTCCGGG
CAGCGTAGGCGGGCGACTCAGATCCCAGCCAGTGGACTTAGCCCCTGTTTGCTCCTCCGATAAC
TGGGGTGACCTTGGTTAATATTCACCAGCAGCCTCCCCCGTTGCCCCTCTGGATCCACTGCTTAA
ATACGGACGAGGACAGGGCCCTGTCTCCTCAGCTTCAGGCACCACCACTGACCTGGGACAGTG
AATCCGGACTCTAAGAGAATTCCCCGGACCGGTGGATCCGCCACCATGGGCTGGTCCTGCATCA
TTCTGTTTCTGGTGGCCACAGCCACCGGCGTGCACTCTATTTGGGAGCTGAAGAAAGACGTGTA
CGTGGTCGAGCTGGACTGGTATCCTGATGCTCCCGGCGAAATGGTGGTGCTGACCTGTGATAC
ACCCGAAGAGGACGGCATCACATGGACACTGGATCAGTCTAGCGAGGTGCTCGGCAGCGGCA
AGACACTGACCATCCAAGTGAAAGAGTTTGGCGACGCCGGCCAGTACACATGTCACAAAGGCG
GAGAGGTGCTGAGCCATTCTCTGCTGCTGCTCCACAAGAAAGAGGATGGCATTTGGAGCACCG
ACATCCTGAAGGACCAGAAAGAGCCCAAGAACAAGACCTTCCTGAGATGCGAGGCCAAGAACT
ACAGCGGCAGATTCACCTGTTGGTGGCTGACCACCATCAGCACCGATCTGACCTTCAGCGTGAA
GTCCAGCAGAGGCAGCTCTGATCCTCAAGGCGTTACATGTGGCGCCGCTACACTGTCTGCCGAA
AGAGTGCGGGGCGACAACAAAGAGTACGAGTACAGCGTCGAGTGCCAAGAGGATTCTGCCTG
TCCTGCCGCCGAGGAATCTCTGCCTATCGAAGTGATGGTGGACGCCGTGCACAAGCTGAAGTAC
GAGAACTACACCAGCAGCTTTTTCATCCGGGACATCATCAAGCCCGATCCTCCAAAGAACCTGC
AGCTGAAGCCCCTGAAGAACAGCAGACAGGTGGAAGTGTCTTGGGAGTACCCCGACACATGGT
CTACCCCTCACAGCTACTTCAGCCTGACCTTCTGTGTGCAAGTGCAGGGCAAGTCCAAGCGCGA
GAAGAAAGATCGGGTGTTCACCGACAAGACCAGCGCCACCGTGATCTGCAGAAAGAACGCCAG
CATCAGCGTGCGCGCTCAGGATAGGTACTACAGCAGCTCTTGGAGCGAGTGGGCCTCTGTTCCT
TGTTCTGGCGGCGGAGGAAGCGGAGGCGGAGGATCTGGTGGTGGTGGAAGTAGAAACCTGCC
AGTGGCTACCCCTGATCCGGGCATGTTTCCTTGTCTGCACCACAGCCAGAACCTGCTGAGAGCC
GTGTCCAATATGCTGCAGAAGGCCCGGCAGACCCTTGAGTTCTACCCTTGCACCAGCGAGGAAA
TCGACCACGAGGACATCACCAAGGATAAGACCAGCACCGTGGAAGCCTGCCTGCCTCTGGAAC
TGACCAAGAACGAGTCCTGCCTGAACAGCCGGGAAACCAGCTTCATCACCAATGGCAGCTGTCT
GGCCAGCAGAAAGACCTCCTTCATGATGGCCCTGTGCCTGAGCAGCATCTACGAGGACCTGAA
GATGTATCAGGTCGAGTTCAAGACCATGAACGCCAAGCTGCTGATGGACCCCAAGAGACAGAT
TTTCCTCGACCAGAACATGCTGGCCGTGATCGATGAACTGATGCAGGCCCTGAACTTCAACAGC
GAGACAGTGCCCCAGAAGTCCTCTCTGGAAGAACCCGACTTCTACAAGACCAAGATCAAGCTGT
GCATCCTGCTGCACGCCTTCAGAATCAGGGCCGTGACCATCGACAGAGTGATGAGCTATCTGAA
CGCCAGCTGAAAGCTTGGTACCCAAACAAACAAAGGCGCGTCCTGGATTCGTGGTAAAACATA
CCAGATTTCGATCTGGAGAGGTGAAGAATACGACCACCTACTACATCCAGCTGATGAGTCCCAA
ATAGGACGAAACGCGCTCAAACAAACAAAAGATCTCTTGTTTATTGCAGCTTATAATGGTTACA
AATAAAGCAATAGCATCACAAATTTCACAAATAAAGCATTTTTTTCACTGCATTCTAGTTGTGGTT
TGTCCAAACTCATCAATGTATCTTAACGCGGCCGCACGTGCGGACCGAGCGGCCGCAGGAACCC
CTAGTGATGGAGTTGGCCACTCCCTCTCTGCGCGCTCGCTCGCTCACTGAGGCCGCCCGGGCAA
AGCCCGGGCGTCGGGCGACCTTTGGTCGCCCGGCCTCAGTGAGCGAGCGAGCGCGCAGAGAG
GGAGTGGCCAA
> pAAV.LP1-hIL-12-3′-riboswitch (Seq ID No. 58); signal peptide, IL12 p35-(G4S)3-p40;
AAV2-WT ITR
ttggccactccctctctgcgcgctcgctcgctcactgaggccgggcgaccaaaggtcgcccgacgcccgggctttgcccgggc
ggcctcagtgagcgagcgagcgcgcagagagggagtggccaactccatcactaggggttccTGCGGCCGCACGCGTT
CGACCCCCTAAAATGGGCAAACATTGCAAGCAAACAGCAAACACACAGCCCTCCCTGCCTGCTG
ACCTTGGAGCTGGGGCAGAGGTCAGAGACCTCTCTGGGCCCATGCCACCTCCAACATCCACTCG
ACCCCTTGGAATTTCGGTGGAGAGGAGCAGAGGTTGTCCTGGCGTGGTTTAGGTAGTGTGAGA
GGGGAATGACTCCTTTCGGTAAGTGCAGTGGAAGCTGTACACTGCCCAGGCAAAGCGTCCGGG
CAGCGTAGGCGGGCGACTCAGATCCCAGCCAGTGGACTTAGCCCCTGTTTGCTCCTCCGATAAC
TGGGGTGACCTTGGTTAATATTCACCAGCAGCCTCCCCCGTTGCCCCTCTGGATCCACTGCTTAA
ATACGGACGAGGACAGGGCCCTGTCTCCTCAGCTTCAGGCACCACCACTGACCTGGGACAGTG
AATCCGGACTCTAAGAGAATTCCCCGGACCGGTGGATCCGCCACCATGGGCTGGTCCTGCATCA
TTCTGTTTCTGGTGGCCACAGCCACCGGCGTGCACTCTAGAAACCTGCCAGTGGCTACCCCTGAT
CCGGGCATGTTTCCTTGTCTGCACCACAGCCAGAACCTGCTGAGAGCCGTGTCCAATATGCTGC
AGAAGGCCCGGCAGACCCTTGAGTTCTACCCTTGCACCAGCGAGGAAATCGACCACGAGGACA
TCACCAAGGATAAGACCAGCACCGTGGAAGCCTGCCTGCCTCTGGAACTGACCAAGAACGAGT
CCTGCCTGAACAGCCGGGAAACCAGCTTCATCACCAATGGCAGCTGTCTGGCCAGCAGAAAGA
CCTCCTTCATGATGGCCCTGTGCCTGAGCAGCATCTACGAGGACCTGAAGATGTATCAGGTCGA
GTTCAAGACCATGAACGCCAAGCTGCTGATGGACCCCAAGAGACAGATTTTCCTCGACCAGAAC
ATGCTGGCCGTGATCGATGAACTGATGCAGGCCCTGAACTTCAACAGCGAGACAGTGCCCCAG
AAGTCCTCTCTGGAAGAACCCGACTTCTACAAGACCAAGATCAAGCTGTGCATCCTGCTGCACG
CCTTCAGAATCAGGGCCGTGACCATCGACAGAGTGATGAGCTATCTGAACGCCAGCGGCGGCG
GAGGAAGCGGAGGCGGAGGATCTGGTGGTGGTGGAAGTATTTGGGAGCTGAAGAAAGACGT
GTACGTGGTCGAGCTGGACTGGTATCCTGATGCTCCCGGCGAAATGGTGGTGCTGACCTGTGA
TACACCCGAAGAGGACGGCATCACATGGACACTGGATCAGTCTAGCGAGGTGCTCGGCAGCGG
CAAGACACTGACCATCCAAGTGAAAGAGTTTGGCGACGCCGGCCAGTACACATGTCACAAAGG
CGGAGAGGTGCTGAGCCATTCTCTGCTGCTGCTCCACAAGAAAGAGGATGGCATTTGGAGCAC
CGACATCCTGAAGGACCAGAAAGAGCCCAAGAACAAGACCTTCCTGAGATGCGAGGCCAAGAA
CTACAGCGGCAGATTCACCTGTTGGTGGCTGACCACCATCAGCACCGATCTGACCTTCAGCGTG
AAGTCCAGCAGAGGCAGCTCTGATCCTCAAGGCGTTACATGTGGCGCCGCTACACTGTCTGCCG
AAAGAGTGCGGGGCGACAACAAAGAGTACGAGTACAGCGTCGAGTGCCAAGAGGATTCTGCC
TGTCCTGCCGCCGAGGAATCTCTGCCTATCGAAGTGATGGTGGACGCCGTGCACAAGCTGAAG
TACGAGAACTACACCAGCAGCTTTTTCATCCGGGACATCATCAAGCCCGATCCTCCAAAGAACCT
GCAGCTGAAGCCCCTGAAGAACAGCAGACAGGTGGAAGTGTCTTGGGAGTACCCCGACACATG
GTCTACCCCTCACAGCTACTTCAGCCTGACCTTCTGTGTGCAAGTGCAGGGCAAGTCCAAGCGC
GAGAAGAAAGATCGGGTGTTCACCGACAAGACCAGCGCCACCGTGATCTGCAGAAAGAACGCC
AGCATCAGCGTGCGCGCTCAGGATAGGTACTACAGCAGCTCTTGGAGCGAGTGGGCCTCTGTT
CCTTGTTCTTGAAAGCTTGGTACCCAAACAAACAAAGGCGCGTCCTGGATTCGTGGTAAAACAT
ACCAGATTTCGATCTGGAGAGGTGAAGAATACGACCACCTACTACATCCAGCTGATGAGTCCCA
AATAGGACGAAACGCGCTCAAACAAACAAAAGATCTCTTGTTTATTGCAGCTTATAATGGTTAC
AAATAAAGCAATAGCATCACAAATTTCACAAATAAAGCATTTTTTTCACTGCATTCTAGTTGTGG
TTTGTCCAAACTCATCAATGTATCTTAACGCGGCCGCACGTGCGGACCGAGCGGCCGCAGGAAC
CCCTAGTGATGGAGTTGGCCACTCCCTCTCTGCGCGCTCGCTCGCTCACTGAGGCCGCCCGGGC
AAAGCCCGGGCGTCGGGCGACCTTTGGTCGCCCGGCCTCAGTGAGCGAGCGAGCGCGCAGAG
AGGGAGTGGCCAA
> AAV.LP1_SV40-hIL-12-3′-riboswitch (Seq ID No. 59): signal peptide, IL12 p40-(G4S)3-
p35; AAV2-WT ITR
ttggccactccctctctgcgcgctcgctcgctcactgaggccgggcgaccaaaggtcgcccgacgcccgggctttgcccgggc
ggcctcagtgagcgagcgagcgcgcagagagggagtggccaactccatcactaggggttccTGCGGCCGCACGCGTC
CCTAAAATGGGCAAACATTGCAAGCAGCAAACAGCAAACACACAGCCCTCCCTGCCTGCTGACC
TTGGAGCTGGGGCAGAGGTCAGAGACCTCTCTGGGCCCATGCCACCTCCAACATCCACTCGACC
CCTTGGAATTTCGGTGGAGAGGAGCAGAGGTTGTCCTGGCGTGGTTTAGGTAGTGTGAGAGG
GTCCGGGTTCAAAACCACTTGCTGGGTGGGGAGTCGTCAGTAAGTGGCTATGCCCCGACCCCG
AAGCCTGTTTCCCCATCTGTACAATGGAAATGATAAAGACGCCCATCTGATAGGGAATGACTCC
TTTCGGTAAGTGCAGTGGAAGCTGTACACTGCCCAGGCAAAGCGTCCGGGCAGCGTAGGCGGG
CGACTCAGATCCCAGCCAGTGGACTTAGCCCCTGTTTGCTCCTCCGATAACTGGGGTGACCTTG
GTTAATATTCACCAGCAGCCTCCCCCGTTGCCCCTCTGGATCCACTGCTTAAATACGGACGAGGA
CAGGGCCCTGTCTCCTCAGCTTCAGGCACCACCACTGACCTGGGACAGTGAATCCGGACTCTAA
GGTAAATATAAAATTTTTAAGTGTATAATGTGTTAAACTACTGATTCTAATTGTTTCTCTATTTTA
GATTCCAACCTTTGGAACTGAGAATTCCCCGGACCGGTGGATCCGCCACCATGGGCTGGTCCTG
CATCATTCTGTTTCTGGTGGCCACAGCCACCGGCGTGCACTCTATTTGGGAGCTGAAGAAAGAC
GTGTACGTGGTCGAGCTGGACTGGTATCCTGATGCTCCCGGCGAAATGGTGGTGCTGACCTGT
GATACACCCGAAGAGGACGGCATCACATGGACACTGGATCAGTCTAGCGAGGTGCTCGGCAGC
GGCAAGACACTGACCATCCAAGTGAAAGAGTTTGGCGACGCCGGCCAGTACACATGTCACAAA
GGCGGAGAGGTGCTGAGCCATTCTCTGCTGCTGCTCCACAAGAAAGAGGATGGCATTTGGAGC
ACCGACATCCTGAAGGACCAGAAAGAGCCCAAGAACAAGACCTTCCTGAGATGCGAGGCCAAG
AACTACAGCGGCAGATTCACCTGTTGGTGGCTGACCACCATCAGCACCGATCTGACCTTCAGCG
TGAAGTCCAGCAGAGGCAGCTCTGATCCTCAAGGCGTTACATGTGGCGCCGCTACACTGTCTGC
CGAAAGAGTGCGGGGCGACAACAAAGAGTACGAGTACAGCGTCGAGTGCCAAGAGGATTCTG
CCTGTCCTGCCGCCGAGGAATCTCTGCCTATCGAAGTGATGGTGGACGCCGTGCACAAGCTGAA
GTACGAGAACTACACCAGCAGCTTTTTCATCCGGGACATCATCAAGCCCGATCCTCCAAAGAAC
CTGCAGCTGAAGCCCCTGAAGAACAGCAGACAGGTGGAAGTGTCTTGGGAGTACCCCGACACA
TGGTCTACCCCTCACAGCTACTTCAGCCTGACCTTCTGTGTGCAAGTGCAGGGCAAGTCCAAGC
GCGAGAAGAAAGATCGGGTGTTCACCGACAAGACCAGCGCCACCGTGATCTGCAGAAAGAAC
GCCAGCATCAGCGTGCGCGCTCAGGATAGGTACTACAGCAGCTCTTGGAGCGAGTGGGCCTCT
GTTCCTTGTTCTGGCGGCGGAGGAAGCGGAGGCGGAGGATCTGGTGGTGGTGGAAGTAGAAA
CCTGCCAGTGGCTACCCCTGATCCGGGCATGTTTCCTTGTCTGCACCACAGCCAGAACCTGCTGA
GAGCCGTGTCCAATATGCTGCAGAAGGCCCGGCAGACCCTTGAGTTCTACCCTTGCACCAGCGA
GGAAATCGACCACGAGGACATCACCAAGGATAAGACCAGCACCGTGGAAGCCTGCCTGCCTCT
GGAACTGACCAAGAACGAGTCCTGCCTGAACAGCCGGGAAACCAGCTTCATCACCAATGGCAG
CTGTCTGGCCAGCAGAAAGACCTCCTTCATGATGGCCCTGTGCCTGAGCAGCATCTACGAGGAC
CTGAAGATGTATCAGGTCGAGTTCAAGACCATGAACGCCAAGCTGCTGATGGACCCCAAGAGA
CAGATTTTCCTCGACCAGAACATGCTGGCCGTGATCGATGAACTGATGCAGGCCCTGAACTTCA
ACAGCGAGACAGTGCCCCAGAAGTCCTCTCTGGAAGAACCCGACTTCTACAAGACCAAGATCAA
GCTGTGCATCCTGCTGCACGCCTTCAGAATCAGGGCCGTGACCATCGACAGAGTGATGAGCTAT
CTGAACGCCAGCTGAAAGCTTGGTACCCAAACAAACAAAGGCGCGTCCTGGATTCGTGGTAAA
ACATACCAGATTTCGATCTGGAGAGGTGAAGAATACGACCACCTACTACATCCAGCTGATGAGT
CCCAAATAGGACGAAACGCGCTCAAACAAACAAAAGATCTCTTGTTTATTGCAGCTTATAATGG
TTACAAATAAAGCAATAGCATCACAAATTTCACAAATAAAGCATTTTTTTCACTGCATTCTAGTTG
TGGTTTGTCCAAACTCATCAATGTATCTTAACGCGGCCGCACGTGCGGACCGAGCGGCCGCAGG
AACCCCTAGTGATGGAGTTGGCCACTCCCTCTCTGCGCGCTCGCTCGCTCACTGAGGCCGCCCG
GGCAAAGCCCGGGCGTCGGGCGACCTTTGGTCGCCCGGCCTCAGTGAGCGAGCGAGCGCGCA
GAGAGGGAGTGGCCAA
> pAAV.LP1_SV40-hIL-12-3′-riboswitch (Seq ID No. 60): signal peptide, IL12 p35-
(G4S)3-p40; AAV2-WT ITR
ttggccactccctctctgcgcgctcgctcgctcactgaggccgggcgaccaaaggtcgcccgacgcccgggctttgcccgggc
ggcctcagtgagcgagcgagcgcgcagagagggagtggccaactccatcactaggggttccTGCGGCCGCACGCGTC
CCTAAAATGGGCAAACATTGCAAGCAGCAAACAGCAAACACACAGCCCTCCCTGCCTGCTGACC
TTGGAGCTGGGGCAGAGGTCAGAGACCTCTCTGGGCCCATGCCACCTCCAACATCCACTCGACC
CCTTGGAATTTCGGTGGAGAGGAGCAGAGGTTGTCCTGGCGTGGTTTAGGTAGTGTGAGAGG
GTCCGGGTTCAAAACCACTTGCTGGGTGGGGAGTCGTCAGTAAGTGGCTATGCCCCGACCCCG
AAGCCTGTTTCCCCATCTGTACAATGGAAATGATAAAGACGCCCATCTGATAGGGAATGACTCC
TTTCGGTAAGTGCAGTGGAAGCTGTACACTGCCCAGGCAAAGCGTCCGGGCAGCGTAGGCGGG
CGACTCAGATCCCAGCCAGTGGACTTAGCCCCTGTTTGCTCCTCCGATAACTGGGGTGACCTTG
GTTAATATTCACCAGCAGCCTCCCCCGTTGCCCCTCTGGATCCACTGCTTAAATACGGACGAGGA
CAGGGCCCTGTCTCCTCAGCTTCAGGCACCACCACTGACCTGGGACAGTGAATCCGGACTCTAA
GGTAAATATAAAATTTTTAAGTGTATAATGTGTTAAACTACTGATTCTAATTGTTTCTCTATTTTA
GATTCCAACCTTTGGAACTGAGAATTCCCCGGACCGGTGGATCCGCCACCATGGGCTGGTCCTG
CATCATTCTGTTTCTGGTGGCCACAGCCACCGGCGTGCACTCTAGAAACCTGCCAGTGGCTACCC
CTGATCCGGGCATGTTTCCTTGTCTGCACCACAGCCAGAACCTGCTGAGAGCCGTGTCCAATAT
GCTGCAGAAGGCCCGGCAGACCCTTGAGTTCTACCCTTGCACCAGCGAGGAAATCGACCACGA
GGACATCACCAAGGATAAGACCAGCACCGTGGAAGCCTGCCTGCCTCTGGAACTGACCAAGAA
CGAGTCCTGCCTGAACAGCCGGGAAACCAGCTTCATCACCAATGGCAGCTGTCTGGCCAGCAG
AAAGACCTCCTTCATGATGGCCCTGTGCCTGAGCAGCATCTACGAGGACCTGAAGATGTATCAG
GTCGAGTTCAAGACCATGAACGCCAAGCTGCTGATGGACCCCAAGAGACAGATTTTCCTCGACC
AGAACATGCTGGCCGTGATCGATGAACTGATGCAGGCCCTGAACTTCAACAGCGAGACAGTGC
CCCAGAAGTCCTCTCTGGAAGAACCCGACTTCTACAAGACCAAGATCAAGCTGTGCATCCTGCT
GCACGCCTTCAGAATCAGGGCCGTGACCATCGACAGAGTGATGAGCTATCTGAACGCCAGCGG
CGGCGGAGGAAGCGGAGGCGGAGGATCTGGTGGTGGTGGAAGTATTTGGGAGCTGAAGAAA
GACGTGTACGTGGTCGAGCTGGACTGGTATCCTGATGCTCCCGGCGAAATGGTGGTGCTGACC
TGTGATACACCCGAAGAGGACGGCATCACATGGACACTGGATCAGTCTAGCGAGGTGCTCGGC
AGCGGCAAGACACTGACCATCCAAGTGAAAGAGTTTGGCGACGCCGGCCAGTACACATGTCAC
AAAGGCGGAGAGGTGCTGAGCCATTCTCTGCTGCTGCTCCACAAGAAAGAGGATGGCATTTGG
AGCACCGACATCCTGAAGGACCAGAAAGAGCCCAAGAACAAGACCTTCCTGAGATGCGAGGCC
AAGAACTACAGCGGCAGATTCACCTGTTGGTGGCTGACCACCATCAGCACCGATCTGACCTTCA
GCGTGAAGTCCAGCAGAGGCAGCTCTGATCCTCAAGGCGTTACATGTGGCGCCGCTACACTGTC
TGCCGAAAGAGTGCGGGGCGACAACAAAGAGTACGAGTACAGCGTCGAGTGCCAAGAGGATT
CTGCCTGTCCTGCCGCCGAGGAATCTCTGCCTATCGAAGTGATGGTGGACGCCGTGCACAAGCT
GAAGTACGAGAACTACACCAGCAGCTTTTTCATCCGGGACATCATCAAGCCCGATCCTCCAAAG
AACCTGCAGCTGAAGCCCCTGAAGAACAGCAGACAGGTGGAAGTGTCTTGGGAGTACCCCGAC
ACATGGTCTACCCCTCACAGCTACTTCAGCCTGACCTTCTGTGTGCAAGTGCAGGGCAAGTCCAA
GCGCGAGAAGAAAGATCGGGTGTTCACCGACAAGACCAGCGCCACCGTGATCTGCAGAAAGA
ACGCCAGCATCAGCGTGCGCGCTCAGGATAGGTACTACAGCAGCTCTTGGAGCGAGTGGGCCT
CTGTTCCTTGTTCTTGAAAGCTTGGTACCCAAACAAACAAAGGCGCGTCCTGGATTCGTGGTAA
AACATACCAGATTTCGATCTGGAGAGGTGAAGAATACGACCACCTACTACATCCAGCTGATGAG
TCCCAAATAGGACGAAACGCGCTCAAACAAACAAAAGATCTCTTGTTTATTGCAGCTTATAATG
GTTACAAATAAAGCAATAGCATCACAAATTTCACAAATAAAGCATTTTTTTCACTGCATTCTAGTT
GTGGTTTGTCCAAACTCATCAATGTATCTTAACGCGGCCGCACGTGCGGACCGAGCGGCCGCAG
GAACCCCTAGTGATGGAGTTGGCCACTCCCTCTCTGCGCGCTCGCTCGCTCACTGAGGCCGCCC
GGGCAAAGCCCGGGCGTCGGGCGACCTTTGGTCGCCCGGCCTCAGTGAGCGAGCGAGCGCGC
AGAGAGGGAGTGGCCAA
> AAV.LP1_SV40-hIL-12-3′-riboswitch (Seq ID No. 61): signal peptide, IL12 p40-(G4S)3-
p35; carrying the 4 nt deletion in the ITR
CCTGCAGGCAGCTGCGCGCTCGCTCGCTCACTGAGGCCGCCCGGGCAAAGCCCGGGCGTCGGG
CGACCTTTGGTCGCCCGGCCTCAGTGAGCGAGCGAGCGCGCAGAGAGGGAGTGGCCAACTCCA
TCACTAGGGGTTCCTGCGGCCGCACGCGTCCCTAAAATGGGCAAACATTGCAAGCAGCAAACA
GCAAACACACAGCCCTCCCTGCCTGCTGACCTTGGAGCTGGGGCAGAGGTCAGAGACCTCTCTG
GGCCCATGCCACCTCCAACATCCACTCGACCCCTTGGAATTTCGGTGGAGAGGAGCAGAGGTTG
TCCTGGCGTGGTTTAGGTAGTGTGAGAGGGTCCGGGTTCAAAACCACTTGCTGGGTGGGGAGT
CGTCAGTAAGTGGCTATGCCCCGACCCCGAAGCCTGTTTCCCCATCTGTACAATGGAAATGATA
AAGACGCCCATCTGATAGGGAATGACTCCTTTCGGTAAGTGCAGTGGAAGCTGTACACTGCCCA
GGCAAAGCGTCCGGGCAGCGTAGGCGGGCGACTCAGATCCCAGCCAGTGGACTTAGCCCCTGT
TTGCTCCTCCGATAACTGGGGTGACCTTGGTTAATATTCACCAGCAGCCTCCCCCGTTGCCCCTC
TGGATCCACTGCTTAAATACGGACGAGGACAGGGCCCTGTCTCCTCAGCTTCAGGCACCACCAC
TGACCTGGGACAGTGAATCCGGACTCTAAGGTAAATATAAAATTTTTAAGTGTATAATGTGTTA
AACTACTGATTCTAATTGTTTCTCTATTTTAGATTCCAACCTTTGGAACTGAGAATTCCCCGGACC
GGTGGATCCGCCACCATGGGCTGGTCCTGCATCATTCTGTTTCTGGTGGCCACAGCCACCGGCG
TGCACTCTATTTGGGAGCTGAAGAAAGACGTGTACGTGGTCGAGCTGGACTGGTATCCTGATG
CTCCCGGCGAAATGGTGGTGCTGACCTGTGATACACCCGAAGAGGACGGCATCACATGGACAC
TGGATCAGTCTAGCGAGGTGCTCGGCAGCGGCAAGACACTGACCATCCAAGTGAAAGAGTTTG
GCGACGCCGGCCAGTACACATGTCACAAAGGCGGAGAGGTGCTGAGCCATTCTCTGCTGCTGC
TCCACAAGAAAGAGGATGGCATTTGGAGCACCGACATCCTGAAGGACCAGAAAGAGCCCAAGA
ACAAGACCTTCCTGAGATGCGAGGCCAAGAACTACAGCGGCAGATTCACCTGTTGGTGGCTGA
CCACCATCAGCACCGATCTGACCTTCAGCGTGAAGTCCAGCAGAGGCAGCTCTGATCCTCAAGG
CGTTACATGTGGCGCCGCTACACTGTCTGCCGAAAGAGTGCGGGGCGACAACAAAGAGTACGA
GTACAGCGTCGAGTGCCAAGAGGATTCTGCCTGTCCTGCCGCCGAGGAATCTCTGCCTATCGAA
GTGATGGTGGACGCCGTGCACAAGCTGAAGTACGAGAACTACACCAGCAGCTTTTTCATCCGG
GACATCATCAAGCCCGATCCTCCAAAGAACCTGCAGCTGAAGCCCCTGAAGAACAGCAGACAG
GTGGAAGTGTCTTGGGAGTACCCCGACACATGGTCTACCCCTCACAGCTACTTCAGCCTGACCTT
CTGTGTGCAAGTGCAGGGCAAGTCCAAGCGCGAGAAGAAAGATCGGGTGTTCACCGACAAGA
CCAGCGCCACCGTGATCTGCAGAAAGAACGCCAGCATCAGCGTGCGCGCTCAGGATAGGTACT
ACAGCAGCTCTTGGAGCGAGTGGGCCTCTGTTCCTTGTTCTGGCGGCGGAGGAAGCGGAGGCG
GAGGATCTGGTGGTGGTGGAAGTAGAAACCTGCCAGTGGCTACCCCTGATCCGGGCATGTTTC
CTTGTCTGCACCACAGCCAGAACCTGCTGAGAGCCGTGTCCAATATGCTGCAGAAGGCCCGGCA
GACCCTTGAGTTCTACCCTTGCACCAGCGAGGAAATCGACCACGAGGACATCACCAAGGATAA
GACCAGCACCGTGGAAGCCTGCCTGCCTCTGGAACTGACCAAGAACGAGTCCTGCCTGAACAG
CCGGGAAACCAGCTTCATCACCAATGGCAGCTGTCTGGCCAGCAGAAAGACCTCCTTCATGATG
GCCCTGTGCCTGAGCAGCATCTACGAGGACCTGAAGATGTATCAGGTCGAGTTCAAGACCATG
AACGCCAAGCTGCTGATGGACCCCAAGAGACAGATTTTCCTCGACCAGAACATGCTGGCCGTGA
TCGATGAACTGATGCAGGCCCTGAACTTCAACAGCGAGACAGTGCCCCAGAAGTCCTCTCTGGA
AGAACCCGACTTCTACAAGACCAAGATCAAGCTGTGCATCCTGCTGCACGCCTTCAGAATCAGG
GCCGTGACCATCGACAGAGTGATGAGCTATCTGAACGCCAGCTGAAAGCTTGGTACCCAAACA
AACAAAGGCGCGTCCTGGATTCGTGGTAAAACATACCAGATTTCGATCTGGAGAGGTGAAGAA
TACGACCACCTACTACATCCAGCTGATGAGTCCCAAATAGGACGAAACGCGCTCAAACAAACAA
AAGATCTCTTGTTTATTGCAGCTTATAATGGTTACAAATAAAGCAATAGCATCACAAATTTCACA
AATAAAGCATTTTTTTCACTGCATTCTAGTTGTGGTTTGTCCAAACTCATCAATGTATCTTAACGC
GGCCGCACGTGCGGACCGAGCGGCCGCAGGAACCCCTAGTGATGGAGTTGGCCACTCCCTCTC
TGCGCGCTCGCTCGCTCACTGAGGCCGGGCGACCAAAGGTCGCCCGACGCCCGGGCTTTGCCC
GGGCGGCCTCAGTGAGCGAGCGAGCGCGCAGCTGCCTGCAGG
> pAAV. LP1_SV40-hIL-12-3′riboswitch (SEQ ID No. 62); signal peptide, IL12
p35(G4S)3p40; carrying the 4 nt deletion in the ITR
CCTGCAGGCAGCTGCGCGCTCGCTCGCTCACTGAGGCCGCCCGGGCAAAGCCCGGGCGTCGGG
CGACCTTTGGTCGCCCGGCCTCAGTGAGCGAGCGAGCGCGCAGAGAGGGAGTGGCCAACTCCA
TCACTAGGGGTTCCTGCGGCCGCACGCGTCCCTAAAATGGGCAAACATTGCAAGCAGCAAACA
GCAAACACACAGCCCTCCCTGCCTGCTGACCTTGGAGCTGGGGCAGAGGTCAGAGACCTCTCTG
GGCCCATGCCACCTCCAACATCCACTCGACCCCTTGGAATTTCGGTGGAGAGGAGCAGAGGTTG
TCCTGGCGTGGTTTAGGTAGTGTGAGAGGGTCCGGGTTCAAAACCACTTGCTGGGTGGGGAGT
CGTCAGTAAGTGGCTATGCCCCGACCCCGAAGCCTGTTTCCCCATCTGTACAATGGAAATGATA
AAGACGCCCATCTGATAGGGAATGACTCCTTTCGGTAAGTGCAGTGGAAGCTGTACACTGCCCA
GGCAAAGCGTCCGGGCAGCGTAGGCGGGCGACTCAGATCCCAGCCAGTGGACTTAGCCCCTGT
TTGCTCCTCCGATAACTGGGGTGACCTTGGTTAATATTCACCAGCAGCCTCCCCCGTTGCCCCTC
TGGATCCACTGCTTAAATACGGACGAGGACAGGGCCCTGTCTCCTCAGCTTCAGGCACCACCAC
TGACCTGGGACAGTGAATCCGGACTCTAAGGTAAATATAAAATTTTTAAGTGTATAATGTGTTA
AACTACTGATTCTAATTGTTTCTCTATTTTAGATTCCAACCTTTGGAACTGAGAATTCCCCGGACC
GGTGGATCCGCCACCATGGGCTGGTCCTGCATCATTCTGTTTCTGGTGGCCACAGCCACCGGCG
TGCACTCTAGAAACCTGCCAGTGGCTACCCCTGATCCGGGCATGTTTCCTTGTCTGCACCACAGC
CAGAACCTGCTGAGAGCCGTGTCCAATATGCTGCAGAAGGCCCGGCAGACCCTTGAGTTCTACC
CTTGCACCAGCGAGGAAATCGACCACGAGGACATCACCAAGGATAAGACCAGCACCGTGGAAG
CCTGCCTGCCTCTGGAACTGACCAAGAACGAGTCCTGCCTGAACAGCCGGGAAACCAGCTTCAT
CACCAATGGCAGCTGTCTGGCCAGCAGAAAGACCTCCTTCATGATGGCCCTGTGCCTGAGCAGC
ATCTACGAGGACCTGAAGATGTATCAGGTCGAGTTCAAGACCATGAACGCCAAGCTGCTGATG
GACCCCAAGAGACAGATTTTCCTCGACCAGAACATGCTGGCCGTGATCGATGAACTGATGCAG
GCCCTGAACTTCAACAGCGAGACAGTGCCCCAGAAGTCCTCTCTGGAAGAACCCGACTTCTACA
AGACCAAGATCAAGCTGTGCATCCTGCTGCACGCCTTCAGAATCAGGGCCGTGACCATCGACAG
AGTGATGAGCTATCTGAACGCCAGCGGCGGCGGAGGAAGCGGAGGCGGAGGATCTGGTGGT
GGTGGAAGTATTTGGGAGCTGAAGAAAGACGTGTACGTGGTCGAGCTGGACTGGTATCCTGAT
GCTCCCGGCGAAATGGTGGTGCTGACCTGTGATACACCCGAAGAGGACGGCATCACATGGACA
CTGGATCAGTCTAGCGAGGTGCTCGGCAGCGGCAAGACACTGACCATCCAAGTGAAAGAGTTT
GGCGACGCCGGCCAGTACACATGTCACAAAGGCGGAGAGGTGCTGAGCCATTCTCTGCTGCTG
CTCCACAAGAAAGAGGATGGCATTTGGAGCACCGACATCCTGAAGGACCAGAAAGAGCCCAAG
AACAAGACCTTCCTGAGATGCGAGGCCAAGAACTACAGCGGCAGATTCACCTGTTGGTGGCTG
ACCACCATCAGCACCGATCTGACCTTCAGCGTGAAGTCCAGCAGAGGCAGCTCTGATCCTCAAG
GCGTTACATGTGGCGCCGCTACACTGTCTGCCGAAAGAGTGCGGGGCGACAACAAAGAGTACG
AGTACAGCGTCGAGTGCCAAGAGGATTCTGCCTGTCCTGCCGCCGAGGAATCTCTGCCTATCGA
AGTGATGGTGGACGCCGTGCACAAGCTGAAGTACGAGAACTACACCAGCAGCTTTTTCATCCG
GGACATCATCAAGCCCGATCCTCCAAAGAACCTGCAGCTGAAGCCCCTGAAGAACAGCAGACA
GGTGGAAGTGTCTTGGGAGTACCCCGACACATGGTCTACCCCTCACAGCTACTTCAGCCTGACC
TTCTGTGTGCAAGTGCAGGGCAAGTCCAAGCGCGAGAAGAAAGATCGGGTGTTCACCGACAAG
ACCAGCGCCACCGTGATCTGCAGAAAGAACGCCAGCATCAGCGTGCGCGCTCAGGATAGGTAC
TACAGCAGCTCTTGGAGCGAGTGGGCCTCTGTTCCTTGTTCTTGAAAGCTTGGTACCCAAACAA
ACAAAGGCGCGTCCTGGATTCGTGGTAAAACATACCAGATTTCGATCTGGAGAGGTGAAGAAT
ACGACCACCTACTACATCCAGCTGATGAGTCCCAAATAGGACGAAACGCGCTCAAACAAACAAA
AGATCTCTTGTTTATTGCAGCTTATAATGGTTACAAATAAAGCAATAGCATCACAAATTTCACAA
ATAAAGCATTTTTTTCACTGCATTCTAGTTGTGGTTTGTCCAAACTCATCAATGTATCTTAACGCG
GCCGCACGTGCGGACCGAGCGGCCGCAGGAACCCCTAGTGATGGAGTTGGCCACTCCCTCTCT
GCGCGCTCGCTCGCTCACTGAGGCCGGGCGACCAAAGGTCGCCCGACGCCCGGGCTTTGCCCG
GGCGGCCTCAGTGAGCGAGCGAGCGCGCAGCTGCCTGCAGG
> AAV.LP1_SV40-hIL-12-3′-riboswitch (Seq ID No. 63): signal peptide, IL12 p40-(G4S)3-
p35; carrying the 11 nt and 4 nt deletion in the left ITR, and the 4 nt deletion in the right
ITR
aggaacccctagtgatggagttggccactccctctctgcgcgctcgctcgctcactgaggccgggcgaccaaaggtcgcccga
cgcccgggcggcctcagtgagcgagcgagcgcgcagctgcctgcaggGCGGCCGCACGCGTCCCTAAAATGGG
CAAACATTGCAAGCAGCAAACAGCAAACACACAGCCCTCCCTGCCTGCTGACCTTGGAGCTGGG
GCAGAGGTCAGAGACCTCTCTGGGCCCATGCCACCTCCAACATCCACTCGACCCCTTGGAATTTC
GGTGGAGAGGAGCAGAGGTTGTCCTGGCGTGGTTTAGGTAGTGTGAGAGGGTCCGGGTTCAA
AACCACTTGCTGGGTGGGGAGTCGTCAGTAAGTGGCTATGCCCCGACCCCGAAGCCTGTTTCCC
CATCTGTACAATGGAAATGATAAAGACGCCCATCTGATAGGGAATGACTCCTTTCGGTAAGTGC
AGTGGAAGCTGTACACTGCCCAGGCAAAGCGTCCGGGCAGCGTAGGCGGGCGACTCAGATCCC
AGCCAGTGGACTTAGCCCCTGTTTGCTCCTCCGATAACTGGGGTGACCTTGGTTAATATTCACCA
GCAGCCTCCCCCGTTGCCCCTCTGGATCCACTGCTTAAATACGGACGAGGACAGGGCCCTGTCT
CCTCAGCTTCAGGCACCACCACTGACCTGGGACAGTGAATCCGGACTCTAAGGTAAATATAAAA
TTTTTAAGTGTATAATGTGTTAAACTACTGATTCTAATTGTTTCTCTATTTTAGATTCCAACCTTTG
GAACTGAGAATTCCCCGGACCGGTGGATCCGCCACCATGGGCTGGTCCTGCATCATTCTGTTTC
TGGTGGCCACAGCCACCGGCGTGCACTCTATTTGGGAGCTGAAGAAAGACGTGTACGTGGTCG
AGCTGGACTGGTATCCTGATGCTCCCGGCGAAATGGTGGTGCTGACCTGTGATACACCCGAAG
AGGACGGCATCACATGGACACTGGATCAGTCTAGCGAGGTGCTCGGCAGCGGCAAGACACTGA
CCATCCAAGTGAAAGAGTTTGGCGACGCCGGCCAGTACACATGTCACAAAGGCGGAGAGGTGC
TGAGCCATTCTCTGCTGCTGCTCCACAAGAAAGAGGATGGCATTTGGAGCACCGACATCCTGAA
GGACCAGAAAGAGCCCAAGAACAAGACCTTCCTGAGATGCGAGGCCAAGAACTACAGCGGCA
GATTCACCTGTTGGTGGCTGACCACCATCAGCACCGATCTGACCTTCAGCGTGAAGTCCAGCAG
AGGCAGCTCTGATCCTCAAGGCGTTACATGTGGCGCCGCTACACTGTCTGCCGAAAGAGTGCG
GGGCGACAACAAAGAGTACGAGTACAGCGTCGAGTGCCAAGAGGATTCTGCCTGTCCTGCCGC
CGAGGAATCTCTGCCTATCGAAGTGATGGTGGACGCCGTGCACAAGCTGAAGTACGAGAACTA
CACCAGCAGCTTTTTCATCCGGGACATCATCAAGCCCGATCCTCCAAAGAACCTGCAGCTGAAG
CCCCTGAAGAACAGCAGACAGGTGGAAGTGTCTTGGGAGTACCCCGACACATGGTCTACCCCTC
ACAGCTACTTCAGCCTGACCTTCTGTGTGCAAGTGCAGGGCAAGTCCAAGCGCGAGAAGAAAG
ATCGGGTGTTCACCGACAAGACCAGCGCCACCGTGATCTGCAGAAAGAACGCCAGCATCAGCG
TGCGCGCTCAGGATAGGTACTACAGCAGCTCTTGGAGCGAGTGGGCCTCTGTTCCTTGTTCTGG
CGGCGGAGGAAGCGGAGGCGGAGGATCTGGTGGTGGTGGAAGTAGAAACCTGCCAGTGGCT
ACCCCTGATCCGGGCATGTTTCCTTGTCTGCACCACAGCCAGAACCTGCTGAGAGCCGTGTCCA
ATATGCTGCAGAAGGCCCGGCAGACCCTTGAGTTCTACCCTTGCACCAGCGAGGAAATCGACCA
CGAGGACATCACCAAGGATAAGACCAGCACCGTGGAAGCCTGCCTGCCTCTGGAACTGACCAA
GAACGAGTCCTGCCTGAACAGCCGGGAAACCAGCTTCATCACCAATGGCAGCTGTCTGGCCAG
CAGAAAGACCTCCTTCATGATGGCCCTGTGCCTGAGCAGCATCTACGAGGACCTGAAGATGTAT
CAGGTCGAGTTCAAGACCATGAACGCCAAGCTGCTGATGGACCCCAAGAGACAGATTTTCCTCG
ACCAGAACATGCTGGCCGTGATCGATGAACTGATGCAGGCCCTGAACTTCAACAGCGAGACAG
TGCCCCAGAAGTCCTCTCTGGAAGAACCCGACTTCTACAAGACCAAGATCAAGCTGTGCATCCT
GCTGCACGCCTTCAGAATCAGGGCCGTGACCATCGACAGAGTGATGAGCTATCTGAACGCCAG
CTGAAAGCTTGGTACCCAAACAAACAAAGGCGCGTCCTGGATTCGTGGTAAAACATACCAGATT
TCGATCTGGAGAGGTGAAGAATACGACCACCTACTACATCCAGCTGATGAGTCCCAAATAGGAC
GAAACGCGCTCAAACAAACAAAAGATCTCTTGTTTATTGCAGCTTATAATGGTTACAAATAAAG
CAATAGCATCACAAATTTCACAAATAAAGCATTTTTTTCACTGCATTCTAGTTGTGGTTTGTCCAA
ACTCATCAATGTATCTTAACGCGGCCGCACGTGCGGACCGAGCGGCCGCAGGAACCCCTAGTG
ATGGAGTTGGCCACTCCCTCTCTGCGCGCTCGCTCGCTCACTGAGGCCGGGCGACCAAAGGTCG
CCCGACGCCCGGGCTTTGCCCGGGCGGCCTCAGTGAGCGAGCGAGCGCGCAGCTGCCTGCAG
G
> AAV. LP1_SV40-hIL-12-3′riboswitch (SEQ ID No. 64); signal peptide, IL12
p35(G4S)3p40; carrying the 11 nt and 4 nt deletion in the left ITR, and the 4 nt deletion
in the right ITR
aggaacccctagtgatggagttggccactccctctctgcgcgctcgctcgctcactgaggccgggcgaccaaaggtcgcccgacgcccgg
gcggcctcagtgagcgagcgagcgcgcagctgcctgcaggGCGGCCGCACGCGTCCCTAAAATGGGCAAACATTGCAA
GCAGCAAACAGCAAACACACAGCCCTCCCTGCCTGCTGACCTTGGAGCTGGGGCAGAGGTCAGAGACCT
CTCTGGGCCCATGCCACCTCCAACATCCACTCGACCCCTTGGAATTTCGGTGGAGAGGAGCAGAGGTTGT
CCTGGCGTGGTTTAGGTAGTGTGAGAGGGTCCGGGTTCAAAACCACTTGCTGGGTGGGGAGTCGTCAGT
AAGTGGCTATGCCCCGACCCCGAAGCCTGTTTCCCCATCTGTACAATGGAAATGATAAAGACGCCCATCT
GATAGGGAATGACTCCTTTCGGTAAGTGCAGTGGAAGCTGTACACTGCCCAGGCAAAGCGTCCGGGCAG
CGTAGGCGGGCGACTCAGATCCCAGCCAGTGGACTTAGCCCCTGTTTGCTCCTCCGATAACTGGGGTGAC
CTTGGTTAATATTCACCAGCAGCCTCCCCCGTTGCCCCTCTGGATCCACTGCTTAAATACGGACGAGGACA
GGGCCCTGTCTCCTCAGCTTCAGGCACCACCACTGACCTGGGACAGTGAATCCGGACTCTAAGGTAAATA
TAAAATTTTTAAGTGTATAATGTGTTAAACTACTGATTCTAATTGTTTCTCTATTTTAGATTCCAACCTTTGG
AACTGAGAATTCCCCGGACCGGTGGATCCGCCACCATGGGCTGGTCCTGCATCATTCTGTTTCTGGTGGC
CACAGCCACCGGCGTGCACTCTAGAAACCTGCCAGTGGCTACCCCTGATCCGGGCATGTTTCCTTGTCTG
CACCACAGCCAGAACCTGCTGAGAGCCGTGTCCAATATGCTGCAGAAGGCCCGGCAGACCCTTGAGTTC
TACCCTTGCACCAGCGAGGAAATCGACCACGAGGACATCACCAAGGATAAGACCAGCACCGTGGAAGCC
TGCCTGCCTCTGGAACTGACCAAGAACGAGTCCTGCCTGAACAGCCGGGAAACCAGCTTCATCACCAATG
GCAGCTGTCTGGCCAGCAGAAAGACCTCCTTCATGATGGCCCTGTGCCTGAGCAGCATCTACGAGGACCT
GAAGATGTATCAGGTCGAGTTCAAGACCATGAACGCCAAGCTGCTGATGGACCCCAAGAGACAGATTTT
CCTCGACCAGAACATGCTGGCCGTGATCGATGAACTGATGCAGGCCCTGAACTTCAACAGCGAGACAGT
GCCCCAGAAGTCCTCTCTGGAAGAACCCGACTTCTACAAGACCAAGATCAAGCTGTGCATCCTGCTGCAC
GCCTTCAGAATCAGGGCCGTGACCATCGACAGAGTGATGAGCTATCTGAACGCCAGCGGCGGCGGAGG
AAGCGGAGGCGGAGGATCTGGTGGTGGTGGAAGTATTTGGGAGCTGAAGAAAGACGTGTACGTGGTC
GAGCTGGACTGGTATCCTGATGCTCCCGGCGAAATGGTGGTGCTGACCTGTGATACACCCGAAGAGGAC
GGCATCACATGGACACTGGATCAGTCTAGCGAGGTGCTCGGCAGCGGCAAGACACTGACCATCCAAGTG
AAAGAGTTTGGCGACGCCGGCCAGTACACATGTCACAAAGGCGGAGAGGTGCTGAGCCATTCTCTGCTG
CTGCTCCACAAGAAAGAGGATGGCATTTGGAGCACCGACATCCTGAAGGACCAGAAAGAGCCCAAGAA
CAAGACCTTCCTGAGATGCGAGGCCAAGAACTACAGCGGCAGATTCACCTGTTGGTGGCTGACCACCAT
CAGCACCGATCTGACCTTCAGCGTGAAGTCCAGCAGAGGCAGCTCTGATCCTCAAGGCGTTACATGTGGC
GCCGCTACACTGTCTGCCGAAAGAGTGCGGGGCGACAACAAAGAGTACGAGTACAGCGTCGAGTGCCA
AGAGGATTCTGCCTGTCCTGCCGCCGAGGAATCTCTGCCTATCGAAGTGATGGTGGACGCCGTGCACAA
GCTGAAGTACGAGAACTACACCAGCAGCTTTTTCATCCGGGACATCATCAAGCCCGATCCTCCAAAGAAC
CTGCAGCTGAAGCCCCTGAAGAACAGCAGACAGGTGGAAGTGTCTTGGGAGTACCCCGACACATGGTCT
ACCCCTCACAGCTACTTCAGCCTGACCTTCTGTGTGCAAGTGCAGGGCAAGTCCAAGCGCGAGAAGAAA
GATCGGGTGTTCACCGACAAGACCAGCGCCACCGTGATCTGCAGAAAGAACGCCAGCATCAGCGTGCGC
GCTCAGGATAGGTACTACAGCAGCTCTTGGAGCGAGTGGGCCTCTGTTCCTTGTTCTTGAAAGCTTGGTA
CCCAAACAAACAAAGGCGCGTCCTGGATTCGTGGTAAAACATACCAGATTTCGATCTGGAGAGGTGAAG
AATACGACCACCTACTACATCCAGCTGATGAGTCCCAAATAGGACGAAACGCGCTCAAACAAACAAAAG
ATCTCTTGTTTATTGCAGCTTATAATGGTTACAAATAAAGCAATAGCATCACAAATTTCACAAATAAAGCA
TTTTTTTCACTGCATTCTAGTTGTGGTTTGTCCAAACTCATCAATGTATCTTAACGCGGCCGCACGTGCGGA
CCGAGCGGCCGCAGGAACCCCTAGTGATGGAGTTGGCCACTCCCTCTCTGCGCGCTCGCTCGCTCACTGA
GGCCGGGCGACCAAAGGTCGCCCGACGCCCGGGCTTTGCCCGGGCGGCCTCAGTGAGCGAGCGAGCGC
GCAGCTGCCTGCAGG
> AAV.LP1-hIL-12-3′-riboswitch (Seq ID No. 65): signal peptide, IL12 p40-(G4S)3-p35;
carrying the 11 nt and 4 nt deletion in the left ITR, and the 4 nt deletion in the right ITR
aggaacccctagtgatggagttggccactccctctctgcgcgctcgctcgctcactgaggccgggcgaccaaaggtcgcccga
cgcccgggcggcctcagtgagcgagcgagcgcgcagctgcctgcaggGCGGCCGCACGCGTTCGACCCCCTAAA
ATGGGCAAACATTGCAAGCAAACAGCAAACACACAGCCCTCCCTGCCTGCTGACCTTGGAGCTG
GGGCAGAGGTCAGAGACCTCTCTGGGCCCATGCCACCTCCAACATCCACTCGACCCCTTGGAAT
TTCGGTGGAGAGGAGCAGAGGTTGTCCTGGCGTGGTTTAGGTAGTGTGAGAGGGGAATGACT
CCTTTCGGTAAGTGCAGTGGAAGCTGTACACTGCCCAGGCAAAGCGTCCGGGCAGCGTAGGCG
GGCGACTCAGATCCCAGCCAGTGGACTTAGCCCCTGTTTGCTCCTCCGATAACTGGGGTGACCT
TGGTTAATATTCACCAGCAGCCTCCCCCGTTGCCCCTCTGGATCCACTGCTTAAATACGGACGAG
GACAGGGCCCTGTCTCCTCAGCTTCAGGCACCACCACTGACCTGGGACAGTGAATCCGGACTCT
AAGAGAATTCCCCGGACCGGTGGATCCGCCACCATGGGCTGGTCCTGCATCATTCTGTTTCTGG
TGGCCACAGCCACCGGCGTGCACTCTATTTGGGAGCTGAAGAAAGACGTGTACGTGGTCGAGC
TGGACTGGTATCCTGATGCTCCCGGCGAAATGGTGGTGCTGACCTGTGATACACCCGAAGAGG
ACGGCATCACATGGACACTGGATCAGTCTAGCGAGGTGCTCGGCAGCGGCAAGACACTGACCA
TCCAAGTGAAAGAGTTTGGCGACGCCGGCCAGTACACATGTCACAAAGGCGGAGAGGTGCTGA
GCCATTCTCTGCTGCTGCTCCACAAGAAAGAGGATGGCATTTGGAGCACCGACATCCTGAAGGA
CCAGAAAGAGCCCAAGAACAAGACCTTCCTGAGATGCGAGGCCAAGAACTACAGCGGCAGATT
CACCTGTTGGTGGCTGACCACCATCAGCACCGATCTGACCTTCAGCGTGAAGTCCAGCAGAGGC
AGCTCTGATCCTCAAGGCGTTACATGTGGCGCCGCTACACTGTCTGCCGAAAGAGTGCGGGGC
GACAACAAAGAGTACGAGTACAGCGTCGAGTGCCAAGAGGATTCTGCCTGTCCTGCCGCCGAG
GAATCTCTGCCTATCGAAGTGATGGTGGACGCCGTGCACAAGCTGAAGTACGAGAACTACACC
AGCAGCTTTTTCATCCGGGACATCATCAAGCCCGATCCTCCAAAGAACCTGCAGCTGAAGCCCCT
GAAGAACAGCAGACAGGTGGAAGTGTCTTGGGAGTACCCCGACACATGGTCTACCCCTCACAG
CTACTTCAGCCTGACCTTCTGTGTGCAAGTGCAGGGCAAGTCCAAGCGCGAGAAGAAAGATCG
GGTGTTCACCGACAAGACCAGCGCCACCGTGATCTGCAGAAAGAACGCCAGCATCAGCGTGCG
CGCTCAGGATAGGTACTACAGCAGCTCTTGGAGCGAGTGGGCCTCTGTTCCTTGTTCTGGCGGC
GGAGGAAGCGGAGGCGGAGGATCTGGTGGTGGTGGAAGTAGAAACCTGCCAGTGGCTACCCC
TGATCCGGGCATGTTTCCTTGTCTGCACCACAGCCAGAACCTGCTGAGAGCCGTGTCCAATATG
CTGCAGAAGGCCCGGCAGACCCTTGAGTTCTACCCTTGCACCAGCGAGGAAATCGACCACGAG
GACATCACCAAGGATAAGACCAGCACCGTGGAAGCCTGCCTGCCTCTGGAACTGACCAAGAAC
GAGTCCTGCCTGAACAGCCGGGAAACCAGCTTCATCACCAATGGCAGCTGTCTGGCCAGCAGA
AAGACCTCCTTCATGATGGCCCTGTGCCTGAGCAGCATCTACGAGGACCTGAAGATGTATCAGG
TCGAGTTCAAGACCATGAACGCCAAGCTGCTGATGGACCCCAAGAGACAGATTTTCCTCGACCA
GAACATGCTGGCCGTGATCGATGAACTGATGCAGGCCCTGAACTTCAACAGCGAGACAGTGCC
CCAGAAGTCCTCTCTGGAAGAACCCGACTTCTACAAGACCAAGATCAAGCTGTGCATCCTGCTG
CACGCCTTCAGAATCAGGGCCGTGACCATCGACAGAGTGATGAGCTATCTGAACGCCAGCTGA
AAGCTTGGTACCCAAACAAACAAAGGCGCGTCCTGGATTCGTGGTAAAACATACCAGATTTCGA
TCTGGAGAGGTGAAGAATACGACCACCTACTACATCCAGCTGATGAGTCCCAAATAGGACGAA
ACGCGCTCAAACAAACAAAAGATCTCTTGTTTATTGCAGCTTATAATGGTTACAAATAAAGCAAT
AGCATCACAAATTTCACAAATAAAGCATTTTTTTCACTGCATTCTAGTTGTGGTTTGTCCAAACTC
ATCAATGTATCTTAACGCGGCCGCACGTGCGGACCGAGCGGCCGCAGGAACCCCTAGTGATGG
AGTTGGCCACTCCCTCTCTGCGCGCTCGCTCGCTCACTGAGGCCGGGCGACCAAAGGTCGCCCG
ACGCCCGGGCTTTGCCCGGGCGGCCTCAGTGAGCGAGCGAGCGCGCAGCTGCCTGCAGG
> AAV. LP1-hIL-12-3′riboswitch (SEQ ID No. 66); signal peptide, IL12 p35(G4S)3p40;
carrying the 11 nt and 4 nt deletion in the left ITR, and the 4 nt deletion in the right ITR
aggaacccctagtgatggagttggccactccctctctgcgcgctcgctcgctcactgaggccgggcgaccaaaggtcgcccga
cgcccgggcggcctcagtgagcgagcgagcgcgcagctgcctgcaggGCGGCCGCACGCGTTCGACCCCCTAAA
ATGGGCAAACATTGCAAGCAAACAGCAAACACACAGCCCTCCCTGCCTGCTGACCTTGGAGCTG
GGGCAGAGGTCAGAGACCTCTCTGGGCCCATGCCACCTCCAACATCCACTCGACCCCTTGGAAT
TTCGGTGGAGAGGAGCAGAGGTTGTCCTGGCGTGGTTTAGGTAGTGTGAGAGGGGAATGACT
CCTTTCGGTAAGTGCAGTGGAAGCTGTACACTGCCCAGGCAAAGCGTCCGGGCAGCGTAGGCG
GGCGACTCAGATCCCAGCCAGTGGACTTAGCCCCTGTTTGCTCCTCCGATAACTGGGGTGACCT
TGGTTAATATTCACCAGCAGCCTCCCCCGTTGCCCCTCTGGATCCACTGCTTAAATACGGACGAG
GACAGGGCCCTGTCTCCTCAGCTTCAGGCACCACCACTGACCTGGGACAGTGAATCCGGACTCT
AAGAGAATTCCCCGGACCGGTGGATCCGCCACCATGGGCTGGTCCTGCATCATTCTGTTTCTGG
TGGCCACAGCCACCGGCGTGCACTCTAGAAACCTGCCAGTGGCTACCCCTGATCCGGGCATGTT
TCCTTGTCTGCACCACAGCCAGAACCTGCTGAGAGCCGTGTCCAATATGCTGCAGAAGGCCCGG
CAGACCCTTGAGTTCTACCCTTGCACCAGCGAGGAAATCGACCACGAGGACATCACCAAGGATA
AGACCAGCACCGTGGAAGCCTGCCTGCCTCTGGAACTGACCAAGAACGAGTCCTGCCTGAACA
GCCGGGAAACCAGCTTCATCACCAATGGCAGCTGTCTGGCCAGCAGAAAGACCTCCTTCATGAT
GGCCCTGTGCCTGAGCAGCATCTACGAGGACCTGAAGATGTATCAGGTCGAGTTCAAGACCAT
GAACGCCAAGCTGCTGATGGACCCCAAGAGACAGATTTTCCTCGACCAGAACATGCTGGCCGT
GATCGATGAACTGATGCAGGCCCTGAACTTCAACAGCGAGACAGTGCCCCAGAAGTCCTCTCTG
GAAGAACCCGACTTCTACAAGACCAAGATCAAGCTGTGCATCCTGCTGCACGCCTTCAGAATCA
GGGCCGTGACCATCGACAGAGTGATGAGCTATCTGAACGCCAGCGGCGGCGGAGGAAGCGGA
GGCGGAGGATCTGGTGGTGGTGGAAGTATTTGGGAGCTGAAGAAAGACGTGTACGTGGTCGA
GCTGGACTGGTATCCTGATGCTCCCGGCGAAATGGTGGTGCTGACCTGTGATACACCCGAAGA
GGACGGCATCACATGGACACTGGATCAGTCTAGCGAGGTGCTCGGCAGCGGCAAGACACTGAC
CATCCAAGTGAAAGAGTTTGGCGACGCCGGCCAGTACACATGTCACAAAGGCGGAGAGGTGCT
GAGCCATTCTCTGCTGCTGCTCCACAAGAAAGAGGATGGCATTTGGAGCACCGACATCCTGAAG
GACCAGAAAGAGCCCAAGAACAAGACCTTCCTGAGATGCGAGGCCAAGAACTACAGCGGCAG
ATTCACCTGTTGGTGGCTGACCACCATCAGCACCGATCTGACCTTCAGCGTGAAGTCCAGCAGA
GGCAGCTCTGATCCTCAAGGCGTTACATGTGGCGCCGCTACACTGTCTGCCGAAAGAGTGCGG
GGCGACAACAAAGAGTACGAGTACAGCGTCGAGTGCCAAGAGGATTCTGCCTGTCCTGCCGCC
GAGGAATCTCTGCCTATCGAAGTGATGGTGGACGCCGTGCACAAGCTGAAGTACGAGAACTAC
ACCAGCAGCTTTTTCATCCGGGACATCATCAAGCCCGATCCTCCAAAGAACCTGCAGCTGAAGC
CCCTGAAGAACAGCAGACAGGTGGAAGTGTCTTGGGAGTACCCCGACACATGGTCTACCCCTC
ACAGCTACTTCAGCCTGACCTTCTGTGTGCAAGTGCAGGGCAAGTCCAAGCGCGAGAAGAAAG
ATCGGGTGTTCACCGACAAGACCAGCGCCACCGTGATCTGCAGAAAGAACGCCAGCATCAGCG
TGCGCGCTCAGGATAGGTACTACAGCAGCTCTTGGAGCGAGTGGGCCTCTGTTCCTTGTTCTTG
AAAGCTTGGTACCCAAACAAACAAAGGCGCGTCCTGGATTCGTGGTAAAACATACCAGATTTCG
ATCTGGAGAGGTGAAGAATACGACCACCTACTACATCCAGCTGATGAGTCCCAAATAGGACGA
AACGCGCTCAAACAAACAAAAGATCTCTTGTTTATTGCAGCTTATAATGGTTACAAATAAAGCAA
TAGCATCACAAATTTCACAAATAAAGCATTTTTTTCACTGCATTCTAGTTGTGGTTTGTCCAAACT
CATCAATGTATCTTAACGCGGCCGCACGTGCGGACCGAGCGGCCGCAGGAACCCCTAGTGATG
GAGTTGGCCACTCCCTCTCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGCAGCTGCCTGCAGG
human CEA promoter (Seq ID No. 67)
GCCCTGGAGAGCATGGGGAGACCCGGGACCCTGCTGGGTTTCTCTGTCACAAAGGAAAATAAT
CCCCCTGGTGTGACAGACCCAAGGACAGAACACAGCAGAGGTCAGCACTGGGGAAGACAGGT
TGTCCTCCCAGGGGATGGGGGTCCATCCACCTTGCCGAAAAGATTTGTCTGAGGAACTGAAAAT
AGAAGGGAAAAAAGAGGAGGGACAAAAGAGGCAGAAATGAGAGGGGAGGGGACAGAGGAC
ACCTGAATAAAGACCACACCCATGACCCACGTGATGCTGAGAAGTACTCCTGCCCTAGGAAGAG
ACTCAGGGCAGAGGGAGGAAGGACAGCAGACCAGACAGTCACAGCAGCCTTGACAAAACGTT
CCTGGAACTCAAGCTCTTCTCCACAGAGGAGGACAGAGCAGACAGCAGAGACC
human Muc1 promoter (Seq ID No. 68)
CCTGCAGGGCCCaCTAGtGTTCATCGGAGCCCAGGTTTACTCCCTTAAGTGGAAATTTCTTCCCC
CACTCCCTCCTTGGCTTTCTCCAAGGAGGGAACCCAGGCTACTGGAAAGTCCGGCTGGGGCGG
GGACTGTGGGTTTCAGGGTAGAACTGCGTGTGGAACGGGACAGGGAGCGGTTAGAAGGGTG
GGGCTATTCCGGGAAGTGGTGGGGGGAGGGAGCCCAAAACTAGCACCTAGTCCACTCATTATC
CAGCCCTCTTATTTCTCGGCCCCGCTCTGCTTCAGTGGACCCGGGGAGGGCGGGGAAGTGGAG
TGGGAGACCTAGGGGTGGGCTTCCCGACCTTGCTGTACAGGACCTCGACCTAGCTGGCTTTGTT
CCCCATCCCCACGTTAGTTGTTGCCCTGAGGCTAAAACTAGAGCCCAGGGGCCCCAAGTTCCAG
ACTGCCCCTCCCCCCTCCCCCGGAGCCAGGGAGTGGTTGGTGAAAGGGGGAGGCCAGCTGGAG
AACAAACGGGTAGTCAGGGGGTTGAGCGATTAGAGCCCTTGTACCCTACCCAGGAATGGTTGG
GGAGGAGGAGGAAGAGGTAGGAGGTAGGGGAGGGGGCGGGGTTTTGTCACCTGTCACCTGC
TCCGGCTGTGCCTAGGGCGGGCGGGGGGGGAGTGGGGGGACCGGTATAAAGCGGTAGGCGC
CTGTGCCCGCTCCACCTCTCAAGCAGCCAGCGCCTGCCTGAATCTGTTCTGCCCCCTCCCCACCC
ATTTCACCACCACC
human AFP promoter (Seq ID No. 69)
attctgtagtttgaggagaatatttgttatatttgcaaaataaaataagtttgcaagttttttttttctgccccaaagagctctgtg
tccttgaacataaaatacaaataaccgctatgctgttaattattggcaaatgtcccattttcaacctaaggaaataccataaag
taacagatataccaacaaaaggttactagttaacaggcattgcctgaaaagagtataaaagaatttcagcatgattttccata
ttgtgcttccaccactgccaat
GenBank: AAN03857.1 Capsid Protein adeno-associated Virus 8, AAV8 VP1 Sequence (SEQ ID
No. 70)
MAADGYLPDW LEDNLSEGIR EWWALKPGAP KPKANQQKQD DGRGLVLPGY KYLGPENGLD
KGEPVNAADA AALEHDKAYD QQLQAGDNPY LRYNHADAEF QERLQEDTSF GGNLGRAVFQ
AKKRVLEPLG LVEEGAKTAP GKKRPVEPSP QRSPDSSTGI GKKGQQPARK RLNFGQTGDS
ESVPDPQPLG EPPAAPSGVG PNTMAAGGGA PMADNNEGAD GVGSSSGNWH CDSTWLGDRV
ITTSTRTWAL PTYNNHLYKQ ISNGTSGGAT NDNTYFGYST PWGYFDFNRF HCHFSPRDWQ
RLINNNWGFR PKRLSFKLEN IQVKEVTQNE GTKTIANNLT STIQVFTDSE YQLPYVLGSA
HQGCLPPFPA DVFMIPQYGY LTLNNGSQAV GRSSFYCLEY FPSQMLRTGN NFQFTYTFED
VPFHSSYAHS QSLDRLMNPL IDQYLYYLSR TQTTGGTANT QTLGFSQGGP NTMANQAKNW
LPGPCYRQQR VSTTTGQNNN SNFAWTAGTK YHLNGRNSLA NPGIAMATHK DDEERFFPSN
GILIFGKQNA ARDNADYSDV MLTSEEEIKT TNPVATEEYG IVADNLQQQN TAPQIGTVNS
QGALPGMVWQ NRDVYLQGPI WAKIPHTDGN FHPSPLMGGF GLKHPPPQIL IKNTPVPADP
PTTFNQSKLN SFITQYSTGQ VSVEIEWELQ KENSKRWNPE IQYTSNYYKS TSVDFAVNTE
GVYSEPRPIG TRYLTRNL
GenBank: AF513852.1 AAV8 VP1 Capsid CDS Sequence (Sequence ID No. 71)
atggctgccgatggttatcttccagattggctcgaggacaacctctctgagggcattcgcgagtggtgggcgctgaaacctgg
Agccccgaagcccaaagccaaccagcaaaagcaggacgacggccggggtctggtgcttcctggctacaagtacctcggacc
Cttcaacggactcgacaagggggagcccgtcaacgcggcggacgcagcggccctcgagcacgacaaggcctacgaccagc
Agctgcaggcgggtgacaatccgtacctgcggtataaccacgccgacgccgagtttcaggagcgtctgcaagaagatacgtc
Ttttgggggcaacctcgggcgagcagtcttccaggccaagaagcgggttctcgaacctctcggtctggttgaggaaggcgcta
Agacggctcctggaaagaagagaccggtagagccatcaccccagcgttctccagactcctctacgggcatcggcaagaaagg
Ccaacagcccgccagaaaaagactcaattttggtcagactggcgactcagagtcagttccagaccctcaacctctcggagaac
Ctccagcagcgccctctggtgtgggacctaatacaatggctgcaggcggtggcgcaccaatggcagacaataacgaaggcgc
Cgacggagtgggtagttcctcgggaaattggcattgcgattccacatggctgggcgacagagtcatcaccaccagcacccgaa
Cctgggccctgcccacctacaacaaccacctctacaagcaaatctccaacgggacatcgggaggagccaccaacgacaacac
Ctacttcggctacagcaccccctgggggtattttgactttaacagattccactgccacttttcaccacgtgactggcagcgactca
Tcaacaacaactggggattccggcccaagagactcagcttcaagctcttcaacatccaggtcaaggaggtcacgcagaatgaa
Ggcaccaagaccatcgccaataacctcaccagcaccatccaggtgtttacggactcggagtaccagctgccgtacgttctcggct
Ctgcccaccagggctgcctgcctccgttcccggcggacgtgttcatgattccccagtacggctacctaacactcaacaacggtag
Tcaggccgtgggacgctcctccttctactgcctggaatactttccttcgcagatgctgagaaccggcaacaacttccagtttactt
Acaccttcgaggacgtgcctttccacagcagctacgcccacagccagagcttggaccggctgatgaatcctctgattgaccagt
Acctgtactacttgtctcggactcaaacaacaggaggcacggcaaatacgcagactctgggcttcagccaaggtgggcctaat
Acaatggccaatcaggcaaagaactggctgccaggaccctgttaccgccaacaacgcgtctcaacgacaaccgggcaaaaca
Acaatagcaactttgcctggactgctgggaccaaataccatctgaatggaagaaattcattggctaatcctggcatcgctatggc
Aacacacaaagacgacgaggagcgtttttttcccagtaacgggatcctgatttttggcaaacaaaatgctgccagagacaatgc
Ggattacagcgatgtcatgctcaccagcgaggaagaaatcaaaaccactaaccctgtggctacagaggaatacggtatcgtgg
Cagataacttgcagcagcaaaacacggctcctcaaattggaactgtcaacagccagggggccttacccggtatggtctggcaga
Accgggacgtgtacctgcagggtcccatctgggccaagattcctcacacggacggcaacttccacccgtctccgctgatgggcg
Gctttggcctgaaacatcctccgcctcagatcctgatcaagaacacgcctgtacctgcggatcctccgaccaccttcaaccagtca
Aagctgaactctttcatcacgcaatacagcaccggacaggtcagcgtggaaattgaatgggagctgcagaaggaaaacagcaa
Gcgctggaaccccgagatccagtacacctccaactactacaaatctacaagtgtggactttgctgttaatacagaaggcgtgtact
Ctgaaccccgccccattggcacccgttacctcacccgtaatctgtaa
>LP1 promotor without SV40 intron (Seq ID 72)
TCGACCCCCTAAAATGGGCAAACATTGCAAGCAAACAGCAAACACACAGCCCTCCCTGCCTGCTGACCTT
GGAGCTGGGGCAG
AGGTCAGAGACCTCTCTGGGCCCATGCCACCTCCAACATCCACTCGACCCCTTGGAATTTCGGTGGAGAG
GAGCAGAGGTTGT
CCTGGCGTGGTTTAGGTAGTGTGAGAGGGGAATGACTCCTTTCGGTAAGTGCAGTGGAAGCTGTACACT
GCCCAGGCAAAGC
GTCCGGGCAGCGTAGGCGGGCGACTCAGATCCCAGCCAGTGGACTTAGCCCCTGTTTGCTCCTCCGATAA
CTGGGGTGACCTT
GGTTAATATTCACCAGCAGCCTCCCCCGTTGCCCCTCTGGATCCACTGCTTAAATACGGACGAGGACAGG
GCCCTGTCTCCTCA
GCTTCAGGCACCACCACTGACCTGGGACAGTGAATCCGGACTCTAAGA
> LP1-hIL-12-3′-riboswitch (Seq ID No. 73): signal peptide, IL12 p40-(G4S)3-p35; ex-
pression cassette
TCGACCCCCTAAAATGGGCAAACATTGCAAGCAAACAGCAAACACACAGCCCTCCCTGCCTGCT
GACCTTGGAGCTGGGGCAGAGGTCAGAGACCTCTCTGGGCCCATGCCACCTCCAACATCCACTC
GACCCCTTGGAATTTCGGTGGAGAGGAGCAGAGGTTGTCCTGGCGTGGTTTAGGTAGTGTGAG
AGGGGAATGACTCCTTTCGGTAAGTGCAGTGGAAGCTGTACACTGCCCAGGCAAAGCGTCCGG
GCAGCGTAGGCGGGCGACTCAGATCCCAGCCAGTGGACTTAGCCCCTGTTTGCTCCTCCGATAA
CTGGGGTGACCTTGGTTAATATTCACCAGCAGCCTCCCCCGTTGCCCCTCTGGATCCACTGCTTA
AATACGGACGAGGACAGGGCCCTGTCTCCTCAGCTTCAGGCACCACCACTGACCTGGGACAGT
GAATCCGGACTCTAAGAGAATTCCCCGGACCGGTGGATCCGCCACCATGGGCTGGTCCTGCATC
ATTCTGTTTCTGGTGGCCACAGCCACCGGCGTGCACTCTATTTGGGAGCTGAAGAAAGACGTGT
ACGTGGTCGAGCTGGACTGGTATCCTGATGCTCCCGGCGAAATGGTGGTGCTGACCTGTGATA
CACCCGAAGAGGACGGCATCACATGGACACTGGATCAGTCTAGCGAGGTGCTCGGCAGCGGCA
AGACACTGACCATCCAAGTGAAAGAGTTTGGCGACGCCGGCCAGTACACATGTCACAAAGGCG
GAGAGGTGCTGAGCCATTCTCTGCTGCTGCTCCACAAGAAAGAGGATGGCATTTGGAGCACCG
ACATCCTGAAGGACCAGAAAGAGCCCAAGAACAAGACCTTCCTGAGATGCGAGGCCAAGAACT
ACAGCGGCAGATTCACCTGTTGGTGGCTGACCACCATCAGCACCGATCTGACCTTCAGCGTGAA
GTCCAGCAGAGGCAGCTCTGATCCTCAAGGCGTTACATGTGGCGCCGCTACACTGTCTGCCGAA
AGAGTGCGGGGCGACAACAAAGAGTACGAGTACAGCGTCGAGTGCCAAGAGGATTCTGCCTG
TCCTGCCGCCGAGGAATCTCTGCCTATCGAAGTGATGGTGGACGCCGTGCACAAGCTGAAGTAC
GAGAACTACACCAGCAGCTTTTTCATCCGGGACATCATCAAGCCCGATCCTCCAAAGAACCTGC
AGCTGAAGCCCCTGAAGAACAGCAGACAGGTGGAAGTGTCTTGGGAGTACCCCGACACATGGT
CTACCCCTCACAGCTACTTCAGCCTGACCTTCTGTGTGCAAGTGCAGGGCAAGTCCAAGCGCGA
GAAGAAAGATCGGGTGTTCACCGACAAGACCAGCGCCACCGTGATCTGCAGAAAGAACGCCAG
CATCAGCGTGCGCGCTCAGGATAGGTACTACAGCAGCTCTTGGAGCGAGTGGGCCTCTGTTCCT
TGTTCTGGCGGCGGAGGAAGCGGAGGCGGAGGATCTGGTGGTGGTGGAAGTAGAAACCTGCC
AGTGGCTACCCCTGATCCGGGCATGTTTCCTTGTCTGCACCACAGCCAGAACCTGCTGAGAGCC
GTGTCCAATATGCTGCAGAAGGCCCGGCAGACCCTTGAGTTCTACCCTTGCACCAGCGAGGAAA
TCGACCACGAGGACATCACCAAGGATAAGACCAGCACCGTGGAAGCCTGCCTGCCTCTGGAAC
TGACCAAGAACGAGTCCTGCCTGAACAGCCGGGAAACCAGCTTCATCACCAATGGCAGCTGTCT
GGCCAGCAGAAAGACCTCCTTCATGATGGCCCTGTGCCTGAGCAGCATCTACGAGGACCTGAA
GATGTATCAGGTCGAGTTCAAGACCATGAACGCCAAGCTGCTGATGGACCCCAAGAGACAGAT
TTTCCTCGACCAGAACATGCTGGCCGTGATCGATGAACTGATGCAGGCCCTGAACTTCAACAGC
GAGACAGTGCCCCAGAAGTCCTCTCTGGAAGAACCCGACTTCTACAAGACCAAGATCAAGCTGT
GCATCCTGCTGCACGCCTTCAGAATCAGGGCCGTGACCATCGACAGAGTGATGAGCTATCTGAA
CGCCAGCTGAAAGCTTGGTACCCAAACAAACAAAGGCGCGTCCTGGATTCGTGGTAAAACATA
CCAGATTTCGATCTGGAGAGGTGAAGAATACGACCACCTACTACATCCAGCTGATGAGTCCCAA
ATAGGACGAAACGCGCTCAAACAAACAAAAGATCTCTTGTTTATTGCAGCTTATAATGGTTACA
AATAAAGCAATAGCATCACAAATTTCACAAATAAAGCATTTTTTTCACTGCATTCTAGTTGTGGTT
TGTCCAAACTCATCAATGTATCTTAACGCGGCCGCACGTGCGGACCGAGCGGCCGC
> LP1_SV40-hIL-12-3′-riboswitch (Seq ID No. 74): signal peptide, IL12 p40-(G4S)3-p35;
expression cassette
CCCTAAAATGGGCAAACATTGCAAGCAGCAAACAGCAAACACACAGCCCTCCCTGCCTGCTGAC
CTTGGAGCTGGGGCAGAGGTCAGAGACCTCTCTGGGCCCATGCCACCTCCAACATCCACTCGAC
CCCTTGGAATTTCGGTGGAGAGGAGCAGAGGTTGTCCTGGCGTGGTTTAGGTAGTGTGAGAGG
GTCCGGGTTCAAAACCACTTGCTGGGTGGGGAGTCGTCAGTAAGTGGCTATGCCCCGACCCCG
AAGCCTGTTTCCCCATCTGTACAATGGAAATGATAAAGACGCCCATCTGATAGGGAATGACTCC
TTTCGGTAAGTGCAGTGGAAGCTGTACACTGCCCAGGCAAAGCGTCCGGGCAGCGTAGGCGGG
CGACTCAGATCCCAGCCAGTGGACTTAGCCCCTGTTTGCTCCTCCGATAACTGGGGTGACCTTG
GTTAATATTCACCAGCAGCCTCCCCCGTTGCCCCTCTGGATCCACTGCTTAAATACGGACGAGGA
CAGGGCCCTGTCTCCTCAGCTTCAGGCACCACCACTGACCTGGGACAGTGAATCCGGACTCTAA
GGTAAATATAAAATTTTTAAGTGTATAATGTGTTAAACTACTGATTCTAATTGTTTCTCTATTTTA
GATTCCAACCTTTGGAACTGAGAATTCCCCGGACCGGTGGATCCGCCACCATGGGCTGGTCCTG
CATCATTCTGTTTCTGGTGGCCACAGCCACCGGCGTGCACTCTATTTGGGAGCTGAAGAAAGAC
GTGTACGTGGTCGAGCTGGACTGGTATCCTGATGCTCCCGGCGAAATGGTGGTGCTGACCTGT
GATACACCCGAAGAGGACGGCATCACATGGACACTGGATCAGTCTAGCGAGGTGCTCGGCAGC
GGCAAGACACTGACCATCCAAGTGAAAGAGTTTGGCGACGCCGGCCAGTACACATGTCACAAA
GGCGGAGAGGTGCTGAGCCATTCTCTGCTGCTGCTCCACAAGAAAGAGGATGGCATTTGGAGC
ACCGACATCCTGAAGGACCAGAAAGAGCCCAAGAACAAGACCTTCCTGAGATGCGAGGCCAAG
AACTACAGCGGCAGATTCACCTGTTGGTGGCTGACCACCATCAGCACCGATCTGACCTTCAGCG
TGAAGTCCAGCAGAGGCAGCTCTGATCCTCAAGGCGTTACATGTGGCGCCGCTACACTGTCTGC
CGAAAGAGTGCGGGGCGACAACAAAGAGTACGAGTACAGCGTCGAGTGCCAAGAGGATTCTG
CCTGTCCTGCCGCCGAGGAATCTCTGCCTATCGAAGTGATGGTGGACGCCGTGCACAAGCTGAA
GTACGAGAACTACACCAGCAGCTTTTTCATCCGGGACATCATCAAGCCCGATCCTCCAAAGAAC
CTGCAGCTGAAGCCCCTGAAGAACAGCAGACAGGTGGAAGTGTCTTGGGAGTACCCCGACACA
TGGTCTACCCCTCACAGCTACTTCAGCCTGACCTTCTGTGTGCAAGTGCAGGGCAAGTCCAAGC
GCGAGAAGAAAGATCGGGTGTTCACCGACAAGACCAGCGCCACCGTGATCTGCAGAAAGAAC
GCCAGCATCAGCGTGCGCGCTCAGGATAGGTACTACAGCAGCTCTTGGAGCGAGTGGGCCTCT
GTTCCTTGTTCTGGCGGCGGAGGAAGCGGAGGCGGAGGATCTGGTGGTGGTGGAAGTAGAAA
CCTGCCAGTGGCTACCCCTGATCCGGGCATGTTTCCTTGTCTGCACCACAGCCAGAACCTGCTGA
GAGCCGTGTCCAATATGCTGCAGAAGGCCCGGCAGACCCTTGAGTTCTACCCTTGCACCAGCGA
GGAAATCGACCACGAGGACATCACCAAGGATAAGACCAGCACCGTGGAAGCCTGCCTGCCTCT
GGAACTGACCAAGAACGAGTCCTGCCTGAACAGCCGGGAAACCAGCTTCATCACCAATGGCAG
CTGTCTGGCCAGCAGAAAGACCTCCTTCATGATGGCCCTGTGCCTGAGCAGCATCTACGAGGAC
CTGAAGATGTATCAGGTCGAGTTCAAGACCATGAACGCCAAGCTGCTGATGGACCCCAAGAGA
CAGATTTTCCTCGACCAGAACATGCTGGCCGTGATCGATGAACTGATGCAGGCCCTGAACTTCA
ACAGCGAGACAGTGCCCCAGAAGTCCTCTCTGGAAGAACCCGACTTCTACAAGACCAAGATCAA
GCTGTGCATCCTGCTGCACGCCTTCAGAATCAGGGCCGTGACCATCGACAGAGTGATGAGCTAT
CTGAACGCCAGCTGAAAGCTTGGTACCCAAACAAACAAAGGCGCGTCCTGGATTCGTGGTAAA
ACATACCAGATTTCGATCTGGAGAGGTGAAGAATACGACCACCTACTACATCCAGCTGATGAGT
CCCAAATAGGACGAAACGCGCTCAAACAAACAAAAGATCTCTTGTTTATTGCAGCTTATAATGG
TTACAAATAAAGCAATAGCATCACAAATTTCACAAATAAAGCATTTTTTTCACTGCATTCTAGTTG
TGGTTTGTCCAAACTCATCAATGTATCTTAACGCGGCCGCACGTGCGGACCGAGCGGCCGC
> single chain human IL12 - with SP according to SEQ ID NO: 52, long linker (Seq ID No.
75)
mgwsciilflvatatgvhsIWELKKDVYVVELDWYPDAPGEMVVLTCDTPEEDGITWTLDQSSEVLGSGK
TLTIQVKEFGDAGQYTCHKGGEVLSHSLLLLHKKEDGIWSTDILKDQKEPKNKTFLRCEAKNYSGRFT
CWWLTTISTDLTFSVKSSRGSSDPQGVTCGAATLSAERVRGDNKEYEYSVECQEDSACPAAEESLPIE
VMVDAVHKLKYENYTSSFFIRDIIKPDPPKNLQLKPLKNSRQVEVSWEYPDTWSTPHSYFSLTFCVQV
QGKSKREKKDRVFTDKTSATVICRKNASISVRAQDRYYSSSWSEWASVPCSGGGGSGGGGSGGGG
SRNLPVATPDPGMFPCLHHSQNLLRAVSNMLQKARQTLEFYPCTSEEIDHEDITKDKTSTVEACLPLE
LTKNESCLNSRETSFITNGSCLASRKTSFMMALCLSSIYEDLKMYQVEFKTMNAKLLMDPKRQIFLDQ
NMLAVIDELMQALNFNSETVPQKSSLEEPDFYKTKIKLCILLHAFRIRAVTIDRVMSYLNAS

TABLE 3

| Seq ID No | Comment | Sequence |
|---|---|---|
| 1 | human fusion protein (single chain human IL12 - without SP, | See Table 1 |
| | long linker) | |
| 2 | single chain human IL12 - with SP, long linker | |
| 3 | human P40 without SP (IL12b) | |
| 4 | human p35 without SP (IL12a) | |
| 5 | human single chain IL12 - without SP, short linker | |
| 6 | human single chain IL12 - with SP, short linker | |
| 7 | SV40 poly(A) | See Table 2 |
| 8 | AAV2 ITR (upstream lead strand) | |
| 9 | K19 riboswitch DNA (described in Beilstein et al., 2014) | |
| 10 | K19 riboswitch RNA | |
| 11 | Nucleotide sequence encoding functional muIL12 single | |
| | chain (human IgG signal peptide, p40 subunit, (Gly4Ser)3 | |
| | linker, p35 subunit | |
| 12 | Protein sequence of functional muIL12 single chain (p40 sub- | |
| | unit, (Gly4Ser)3 linker, p35 subunit) without signal peptide | |
| 13 | DNA coding for human IgG signal peptide | |
| 14 | murine IL-12 beta chain (p40 subunit) | |
| 15 | murine IL-12 alpha chain (p35 subunit) | |
| 16 | (Gly4Ser)3 linker | |
| 17 | Primer - K19-riboswitch FW | |
| 18 | Primer - K19-riboswitch RV | |
| 19 | K19-riboswitch probe | |
| 20 | Primer-anti-FITC gene FW | |
| 21 | Primer-anti-FITC gene RV | |
| 22 | anti-FITC gene RV | |
| 23 | Primer - polr2a FW | |
| 24 | Primer - polr2a FW | |
| 25 | polr2a probe | |
| 26 | Primer - POLR2A FW | |
| 27 | Primer - POLR2A RV | |
| 28 | Primer - POLR2A probe | |
| 29 | pAAV.LP1-mIL-12-3′-riboswitch | |
| 30 | pAAV.LP1-aFITC-3′-riboswitch | |
| 31 | pAAV.CMV-cNluc-3′-riboswitch | |
| 32 | pAAV.CMV-GFP-3′-riboswitch | |
| 33 | human IL-12 SP | |
| 34 | human single chain IL12 - without SP, G4S linker | See Table 1 |
| 35 | human single chain IL12 - with SP, G4S linker | |
| 36 | hIL-12 p35-p40, (G4S)3 linker | |
| 37 | hIL-12 p35-p40, (G4S)3 linker with signal peptide | |
| 38 | hIL-12 p35-p40, G6S linker | |
| 39 | hIL-12 p35-p40, G6S linker, with signal peptide | |
| 40 | hIL-12 p35-p40, G4S linker | |
| 41 | hIL-12 p35-p40, G4S linker with signal peptide | |
| 42 | LP1-Promotor/SV40-intron | See Table 2 |
| 43 | WT AAV2 ITR (reverse complement of the conventional view) | |
| 44 | AAV ITR delta(C) lacks 4nt at the 3′end and 11nt in the so | |
| | called c-region | |
| 45 | K19 riboswitch-inactive DNA | |
| 46 | sequence according to Seq ID 29 as confirmed by confirmatory | |
| | sequencing having an 11nt deletion in the left ITR | |
| 47 | Sequence according to Seq ID 29 but with the wt AAV2 ITR | |
| | sequences | |
| 48 | AAV2 ITR conventional view, see Wilmot et al, see Seq ID 43 | See sequence |
| | for its reverse complement | listing |
| 49 | AAV ITR according to Stratagene (reverse complement of the | |
| | conventional view) | |
| 50 | AAV.LP1-hIL-12-3′-riboswitch: signal peptide, IL12 p40- | Table 2 |
| | (G4S)3-p35; carrying the 4nt deletion in the ITR | |
| 51 | AAV.LP1-hIL-12-3′-riboswitch; signal peptide, IL12 p35- | |
| | (G4S)3-p40; carrying the 4nt deletion in the ITR | |
| 52 | human-immunoglobulin-signal-sequence | |
| 53 | signal-sequence-p35-p40-huIL12 | |
| 54 | p35-p40-huIL12 (after signal sequence cleavage) | |
| 55 | signal-sequence-p35-p40-huIL12-GS_linker | |
| 56 | p35-p40-huIL12-GS_linker (after signal sequence cleavage) | |
| 57 | AAV.LP1-hIL-12-3′-riboswitch: signal peptide, IL12 p40- | |
| | (G4S)3-p35; AAV2-WT ITR | |
| 58 | AAV.LP1-hIL-12-3′-riboswitch; signal peptide, IL12 p35- | |
| | (G4S)3-p40; AAV2-WT ITR | |
| 59 | AAV.LP1_SV40-hIL-12-3′-riboswitch: signal peptide, IL12 p40- | |
| | (G4S)3-p35; AAV2-WT ITR | |
| 60 | pAAV.LP1_SV40-hIL-12-3′-riboswitch: signal peptide, IL12 | |
| | p35-(G4S)3-p40; AAV2-WT ITR | |
| 61 | AAV.LP1_SV40-hIL-12-3′-riboswitch: signal peptide, IL12 p40- | |
| | (G4S)3-p35; carrying the 4nt deletion in the ITR | |
| 62 | AAV. LP1_SV40-hIL-12-3′riboswitch; signal peptide, IL12 | |
| | p35(G4S)3p40; carrying the 4nt deletion in the ITR | |
| 63 | AAV.LP1_SV40-hIL-12-3′-riboswitch: signal peptide, IL12 p40- | |
| | (G4S)3-p35; carrying the 11nt and 4nt deletion in the left ITR, | |
| | and the 4nt deletion in the right ITR | |
| 64 | AAV. LP1_SV40-hIL-12-3′riboswitch; signal peptide, IL12 | |
| | p35(G4S)3p40; carrying the 11nt and 4nt deletion in the left | |
| | ITR, and the 4nt deletion in the right ITR | |
| 65 | AAV.LP1-hIL-12-3′-riboswitch: signal peptide, IL12 p40- | |
| | (G4S)3-p35; carrying the 11nt and 4nt deletion in the left ITR, | |
| | and the 4nt deletion in the right ITR | |
| 66 | AAV. LP1-hIL-12-3′riboswitch: signal peptide, IL12 | |
| | p35(G4S)3p40; carrying the 11nt and 4nt deletion in the left | |
| | ITR, and the 4nt deletion in the right ITR | |
| 67 | human CEA promoter | |
| 68 | human Muc1 promoter | |
| 69 | human AFP promoter | |
| 70 | GenBank: AAN03857.1 Capsid Protein adeno-associated | |
| | Virus 8, AAV8 VP1 Sequence | |
| 71 | GenBank: AF513852.1 AAV8 VP1 Capsid CDS Sequence | |
| 72 | LP1 promotor without SV40 intron | |
| 73 | LP1-hIL-12-3′-riboswitch: signal peptide, IL12 p40-(G4S)3- | |
| | p35; expression cassette | |
| 74 | LP1_SV40-hIL-12-3′-riboswitch: signal peptide, IL12 p40- | |
| | (G4S)3-p35; expression cassette | |
| 75 | >single chain human IL12 - with SP according to SEQ ID NO: | |
| | 52, long linker (Seq ID No. 75) | |

TABLE 4
Alternative human single chain IL12 sequences can be
selected from the group consisting of a.1 to a.20:

| | Origin | Accession No. |
|---|---|---|
| a.1 | Sequence 8 from U.S. Pat. No. 9,580,485 | ATK71739.1 |
| a.2 | Sequence 105 from U.S. Pat. No. 8,945,571 | AKW40508.1 |
| a.3 | Sequence 4 from U.S. Pat. No. 9,447,159 | ARH47846.1 |
| a.4 | Sequence 46 from U.S. Pat. No. 10,232,053 | QCB09470.1 |
| a.5 | Sequence 45 from U.S. Pat. No. 10,696,723 | QPU05219.1 |
| a.6 | Sequence 8 from U.S. Pat. No. 5,994,104 | AAE38446.1 |
| a.7 | Sequence 12 from U.S. Pat. No. 5,994,104 | AAE38448.1 |
| a.8 | Sequence 3 from U.S. Pat. No. 10,723,782 | QPV46331.1 |
| a.9 | Sequence 5 from U.S. Pat. No. 10,723,782 | QPV46333.1 |
| a.10 | Sequence 49 from U.S. Pat. No. 10,646,549 | QNB69386.1 |
| a.11 | Sequence 1259 from U.S. Pat. No. 10,335,486 | QFN60269.1 |
| a.12 | Sequence 12 from U.S. Pat. No. 10,344,067 | QFO42313.1 |
| a.13 | Sequence 312 from U.S. Pat. No. 10,918,667 | QTW21904.1 |
| a.14 | Sequence 313 from U.S. Pat. No. 10,918,667 | QTW21905.1 |
| a.15 | Sequence 35 from U.S. Pat. No. 10,716,818 | QPV23492.1 |
| a.16 | Sequence 10 from U.S. Pat. No. 10,669,322 | QPT65568.1 |
| a.17 | Sequence 1 from U.S. Pat. No. 10,201,592 | QCA50445.1 |
| a.18 | Sequence 44 from U.S. Pat. No. 6,818,444 | AAW13832.1 |
| a.19 | Sequence 39 from U.S. Pat. No. 6,818,444 | AAW13831.1 |
| a.20 | Sequence 62 from U.S. Pat. No. 6,818,444 | AAW13838.1 |

1. Nucleic acid construct comprising a transgene encoding one or more therapeutic proteins, at least one tetracycline-responsive aptazyme sequence, and inverted terminal repeats (ITRs).

2. Nucleic acid construct according to claim 1, further comprising a promoter, such as a liver-specific promoter or a tumor-specific promoter.

3. Nucleic acid construct according to claim 1, wherein said promoter is selected from the group of the human cytomegalovirus (CMV) promoter, the liver-specific promoter LP1, the tumor-specific alpha fetoprotein (AFP) promoter, the human telomerase reverse transcriptase (hTERT) promoter, the CEA promoter and the Mud promoter.

4. Nucleic acid construct according to claim 1, further comprising a poly(A) signal, such as a SV40 poly(A) signal.

5. Nucleic acid construct according to claim 1, wherein said construct comprises single-stranded DNA or double-stranded DNA.

6. (canceled)

7. Nucleic acid construct according to claim 1, wherein said ITRs (a) flank the transgene and the aptazyme sequence, or (b) are derived from AAV2.

8. (canceled)

9. Nucleic acid construct according to claim 1, comprising transgene expression cassette, said transgene expression cassette comprising a promoter, a transgene encoding a therapeutic protein, a polyadenylation signal, and ITRs.

10.-11. (canceled)

12. Nucleic acid construct according to claim 1, wherein said transgene encodes one or more immunoregulatory proteins, wherein said immunoregulatory protein is selected from the group consisting of an interleukin, an interferon, an antibody, an antibody fragment and pro-inflammatory or pro-apoptotic members of the TNF/TNFR superfamily.

13. (canceled)

14. Nucleic acid construct according to claim 12, wherein said immunoregulatory protein is an interleukin selected from the group consisting of IL-4, IL-6, IL-10, IL-11, IL-12, IL-13 IL-23, IL-27, and IL-33.

15. Nucleic acid construct according to claim 14, wherein said interleukin is single chain IL-12, preferably a single chain IL-12 comprising one or more of the sequences selected from the group consisting of SEQ ID Nos. 1-6.

16. Nucleic acid construct according to claim 1, wherein said at least one tetracycline-responsive aptazyme sequence:
(a) is located 3′ of the transgene,
(b) induces or enhances expression of the transgene upon tetracycline binding, or
(c) comprises the sequence of SEQ ID NO:9.

17.-18. (canceled)

19. Nucleic acid construct according to claim 1, wherein said construct:
(a) comprises more than one tetracycline-responsive aptazyme sequence,
(b) is a plasmid, or
(c) comprises a transgene encoding single chain IL-12, at least one tetracycline responsive aptazyme sequence which comprises the sequence of SEQ ID NO:9, ITRs derived from AAV2, and optionally the liver-specific promoter LP1.

20.-21. (canceled)

22. Nucleic acid construct according to claim 19, which comprises any of the sequences set forth in SEQ ID NOs:50, 51, 57, 58, 59, 60, 61, 62, 63, 64, 65 or 66 or a complement thereof or a double stranded version thereof.

23. Nucleic acid construct according to claim 1, wherein the nucleic acid construct of the invention, after delivery into a test subject, results in an at least 4-fold, preferably at least 6-fold, at least 8-fold, or at least 9-fold, higher expression level of the transgene compared to baseline level 8 hours after administration of 30 mg tetracycline per kg bodyweight to said subject, wherein said test subject is preferably a mouse.

24. Transgene expression cassette comprising a promoter, a transgene encoding one or more therapeutic proteins, and at least one tetracycline-responsive aptazyme sequence.

25. Transgene expression cassette according to claim 24, wherein said promoter is a liver-specific promoter or a tumor-specific promoter.

26. Transgene expression cassette according to claim 24, further comprising a poly(A) signal, such as a SV40 poly(A) signal.

27. Transgene expression cassette according to claim 24, wherein said construct comprises single-stranded DNA or double-stranded DNA.

28. Transgene expression cassette according to claim 24, wherein said transgene encodes one or more immunoregulatory proteins, wherein said immunoregulatory protein is selected from the group consisting of an interleukin, an interferon, an antibody, an antibody fragment and pro-inflammatory or pro-apoptotic members of the TNF/TNFR superfamily.

29. (canceled)

30. Transgene expression cassette according to claim 28, wherein said immunoregulatory protein is an interleukin selected from the group consisting of IL-4, IL-6, IL-10, IL-11, IL-12, IL-13 IL-23, IL-27, and IL-33.

31. Transgene expression cassette according to claim 30, wherein said interleukin is single chain IL-12, preferably a single chain IL-12 comprising one or more of the sequences selected from the group consisting of SEQ ID Nos. 1-6.

32. Transgene expression cassette according to claim 24, wherein said at least one tetracycline-responsive aptazyme sequence:
(a) is located 3′ of the transgene,
(b) induces or enhances expression of the transgene upon tetracycline binding, or
(c) comprises the sequence of SEQ ID NO:9.

33.-34. (canceled)

35. Transgene expression cassette according to claim 24, wherein said cassette comprises more than one tetracycline-responsive aptazyme sequence.

36. Transgene expression cassette according to claim 24, wherein said construct comprises a transgene encoding single chain IL-12, at least one tetracycline-responsive aptazyme sequence which comprises the sequence of SEQ ID NO:9, and optionally the liver-specific promoter LP1.

37. Viral vector comprising a capsid and a packaged nucleic acid, wherein the packaged nucleic acid comprises a nucleic acid construct according to claim 1.

38. Viral vector according to claim 37, wherein said vector is a recombinant AAV vector.

39. Viral vector according to claim 38, wherein the vector is a recombinant AAV vector having the AAV-2, AAV-8 or AAV-9 serotype.

40. Viral vector according to claim 37, wherein said capsid comprises an amino acid sequence that provides for selective binding to a target tissue, such as liver tissue.

41. Viral vector according to claim 37, wherein said vector is a recombinant AAV vector having the AAV-8 serotype.

42. Viral vector according to claim 41, wherein said nucleic acid construct comprises a transgene encoding single chain IL-12, at least one tetracycline responsive aptazyme sequence which comprises the sequence of SEQ ID NO:9, ITRs derived from AAV2, and optionally the liver-specific promoter LP1.

43. Viral vector having the AAV-8 serotype, which comprises any of the sequences set forth in SEQ ID NOs:50, 51, 57, 58, 59, 60, 61, 62, 63, 64, 65 or 66 or a complement thereof or a double stranded version thereof.

44. Viral vector according to claim 43, which comprises any of the sequences set forth in SEQ ID NOs:57 and 59.

45.-53. (canceled)

54. Cell which comprises the nucleic acid construct according to claim 1.

55. Pharmaceutical composition comprising the nucleic acid construct according to claim 1 and a pharmaceutical-acceptable carrier or diluent.

56. Method of treating a proliferative disease comprising administering to a patient in need thereof a therapeutically effective amount of the nucleic acid construct according to claim 1.

57. Method according to claim 56, wherein said proliferative disease is a fibrosis or cancer disease.

58. Method according to claim 57, wherein said cancer disease is selected from the group of liver cancer brain cancer, pancreatic cancer, colorectal cancer, esophageal cancer, gastric cancer, hepatocellular cancer, anal cancer, breast cancer, cervical cancer, ovarian cancer, endometrial cancer, prostate cancer, testicular cancer, vulvar cancer, skin cancer, urogenital cancer, renal cancer, bladder cancer, head and neck cancer, oropharyngeal cancer, laryngeal cancer, non-small cell lung cancer, small cell lung cancer.

59. Method according to claim 58, wherein said cancer disease is liver cancer.

60. Method according to claim 59, wherein said liver cancer is hepatocellular carcinoma (HCC) or cholangiocarcinoma.

61. Method according to claim 56, wherein the patient to be treated has one or more cancer lesions located in the liver.

62.-63. (canceled)


SEQUENCE LISTING (80 sequences)

SEQ ID NO: 1 - protein, 518 aa, Artificial Sequence
  human fusion protein (single chain human IL12 - without SP, long linker)
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 2 - protein, 540 aa, Artificial Sequence
  single chain human IL12 - with SP, long linker
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 3 - protein, 306 aa, Artificial Sequence
  human P40 without SP (IL12b)
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 4 - protein, 197 aa, Artificial Sequence
  human p35 without SP (IL12a)
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 5 - protein, 510 aa, Artificial Sequence
  human single chain IL12 - without SP, short linker
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 6 - protein, 532 aa, Artificial Sequence
  human single chain IL12 - with SP, short linker
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 7 - DNA, 129 bp, Artificial Sequence
  SV40 poly(A)
cttgtttattgcagcttataatggttacaaataaagcaatagcatcacaaatttcacaaataaagcatttttttcactgcattctagttgtggtttgtccaaactcatcaatgtatcttaacgcggccg

SEQ ID NO: 8 - DNA, 141 bp, Artificial Sequence
  AAV2 ITR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 9 - DNA, 110 bp, Artificial Sequence
  K19 riboswitch DNA (described in Beilstein et al., 2014)
ggcgcgtcctggattcgtggtaaaacataccagatttcgatctggagaggtgaagaatacgaccacctactacatccagctgatgagtcccaaataggacgaaacgcgct

SEQ ID NO: 10 - RNA, 110 nt, Artificial Sequence
  K19 riboswitch RNA
ggcgcguccuggauucgugguaaaacauaccagauuucgaucuggagaggugaagaauacgaccaccuacuacauccagcugaugagucccaaauaggacgaaacgcgcu

SEQ ID NO: 11 - DNA, 1623 bp, Artificial Sequence
  Nucleotide sequence encoding functional muIL12 single chain (human IgG signal peptide, p40 subunit, (Gly4Ser)3 linker, p35 subunit
atgggctggtcctgcatcattctgtttctggtggccacagccaccggtgtccactctatgtgggaactcgagaaggacgtgtacgtggtggaagtggactggacacctgatgctccaggcgagacagtgaacctgacctgtgacacacccgaagaggacgacatcacctggacaagcgatcagagacacggcgtgatcggcagcggcaagaccctgacaatcaccgtgaaagagtttctggacgccggccagtacacctgtcacaaaggcggagagacactgtcccacagccatctgctgctgcacaagaaagagaacggcatctggtccaccgagatcctgaagaacttcaagaacaagaccttcctgaagtgcgaggcccctaactacagcggcagattcacatgtagctggctggtgcagagaaacatggacctgaagttcaacatcaagtcctccagcagcagccccgacagcagagctgttacatgtggcatggctagcctgagcgccgagaaagtgacactggaccagagagactacgagaagtacagcgtgtcctgccaagaggacgtgacctgtcctacagccgaggaaacactgcctatcgagctggccctggaagccagacagcagaacaaatacgagaactactctaccagcttcttcatccgggacatcatcaagcccgatcctccaaagaacctgcagatgaagcctctgaagaacagccaggtcgaggtgtcctgggagtaccctgactcttggagcacccctcacagctacttcagcctgaaattcttcgtgcgcatccagcgcaagaaagaaaagatgaaggaaaccgaggaaggctgcaaccagaagggcgccttcctggtcgaaaagacctctaccgaggtgcagtgcaaaggcggcaatgtctgtgtgcaggcccaggataggtactacaacagcagctgcagcaagtgggcctgcgtgccatgtagagttagaagcggaggcggaggaagtggtggcggaggttctggcggcggtggaagtagagttatccctgtgtctggccctgccagatgcctgtctcagagcagaaacctgctgaaaaccaccgacgacatggtcaagaccgccagagagaagctgaagcactacagctgcaccgccgaggacatcgaccacgaggatatcacaagggaccagaccagcacactgaaaacctgcctgcctctggaactgcataagaacgagagctgcctggccacaagagagacaagcagcaccacaagaggcagctgtctgcctcctcagaaaaccagcctgatgatgacactgtgcctgggcagcatctacgaggatctgaagatgtaccagaccgagttccaggccatcaacgccgctctgcagaaccacaaccaccagcagatcatcctggataagggcatgctggtggctatcgacgagctgatgcagagcctgaaccacaatggcgagacactgagacagaagcctccagtcggagaggccgatccttacagagtgaagatgaagctgtgcatcctgctgcacgccttcagcaccagagtggtcaccatcaacagagtgatgggctacctgagtagtgcatga

SEQ ID NO: 12 - protein, 521 aa, Artificial Sequence
  Protein sequence of functional muIL12 single chain (p40 subunit, (Gly4Ser)3 linker, p35 subunit) without signal peptide
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 13 - DNA, 57 bp, Artificial Sequence
  DNA coding for human IgG signal peptide
atgggctggtcctgcatcattctgtttctggtggccacagccaccggtgtccactct

SEQ ID NO: 14 - DNA, 936 bp, Artificial Sequence
  murine IL-12 beta chain (p40 subunit)
atgtgggaactcgagaaggacgtgtacgtggtggaagtggactggacacctgatgctccaggcgagacagtgaacctgacctgtgacacacccgaagaggacgacatcacctggacaagcgatcagagacacggcgtgatcggcagcggcaagaccctgacaatcaccgtgaaagagtttctggacgccggccagtacacctgtcacaaaggcggagagacactgtcccacagccatctgctgctgcacaagaaagagaacggcatctggtccaccgagatcctgaagaacttcaagaacaagaccttcctgaagtgcgaggcccctaactacagcggcagattcacatgtagctggctggtgcagagaaacatggacctgaagttcaacatcaagtcctccagcagcagccccgacagcagagctgttacatgtggcatggctagcctgagcgccgagaaagtgacactggaccagagagactacgagaagtacagcgtgtcctgccaagaggacgtgacctgtcctacagccgaggaaacactgcctatcgagctggccctggaagccagacagcagaacaaatacgagaactactctaccagcttcttcatccgggacatcatcaagcccgatcctccaaagaacctgcagatgaagcctctgaagaacagccaggtcgaggtgtcctgggagtaccctgactcttggagcacccctcacagctacttcagcctgaaattcttcgtgcgcatccagcgcaagaaagaaaagatgaaggaaaccgaggaaggctgcaaccagaagggcgccttcctggtcgaaaagacctctaccgaggtgcagtgcaaaggcggcaatgtctgtgtgcaggcccaggataggtactacaacagcagctgcagcaagtgggcctgcgtgccatgtagagttaga

SEQ ID NO: 15 - DNA, 582 bp, Artificial Sequence
  murine IL-12 alpha chain (p35 subunit)
agagttatccctgtgtctggccctgccagatgcctgtctcagagcagaaacctgctgaaaaccaccgacgacatggtcaagaccgccagagagaagctgaagcactacagctgcaccgccgaggacatcgaccacgaggatatcacaagggaccagaccagcacactgaaaacctgcctgcctctggaactgcataagaacgagagctgcctggccacaagagagacaagcagcaccacaagaggcagctgtctgcctcctcagaaaaccagcctgatgatgacactgtgcctgggcagcatctacgaggatctgaagatgtaccagaccgagttccaggccatcaacgccgctctgcagaaccacaaccaccagcagatcatcctggataagggcatgctggtggctatcgacgagctgatgcagagcctgaaccacaatggcgagacactgagacagaagcctccagtcggagaggccgatccttacagagtgaagatgaagctgtgcatcctgctgcacgccttcagcaccagagtggtcaccatcaacagagtgatgggctacctgagtagtgcatga

SEQ ID NO: 16 - DNA, 48 bp, Artificial Sequence
  (Gly4Ser)3 linker
agcggaggcggaggaagtggtggcggaggttctggcggcggtggaagt

SEQ ID NO: 17 - DNA, 20 bp, Artificial Sequence
  Primer -K19-riboswitch FW
gcgtcctggattcgtggtaa

SEQ ID NO: 18 - DNA, 25 bp, Artificial Sequence
  Primer - K19-riboswitch RV
gctggatgtagtaggtggtcgtatt

SEQ ID NO: 19 - DNA, 19 bp, Artificial Sequence
  K19-riboswitch probe
atttcgatctggagaggtg

SEQ ID NO: 20 - DNA, 20 bp, Artificial Sequence
  Primer anti-FITC gene FW
tctgcgcgtggaagatacag

SEQ ID NO: 21 - DNA, 23 bp, Artificial Sequence
  Primer - anti-FITC gene RV
caatatccggaggagtcgtagct

SEQ ID NO: 22 - DNA, 20 bp, Artificial Sequence
  anti-FITC gene RV
tgtgtattattgcgctaggc

SEQ ID NO: 23 - DNA, 24 bp, Artificial Sequence
  Primer - polr2a FW
gccaaagactccttcactcactgt

SEQ ID NO: 24 - DNA, 20 bp, Artificial Sequence
  Primer - polr2a FW
ttccaagcggcaaagaatgt

SEQ ID NO: 25 - DNA, 27 bp, Artificial Sequence
  polr2a probe
tggctctttcagcatctcgtgcagatt

SEQ ID NO: 26 - DNA, 19 bp, Artificial Sequence
  Primer - POLR2A FW
gcaagcggattccatttgg

SEQ ID NO: 27 - DNA, 21 bp, Artificial Sequence
  Primer - POLR2A RV
tctcaggcccgtagtcatcct

SEQ ID NO: 28 - DNA, 27 bp, Artificial Sequence
  Primer - POLR2A probe
aagcaccggactctgcctcacttcatc

SEQ ID NO: 29 - DNA, 2714 bp, Artificial Sequence
  pAAV.LP1-mIL-12-3′-riboswitch
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagagaattccccggaccggtggatccgccaccatgggctggtcctgcatcattctgtttctggtggccacagccaccggtgtccactctatgtgggaactcgagaaggacgtgtacgtggtggaagtggactggacacctgatgctccaggcgagacagtgaacctgacctgtgacacacccgaagaggacgacatcacctggacaagcgatcagagacacggcgtgatcggcagcggcaagaccctgacaatcaccgtgaaagagtttctggacgccggccagtacacctgtcacaaaggcggagagacactgtcccacagccatctgctgctgcacaagaaagagaacggcatctggtccaccgagatcctgaagaacttcaagaacaagaccttcctgaagtgcgaggcccctaactacagcggcagattcacatgtagctggctggtgcagagaaacatggacctgaagttcaacatcaagtcctccagcagcagccccgacagcagagctgttacatgtggcatggctagcctgagcgccgagaaagtgacactggaccagagagactacgagaagtacagcgtgtcctgccaagaggacgtgacctgtcctacagccgaggaaacactgcctatcgagctggccctggaagccagacagcagaacaaatacgagaactactctaccagcttcttcatccgggacatcatcaagcccgatcctccaaagaacctgcagatgaagcctctgaagaacagccaggtcgaggtgtcctgggagtaccctgactcttggagcacccctcacagctacttcagcctgaaattcttcgtgcgcatccagcgcaagaaagaaaagatgaaggaaaccgaggaaggctgcaaccagaagggcgccttcctggtcgaaaagacctctaccgaggtgcagtgcaaaggcggcaatgtctgtgtgcaggcccaggataggtactacaacagcagctgcagcaagtgggcctgcgtgccatgtagagttagaagcggaggcggaggaagtggtggcggaggttctggcggcggtggaagtagagttatccctgtgtctggccctgccagatgcctgtctcagagcagaaacctgctgaaaaccaccgacgacatggtcaagaccgccagagagaagctgaagcactacagctgcaccgccgaggacatcgaccacgaggatatcacaagggaccagaccagcacactgaaaacctgcctgcctctggaactgcataagaacgagagctgcctggccacaagagagacaagcagcaccacaagaggcagctgtctgcctcctcagaaaaccagcctgatgatgacactgtgcctgggcagcatctacgaggatctgaagatgtaccagaccgagttccaggccatcaacgccgctctgcagaaccacaaccaccagcagatcatcctggataagggcatgctggtggctatcgacgagctgatgcagagcctgaaccacaatggcgagacactgagacagaagcctccagtcggagaggccgatccttacagagtgaagatgaagctgtgcatcctgctgcacgccttcagcaccagagtggtcaccatcaacagagtgatgggctacctgagtagtgcatgaaagcttggtacccaaacaaacaaaggcgcgtcctggattcgtggtaaaacataccagatttcgatctggagaggtgaagaatacgaccacctactacatccagctgatgagtcccaaataggacgaaacgcgctcaaacaaacaaaagatctcttgtttattgcagcttataatggttacaaataaagcaatagcatcacaaatttcacaaataaagcatttttttcactgcattctagttgtggtttgtccaaactcatcaatgtatcttaacgcggccgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 30 - DNA, 2747 bp, Artificial Sequence
  pAAV.LP1-aFITC-3′-riboswitch
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagagaattccccggaccggtggatccgccaccatggactggacctggcgggtgttctgtctgctggctgtggctcctggcgcccactctcaggtgcagctggtggaatctggcggcaacctggtgcagcctggcggctctctgagactgtcttgtgccgccagcggcttcaccttcggcagcttcagcatgagctgggtgcgacaggctccaggcggaggactggaatgggtggcaggcctgagcgccagaagcagcctgacacactacgccgacagcgtgaagggcagattcaccatcagcagagacaacgccaagaacagcgtgtacctgcagatgaacagcctgagggtggaagataccgccgtgtactactgtgccagaagaagctacgacagcagcggctactggggccacttctacagctacatggacgtgtggggccagggcaccctcgtgacagtgtctagtggcggaggcggaagtggcggcggaggatcagggggaggcggatctcagtctgtgctgacccagcctagcagcgtgtccgctgctcctggccagaaagtgaccatcagctgcagcggcagcaccagcaacatcggcaacaactacgtgtcctggtatcagcagcaccccggcaaggcccccaagctgatgatctacgacgtgtccaagaggcccagcggcgtgcccgatagattcagcggctctaagagcggcaacagcgccagcctggacatcagcggcctgcagtctgaggacgaggccgactattactgcgccgcctgggacgacagcctgtccgagttcctgttcggcaccggcaccaagctgacagtgctgggagggggaggatctggcgggggaggctcacaggtgcagctggtggaaagcggcggaaatctggtgcagccagggggcagcctgagactgagctgtgccgcttccggctttacctttggctccttctccatgtcctgggtgcgccaggcacctgggggcggactggaatgggtggccggactgtctgccagaagctctctgacccactatgctgactctgtgaagggccggttcacaatctcccgggataacgctaagaactctgtgtacctgcagatgaactctctgcgcgtggaagatacagctgtgtattattgcgctaggcggagctacgactcctccggatattggggacacttttactcttatatggatgtgtgggggcagggaacactcgtgaccgtgtcaagcggaggcggcggaagcgggggagggggatctgggggcggaggcagtcagagtgtgctgacacagcccagctccgtgtctgccgccccaggacagaaagtgacaatctcctgctccggctccacctccaatatcggaaacaattatgtgtcttggtatcagcagcatcctgggaaggctcctaaactgatgatctatgatgtgtctaaacggccttccggcgtgccagacaggttctccggaagcaagtccggcaactccgcctctctggacatctccggactgcagagcgaggatgaggctgactactattgtgctgcttgggacgactccctgagcgagtttctgtttggaacagggacaaaactgaccgtgctgggcggcagcggaggcaagcctatccctaatcctctgctgggcctggacagcacctgaaagcttggtacccaaacaaacaaaggcgcgtcctggattcgtggtaaaacataccagatttcgatctggagaggtgaagaatacgaccacctactacatccagctgatgagtcccaaataggacgaaacgcgctcaaacaaacaaaagatctcttgtttattgcagcttataatggttacaaataaagcaatagcatcacaaatttcacaaataaagcatttttttcactgcattctagttgtggtttgtccaaactcatcaatgtatcttaacgcggccgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 31 - DNA, 1816 bp, Artificial Sequence
  pAAV.CMV-cNluc-3′-riboswitch
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaacatcgattgaattccccggaccggtggatccgccaccatggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaaaagcttggtacccaaacaaacaaaggcgcgtcctggattcgtggtaaaacataccagatttcgatctggagaggtgaagaatacgaccacctactacatccagctgatgagtcccaaataggacgaaacgcgctcaaacaaacaaaagatctcttgtttattgcagcttataatggttacaaataaagcaatagcatcacaaatttcacaaataaagcatttttttcactgcattctagttgtggtttgtccaaactcatcaatgtatcttaacgcggccgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 32 - DNA, 2059 bp, Artificial Sequence
  pAAV.CMV-GFP-3′-riboswitch
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaacatcgattgaattccccggaccggtggatccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggccggactcagatttcgagctcaagttttgaattttagaagcttggtacccaaacaaacaaaggcgcgtcctggattcgtggtaaaacataccagatttcgatctggagaggtgaagaatacgaccacctactacatccagctgatgagtcccaaataggacgaaacgcgctcaaacaaacaaaagatctcttgtttattgcagcttataatggttacaaataaagcaatagcatcacaaatttcacaaataaagcatttttttcactgcattctagttgtggtttgtccaaactcatcaatgtatcttaacgcggccgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 33 - protein, 22 aa, Homo sapiens
  MISC_FEATURE (1)..(11): human IL-12 SP
MCHQQLVISWFSLVFLASPLVA

SEQ ID NO: 34 - protein, 508 aa, Artificial Sequence
  human single chain IL12 - without SP, G4S linker
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 35 - protein, 530 aa, Artificial Sequence
  human single chain IL12 - with SP, G4S linker
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 36 - protein, 518 aa, Artificial Sequence
  hIL-12 p35-p40, (G4S)3 linker
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 37 - protein, 540 aa, Artificial Sequence
  hIL-12 p35-p40, (G4S)3 linker with signal peptide
MCHQQLVISWFSLVFLASPLVARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 38 - protein, 510 aa, Artificial Sequence
  hIL-12 p35-p40, G6S linker
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 39 - protein, 532 aa, Artificial Sequence
  hIL-12 p35-p40, G6S linker, with signal peptide
MCHQQLVISWFSLVFLASPLVARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 40 - protein, 508 aa, Artificial Sequence
  hIL-12 p35-p40, G4S linker #
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 41 - protein, 530 aa, Artificial Sequence
  hIL-12 p35-p40, G4S linker with signal peptide
MCHQQLVISWFSLVFLASPLVARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 42 - DNA, 661 bp, Artificial Sequence
  LP1-Promotor/SV40-intron
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctattttagattccaacctttggaactga

SEQ ID NO: 43 - DNA, 145 bp, Artificial Sequence
  WT AAV2 ITR (reverse complement of the conventional view)
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 44 - DNA, 130 bp, Artificial Sequence
  AAV ITR delta(C) lacks 4nt at the 3′end and 11nt in the so called c-region
aggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 45 - DNA, 110 bp, Artificial Sequence
  K19 riboswitch-inactive DNA
ggcgcgtcctggattcgtggtaaaacataccagatttcgatctggagaggtgaagaatacgaccacctactacatccagctgatgagtcccaaataggacgagacgcgct

SEQ ID NO: 46 - DNA, 2703 bp, Artificial Sequence
  sequence according to Seq ID 29 as confirmed by confirmatory sequencing having an 11nt deletion in the left ITR
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgttcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagagaattccccggaccggtggatccgccaccatgggctggtcctgcatcattctgtttctggtggccacagccaccggtgtccactctatgtgggaactcgagaaggacgtgtacgtggtggaagtggactggacacctgatgctccaggcgagacagtgaacctgacctgtgacacacccgaagaggacgacatcacctggacaagcgatcagagacacggcgtgatcggcagcggcaagaccctgacaatcaccgtgaaagagtttctggacgccggccagtacacctgtcacaaaggcggagagacactgtcccacagccatctgctgctgcacaagaaagagaacggcatctggtccaccgagatcctgaagaacttcaagaacaagaccttcctgaagtgcgaggcccctaactacagcggcagattcacatgtagctggctggtgcagagaaacatggacctgaagttcaacatcaagtcctccagcagcagccccgacagcagagctgttacatgtggcatggctagcctgagcgccgagaaagtgacactggaccagagagactacgagaagtacagcgtgtcctgccaagaggacgtgacctgtcctacagccgaggaaacactgcctatcgagctggccctggaagccagacagcagaacaaatacgagaactactctaccagcttcttcatccgggacatcatcaagcccgatcctccaaagaacctgcagatgaagcctctgaagaacagccaggtcgaggtgtcctgggagtaccctgactcttggagcacccctcacagctacttcagcctgaaattcttcgtgcgcatccagcgcaagaaagaaaagatgaaggaaaccgaggaaggctgcaaccagaagggcgccttcctggtcgaaaagacctctaccgaggtgcagtgcaaaggcggcaatgtctgtgtgcaggcccaggataggtactacaacagcagctgcagcaagtgggcctgcgtgccatgtagagttagaagcggaggcggaggaagtggtggcggaggttctggcggcggtggaagtagagttatccctgtgtctggccctgccagatgcctgtctcagagcagaaacctgctgaaaaccaccgacgacatggtcaagaccgccagagagaagctgaagcactacagctgcaccgccgaggacatcgaccacgaggatatcacaagggaccagaccagcacactgaaaacctgcctgcctctggaactgcataagaacgagagctgcctggccacaagagagacaagcagcaccacaagaggcagctgtctgcctcctcagaaaaccagcctgatgatgacactgtgcctgggcagcatctacgaggatctgaagatgtaccagaccgagttccaggccatcaacgccgctctgcagaaccacaaccaccagcagatcatcctggataagggcatgctggtggctatcgacgagctgatgcagagcctgaaccacaatggcgagacactgagacagaagcctccagtcggagaggccgatccttacagagtgaagatgaagctgtgcatcctgctgcacgccttcagcaccagagtggtcaccatcaacagagtgatgggctacctgagtagtgcatgaaagcttggtacccaaacaaacaaaggcgcgtcctggattcgtggtaaaacataccagatttcgatctggagaggtgaagaatacgaccacctactacatccagctgatgagtcccaaataggacgaaacgcgctcaaacaaacaaaagatctcttgtttattgcagcttataatggttacaaataaagcaatagcatcacaaatttcacaaataaagcatttttttcactgcattctagttgtggtttgtccaaactcatcaatgtatcttaacgcggccgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 47 - DNA, 2722 bp, Artificial Sequence
  Sequence accoding to Seq ID 29 with the wt AAV2 ITR sequences
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggccgcacgcgttcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagagaattccccggaccggtggatccgccaccatgggctggtcctgcatcattctgtttctggtggccacagccaccggtgtccactctatgtgggaactcgagaaggacgtgtacgtggtggaagtggactggacacctgatgctccaggcgagacagtgaacctgacctgtgacacacccgaagaggacgacatcacctggacaagcgatcagagacacggcgtgatcggcagcggcaagaccctgacaatcaccgtgaaagagtttctggacgccggccagtacacctgtcacaaaggcggagagacactgtcccacagccatctgctgctgcacaagaaagagaacggcatctggtccaccgagatcctgaagaacttcaagaacaagaccttcctgaagtgcgaggcccctaactacagcggcagattcacatgtagctggctggtgcagagaaacatggacctgaagttcaacatcaagtcctccagcagcagccccgacagcagagctgttacatgtggcatggctagcctgagcgccgagaaagtgacactggaccagagagactacgagaagtacagcgtgtcctgccaagaggacgtgacctgtcctacagccgaggaaacactgcctatcgagctggccctggaagccagacagcagaacaaatacgagaactactctaccagcttcttcatccgggacatcatcaagcccgatcctccaaagaacctgcagatgaagcctctgaagaacagccaggtcgaggtgtcctgggagtaccctgactcttggagcacccctcacagctacttcagcctgaaattcttcgtgcgcatccagcgcaagaaagaaaagatgaaggaaaccgaggaaggctgcaaccagaagggcgccttcctggtcgaaaagacctctaccgaggtgcagtgcaaaggcggcaatgtctgtgtgcaggcccaggataggtactacaacagcagctgcagcaagtgggcctgcgtgccatgtagagttagaagcggaggcggaggaagtggtggcggaggttctggcggcggtggaagtagagttatccctgtgtctggccctgccagatgcctgtctcagagcagaaacctgctgaaaaccaccgacgacatggtcaagaccgccagagagaagctgaagcactacagctgcaccgccgaggacatcgaccacgaggatatcacaagggaccagaccagcacactgaaaacctgcctgcctctggaactgcataagaacgagagctgcctggccacaagagagacaagcagcaccacaagaggcagctgtctgcctcctcagaaaaccagcctgatgatgacactgtgcctgggcagcatctacgaggatctgaagatgtaccagaccgagttccaggccatcaacgccgctctgcagaaccacaaccaccagcagatcatcctggataagggcatgctggtggctatcgacgagctgatgcagagcctgaaccacaatggcgagacactgagacagaagcctccagtcggagaggccgatccttacagagtgaagatgaagctgtgcatcctgctgcacgccttcagcaccagagtggtcaccatcaacagagtgatgggctacctgagtagtgcatgaaagcttggtacccaaacaaacaaaggcgcgtcctggattcgtggtaaaacataccagatttcgatctggagaggtgaagaatacgaccacctactacatccagctgatgagtcccaaataggacgaaacgcgctcaaacaaacaaaagatctcttgtttattgcagcttataatggttacaaataaagcaatagcatcacaaatttcacaaataaagcatttttttcactgcattctagttgtggtttgtccaaactcatcaatgtatcttaacgcggccgcacgtgcggaccgagcggccgcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 48 - DNA, 145 bp, Artificial Sequence
  AAV2 ITR conventional view, see Wilmot et al, see Seq ID 43 for its reverse complement
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 49 - DNA, 145 bp, Artificial Sequence
  AAV ITR according to Stratagene (reverse complement of the conventional view)
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcgg

SEQ ID NO: 50 - DNA, 2705 bp, Artificial Sequence
  pAAV.LP1-hIL-12-3′-riboswitch signalpeptide, IL12 p40-(G4S)3-p35; carrying the 4nt deletion in the ITR, as confimed by sequencing
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagagaattccccggaccggtggatccgccaccatgggctggtcctgcatcattctgtttctggtggccacagccaccggcgtgcactctatttgggagctgaagaaagacgtgtacgtggtcgagctggactggtatcctgatgctcccggcgaaatggtggtgctgacctgtgatacacccgaagaggacggcatcacatggacactggatcagtctagcgaggtgctcggcagcggcaagacactgaccatccaagtgaaagagtttggcgacgccggccagtacacatgtcacaaaggcggagaggtgctgagccattctctgctgctgctccacaagaaagaggatggcatttggagcaccgacatcctgaaggaccagaaagagcccaagaacaagaccttcctgagatgcgaggccaagaactacagcggcagattcacctgttggtggctgaccaccatcagcaccgatctgaccttcagcgtgaagtccagcagaggcagctctgatcctcaaggcgttacatgtggcgccgctacactgtctgccgaaagagtgcggggcgacaacaaagagtacgagtacagcgtcgagtgccaagaggattctgcctgtcctgccgccgaggaatctctgcctatcgaagtgatggtggacgccgtgcacaagctgaagtacgagaactacaccagcagctttttcatccgggacatcatcaagcccgatcctccaaagaacctgcagctgaagcccctgaagaacagcagacaggtggaagtgtcttgggagtaccccgacacatggtctacccctcacagctacttcagcctgaccttctgtgtgcaagtgcagggcaagtccaagcgcgagaagaaagatcgggtgttcaccgacaagaccagcgccaccgtgatctgcagaaagaacgccagcatcagcgtgcgcgctcaggataggtactacagcagctcttggagcgagtgggcctctgttccttgttctggcggcggaggaagcggaggcggaggatctggtggtggtggaagtagaaacctgccagtggctacccctgatccgggcatgtttccttgtctgcaccacagccagaacctgctgagagccgtgtccaatatgctgcagaaggcccggcagacccttgagttctacccttgcaccagcgaggaaatcgaccacgaggacatcaccaaggataagaccagcaccgtggaagcctgcctgcctctggaactgaccaagaacgagtcctgcctgaacagccgggaaaccagcttcatcaccaatggcagctgtctggccagcagaaagacctccttcatgatggccctgtgcctgagcagcatctacgaggacctgaagatgtatcaggtcgagttcaagaccatgaacgccaagctgctgatggaccccaagagacagattttcctcgaccagaacatgctggccgtgatcgatgaactgatgcaggccctgaacttcaacagcgagacagtgccccagaagtcctctctggaagaacccgacttctacaagaccaagatcaagctgtgcatcctgctgcacgccttcagaatcagggccgtgaccatcgacagagtgatgagctatctgaacgccagctgaaagcttggtacccaaacaaacaaaggcgcgtcctggattcgtggtaaaacataccagatttcgatctggagaggtgaagaatacgaccacctactacatccagctgatgagtcccaaataggacgaaacgcgctcaaacaaacaaaagatctcttgtttattgcagcttataatggttacaaataaagcaatagcatcacaaatttcacaaataaagcatttttttcactgcattctagttgtggtttgtccaaactcatcaatgtatcttaacgcggccgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 51 - DNA, 2705 bp, Artificial Sequence
  pAAV.LP1-hIL-12-3′-riboswitch - Sequence with 4nt deletion in ITRs; signalpeptide p35-(G4S)3-p40
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagagaattccccggaccggtggatccgccaccatgggctggtcctgcatcattctgtttctggtggccacagccaccggcgtgcactctagaaacctgccagtggctacccctgatccgggcatgtttccttgtctgcaccacagccagaacctgctgagagccgtgtccaatatgctgcagaaggcccggcagacccttgagttctacccttgcaccagcgaggaaatcgaccacgaggacatcaccaaggataagaccagcaccgtggaagcctgcctgcctctggaactgaccaagaacgagtcctgcctgaacagccgggaaaccagcttcatcaccaatggcagctgtctggccagcagaaagacctccttcatgatggccctgtgcctgagcagcatctacgaggacctgaagatgtatcaggtcgagttcaagaccatgaacgccaagctgctgatggaccccaagagacagattttcctcgaccagaacatgctggccgtgatcgatgaactgatgcaggccctgaacttcaacagcgagacagtgccccagaagtcctctctggaagaacccgacttctacaagaccaagatcaagctgtgcatcctgctgcacgccttcagaatcagggccgtgaccatcgacagagtgatgagctatctgaacgccagcggcggcggaggaagcggaggcggaggatctggtggtggtggaagtatttgggagctgaagaaagacgtgtacgtggtcgagctggactggtatcctgatgctcccggcgaaatggtggtgctgacctgtgatacacccgaagaggacggcatcacatggacactggatcagtctagcgaggtgctcggcagcggcaagacactgaccatccaagtgaaagagtttggcgacgccggccagtacacatgtcacaaaggcggagaggtgctgagccattctctgctgctgctccacaagaaagaggatggcatttggagcaccgacatcctgaaggaccagaaagagcccaagaacaagaccttcctgagatgcgaggccaagaactacagcggcagattcacctgttggtggctgaccaccatcagcaccgatctgaccttcagcgtgaagtccagcagaggcagctctgatcctcaaggcgttacatgtggcgccgctacactgtctgccgaaagagtgcggggcgacaacaaagagtacgagtacagcgtcgagtgccaagaggattctgcctgtcctgccgccgaggaatctctgcctatcgaagtgatggtggacgccgtgcacaagctgaagtacgagaactacaccagcagctttttcatccgggacatcatcaagcccgatcctccaaagaacctgcagctgaagcccctgaagaacagcagacaggtggaagtgtcttgggagtaccccgacacatggtctacccctcacagctacttcagcctgaccttctgtgtgcaagtgcagggcaagtccaagcgcgagaagaaagatcgggtgttcaccgacaagaccagcgccaccgtgatctgcagaaagaacgccagcatcagcgtgcgcgctcaggataggtactacagcagctcttggagcgagtgggcctctgttccttgttcttgaaagcttggtacccaaacaaacaaaggcgcgtcctggattcgtggtaaaacataccagatttcgatctggagaggtgaagaatacgaccacctactacatccagctgatgagtcccaaataggacgaaacgcgctcaaacaaacaaaagatctcttgtttattgcagcttataatggttacaaataaagcaatagcatcacaaatttcacaaataaagcatttttttcactgcattctagttgtggtttgtccaaactcatcaatgtatcttaacgcggccgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 52 - protein, 19 aa, Artificial Sequence
  human-immunoglobulin-signal-sequence
MGWSCIILFLVATATGVHS

SEQ ID NO: 53 - protein, 537 aa, Artificial Sequence
  signal-sequence-p35-p40-huIL12
MGWSCIILFLVATATGVHSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 54 - protein, 518 aa, Artificial Sequence; identical to SEQ ID NO: 36
  p35-p40-huIL12 (after signal sequence cleavage)

SEQ ID NO: 55 - protein, 546 aa, Artificial Sequence
  >signal-sequence-p35-p40-huIL12-GS_linker
MGWSCIILFLVATATGVHSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGS

SEQ ID NO: 56 - protein, 527 aa, Artificial Sequence
  >p35-p40-huIL12-GS_linker (after signal sequence cleavage)
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGS

SEQ ID NO: 57 - DNA, 2713 bp, Artificial Sequence
  > AAV.LP1-hIL-12-3-riboswitch signalpeptide, IL12 p40-(G4S)3-p35; AAV2-WT ITR
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggccgcacgcgttcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagagaattccccggaccggtggatccgccaccatgggctggtcctgcatcattctgtttctggtggccacagccaccggcgtgcactctatttgggagctgaagaaagacgtgtacgtggtcgagctggactggtatcctgatgctcccggcgaaatggtggtgctgacctgtgatacacccgaagaggacggcatcacatggacactggatcagtctagcgaggtgctcggcagcggcaagacactgaccatccaagtgaaagagtttggcgacgccggccagtacacatgtcacaaaggcggagaggtgctgagccattctctgctgctgctccacaagaaagaggatggcatttggagcaccgacatcctgaaggaccagaaagagcccaagaacaagaccttcctgagatgcgaggccaagaactacagcggcagattcacctgttggtggctgaccaccatcagcaccgatctgaccttcagcgtgaagtccagcagaggcagctctgatcctcaaggcgttacatgtggcgccgctacactgtctgccgaaagagtgcggggcgacaacaaagagtacgagtacagcgtcgagtgccaagaggattctgcctgtcctgccgccgaggaatctctgcctatcgaagtgatggtggacgccgtgcacaagctgaagtacgagaactacaccagcagctttttcatccgggacatcatcaagcccgatcctccaaagaacctgcagctgaagcccctgaagaacagcagacaggtggaagtgtcttgggagtaccccgacacatggtctacccctcacagctacttcagcctgaccttctgtgtgcaagtgcagggcaagtccaagcgcgagaagaaagatcgggtgttcaccgacaagaccagcgccaccgtgatctgcagaaagaacgccagcatcagcgtgcgcgctcaggataggtactacagcagctcttggagcgagtgggcctctgttccttgttctggcggcggaggaagcggaggcggaggatctggtggtggtggaagtagaaacctgccagtggctacccctgatccgggcatgtttccttgtctgcaccacagccagaacctgctgagagccgtgtccaatatgctgcagaaggcccggcagacccttgagttctacccttgcaccagcgaggaaatcgaccacgaggacatcaccaaggataagaccagcaccgtggaagcctgcctgcctctggaactgaccaagaacgagtcctgcctgaacagccgggaaaccagcttcatcaccaatggcagctgtctggccagcagaaagacctccttcatgatggccctgtgcctgagcagcatctacgaggacctgaagatgtatcaggtcgagttcaagaccatgaacgccaagctgctgatggaccccaagagacagattttcctcgaccagaacatgctggccgtgatcgatgaactgatgcaggccctgaacttcaacagcgagacagtgccccagaagtcctctctggaagaacccgacttctacaagaccaagatcaagctgtgcatcctgctgcacgccttcagaatcagggccgtgaccatcgacagagtgatgagctatctgaacgccagctgaaagcttggtacccaaacaaacaaaggcgcgtcctggattcgtggtaaaacataccagatttcgatctggagaggtgaagaatacgaccacctactacatccagctgatgagtcccaaataggacgaaacgcgctcaaacaaacaaaagatctcttgtttattgcagcttataatggttacaaataaagcaatagcatcacaaatttcacaaataaagcatttttttcactgcattctagttgtggtttgtccaaactcatcaatgtatcttaacgcggccgcacgtgcggaccgagcggccgcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 58 - DNA, 2713 bp, Artificial Sequence
  > pAAV.LP1-hIL-12-3-riboswitch; signalpeptide, IL12 p35-(G4S)3-p40; AAV2-WT ITR
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggccgcacgcgttcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagagaattccccggaccggtggatccgccaccatgggctggtcctgcatcattctgtttctggtggccacagccaccggcgtgcactctagaaacctgccagtggctacccctgatccgggcatgtttccttgtctgcaccacagccagaacctgctgagagccgtgtccaatatgctgcagaaggcccggcagacccttgagttctacccttgcaccagcgaggaaatcgaccacgaggacatcaccaaggataagaccagcaccgtggaagcctgcctgcctctggaactgaccaagaacgagtcctgcctgaacagccgggaaaccagcttcatcaccaatggcagctgtctggccagcagaaagacctccttcatgatggccctgtgcctgagcagcatctacgaggacctgaagatgtatcaggtcgagttcaagaccatgaacgccaagctgctgatggaccccaagagacagattttcctcgaccagaacatgctggccgtgatcgatgaactgatgcaggccctgaacttcaacagcgagacagtgccccagaagtcctctctggaagaacccgacttctacaagaccaagatcaagctgtgcatcctgctgcacgccttcagaatcagggccgtgaccatcgacagagtgatgagctatctgaacgccagcggcggcggaggaagcggaggcggaggatctggtggtggtggaagtatttgggagctgaagaaagacgtgtacgtggtcgagctggactggtatcctgatgctcccggcgaaatggtggtgctgacctgtgatacacccgaagaggacggcatcacatggacactggatcagtctagcgaggtgctcggcagcggcaagacactgaccatccaagtgaaagagtttggcgacgccggccagtacacatgtcacaaaggcggagaggtgctgagccattctctgctgctgctccacaagaaagaggatggcatttggagcaccgacatcctgaaggaccagaaagagcccaagaacaagaccttcctgagatgcgaggccaagaactacagcggcagattcacctgttggtggctgaccaccatcagcaccgatctgaccttcagcgtgaagtccagcagaggcagctctgatcctcaaggcgttacatgtggcgccgctacactgtctgccgaaagagtgcggggcgacaacaaagagtacgagtacagcgtcgagtgccaagaggattctgcctgtcctgccgccgaggaatctctgcctatcgaagtgatggtggacgccgtgcacaagctgaagtacgagaactacaccagcagctttttcatccgggacatcatcaagcccgatcctccaaagaacctgcagctgaagcccctgaagaacagcagacaggtggaagtgtcttgggagtaccccgacacatggtctacccctcacagctacttcagcctgaccttctgtgtgcaagtgcagggcaagtccaagcgcgagaagaaagatcgggtgttcaccgacaagaccagcgccaccgtgatctgcagaaagaacgccagcatcagcgtgcgcgctcaggataggtactacagcagctcttggagcgagtgggcctctgttccttgttcttgaaagcttggtacccaaacaaacaaaggcgcgtcctggattcgtggtaaaacataccagatttcgatctggagaggtgaagaatacgaccacctactacatccagctgatgagtcccaaataggacgaaacgcgctcaaacaaacaaaagatctcttgtttattgcagcttataatggttacaaataaagcaatagcatcacaaatttcacaaataaagcatttttttcactgcattctagttgtggtttgtccaaactcatcaatgtatcttaacgcggccgcacgtgcggaccgagcggccgcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 59 - DNA, 2911 bp, Artificial Sequence
  > AAV.LP1_SV40-hIL-12-3-riboswitch signalpeptide, IL12 p40-(G4S)3-p35; AAV2-WT ITR
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggccgcacgcgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctattttagattccaacctttggaactgagaattccccggaccggtggatccgccaccatgggctggtcctgcatcattctgtttctggtggccacagccaccggcgtgcactctatttgggagctgaagaaagacgtgtacgtggtcgagctggactggtatcctgatgctcccggcgaaatggtggtgctgacctgtgatacacccgaagaggacggcatcacatggacactggatcagtctagcgaggtgctcggcagcggcaagacactgaccatccaagtgaaagagtttggcgacgccggccagtacacatgtcacaaaggcggagaggtgctgagccattctctgctgctgctccacaagaaagaggatggcatttggagcaccgacatcctgaaggaccagaaagagcccaagaacaagaccttcctgagatgcgaggccaagaactacagcggcagattcacctgttggtggctgaccaccatcagcaccgatctgaccttcagcgtgaagtccagcagaggcagctctgatcctcaaggcgttacatgtggcgccgctacactgtctgccgaaagagtgcggggcgacaacaaagagtacgagtacagcgtcgagtgccaagaggattctgcctgtcctgccgccgaggaatctctgcctatcgaagtgatggtggacgccgtgcacaagctgaagtacgagaactacaccagcagctttttcatccgggacatcatcaagcccgatcctccaaagaacctgcagctgaagcccctgaagaacagcagacaggtggaagtgtcttgggagtaccccgacacatggtctacccctcacagctacttcagcctgaccttctgtgtgcaagtgcagggcaagtccaagcgcgagaagaaagatcgggtgttcaccgacaagaccagcgccaccgtgatctgcagaaagaacgccagcatcagcgtgcgcgctcaggataggtactacagcagctcttggagcgagtgggcctctgttccttgttctggcggcggaggaagcggaggcggaggatctggtggtggtggaagtagaaacctgccagtggctacccctgatccgggcatgtttccttgtctgcaccacagccagaacctgctgagagccgtgtccaatatgctgcagaaggcccggcagacccttgagttctacccttgcaccagcgaggaaatcgaccacgaggacatcaccaaggataagaccagcaccgtggaagcctgcctgcctctggaactgaccaagaacgagtcctgcctgaacagccgggaaaccagcttcatcaccaatggcagctgtctggccagcagaaagacctccttcatgatggccctgtgcctgagcagcatctacgaggacctgaagatgtatcaggtcgagttcaagaccatgaacgccaagctgctgatggaccccaagagacagattttcctcgaccagaacatgctggccgtgatcgatgaactgatgcaggccctgaacttcaacagcgagacagtgccccagaagtcctctctggaagaacccgacttctacaagaccaagatcaagctgtgcatcctgctgcacgccttcagaatcagggccgtgaccatcgacagagtgatgagctatctgaacgccagctgaaagcttggtacccaaacaaacaaaggcgcgtcctggattcgtggtaaaacataccagatttcgatctggagaggtgaagaatacgaccacctactacatccagctgatgagtcccaaataggacgaaacgcgctcaaacaaacaaaagatctcttgtttattgcagcttataatggttacaaataaagcaatagcatcacaaatttcacaaataaagcatttttttcactgcattctagttgtggtttgtccaaactcatcaatgtatcttaacgcggccgcacgtgcggaccgagcggccgcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 60 - DNA, 2911 bp, Artificial Sequence
  > pAAV.LP1_SV40-hIL-12-3-riboswitch signalpeptide, IL12 p35-(G4S)3-p40; AAV2-WT ITR
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggccgcacgcgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctattttagattccaacctttggaactgagaattccccggaccggtggatccgccaccatgggctggtcctgcatcattctgtttctggtggccacagccaccggcgtgcactctagaaacctgccagtggctacccctgatccgggcatgtttccttgtctgcaccacagccagaacctgctgagagccgtgtccaatatgctgcagaaggcccggcagacccttgagttctacccttgcaccagcgaggaaatcgaccacgaggacatcaccaaggataagaccagcaccgtggaagcctgcctgcctctggaactgaccaagaacgagtcctgcctgaacagccgggaaaccagcttcatcaccaatggcagctgtctggccagcagaaagacctccttcatgatggccctgtgcctgagcagcatctacgaggacctgaagatgtatcaggtcgagttcaagaccatgaacgccaagctgctgatggaccccaagagacagattttcctcgaccagaacatgctggccgtgatcgatgaactgatgcaggccctgaacttcaacagcgagacagtgccccagaagtcctctctggaagaacccgacttctacaagaccaagatcaagctgtgcatcctgctgcacgccttcagaatcagggccgtgaccatcgacagagtgatgagctatctgaacgccagcggcggcggaggaagcggaggcggaggatctggtggtggtggaagtatttgggagctgaagaaagacgtgtacgtggtcgagctggactggtatcctgatgctcccggcgaaatggtggtgctgacctgtgatacacccgaagaggacggcatcacatggacactggatcagtctagcgaggtgctcggcagcggcaagacactgaccatccaagtgaaagagtttggcgacgccggccagtacacatgtcacaaaggcggagaggtgctgagccattctctgctgctgctccacaagaaagaggatggcatttggagcaccgacatcctgaaggaccagaaagagcccaagaacaagaccttcctgagatgcgaggccaagaactacagcggcagattcacctgttggtggctgaccaccatcagcaccgatctgaccttcagcgtgaagtccagcagaggcagctctgatcctcaaggcgttacatgtggcgccgctacactgtctgccgaaagagtgcggggcgacaacaaagagtacgagtacagcgtcgagtgccaagaggattctgcctgtcctgccgccgaggaatctctgcctatcgaagtgatggtggacgccgtgcacaagctgaagtacgagaactacaccagcagctttttcatccgggacatcatcaagcccgatcctccaaagaacctgcagctgaagcccctgaagaacagcagacaggtggaagtgtcttgggagtaccccgacacatggtctacccctcacagctacttcagcctgaccttctgtgtgcaagtgcagggcaagtccaagcgcgagaagaaagatcgggtgttcaccgacaagaccagcgccaccgtgatctgcagaaagaacgccagcatcagcgtgcgcgctcaggataggtactacagcagctcttggagcgagtgggcctctgttccttgttcttgaaagcttggtacccaaacaaacaaaggcgcgtcctggattcgtggtaaaacataccagatttcgatctggagaggtgaagaatacgaccacctactacatccagctgatgagtcccaaataggacgaaacgcgctcaaacaaacaaaagatctcttgtttattgcagcttataatggttacaaataaagcaatagcatcacaaatttcacaaataaagcatttttttcactgcattctagttgtggtttgtccaaactcatcaatgtatcttaacgcggccgcacgtgcggaccgagcggccgcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 61 - DNA, 2903 bp, Artificial Sequence
  > AAV.LP1_SV40-hIL-12-3-riboswitch signalpeptide, IL12 p40-(G4S)3-p35; carrying the 4nt deletion in the ITR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctattttagattccaacctttggaactgagaattccccggaccggtggatccgccaccatgggctggtcctgcatcattctgtttctggtggccacagccaccggcgtgcactctatttgggagctgaagaaagacgtgtacgtggtcgagctggactggtatcctgatgctcccggcgaaatggtggtgctgacctgtgatacacccgaagaggacggcatcacatggacactggatcagtctagcgaggtgctcggcagcggcaagacactgaccatccaagtgaaagagtttggcgacgccggccagtacacatgtcacaaaggcggagaggtgctgagccattctctgctgctgctccacaagaaagaggatggcatttggagcaccgacatcctgaaggaccagaaagagcccaagaacaagaccttcctgagatgcgaggccaagaactacagcggcagattcacctgttggtggctgaccaccatcagcaccgatctgaccttcagcgtgaagtccagcagaggcagctctgatcctcaaggcgttacatgtggcgccgctacactgtctgccgaaagagtgcggggcgacaacaaagagtacgagtacagcgtcgagtgccaagaggattctgcctgtcctgccgccgaggaatctctgcctatcgaagtgatggtggacgccgtgcacaagctgaagtacgagaactacaccagcagctttttcatccgggacatcatcaagcccgatcctccaaagaacctgcagctgaagcccctgaagaacagcagacaggtggaagtgtcttgggagtaccccgacacatggtctacccctcacagctacttcagcctgaccttctgtgtgcaagtgcagggcaagtccaagcgcgagaagaaagatcgggtgttcaccgacaagaccagcgccaccgtgatctgcagaaagaacgccagcatcagcgtgcgcgctcaggataggtactacagcagctcttggagcgagtgggcctctgttccttgttctggcggcggaggaagcggaggcggaggatctggtggtggtggaagtagaaacctgccagtggctacccctgatccgggcatgtttccttgtctgcaccacagccagaacctgctgagagccgtgtccaatatgctgcagaaggcccggcagacccttgagttctacccttgcaccagcgaggaaatcgaccacgaggacatcaccaaggataagaccagcaccgtggaagcctgcctgcctctggaactgaccaagaacgagtcctgcctgaacagccgggaaaccagcttcatcaccaatggcagctgtctggccagcagaaagacctccttcatgatggccctgtgcctgagcagcatctacgaggacctgaagatgtatcaggtcgagttcaagaccatgaacgccaagctgctgatggaccccaagagacagattttcctcgaccagaacatgctggccgtgatcgatgaactgatgcaggccctgaacttcaacagcgagacagtgccccagaagtcctctctggaagaacccgacttctacaagaccaagatcaagctgtgcatcctgctgcacgccttcagaatcagggccgtgaccatcgacagagtgatgagctatctgaacgccagctgaaagcttggtacccaaacaaacaaaggcgcgtcctggattcgtggtaaaacataccagatttcgatctggagaggtgaagaatacgaccacctactacatccagctgatgagtcccaaataggacgaaacgcgctcaaacaaacaaaagatctcttgtttattgcagcttataatggttacaaataaagcaatagcatcacaaatttcacaaataaagcatttttttcactgcattctagttgtggtttgtccaaactcatcaatgtatcttaacgcggccgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 62 - DNA, 2903 bp, Artificial Sequence
  > pAAV. LP1_SV40-hIL-12-3riboswitch; signalpeptide, IL12 p35(G4S)3p40; carrying the 4nt deletion in the ITR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctattttagattccaacctttggaactgagaattccccggaccggtggatccgccaccatgggctggtcctgcatcattctgtttctggtggccacagccaccggcgtgcactctagaaacctgccagtggctacccctgatccgggcatgtttccttgtctgcaccacagccagaacctgctgagagccgtgtccaatatgctgcagaaggcccggcagacccttgagttctacccttgcaccagcgaggaaatcgaccacgaggacatcaccaaggataagaccagcaccgtggaagcctgcctgcctctggaactgaccaagaacgagtcctgcctgaacagccgggaaaccagcttcatcaccaatggcagctgtctggccagcagaaagacctccttcatgatggccctgtgcctgagcagcatctacgaggacctgaagatgtatcaggtcgagttcaagaccatgaacgccaagctgctgatggaccccaagagacagattttcctcgaccagaacatgctggccgtgatcgatgaactgatgcaggccctgaacttcaacagcgagacagtgccccagaagtcctctctggaagaacccgacttctacaagaccaagatcaagctgtgcatcctgctgcacgccttcagaatcagggccgtgaccatcgacagagtgatgagctatctgaacgccagcggcggcggaggaagcggaggcggaggatctggtggtggtggaagtatttgggagctgaagaaagacgtgtacgtggtcgagctggactggtatcctgatgctcccggcgaaatggtggtgctgacctgtgatacacccgaagaggacggcatcacatggacactggatcagtctagcgaggtgctcggcagcggcaagacactgaccatccaagtgaaagagtttggcgacgccggccagtacacatgtcacaaaggcggagaggtgctgagccattctctgctgctgctccacaagaaagaggatggcatttggagcaccgacatcctgaaggaccagaaagagcccaagaacaagaccttcctgagatgcgaggccaagaactacagcggcagattcacctgttggtggctgaccaccatcagcaccgatctgaccttcagcgtgaagtccagcagaggcagctctgatcctcaaggcgttacatgtggcgccgctacactgtctgccgaaagagtgcggggcgacaacaaagagtacgagtacagcgtcgagtgccaagaggattctgcctgtcctgccgccgaggaatctctgcctatcgaagtgatggtggacgccgtgcacaagctgaagtacgagaactacaccagcagctttttcatccgggacatcatcaagcccgatcctccaaagaacctgcagctgaagcccctgaagaacagcagacaggtggaagtgtcttgggagtaccccgacacatggtctacccctcacagctacttcagcctgaccttctgtgtgcaagtgcagggcaagtccaagcgcgagaagaaagatcgggtgttcaccgacaagaccagcgccaccgtgatctgcagaaagaacgccagcatcagcgtgcgcgctcaggataggtactacagcagctcttggagcgagtgggcctctgttccttgttcttgaaagcttggtacccaaacaaacaaaggcgcgtcctggattcgtggtaaaacataccagatttcgatctggagaggtgaagaatacgaccacctactacatccagctgatgagtcccaaataggacgaaacgcgctcaaacaaacaaaagatctcttgtttattgcagcttataatggttacaaataaagcaatagcatcacaaatttcacaaataaagcatttttttcactgcattctagttgtggtttgtccaaactcatcaatgtatcttaacgcggccgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 63 - DNA, 2892 bp, Artificial Sequence
  > AAV.LP1_SV40-hIL-12-3-riboswitch signalpeptide, IL12 p40-(G4S)3-p35; carrying the 11nt deletion in the left ITR, and the 4nt deletion in the right ITR
aggaacccctagtgatggagttggccactccctctctgcgcgctcgctcgctcactgaggccgggcgaccaaaggtcgcccgacgcccgggcggcctcagtgagcgagcgagcgcgcagctgcctgcagggcggccgcacgcgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctattttagattccaacctttggaactgagaattccccggaccggtggatccgccaccatgggctggtcctgcatcattctgtttctggtggccacagccaccggcgtgcactctatttgggagctgaagaaagacgtgtacgtggtcgagctggactggtatcctgatgctcccggcgaaatggtggtgctgacctgtgatacacccgaagaggacggcatcacatggacactggatcagtctagcgaggtgctcggcagcggcaagacactgaccatccaagtgaaagagtttggcgacgccggccagtacacatgtcacaaaggcggagaggtgctgagccattctctgctgctgctccacaagaaagaggatggcatttggagcaccgacatcctgaaggaccagaaagagcccaagaacaagaccttcctgagatgcgaggccaagaactacagcggcagattcacctgttggtggctgaccaccatcagcaccgatctgaccttcagcgtgaagtccagcagaggcagctctgatcctcaaggcgttacatgtggcgccgctacactgtctgccgaaagagtgcggggcgacaacaaagagtacgagtacagcgtcgagtgccaagaggattctgcctgtcctgccgccgaggaatctctgcctatcgaagtgatggtggacgccgtgcacaagctgaagtacgagaactacaccagcagctttttcatccgggacatcatcaagcccgatcctccaaagaacctgcagctgaagcccctgaagaacagcagacaggtggaagtgtcttgggagtaccccgacacatggtctacccctcacagctacttcagcctgaccttctgtgtgcaagtgcagggcaagtccaagcgcgagaagaaagatcgggtgttcaccgacaagaccagcgccaccgtgatctgcagaaagaacgccagcatcagcgtgcgcgctcaggataggtactacagcagctcttggagcgagtgggcctctgttccttgttctggcggcggaggaagcggaggcggaggatctggtggtggtggaagtagaaacctgccagtggctacccctgatccgggcatgtttccttgtctgcaccacagccagaacctgctgagagccgtgtccaatatgctgcagaaggcccggcagacccttgagttctacccttgcaccagcgaggaaatcgaccacgaggacatcaccaaggataagaccagcaccgtggaagcctgcctgcctctggaactgaccaagaacgagtcctgcctgaacagccgggaaaccagcttcatcaccaatggcagctgtctggccagcagaaagacctccttcatgatggccctgtgcctgagcagcatctacgaggacctgaagatgtatcaggtcgagttcaagaccatgaacgccaagctgctgatggaccccaagagacagattttcctcgaccagaacatgctggccgtgatcgatgaactgatgcaggccctgaacttcaacagcgagacagtgccccagaagtcctctctggaagaacccgacttctacaagaccaagatcaagctgtgcatcctgctgcacgccttcagaatcagggccgtgaccatcgacagagtgatgagctatctgaacgccagctgaaagcttggtacccaaacaaacaaaggcgcgtcctggattcgtggtaaaacataccagatttcgatctggagaggtgaagaatacgaccacctactacatccagctgatgagtcccaaataggacgaaacgcgctcaaacaaacaaaagatctcttgtttattgcagcttataatggttacaaataaagcaatagcatcacaaatttcacaaataaagcatttttttcactgcattctagttgtggtttgtccaaactcatcaatgtatcttaacgcggccgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 64 - DNA, 2694 bp, Artificial Sequence
  > AAV. LP1_SV40-hIL-12-3riboswitch; signalpeptide, IL12 p35(G4S)3p40; carrying the 11nt deletion in the left ITR, and the 4nt deletion in the right ITR
aggaacccctagtgatggagttggccactccctctctgcgcgctcgctcgctcactgaggccgggcgaccaaaggtcgcccgacgcccgggcggcctcagtgagcgagcgagcgcgcagctgcctgcagggcggccgcacgcgttcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagagaattccccggaccggtggatccgccaccatgggctggtcctgcatcattctgtttctggtggccacagccaccggcgtgcactctatttgggagctgaagaaagacgtgtacgtggtcgagctggactggtatcctgatgctcccggcgaaatggtggtgctgacctgtgatacacccgaagaggacggcatcacatggacactggatcagtctagcgaggtgctcggcagcggcaagacactgaccatccaagtgaaagagtttggcgacgccggccagtacacatgtcacaaaggcggagaggtgctgagccattctctgctgctgctccacaagaaagaggatggcatttggagcaccgacatcctgaaggaccagaaagagcccaagaacaagaccttcctgagatgcgaggccaagaactacagcggcagattcacctgttggtggctgaccaccatcagcaccgatctgaccttcagcgtgaagtccagcagaggcagctctgatcctcaaggcgttacatgtggcgccgctacactgtctgccgaaagagtgcggggcgacaacaaagagtacgagtacagcgtcgagtgccaagaggattctgcctgtcctgccgccgaggaatctctgcctatcgaagtgatggtggacgccgtgcacaagctgaagtacgagaactacaccagcagctttttcatccgggacatcatcaagcccgatcctccaaagaacctgcagctgaagcccctgaagaacagcagacaggtggaagtgtcttgggagtaccccgacacatggtctacccctcacagctacttcagcctgaccttctgtgtgcaagtgcagggcaagtccaagcgcgagaagaaagatcgggtgttcaccgacaagaccagcgccaccgtgatctgcagaaagaacgccagcatcagcgtgcgcgctcaggataggtactacagcagctcttggagcgagtgggcctctgttccttgttctggcggcggaggaagcggaggcggaggatctggtggtggtggaagtagaaacctgccagtggctacccctgatccgggcatgtttccttgtctgcaccacagccagaacctgctgagagccgtgtccaatatgctgcagaaggcccggcagacccttgagttctacccttgcaccagcgaggaaatcgaccacgaggacatcaccaaggataagaccagcaccgtggaagcctgcctgcctctggaactgaccaagaacgagtcctgcctgaacagccgggaaaccagcttcatcaccaatggcagctgtctggccagcagaaagacctccttcatgatggccctgtgcctgagcagcatctacgaggacctgaagatgtatcaggtcgagttcaagaccatgaacgccaagctgctgatggaccccaagagacagattttcctcgaccagaacatgctggccgtgatcgatgaactgatgcaggccctgaacttcaacagcgagacagtgccccagaagtcctctctggaagaacccgacttctacaagaccaagatcaagctgtgcatcctgctgcacgccttcagaatcagggccgtgaccatcgacagagtgatgagctatctgaacgccagctgaaagcttggtacccaaacaaacaaaggcgcgtcctggattcgtggtaaaacataccagatttcgatctggagaggtgaagaatacgaccacctactacatccagctgatgagtcccaaataggacgaaacgcgctcaaacaaacaaaagatctcttgtttattgcagcttataatggttacaaataaagcaatagcatcacaaatttcacaaataaagcatttttttcactgcattctagttgtggtttgtccaaactcatcaatgtatcttaacgcggccgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 65 - DNA, 2694 bp, Artificial Sequence; identical to SEQ ID NO: 64
  > AAV.LP1-hIL-12-3-riboswitch signalpeptide, IL12 p40-(G4S)3-p35; carrying the 11nt deletion in the left ITR, and the 4nt deletion in the right ITR

SEQ ID NO: 66 - DNA, 2694 bp, Artificial Sequence
  > AAV. LP1-hIL-12-3riboswitch; signalpeptide, IL12 p35(G4S)3p40; carrying the 11nt and 4nt deletion in the left ITR, and the 4nt deletion in the right ITR
aggaacccctagtgatggagttggccactccctctctgcgcgctcgctcgctcactgaggccgggcgaccaaaggtcgcccgacgcccgggcggcctcagtgagcgagcgagcgcgcagctgcctgcagggcggccgcacgcgttcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagagaattccccggaccggtggatccgccaccatgggctggtcctgcatcattctgtttctggtggccacagccaccggcgtgcactctagaaacctgccagtggctacccctgatccgggcatgtttccttgtctgcaccacagccagaacctgctgagagccgtgtccaatatgctgcagaaggcccggcagacccttgagttctacccttgcaccagcgaggaaatcgaccacgaggacatcaccaaggataagaccagcaccgtggaagcctgcctgcctctggaactgaccaagaacgagtcctgcctgaacagccgggaaaccagcttcatcaccaatggcagctgtctggccagcagaaagacctccttcatgatggccctgtgcctgagcagcatctacgaggacctgaagatgtatcaggtcgagttcaagaccatgaacgccaagctgctgatggaccccaagagacagattttcctcgaccagaacatgctggccgtgatcgatgaactgatgcaggccctgaacttcaacagcgagacagtgccccagaagtcctctctggaagaacccgacttctacaagaccaagatcaagctgtgcatcctgctgcacgccttcagaatcagggccgtgaccatcgacagagtgatgagctatctgaacgccagcggcggcggaggaagcggaggcggaggatctggtggtggtggaagtatttgggagctgaagaaagacgtgtacgtggtcgagctggactggtatcctgatgctcccggcgaaatggtggtgctgacctgtgatacacccgaagaggacggcatcacatggacactggatcagtctagcgaggtgctcggcagcggcaagacactgaccatccaagtgaaagagtttggcgacgccggccagtacacatgtcacaaaggcggagaggtgctgagccattctctgctgctgctccacaagaaagaggatggcatttggagcaccgacatcctgaaggaccagaaagagcccaagaacaagaccttcctgagatgcgaggccaagaactacagcggcagattcacctgttggtggctgaccaccatcagcaccgatctgaccttcagcgtgaagtccagcagaggcagctctgatcctcaaggcgttacatgtggcgccgctacactgtctgccgaaagagtgcggggcgacaacaaagagtacgagtacagcgtcgagtgccaagaggattctgcctgtcctgccgccgaggaatctctgcctatcgaagtgatggtggacgccgtgcacaagctgaagtacgagaactacaccagcagctttttcatccgggacatcatcaagcccgatcctccaaagaacctgcagctgaagcccctgaagaacagcagacaggtggaagtgtcttgggagtaccccgacacatggtctacccctcacagctacttcagcctgaccttctgtgtgcaagtgcagggcaagtccaagcgcgagaagaaagatcgggtgttcaccgacaagaccagcgccaccgtgatctgcagaaagaacgccagcatcagcgtgcgcgctcaggataggtactacagcagctcttggagcgagtgggcctctgttccttgttcttgaaagcttggtacccaaacaaacaaaggcgcgtcctggattcgtggtaaaacataccagatttcgatctggagaggtgaagaatacgaccacctactacatccagctgatgagtcccaaataggacgaaacgcgctcaaacaaacaaaagatctcttgtttattgcagcttataatggttacaaataaagcaatagcatcacaaatttcacaaataaagcatttttttcactgcattctagttgtggtttgtccaaactcatcaatgtatcttaacgcggccgcacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 67 - DNA, 428 bp, Homo sapiens
  misc_feature (1)..(428): human CEA promoter
gccctggagagcatggggagacccgggaccctgctgggtttctctgtcacaaaggaaaataatccccctggtgtgacagacccaaggacagaacacagcagaggtcagcactggggaagacaggttgtcctcccaggggatgggggtccatccaccttgccgaaaagatttgtctgaggaactgaaaatagaagggaaaaaagaggagggacaaaagaggcagaaatgagaggggaggggacagaggacacctgaataaagaccacacccatgacccacgtgatgctgagaagtactcctgccctaggaagagactcagggcagagggaggaaggacagcagaccagacagtcacagcagccttgacaaaacgttcctggaactcaagctcttctccacagaggaggacagagcagacagcagagacc

SEQ ID NO: 68 - DNA, 770 bp, Homo sapiens
  misc_feature (1)..(770): human Muc1 promoter
cctgcagggcccactagtgttcatcggagcccaggtttactcccttaagtggaaatttcttcccccactccctccttggctttctccaaggagggaacccaggctactggaaagtccggctggggcggggactgtgggtttcagggtagaactgcgtgtggaacgggacagggagcggttagaagggtggggctattccgggaagtggtggggggagggagcccaaaactagcacctagtccactcattatccagccctcttatttctcggccccgctctgcttcagtggacccggggagggcggggaagtggagtgggagacctaggggtgggcttcccgaccttgctgtacaggacctcgacctagctggctttgttccccatccccacgttagttgttgccctgaggctaaaactagagcccaggggccccaagttccagactgcccctcccccctcccccggagccagggagtggttggtgaaagggggaggccagctggagaacaaacgggtagtcagggggttgagcgattagagcccttgtaccctacccaggaatggttggggaggaggaggaagaggtaggaggtaggggagggggcggggttttgtcacctgtcacctgctccggctgtgcctagggcgggcgggcggggagtggggggaccggtataaagcggtaggcgcctgtgcccgctccacctctcaagcagccagcgcctgcctgaatctgttctgccccctccccacccatttcaccaccacc

SEQ ID NO: 69 - DNA, 276 bp, Homo sapiens
  misc_feature (1)..(276): human AFP promoter
attctgtagtttgaggagaatatttgttatatttgcaaaataaaataagtttgcaagttttttttttctgccccaaagagctctgtgtccttgaacataaaatacaaataaccgctatgctgttaattattggcaaatgtcccattttcaacctaaggaaataccataaagtaacagatataccaacaaaaggttactagttaacaggcattgcctgaaaagagtataaaagaatttcagcatgattttccatattgtgcttccaccactgccaat

SEQ ID NO: 70 - protein, 738 aa, Adeno-associated virus - 8
  MISC_FEATURE (1)..(738): GenBank AAN03857.1 Capsid Protein adeno-associated Virus 8, AAV8 VP1 sequence
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 71 - DNA, 2217 bp, Adeno-associated virus - 8
  CDS (1)..(2217): GenBank AF513852.1 AAV8 VP1 Capsid CDS sequence
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 72 - protein, 738 aa, Adeno-associated virus - 8; identical to SEQ ID NO: 70

SEQ ID NO: 73 - DNA, 463 bp, Homo sapiens
  misc_feature (1)..(463): LP1 promotor without SV40 intron
tcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaga

SEQ ID NO: 74 - DNA, 2409 bp, Artificial Sequence
  > LP1-hIL-12-3-riboswitch signalpeptide, IL12 p40-(G4S)3-p35; expression cassette
tcgaccccctaaaatgggcaaacattgcaagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaagagaattccccggaccggtggatccgccaccatgggctggtcctgcatcattctgtttctggtggccacagccaccggcgtgcactctatttgggagctgaagaaagacgtgtacgtggtcgagctggactggtatcctgatgctcccggcgaaatggtggtgctgacctgtgatacacccgaagaggacggcatcacatggacactggatcagtctagcgaggtgctcggcagcggcaagacactgaccatccaagtgaaagagtttggcgacgccggccagtacacatgtcacaaaggcggagaggtgctgagccattctctgctgctgctccacaagaaagaggatggcatttggagcaccgacatcctgaaggaccagaaagagcccaagaacaagaccttcctgagatgcgaggccaagaactacagcggcagattcacctgttggtggctgaccaccatcagcaccgatctgaccttcagcgtgaagtccagcagaggcagctctgatcctcaaggcgttacatgtggcgccgctacactgtctgccgaaagagtgcggggcgacaacaaagagtacgagtacagcgtcgagtgccaagaggattctgcctgtcctgccgccgaggaatctctgcctatcgaagtgatggtggacgccgtgcacaagctgaagtacgagaactacaccagcagctttttcatccgggacatcatcaagcccgatcctccaaagaacctgcagctgaagcccctgaagaacagcagacaggtggaagtgtcttgggagtaccccgacacatggtctacccctcacagctacttcagcctgaccttctgtgtgcaagtgcagggcaagtccaagcgcgagaagaaagatcgggtgttcaccgacaagaccagcgccaccgtgatctgcagaaagaacgccagcatcagcgtgcgcgctcaggataggtactacagcagctcttggagcgagtgggcctctgttccttgttctggcggcggaggaagcggaggcggaggatctggtggtggtggaagtagaaacctgccagtggctacccctgatccgggcatgtttccttgtctgcaccacagccagaacctgctgagagccgtgtccaatatgctgcagaaggcccggcagacccttgagttctacccttgcaccagcgaggaaatcgaccacgaggacatcaccaaggataagaccagcaccgtggaagcctgcctgcctctggaactgaccaagaacgagtcctgcctgaacagccgggaaaccagcttcatcaccaatggcagctgtctggccagcagaaagacctccttcatgatggccctgtgcctgagcagcatctacgaggacctgaagatgtatcaggtcgagttcaagaccatgaacgccaagctgctgatggaccccaagagacagattttcctcgaccagaacatgctggccgtgatcgatgaactgatgcaggccctgaacttcaacagcgagacagtgccccagaagtcctctctggaagaacccgacttctacaagaccaagatcaagctgtgcatcctgctgcacgccttcagaatcagggccgtgaccatcgacagagtgatgagctatctgaacgccagctgaaagcttggtacccaaacaaacaaaggcgcgtcctggattcgtggtaaaacataccagatttcgatctggagaggtgaagaatacgaccacctactacatccagctgatgagtcccaaataggacgaaacgcgctcaaacaaacaaaagatctcttgtttattgcagcttataatggttacaaataaagcaatagcatcacaaatttcacaaataaagcatttttttcactgcattctagttgtggtttgtccaaactcatcaatgtatcttaacgcggccgcacgtgcggaccgagcggccgc

SEQ ID NO: 75 - DNA, 2607 bp, Artificial Sequence
  > LP1_SV40-hIL-12-3-riboswitch signalpeptide, IL12 p40-(G4S)3-p35; expression cassette
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctattttagattccaacctttggaactgagaattccccggaccggtggatccgccaccatgggctggtcctgcatcattctgtttctggtggccacagccaccggcgtgcactctatttgggagctgaagaaagacgtgtacgtggtcgagctggactggtatcctgatgctcccggcgaaatggtggtgctgacctgtgatacacccgaagaggacggcatcacatggacactggatcagtctagcgaggtgctcggcagcggcaagacactgaccatccaagtgaaagagtttggcgacgccggccagtacacatgtcacaaaggcggagaggtgctgagccattctctgctgctgctccacaagaaagaggatggcatttggagcaccgacatcctgaaggaccagaaagagcccaagaacaagaccttcctgagatgcgaggccaagaactacagcggcagattcacctgttggtggctgaccaccatcagcaccgatctgaccttcagcgtgaagtccagcagaggcagctctgatcctcaaggcgttacatgtggcgccgctacactgtctgccgaaagagtgcggggcgacaacaaagagtacgagtacagcgtcgagtgccaagaggattctgcctgtcctgccgccgaggaatctctgcctatcgaagtgatggtggacgccgtgcacaagctgaagtacgagaactacaccagcagctttttcatccgggacatcatcaagcccgatcctccaaagaacctgcagctgaagcccctgaagaacagcagacaggtggaagtgtcttgggagtaccccgacacatggtctacccctcacagctacttcagcctgaccttctgtgtgcaagtgcagggcaagtccaagcgcgagaagaaagatcgggtgttcaccgacaagaccagcgccaccgtgatctgcagaaagaacgccagcatcagcgtgcgcgctcaggataggtactacagcagctcttggagcgagtgggcctctgttccttgttctggcggcggaggaagcggaggcggaggatctggtggtggtggaagtagaaacctgccagtggctacccctgatccgggcatgtttccttgtctgcaccacagccagaacctgctgagagccgtgtccaatatgctgcagaaggcccggcagacccttgagttctacccttgcaccagcgaggaaatcgaccacgaggacatcaccaaggataagaccagcaccgtggaagcctgcctgcctctggaactgaccaagaacgagtcctgcctgaacagccgggaaaccagcttcatcaccaatggcagctgtctggccagcagaaagacctccttcatgatggccctgtgcctgagcagcatctacgaggacctgaagatgtatcaggtcgagttcaagaccatgaacgccaagctgctgatggaccccaagagacagattttcctcgaccagaacatgctggccgtgatcgatgaactgatgcaggccctgaacttcaacagcgagacagtgccccagaagtcctctctggaagaacccgacttctacaagaccaagatcaagctgtgcatcctgctgcacgccttcagaatcagggccgtgaccatcgacagagtgatgagctatctgaacgccagctgaaagcttggtacccaaacaaacaaaggcgcgtcctggattcgtggtaaaacataccagatttcgatctggagaggtgaagaatacgaccacctactacatccagctgatgagtcccaaataggacgaaacgcgctcaaacaaacaaaagatctcttgtttattgcagcttataatggttacaaataaagcaatagcatcacaaatttcacaaataaagcatttttttcactgcattctagttgtggtttgtccaaactcatcaatgtatcttaacgcggccgcacgtgcggaccgagcggccgc

SEQ ID NO: 76 - protein, 537 aa, Artificial Sequence
  >single chain human IL12 - with SP according to SEQ ID NO 52, long linker (Seq ID No. 75)
MGWSCIILFLVATATGVHSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 77 - DNA, 14 bp, Artificial Sequence
  linker sequence
gcggccgcacgcgt

SEQ ID NO: 78 - DNA, 29 bp, Artificial Sequence
  linker sequence
gaattccccggaccggtggatccgccacc

SEQ ID NO: 79 - DNA, 12 bp, Artificial Sequence
  linker sequence
aagcttggtacc

SEQ ID NO: 80 - DNA, 29 bp, Artificial Sequence
  linker sequence
gcggccgcacgtgcggaccgagcggccgc